(19) United States
(12) Patent Application Publication
(10) Pub. No.: US 2022/0305017 A1
(43) Pub. Date: Sep. 29, 2022

(54) COMPOSITIONS AND METHODS FOR THE TREATMENT OF ESTROGEN-DEPENDENT DISORDERS

(71) Applicant: ObsEva S.A., Plan-les-Ouates (CH)

(72) Inventors: Jean-Pierre GOTTELAND, Geneva (CH); Elke BESTEL, Plan-les-Ouates (CH)

(21) Appl. No.: 17/633,479

(22) PCT Filed: Aug. 7, 2020
(86) PCT No.: PCT/EP2020/072302

§ 371 Date: Feb. 7, 2022

Related U.S. Application Data
(60) Provisional application No. 63/048,427, filed on Jul. 6, 2020.
(60) Provisional application No. 63/047,038, filed on Jul. 1, 2020.
(60) Provisional application No. 62/884,426, filed on Aug. 8, 2019.

(51) Int. Cl. A61K 31/519; A61P 15/00; A61K 31/513; A61K 31/4184; A61K 31/4439; A61K 31/565; A61K 31/567; A61K 31/57; A61K 31/585

(52) U.S. Cl. CPC A61K 31/519; A61P 15/00; A61K 31/513; A61K 31/4184; A61K 31/4439; A61K 31/565; A61K 31/567; A61K 31/57; A61K 31/585

(57) ABSTRACT

The present disclosure provides compositions and methods for treating estrogen-dependent disorders, such as disorders of the female reproductive system, including uterine fibroids and endometriosis, among others. Compounds described herein that may be used to treat such indications include gonadotropin-releasing hormone (GnRH) antagonists. Suitable GnRH antagonists useful in conjunction with the compositions and methods described herein include thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid and the choline salt thereof, among others. Using the compositions and methods described herein, GnRH antagonists may be periodically administered to a patient, such as one or more times per day, week, or month. Advantageously, using the dosing schedules of the present disclosure, the periodic administration the GnRH antagonist may be temporarily halted, allowing a patient to recover any lost bone mineral density, without an accompanying return in the patient's symptoms.

### FIELD OF THE INVENTION

The invention relates to the therapeutic treatment of disorders of the female reproductive system, including uterine fibroids and endometriosis, among others, and reduction of symptoms associated therewith.

### BACKGROUND OF THE INVENTION

Estrogen-dependent disorders represent a challenging class of diseases that have a high incidence in the general population and are often associated with particularly severe symptomology. Uterine fibroids, for example, also referred to as leiomyomata, are among the most common benign tumors in women. Symptoms associated with uterine fibroids commonly include heavy or prolonged menstrual bleeding, pelvic pressure and pelvic organ compression, back pain, and adverse reproductive outcomes. Heavy menstrual bleeding may lead to iron deficiency anemia, a key symptom of uterine fibroids and the leading cause of surgical interventions that may include hysterectomy. Endometriosis is another estrogen-dependent gynecological condition, characterized by the presence of endometrial-like tissue outside the uterus. A chronic inflammatory reaction induced by the ectopic endometrial cells, endometriosis may result in infertility and a variety of pain symptoms including dysmenorrhea, dyspareunia, chronic pelvic pain, dysuria, and dyschezia, among others.

Additional examples of estrogen-dependent diseases include adenomyosis and rectovaginal endometriosis, which are particularly severe endometrial growth disorders characterized by the invasion of endometrial tissue into the uterine myometrium and rectovaginal zones, respectively. The term adenomyosis or uterine adenomyosis is used to describe the presence of both endometrial glands and stroma deep within the myometrium. This condition is associated with hypertrophy and hyperplasia of the subjacent muscle cells, which may ultimately result in an altered size and globulous morphology of the uterus. Due to the severity of this disorder, one of the key symptoms is strong menstrual and even non-menstrual pelvic pain with abnormal uterine bleeding. Like adenomyosis, rectovaginal endometriosis patients present with a variety of pain symptoms including dysmenorrhea, dyspareunia, chronic pelvic pain, dysuria, and dyschezia. Treatment options for rectovaginal endometriosis are limited. Since medical therapies are either ineffective or have considerable side effects, rectovaginal endometriosis patients often undergo surgical procedures to reduce the endometrial node, and may even be subject to resection of the bowel if the node infiltrates the rectal or sigmoidal wall.

There exists a need for new and improved therapies for alleviating the symptoms associated with these and other estrogen-dependent disorders, as well as for treating their underlying pathology.

### SUMMARY OF THE INVENTION

The present disclosure relates to compositions and methods for the treatment of estrogen-dependent disorders, such as uterine fibroids and endometriosis, among others. Using the compositions and methods of the disclosure, a patient, such as a female human patient, may be administered a gonadotropin-releasing hormone (GnRH) antagonist so as to treat the underlying biochemical etiology of one or more of these diseases and/or to alleviate one or more symptoms associated with such conditions. Estrogen-dependent diseases, such as uterine fibroids and endometriosis, among others, emerge due to elevated concentrations of circulating β17-estradiol (E2) in a patient. Particularly, endogenous E2 levels that exceed 60 pg/ml can engender the onset of uterine fibroids, endometriosis, and other estrogen-dependent disorders. Without being limited by mechanism, a patient suffering from an estrogen-dependent disorder may be administered a GnRH antagonist so as to reduce the serum concentration of follicle-stimulating hormone (FSH) and/or luteinizing hormone (LH) in the patient, thereby suppressing endogenous levels of E2. The diminished E2 concentration induced by the GnRH antagonist can result in a beneficial effect on symptomology, manifesting in the patient, for example, as a reduction in uterine fibroid volume and/or uterine blood loss. Similarly, in the context of a patient having endometriosis, using the compositions and methods described herein, the patient may be administered a GnRH antagonist so as to reduce endogenous E2 levels, thereby diminishing the volume of endometrial tissue extending outside of the uterus and/or alleviating such symptoms as global pelvic pain, dysmenorrhea, dyspareunia, and dyschezia. The compositions and methods of the disclosure can also be used to treat particularly severe endometrial growth disorders, including adenomyosis and rectovaginal endometriosis, among other pathologies mediated by excessive E2 production.

GnRH antagonists that may be used in conjunction with the compositions and methods described herein include optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof. In some embodiments, the GnRH antagonist is an optionally substituted 3-aminoalkyl pyrimidine-2,4(1H,3H)-dione derivative, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof. The GnRH antagonist may be, for example, an optionally substituted thieno[2,3d]pyrimidine derivative, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof. In some embodiments, the GnRH antagonist is an optionally substituted propane-1,3-dione derivative, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Additional GnRH antagonists that may be used in conjunction with the compositions and methods described herein include SK12670 and BAY-784, as well as derivatives and variants thereof, among others.

Although GnRH antagonist treatment can have the beneficial effect of reducing serum E2 concentration in a patient, and thus, treat the underlying etiology of an estrogen-dependent disease, excessive suppression of endogenous E2 can have harmful side effects. This phenomenon is due, at least in part, to the finding that serum E2 concentration is positively correlated with bone mineral density, but excessive serum E2 concentration can promote the development of an estrogen-dependent disease (Gallagher, Rheumatic Disease Clinics of North America 27:143-162 (2001)). Accordingly, when serum E2 levels in a patient (e.g., a female human patient) are reduced to certain low concentrations, the patient may experience a reduction in bone mineral density. A desired range of serum E2 concentration in a female human patient is from about 10 pg/ml to about 60 pg/ml (Barbieri, The Journal of Reproductive Medicine 43:287-292 (1998)). When serum E2 concentrations are within this range, estrogen-dependent diseases are effectively treated at the molecular level, and the symptoms associated with these disorders, such as uterine blood loss in the case of uterine fibroids and pelvic pain in patients suffering from endometriosis, among others, are ameliorated. Serum E2 concentrations above this range can trigger an estrogen-dependent disorder, but serum concentrations beneath this range can lead to bone mineral density loss.

When a patient (e.g., a female human patient) suffering from an estrogen-dependent disease, such as an estrogen-dependent disease described herein (e.g., uterine fibroids, endometriosis, adenomyosis, and/or rectovaginal endometriosis), is administered a GnRH antagonist, the patient's serum E2 concentration may be reduced so as to fall within the 10 pg/ml-60 pg/ml therapeutic window described above. In some instances, however, administration of a GnRH antagonist may cause the patient's serum E2 concentration to decrease to such an extent that the patient exhibits a reduction in bone mineral density, such as a reduction in bone mineral density of from about 1% to about 5% (e.g., a reduction in bone mineral density of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5%) relative to an assessment of the patient's bone mineral density performed during, or prior to, the onset of GnRH antagonist administration.

Using the compositions and methods of the disclosure, a patient suffering from an estrogen-dependent disease may be administered a GnRH antagonist (e.g., periodically, such as one or more times per day, week, month, or year over the course of a treatment period) so as to reduce serum E2 concentration, thereby targeting the underlying pathology and alleviating the patient's symptoms. In the event that the patient exhibits a reduction in bone mineral density, administration of the GnRH antagonist to the patient may be temporarily ceased. The present disclosure is based, in part, on the discovery that such patients (i) exhibit an increase in serum E2 concentration after cessation of the GnRH antagonist treatment, and, surprisingly, (ii) sustain the therapeutic effect(s) of the GnRH antagonist even after its administration has been halted. As shown in the working examples, below, patients suffering from an estrogen-dependent disease that are periodically treated with a GnRH antagonist, particularly an optionally substituted thieno[3,4d]pyrimidine (e.g., 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof), and that exhibit a reduction in bone mineral density following treatment not only demonstrate a recovery in bone mineral density after the treatment is ceased, but also maintain a reduction in disease symptomology even after the GnRH antagonist treatment. This observation is entirely unexpected, particularly because serum E2 concentration is positively correlated with both bone mineral density and the severity of estrogen-dependent diseases.

This surprising treatment outcome gives rise to a series of beneficial dosing regimens. For example, using the compositions and methods describe herein, a patient suffering from an estrogen-dependent disease may be administered a GnRH antagonist (e.g., one or more times per day, week, or month over the course of a first treatment period) and then monitored for a potential reduction in bone mineral density. If a reduction in bone mineral density is detected (e.g., relative to an assessment of the patient's bone mineral density performed during, or prior to, the onset of GnRH antagonist treatment), periodic administration of the GnRH antagonist may be temporarily stopped. This stoppage period during which a GnRH antagonist is not administered to the patient provides the patient with an opportunity to regain bone mineral density. Moreover, during this stoppage period, the patient may maintain a reduction in disease symptomology. Administration of the GnRH antagonist may then resume during a second treatment period. Collectively, these steps allow a patient to be administered a GnRH antagonist so as to reduce serum E2 concentration to a level sufficient to treat an estrogen-dependent disease while preventing excessive loss of bone mineral density. As a result of these steps, the patient can experience the added benefit of a sustained, continuous alleviation of disease symptoms, even when GnRH antagonist administration is temporarily halted to allow serum E2 recovery.

To achieve the beneficial clinical properties described above, a patient suffering from an estrogen-dependent disease may be administered a GnRH antagonist over the course of a first treatment period, such as a treatment period lasting one or more days, weeks, or months. The patient may then be monitored for a reduction in bone mineral density, and, if one is detected, administration of the GnRH antagonist may be temporarily halted, as described above. Additionally or alternatively, the patient may be administered a reduced dosage of the GnRH antagonist so as to reduce serum E2 concentration to within a therapeutic window (e.g., a level of from about 10 pg/ml to about 60 pg/ml) within which bone mineral density is preserved and disease symptomology is alleviated. In some embodiments, a patient that is being treated, or has previously been treated, with a GnRH antagonist that is not an optionally substituted thieno[3,4d]pyrimidine (e.g., 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof) may be treated with such a compound, for example, in view of such compound's surprising ability to engender a sustained reduction in disease symptoms even after its administration is halted.

In a first aspect, the disclosure features a method of treating an estrogen-dependent disease in a patient (e.g., a mammalian patient, such as a female human patient) in need thereof. The estrogen-dependent disease may be, for example, uterine fibroids, endometriosis, adenomyosis, or rectovaginal endometriosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of treating an estrogen-dependent disease (e.g., uterine fibroids, endometriosis, adenomyosis, or rectovaginal endometriosis) in a human patient in need thereof by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period.

In another aspect, the disclosure features a method of treating an estrogen-dependent disease, such as uterine fibroids, endometriosis, adenomyosis, or rectovaginal endometriosis, in a human patient in need thereof by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period.

In an additional aspect, the disclosure features a method of reducing the volume of menstrual blood loss, inducing amenorrhea, reducing the volume of one or more uterine fibroids, preserving hemoglobin, and/or reducing the concentration of follicle-stimulating hormone (FSH), luteinizing hormone (LH), or β17-estradiol (E2) in a human patient diagnosed as having uterine fibroids. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period.

In another aspect, the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period.

In an additional aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period.

In another aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period.

In an additional aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period.

In another aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period.

In an additional aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period.

In another aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period.

In some embodiments of any of the above aspects of the disclosure, the second treatment period commences at least one day after the end of the first treatment period (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or more, after the end of the first treatment period). For example, the second treatment period may commence at least one week after the end of the first treatment period (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, or more, after the end of the treat treatment period).

In some embodiments of any of the above aspects of the disclosure, the patient is not administered a GnRH antagonist during the time elapsed between the end of the first treatment period and commencement of the second treatment period.

In some embodiments of any of the above aspects of the disclosure, the amount of the GnRH antagonist that is periodically administered to the patient during the second treatment period is less than the amount of the GnRH antagonist that is periodically administered to the patient during the first treatment period.

In some embodiments of any of the above aspects of the disclosure, the GnRH antagonist that is periodically administered to the patient during the first treatment period is not a compound represented by formula (I). In some embodiments, the GnRH antagonist that is periodically administered to the patient during the second treatment period is a compound represented by formula (I)

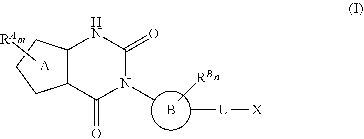

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In another aspect, the disclosure features a method of treating an estrogen-dependent disease in a patient (e.g., a mammalian patient, such as a female human patient) in need thereof. The estrogen-dependent disease may be, for example, uterine fibroids, endometriosis, adenomyosis, or rectovaginal endometriosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of treating an estrogen-dependent disease (e.g., uterine fibroids, endometriosis, adenomyosis, or rectovaginal endometriosis) in a human patient in need thereof by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In another aspect, the disclosure features a method of treating an estrogen-dependent disease, such as uterine fibroids, endometriosis, adenomyosis, or rectovaginal endometriosis, in a human patient in need thereof by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period. Additionally, in accordance with this aspect, the amount of the GnRH antagonist that is periodically administered to the patient during the second treatment period is less than the amount of the GnRH antagonist periodically administered to the patient during the first treatment period.

In an additional aspect, the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In another aspect, the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period. Additionally, in accordance with this aspect, the amount of the GnRH antagonist that is periodically administered to the patient during the second treatment period is less than the amount of the GnRH antagonist periodically administered to the patient during the first treatment period.

In an additional aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In another aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period. Additionally, in accordance with this aspect, the amount of the GnRH antagonist that is periodically administered to the patient during the second treatment period is less than the amount of the GnRH antagonist periodically administered to the patient during the first treatment period.

In an additional aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In another aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period. Additionally, in accordance with this aspect, the amount of the GnRH antagonist that is periodically administered to the patient during the second treatment period is less than the amount of the GnRH antagonist periodically administered to the patient during the first treatment period.

In an additional aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis. The method may include:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In some instances, the patient is identified as exhibiting a reduced bone mineral density at the end of the first treatment period relative to a measurement of the patient's bone mineral density obtained during, or prior to commencement of, the first treatment period. Thus, in another aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis by:
a) periodically administering to the patient a therapeutically effective amount of a GnRH antagonist during a first treatment period,
b) determining that the patient exhibits a reduced bone mineral density at the end of the first treatment period relative to a measurement of the patient's bone mineral density obtained during, or prior to commencement of, the first treatment period, and
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

In another aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis by periodically administering to the patient a therapeutically effective amount of a GnRH antagonist during a second treatment period. In this aspect, the patient has previously been administered a GnRH antagonist during a first treatment period and has been determined to exhibit a reduced bone mineral density at the end of the first treatment period relative to a measurement of the patient's bone mineral density obtained during, or prior to commencement of, the first treatment period. In some embodiments of this aspect, the GnRH antagonist administered to the patient during the first treatment period has been periodically administered to the patient during the first treatment period. Additionally, in accordance with this aspect, the amount of the GnRH antagonist that is periodically administered to the patient during the second treatment period is less than the amount of the GnRH antagonist periodically administered to the patient during the first treatment period.

In some embodiments of any of the foregoing aspects of the disclosure, the second treatment period commences at least one day after the end of the first treatment period (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or more, after the end of the first treatment period). For example, the second treatment period may commence at least one week after the end of the first treatment period (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, or more, after the end of the treat treatment period.

In some embodiments of any of the foregoing aspects of the disclosure, the patient is not administered a GnRH antagonist during the time elapsed between the end of the first treatment period and commencement of the second treatment period.

In some embodiments of any of the foregoing aspects of the disclosure, the GnRH antagonist that is periodically administered to the patient during the first treatment period is not a compound represented by formula (I). In some embodiments, the GnRH antagonist that is periodically administered to the patient during the second treatment period is a compound represented by formula (I)

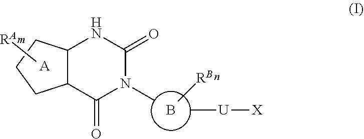

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In a further aspect, the disclosure features a method of treating an estrogen-dependent disease, such as uterine fibroids, endometriosis, adenomyosis, or rectovaginal endometriosis, in a human patient in need thereof by periodically administering to the patient, during a second treatment period, a therapeutically effective amount of a GnRH antagonist represented by formula (I)

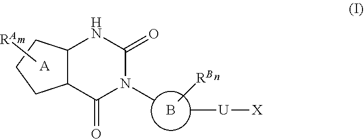

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered, during a first treatment period, a GnRH antagonist (e.g., in a therapeutically effective amount) that is not a compound represented by formula (I).

In another aspect, the disclosure features a method of reducing the volume of menstrual blood loss, inducing amenorrhea, reducing the volume of one or more uterine fibroids, preserving hemoglobin, and/or reducing the concentration of FSH, LH, or E2 in a human patient diagnosed as having uterine fibroids by periodically administering to the patient, during a second treatment period, a therapeutically effective amount of a GnRH antagonist represented by formula (I)

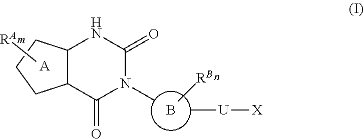

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered, during a first treatment period, a GnRH antagonist (e.g., in a therapeutically effective amount) that is not a compound represented by formula (I).

In an additional aspect, the disclosure features a method of reducing the volume of one or more endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having endometriosis by periodically administering to the patient, during a second treatment period, a therapeutically effective amount of a GnRH antagonist represented by formula (I)

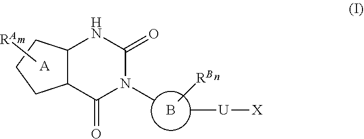

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered, during a first treatment period, a GnRH antagonist (e.g., in a therapeutically effective amount) that is not a compound represented by formula (I).

In another aspect, the disclosure features a method of reducing uterine volume, reducing the thickness of the anterior and/or posterior region of the uterine myometrium, reducing pelvic pain, reducing dysmenorrhea, reducing dyspareunia, reducing dyschezia, reducing uterine tenderness, reducing uterine bleeding, inducing amenorrhea, and/or reducing the concentration of FSH, LH, or E2 in a human patient diagnosed as having adenomyosis by periodically administering to the patient, during a second treatment period, a therapeutically effective amount of a GnRH antagonist represented by formula (I)

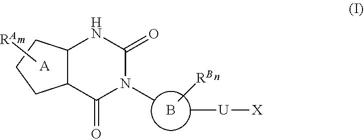

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered, during a first treatment period, a GnRH antagonist (e.g., in a therapeutically effective amount) that is not a compound represented by formula (I).

In another aspect, the disclosure features a method of reducing the volume of one or more rectovaginal endometriosis nodes, reducing the volume of one or more type III rectovaginal endometriosis nodes, reducing pelvic pain, reducing dysmenorrhea, reducing dyspareunia, reducing dyschezia, reducing uterine bleeding, inducing amenorrhea, and/or reducing the concentration of FSH, LH, or E2 in a human patient diagnosed as having rectovaginal endometriosis by periodically administering to the patient, during a second treatment period, a therapeutically effective amount of a GnRH antagonist represented by formula (I)

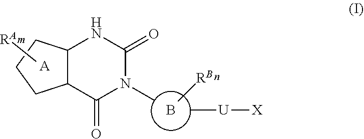

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof,

wherein the patient has previously been administered, during a first treatment period, a GnRH antagonist (e.g., in a therapeutically effective amount) that is not a compound represented by formula (I).

In some embodiments of any of the foregoing aspects of the disclosure, the GnRH antagonist that is not a compound represented by formula (I) has been periodically administered to the patient during the first treatment period, such as in one or more doses per day, week, month, or year.

In some embodiments of any of the foregoing aspects of the disclosure, the patient has been determined to exhibit a reduced bone mineral density at the end of the first treatment period relative to a measurement of the patient's bone mineral density obtained during, or prior to commencement of, the first treatment period.

In some embodiments of any of the foregoing aspects of the disclosure, the second treatment period commences at least one day after the end of the first treatment period (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, or more, after the end of the first treatment period). For example, the second treatment period may commence at least one week after the end of the first treatment period (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, or more, after the end of the treat treatment period.

In some embodiments of any of the foregoing aspects of the disclosure, the patient is not administered a GnRH antagonist during the time elapsed between the end of the first treatment period and commencement of the second treatment period.

In some embodiments of any of the above aspects of the disclosure, the amount of the GnRH antagonist that is periodically administered to the patient during the second treatment period is less than the amount of the GnRH antagonist that is periodically administered to the patient during the first treatment period.

In some embodiments of the disclosure, the GnRH antagonist administered during the first treatment period is a compound represented by formula (I)

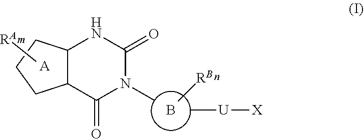

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

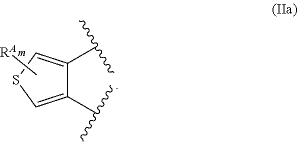

In some embodiments of formula (I) or (IIa), m is 1.

In some embodiments of formula (I) or (IIa), the ring A is an optionally substituted thiophene ring represented by formula (IIb)

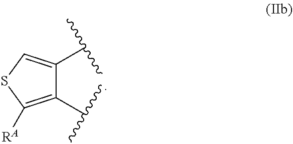

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

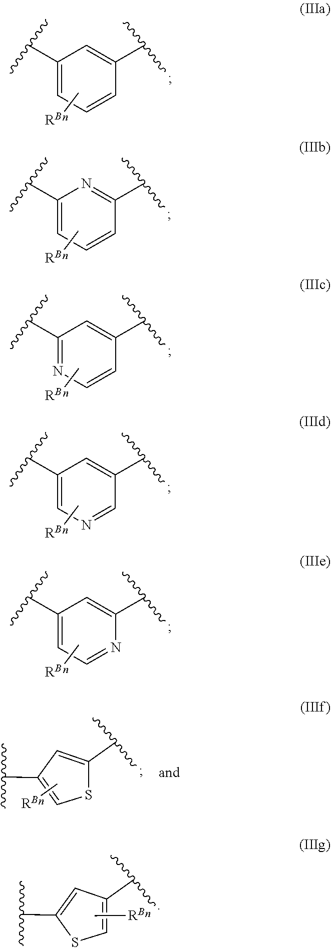

In some embodiments of formula (I), (IIa), (IIb), or any one of (IIIa)-(IIIg), n is 2.

In some embodiments of formula (I), (IIa), (IIb), or any one of (IIIa)-(IIIg), the ring B is represented by a formula selected from the group consisting of:

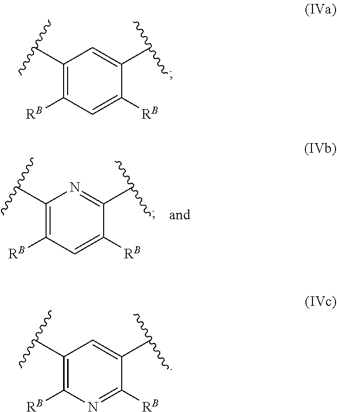

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), U is a single bond.

In some embodiments of formula (I), (IIa), (IIb), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIIa)-(IIIg), or any one of (IVa)-(IVc), Y is an optionally substituted benzene ring represented by formula (V)

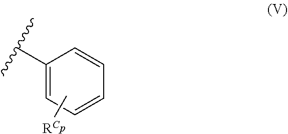

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), Y is a substituted benzene ring represented by formula (Va)

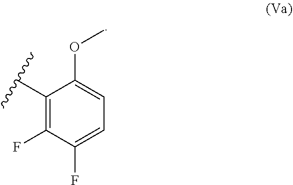 [SMILES: *c1c(OC)ccc(F)c1F]

In some embodiments, the compound is represented by formula (Ia)

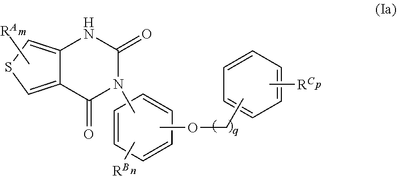

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

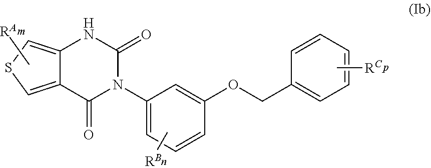

In some embodiments, the compound is represented by formula (Ic)

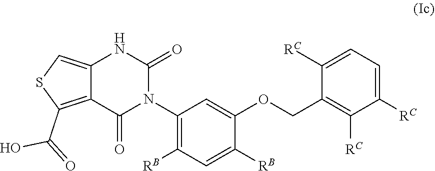

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, represented by formula (VI)

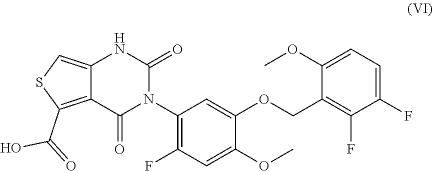 [SMILES: COc1cc(F)c(-n2c(=O)[nH]c3csc(C(=O)O)c3c2=O)cc1OCc1c(OC)ccc(F)c1F]

or a pharmaceutically acceptable salt thereof.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate. It is to be understood that references herein to a compound represented by formula (VI) specifically include the choline salt of compound (VI), which is represented by formula (VIa), below.

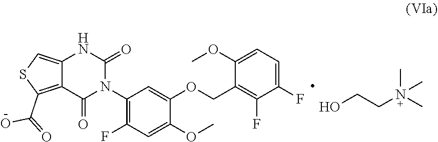

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹⁹F solid-state NMR peaks centered at about −151.8 ppm, −145.2 ppm, and −131.6 ppm.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is orally administered to the patient.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 25 mg to about 400 mg per dose during the first treatment period (e.g., in the recited amount or in an equivalent amount of a pharmaceutically acceptable salt, such as a choline salt), such as an amount of about 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg of the compound of any one of formulas (I), (Ia)-(Ic), (IIa), (IIb), (IIIa)-(IIIg), (IVa)-(IVc), (V), (Va), or (VI) during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 35 mg to about 65 mg per dose during the first treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per dose during the first treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 50 mg per dose during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 60 mg to about 90 mg per dose during the first treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per dose during the first treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 75 mg per dose during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 50 mg to about 150 mg per dose during the first treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per dose during the first treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 100 mg per dose during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 150 mg to about 250 mg per dose during the first treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per dose during the first treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 200 mg per dose during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses (i.e., one or more times) per day, week, or month during the first treatment period, such as from 1 to 10 times per day during the first treatment period (e.g., 1 time, 2 times, 3 times, 4 times, 5 times, 6 times, 7 times, 8 times, 9 times, or 10 times per day, such as 1 time or 2 times per day), 1 to 100 times per week during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the first treatment period, the compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses per day during the first treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more unit dosage forms that collectively constitute a single dose (e.g., during the first treatment period). For example, at a given time within the first treatment period, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, or more (e.g., in the recited amount or in an equivalent amount of a pharmaceutically acceptable salt, such as a choline salt), by administration of one or more unit dosage forms of the compound to the patient. As a non-limiting example, a single dose of 200 mg of the compound may be administered to the subject by way of two individual 100-mg unit dosage forms of the compound. The two 100-mg unit dosage forms collectively constitute a single 200-mg dose of the compound if administered to the patient at substantially the same time.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 35 mg to about 65 mg per day during the first treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the first treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 50 mg per day during the first treatment period (e.g., in the recited amount or in an equivalent amount of a pharmaceutically acceptable salt, such as a choline salt).

In some embodiments, the compound of any one of formulas (i), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 60 mg to about 90 mg per day during the first treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the first treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 75 mg per day during the first treatment period (e.g., in the recited amount or in an equivalent amount of a pharmaceutically acceptable salt, such as a choline salt).

In some embodiments, the compound of any one of formulas (i), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 50 mg to about 150 mg per day during the first treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the first treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 100 mg per day during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 150 mg to about 250 mg per day during the first treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the first treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 200 mg per day during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in a single dose per day during the first treatment period.

For example, the compound may be administered to the patient in an amount (e.g., a single dose) of from about 35 mg to about 65 mg per day during the first treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the first treatment period (e.g., in the recited amount or in an equivalent amount of a pharmaceutically acceptable salt, such as a choline salt). In some embodiments, the compound is administered to the patient in an amount (e.g., a single dose) of about 50 mg per day during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 60 mg to about 90 mg per day during the first treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the first treatment period (e.g., in the recited amount or in an equivalent amount of a pharmaceutically acceptable salt, such as a choline salt). In some embodiments, the compound is administered to the patient in an amount (e.g., a single dose) of about 75 mg per day during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 50 mg to about 150 mg per day during the first treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the first treatment period (e.g., in the recited amount or in an equivalent amount of a pharmaceutically acceptable salt, such as a choline salt). In some embodiments, the compound is administered to the patient in an amount (e.g., a single dose) of about 100 mg per day during the first treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 150 mg to about 250 mg per day during the first treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the first treatment period (e.g., in the recited amount or in an equivalent amount of a pharmaceutically acceptable salt, such as a choline salt). In some embodiments, the compound is administered to the patient in an amount (e.g., a single dose) of about 200 mg per day during the first treatment period (e.g., in the recited amount or in an equivalent amount of a pharmaceutically acceptable salt, such as a choline salt).

In some embodiments of the disclosure, the GnRH antagonist administered during the first treatment period is a compound represented by any one of formulas (VII)-(XIV), below, such as elagolix, relugolix, or opigolix (ASP1707). In some embodiments, the GnRH antagonist administered during the first treatment period is BAY-784 or SK-2706.

For example, in some embodiments, the GnRH antagonist administered during the first treatment period is a compound represented by formula (VII)

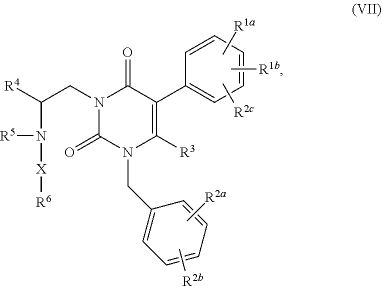

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist administered during the first treatment period is a compound represented by formula (VIII)

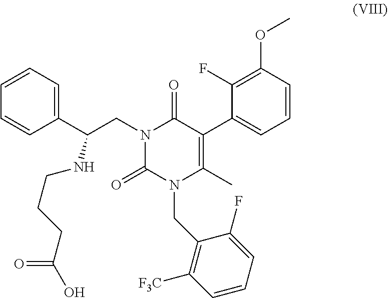 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist administered during the first treatment period is the sodium salt of the compound represented by formula (VIII), which is represented by formula (IX), below.

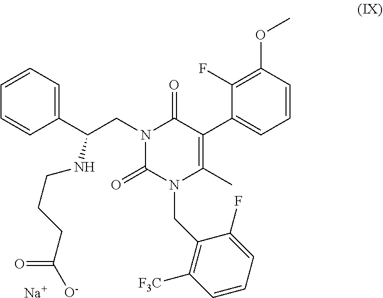 [SMILES: COc1cccc(-c2c(C)n(Cc3c(F)cccc3C(F)(F)F)c(=O)n(C[C@H](NCCCC(=O)[O-])c3ccccc3)c2=O)c1F ; [Na+]]

In some embodiments, the compound of any one of formulas (VII)-(IX) is administered to the patient in an amount of from about 50 mg to about 650 mg per dose during the first treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per dose during the first treatment period (e.g., in the recited amount or in an equivalent amount of a pharmaceutically acceptable salt, such as a sodium salt). In some embodiments, the compound of any one of formulas (VII)-(IX) is administered to the patient in an amount of about 150 mg per dose during the first treatment period, 300 mg per dose during the first treatment period, 400 mg per dose during the first treatment period, or 600 mg per dose during the first treatment period (e.g., in the recited amount or in an equivalent amount of a pharmaceutically acceptable salt, such as a sodium salt).

In some embodiments, the compound of any one of formulas (VII)-(IX) is administered to the patient in one or more doses (i.e., one or more times) per day, week, or month during the first treatment period, such as from 1 to 10 times per day during the first treatment period (e.g., 1 time, 2 times, 3 times, 4 times, 5 times, 6 times, 7 times, 8 times, 9 times, or 10 times per day, such as 1 time or 2 times per day), 1 to 100 times per week during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the first treatment period, the compound of any one of formulas (VII)-(IX) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (VII)-(IX) is administered to the patient in one or more doses per day during the first treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (VII)-(IX) may be administered to the patient in one or more unit dosage forms that collectively constitute a single dose (e.g., during the first treatment period). For example, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, or more (e.g., in the recited amount or in an equivalent amount of a pharmaceutically acceptable salt, such as a sodium salt), by administration of one or more unit dosage forms of the compound to the patient. As a non-limiting example, a single dose of 300 mg of the compound may be administered to the subject by way of two individual 150-mg unit dosage forms of the compound. The two 150-mg unit dosage forms collectively constitute a single 300-mg dose of the compound if administered to the patient at substantially the same time.

In some embodiments, the compound of any one of formulas (VII)-(IX) is administered to the patient in one or more daily doses (e.g., from 1 to 10 doses, such as 1, 2, 3, 4, 5, 6, 7, 8, 9, or 10 doses, or more) totaling from about 50 mg to about 650 mg per day during the first treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per day during the first treatment period (e.g., in the recited amount or in an equivalent amount of a pharmaceutically acceptable salt, such as a sodium salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 150 mg per day during the first treatment period, 300 mg per day during the first treatment period, 400 mg per day during the first treatment period (e.g., 200 mg administered twice daily), or 600 mg per day during the first treatment period (e.g., 300 mg administered twice daily) (e.g., in the recited amount or in an equivalent amount of a pharmaceutically acceptable salt, such as a sodium salt).

In some embodiments, the GnRH antagonist administered during the first treatment period is a compound represented by formula (X)

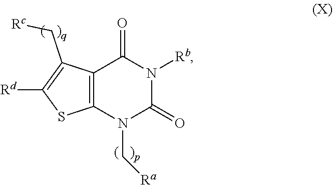

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist administered during the first treatment period is a compound represented by formula (XI)

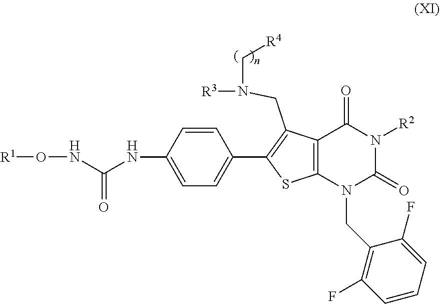

wherein R¹ is C₁₋₄alkyl;

R² is (1) a C₁₋₆alkyl which may have a substituent selected from the group consisting of (1′) a hydroxy group, (2′) a C₁₋₄alkoxy, (3) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist administered during the first treatment period is a compound represented by formula (XII), below.

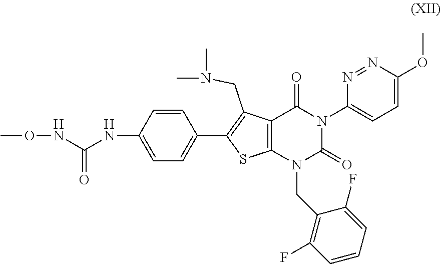

In some embodiments, the compound of any one of formulas (X)-(XII) is administered to the patient in an amount of from about 10 mg to about 60 mg per dose during the first treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per dose during the first treatment period (e.g., in the recited amount or in an equivalent amount of a pharmaceutically acceptable salt, such as a chlorine salt). In some embodiments, the compound of any one of formulas (X)-(XII) is administered to the patient in an amount of about 40 mg per dose during the first treatment period (e.g., in the recited amount or in an equivalent amount of a pharmaceutically acceptable salt, such as a chlorine salt).

In some embodiments, the compound of any one of formulas (X)-(XII) is administered to the patient in one or more doses (i.e., one or more times) per day, week, or month during the first treatment period, such as from 1 to 10 times per day during the first treatment period (e.g., 1 time, 2 times, 3 times, 4 times, 5 times, 6 times, 7 times, 8 times, 9 times, or 10 times per day, such as 1 time or 2 times per day), 1 to 100 times per week during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the first treatment period, the compound of any one of formulas (X)-(XII) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (X)-(XII) is administered to the patient in one or more doses per day during the first treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I)-(XII) may be administered to the patient in one or more unit dosage forms that collectively constitute a single dose (e.g., during the first treatment period). For example, a patient may be administered a single dose of the compound of a specified amount, such as a single dose of 25 mg, 50 mg, 75 mg, 100 mg, or more (e.g., in the recited amount or in an equivalent amount of a pharmaceutically acceptable salt, such as a chlorine salt), by administration of one or more unit dosage forms of the compound to the patient. As a non-limiting example, a single dose of 40 mg of the compound may be administered to the subject by way of two individual 20-mg unit dosage forms of the compound. The two 20-mg unit dosage forms collectively constitute a single 40-mg dose of the compound if administered to the patient at substantially the same time.

In some embodiments, the compound of any one of formulas (X)-(XII) is administered to the patient in one or more daily doses (e.g., from 1 to 10 doses, such as 1, 2, 3, 4, 5, 6, 7, 8, 9, or 10 doses, or more) totaling from about 10 mg to about 60 mg per day during the first treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per day during the first treatment period (e.g., in the recited amount or in an equivalent amount of a pharmaceutically acceptable salt, such as a chlorine salt). In some embodiments, the compound is administered to the patient in one or more daily doses (e.g., from 1 to 10 doses, such as 1, 2, 3, 4, 5, 6, 7, 8, 9, or 10 doses, or more) totaling from about 20 mg to about 50 mg per day during the first treatment period (e.g., in the recited amount or in an equivalent amount of a pharmaceutically acceptable salt, such as a chlorine salt), such as an amount of about 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg per day during the first treatment period (e.g., in the recited amount or in an equivalent amount of a pharmaceutically acceptable salt, such as a chlorine salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 40 mg per day during the first treatment period (e.g., a single daily dose of 40 mg) (e.g., in the recited amount or in an equivalent amount of a pharmaceutically acceptable salt, such as a chlorine salt).

In some embodiments, the GnRH antagonist administered during the first treatment period is a compound represented by formula (XIII)

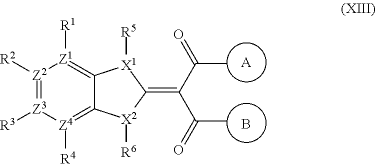

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁- (wherein n₁₀₁ is an integer of 0 to 2), H—S(O)n₁₀₁-, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent; or a pharmaceutically acceptable salt thereof. In some embodiments, the GnRH antagonist is a compound represented by formula (XIV), below.

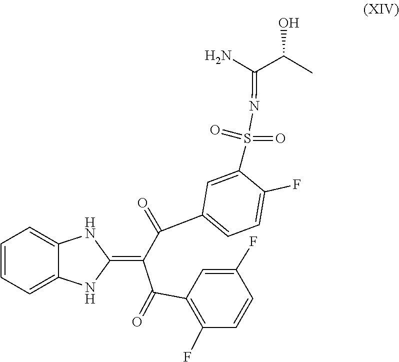 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the compound of any one of formulas (XIII) and (XIV), or SK12670 or BAY-784, is administered to the patient in one or more doses (i.e., one or more times) per day, week, or month during the first treatment period, such as from 1 to 10 times per day during the first treatment period (e.g., 1 time, 2 times, 3 times, 4 times, 5 times, 6 times, 7 times, 8 times, 9 times, or 10 times per day, such as 1 time or 2 times per day), 1 to 100 times per week during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the first treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the first treatment period, the compound of any one of formulas (XIII) and (XIV), or SK12670 or BAY-784, may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (XIII) and (XIV), or SK12670 or BAY-784, is administered to the patient in one or more doses per day during the first treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

In some embodiments of the disclosure, the GnRH antagonist administered during the second treatment period is a compound represented by formula (I)

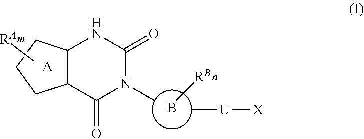

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

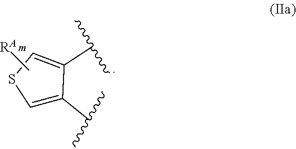

In some embodiments of formula (I) or (IIa), m is 1.

In some embodiments of formula (I) or (IIa), the ring A is an optionally substituted thiophene ring represented by formula (IIb)

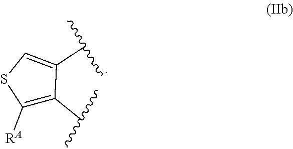

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

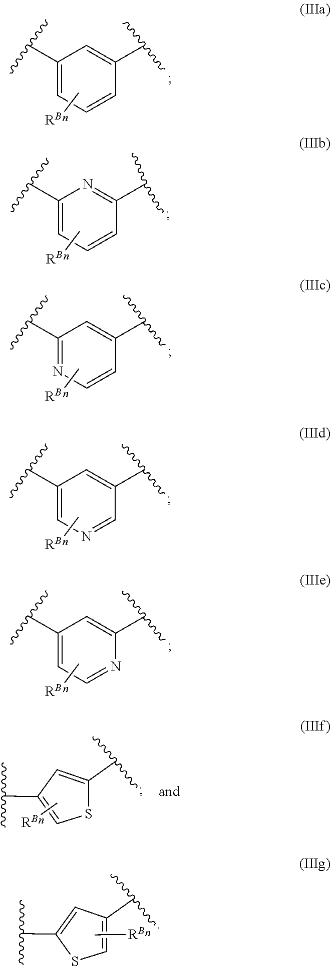

In some embodiments of formula (I), (IIa), (IIb), or any one of (IIIa)-(IIIg), n is 2.

In some embodiments of formula (I), (IIa), (IIb), or any one of (IIIa)-(IIIg), the ring B is represented by a formula selected from the group consisting of:

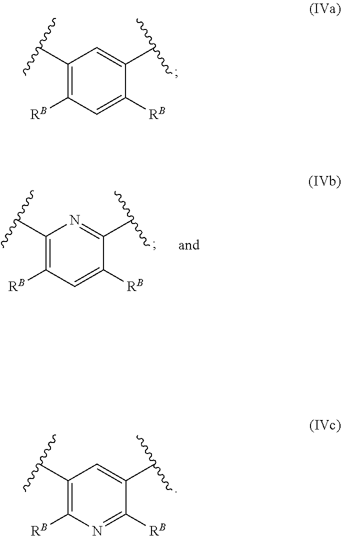

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), U is a single bond.

In some embodiments of formula (I), (IIa), (IIb), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIIa)-(IIIg), or any one of (IVa)-(IVc), Y is an optionally substituted benzene ring represented by formula (V)

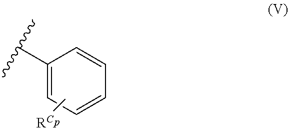

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), Y is a substituted benzene ring represented by formula (Va)

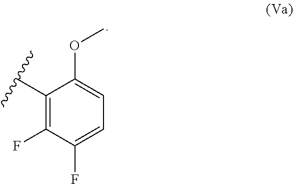 [SMILES: *c1c(OC)ccc(F)c1F]

In some embodiments, the compound is represented by formula (Ia)

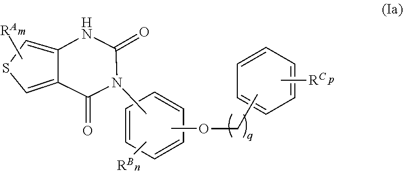

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

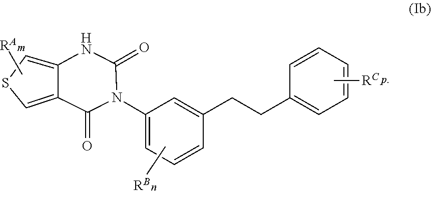

In some embodiments, the compound is represented by formula (Ic)

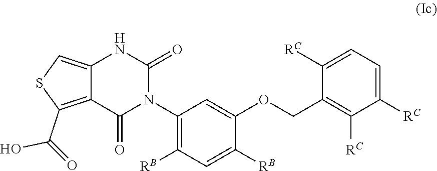

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, represented by formula (VI)

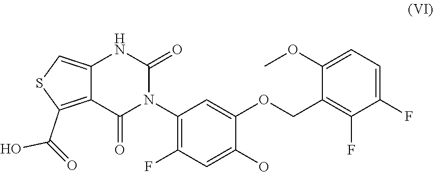 [SMILES: COc1ccc(F)c(F)c1COc1cc(-n2c(=O)[nH]c3csc(C(=O)O)c3c2=O)c(F)cc1O]

or a pharmaceutically acceptable salt thereof.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate. It is to be understood that references herein to a compound represented by formula (VI) specifically include the choline salt of compound (VI), which is represented by formula (VIa), below.

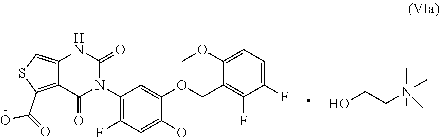

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹⁹F solid-state NMR peaks centered at about −151.8 ppm, −145.2 ppm, and −131.6 ppm.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is orally administered to the patient.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 25 mg to about 400 mg per dose during the second treatment period (e.g., in the recited amount or in an equivalent amount of a pharmaceutically acceptable salt, such as a choline salt), such as an amount of about 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg of the compound of any one of formulas (I), (Ia)-(Ic), (IIa), (IIb), (IIIa)-(IIIg), (IVa)-(IVc), (V), (Va), or (VI) during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 35 mg to about 65 mg per dose during the second treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per dose during the second treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 50 mg per dose during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 60 mg to about 90 mg per dose during the second treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per dose during the second treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 75 mg per dose during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 50 mg to about 150 mg per dose during the second treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per dose during the second treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 100 mg per dose during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 150 mg to about 250 mg per dose during the second treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per dose during the second treatment period (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 200 mg per dose during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses (i.e., one or more times) per day, week, or month during the second treatment period, such as from 1 to 10 times per day during the second treatment period (e.g., 1 time, 2 times, 3 times, 4 times, 5 times, 6 times, 7 times, 8 times, 9 times, or 10 times per day, such as 1 time or 2 times per day), 1 to 100 times per week during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the second treatment period, the compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses per day during the second treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more unit dosage forms that collectively constitute a single dose (e.g., during the second treatment period). For example, at a given time within the second treatment period, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, or more (e.g., in the recited amount or in an equivalent amount of a pharmaceutically acceptable salt, such as a choline salt), by administration of one or more unit dosage forms of the compound to the patient. As a non-limiting example, a single dose of 200 mg of the compound may be administered to the subject by way of two individual 100-mg unit dosage forms of the compound. The two 100-mg unit dosage forms collectively constitute a single 200-mg dose of the compound if administered to the patient at substantially the same time.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 35 mg to about 65 mg per day during the second treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the second treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 50 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 60 mg to about 90 mg per day during the second treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the second treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 75 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 50 mg to about 150 mg per day during the second treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the second treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 100 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 150 mg to about 250 mg per day during the second treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the second treatment period (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 200 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in a single dose per day during the second treatment period.

For example, the compound may be administered to the patient in an amount (e.g., a single dose) of from about 35 mg to about 65 mg per day during the second treatment period (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day during the second treatment period (e.g., in the recited amount or in an equivalent amount of a pharmaceutically acceptable salt, such as a choline salt).

In some embodiments, the compound is administered to the patient in an amount (e.g., a single dose) of about 50 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 60 mg to about 90 mg per day during the second treatment period (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day during the second treatment period (e.g., in the recited amount or in an equivalent amount of a pharmaceutically acceptable salt, such as a choline salt).

In some embodiments, the compound is administered to the patient in an amount (e.g., a single dose) of about 75 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 50 mg to about 150 mg per day during the second treatment period (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day during the second treatment period (e.g., in the recited amount or in an equivalent amount of a pharmaceutically acceptable salt, such as a choline salt). In some embodiments, the compound is administered to the patient in an amount (e.g., a single dose) of about 100 mg per day during the second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount (e.g., a single dose) of from about 150 mg to about 250 mg per day during the second treatment period (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day during the second treatment period (e.g., in the recited amount or in an equivalent amount of a pharmaceutically acceptable salt, such as a choline salt). In some embodiments, the compound is administered to the patient in an amount (e.g., a single dose) of about 200 mg per day during the second treatment period (e.g., in the recited amount or in an equivalent amount of a pharmaceutically acceptable salt, such as a choline salt).

In some embodiments of the disclosure, the GnRH antagonist administered during the second treatment period is a compound represented by any one of formulas (VII)-(XIV), below, such as elagolix, relugolix, or opigolix (ASP1707). In some embodiments, the GnRH antagonist administered during the second treatment period is BAY-784 or SK-2706.

For example, in some embodiments, the GnRH antagonist administered during the second treatment period is a compound represented by formula (VII)

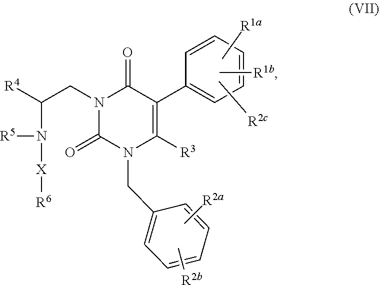

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist administered during the second treatment period is a compound represented by formula (VIII)

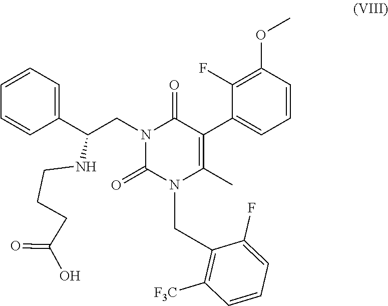 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist administered during the second treatment period is the sodium salt of the compound represented by formula (VIII), which is represented by formula (IX), below.

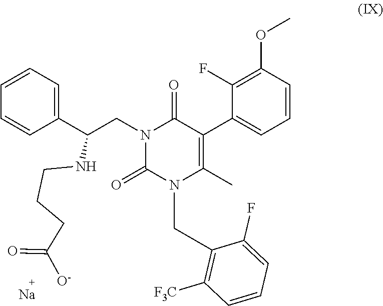

In some embodiments, the compound of any one of formulas (VII)-(IX) is administered to the patient in an amount of from about 50 mg to about 650 mg per dose during the second treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per dose during the second treatment period (e.g., in the recited amount or in an equivalent amount of a pharmaceutically acceptable salt, such as a sodium salt). In some embodiments, the compound of any one of formulas (VII)-(IX) is administered to the patient in an amount of about 150 mg per dose during the second treatment period, 300 mg per dose during the second treatment period, 400 mg per dose during the second treatment period, or 600 mg per dose during the second treatment period (e.g., in the recited amount or in an equivalent amount of a pharmaceutically acceptable salt, such as a sodium salt).

In some embodiments, the compound of any one of formulas (VII)-(IX) is administered to the patient in one or more doses (i.e., one or more times) per day, week, or month during the second treatment period, such as from 1 to 10 times per day during the second treatment period (e.g., 1 time, 2 times, 3 times, 4 times, 5 times, 6 times, 7 times, 8 times, 9 times, or 10 times per day, such as 1 time or 2 times per day), 1 to 100 times per week during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the second treatment period, the compound of any one of formulas (VII)-(IX) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (VII)-(IX) is administered to the patient in one or more doses per day during the second treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (VII)-(IX) may be administered to the patient in one or more unit dosage forms that collectively constitute a single dose (e.g., during the second treatment period). For example, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, or more (e.g., in the recited amount or in an equivalent amount of a pharmaceutically acceptable salt, such as a sodium salt), by administration of one or more unit dosage forms of the compound to the patient. As a non-limiting example, a single dose of 300 mg of the compound may be administered to the subject by way of two individual 150-mg unit dosage forms of the compound. The two 150-mg unit dosage forms collectively constitute a single 300-mg dose of the compound if administered to the patient at substantially the same time.

In some embodiments, the compound of any one of formulas (VII)-(IX) is administered to the patient in one or more daily doses (e.g., from 1 to 10 doses, such as 1, 2, 3, 4, 5, 6, 7, 8, 9, or 10 doses, or more) totaling from about 50 mg to about 650 mg per day during the second treatment period (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per day during the second treatment period (e.g., in the recited amount or in an equivalent amount of a pharmaceutically acceptable salt, such as a sodium salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 150 mg per day during the second treatment period, 300 mg per day during the second treatment period, 400 mg per day during the second treatment period (e.g., 200 mg administered twice daily), or 600 mg per day during the second treatment period (e.g., 300 mg administered twice daily) (e.g., in the recited amount or in an equivalent amount of a pharmaceutically acceptable salt, such as a sodium salt).

In some embodiments, the GnRH antagonist administered during the second treatment period is a compound represented by formula (X)

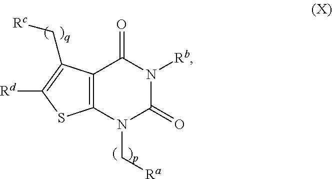

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist administered during the second treatment period is a compound represented by formula (XI)

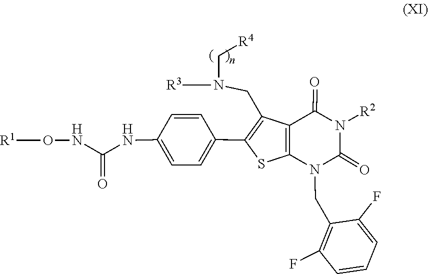

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist administered during the second treatment period is a compound represented by formula (XII), below.

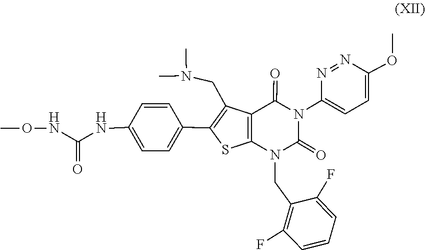

In some embodiments, the compound of any one of formulas (X)-(XII) is administered to the patient in an amount of from about 10 mg to about 60 mg per dose during the second treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per dose during the second treatment period (e.g., in the recited amount or in an equivalent amount of a pharmaceutically acceptable salt, such as a chlorine salt). In some embodiments, the compound of any one of formulas (X)-(XII) is administered to the patient in an amount of about 40 mg per dose during the second treatment period (e.g., in the recited amount or in an equivalent amount of a pharmaceutically acceptable salt, such as a chlorine salt).

In some embodiments, the compound of any one of formulas (X)-(XII) is administered to the patient in one or more doses (i.e., one or more times) per day, week, or month during the second treatment period, such as from 1 to 10 times per day during the second treatment period (e.g., 1 time, 2 times, 3 times, 4 times, 5 times, 6 times, 7 times, 8 times, 9 times, or 10 times per day, such as 1 time or 2 times per day), 1 to 100 times per week during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the second treatment period, the compound of any one of formulas (X)-(XII) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (X)-(XII) is administered to the patient in one or more doses per day during the second treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I)-(XII) may be administered to the patient in one or more unit dosage forms that collectively constitute a single dose (e.g., during the second treatment period). For example, a patient may be administered a single dose of the compound of a specified amount, such as a single dose of 25 mg, 50 mg, 75 mg, 100 mg, or more (e.g., in the recited amount or in an equivalent amount of a pharmaceutically acceptable salt, such as a chlorine salt), by administration of one or more unit dosage forms of the compound to the patient. As a non-limiting example, a single dose of 40 mg of the compound may be administered to the subject by way of two individual 20-mg unit dosage forms of the compound. The two 20-mg unit dosage forms collectively constitute a single 40-mg dose of the compound if administered to the patient at substantially the same time.

In some embodiments, the compound of any one of formulas (X)-(XII) is administered to the patient in one or more daily doses (e.g., from 1 to 10 doses, such as 1, 2, 3, 4, 5, 6, 7, 8, 9, or 10 doses, or more) totaling from about 10 mg to about 60 mg per day during the second treatment period (e.g., in the recited amount or in an equivalent amount of a pharmaceutically acceptable salt, such as a chlorine salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per day during the second treatment period (e.g., in the recited amount or in an equivalent amount of a pharmaceutically acceptable salt, such as a chlorine salt). In some embodiments, the compound is administered to the patient in one or more daily doses (e.g., from 1 to 10 doses, such as 1, 2, 3, 4, 5, 6, 7, 8, 9, or 10 doses, or more) totaling from about 20 mg to about 50 mg per day during the second treatment period (e.g., in the recited amount or in an equivalent amount of a pharmaceutically acceptable salt, such as a chlorine salt), such as an amount of about 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg per day during the second treatment period (e.g., in the recited amount or in an equivalent amount of a pharmaceutically acceptable salt, such as a chlorine salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 40 mg per day during the second treatment period (e.g., a single daily dose of 40 mg) (e.g., in the recited amount or in an equivalent amount of a pharmaceutically acceptable salt, such as a chlorine salt).

In some embodiments, the GnRH antagonist administered during the second treatment period is a compound represented by formula (XIII)

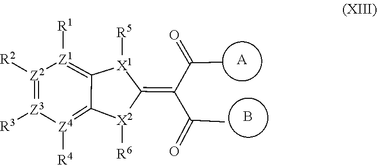

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁- (wherein n₁₀₁ is an integer of 0 to 2), H—S(O)n₁₀₁-, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist is a compound represented by formula (XIV), below.

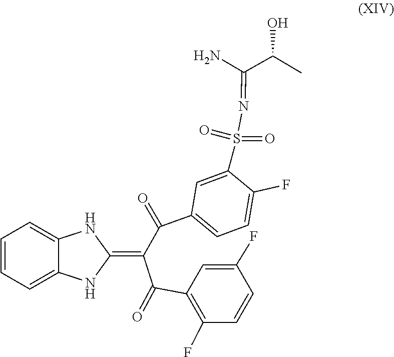 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the compound of any one of formulas (XIII) and (XIV), or SK12670 or BAY-784, is administered to the patient in one or more doses (i.e., one or more times) per day, week, or month during the second treatment period, such as from 1 to 10 times per day during the second treatment period (e.g., 1 time, 2 times, 3 times, 4 times, 5 times, 6 times, 7 times, 8 times, 9 times, or 10 times per day, such as 1 time or 2 times per day), 1 to 100 times per week during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month during the second treatment period (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, during the second treatment period, the compound of any one of formulas (XIII) and (XIV), or SK12670 or BAY-784, may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (XIII) and (XIV), or SK12670 or BAY-784, is administered to the patient in one or more doses per day during the second treatment period, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

In some embodiments of the disclosure, the first treatment period has a duration of at least four weeks (e.g., a duration of about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). For example, the first treatment period may have a duration of at least eight weeks (e.g., a duration of about 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). In some embodiments, the first treatment period has a duration of at least 10 weeks (e.g., a duration of about 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). In some embodiments, the first treatment period has a duration of at least 12 weeks (e.g., a duration of about 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). In some embodiments, the first treatment period has a duration of at least 24 weeks (e.g., a duration of about 24 weeks).

In some embodiments, the first treatment period has a duration of from about four weeks to about 12 months (e.g., a duration of about 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, 6 weeks, 6 1/7 weeks, 6 2/7 weeks, 6 3/7 weeks, 6 4/7 weeks, 6 5/7 weeks, 6 6/7 weeks, 7 weeks, 7 1/7 weeks, 7 2/7 weeks, 7 3/7 weeks, 7 4/7 weeks, 7 5/7 weeks, 7 6/7 weeks, 8 weeks, 8 1/7 weeks, 8 2/7 weeks, 8 3/7 weeks, 8 4/7 weeks, 8 5/7 weeks, 8 6/7 weeks, 9 weeks, 9 1/7 weeks, 9 2/7 weeks, 9 3/7 weeks, 9 4/7 weeks, 9 5/7 weeks, 9 6/7 weeks, 10 weeks, 10 1/7 weeks, 10 2/7 weeks, 10 3/7 weeks, 10 4/7 weeks, 10 5/7 weeks, 10 6/7 weeks, 11 weeks, 11 1/7 weeks, 11 2/7 weeks, 11 3/7 weeks, 11 4/7 weeks, 11 5/7 weeks, 11 6/7 weeks, 12 weeks, 12 1/7 weeks, 12 2/7 weeks, 12 3/7 weeks, 12 4/7 weeks, 12 5/7 weeks, 12 6/7 weeks, 13 weeks, 13 1/7 weeks, 13 2/7 weeks, 13 3/7 weeks, 13 4/7 weeks, 13 5/7 weeks, 13 6/7 weeks, 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, 16 weeks, 16 1/7 weeks, 16 2/7 weeks, 16 3/7 weeks, 16 4/7 weeks, 16 5/7 weeks, 16 6/7 weeks, 17 weeks, 17 1/7 weeks, 17 2/7 weeks, 17 3/7 weeks, 17 4/7 weeks, 17 5/7 weeks, 17 6/7 weeks, 18 weeks, 18 1/7 weeks, 18 2/7 weeks, 18 3/7 weeks, 18 4/7 weeks, 18 5/7 weeks, 18 6/7 weeks, 19 weeks, 19 1/7 weeks, 19 2/7 weeks, 19 3/7 weeks, 19 4/7 weeks, 19 5/7 weeks, 19 6/7 weeks, 20 weeks, 20 1/7 weeks, 20 2/7 weeks, 20 3/7 weeks, 20 4/7 weeks, 20 5/7 weeks, 20 6/7 weeks, 21 weeks, 21 1/7 weeks, 21 2/7 weeks, 21 3/7 weeks, 21 4/7 weeks, 21 5/7 weeks, 21 6/7 weeks, 22 weeks, 22 1/7 weeks, 22 2/7 weeks, 22 3/7 weeks, 22 4/7 weeks, 22 5/7 weeks, 22 6/7 weeks, 23 weeks, 23 1/7 weeks, 23 2/7 weeks, 23 3/7 weeks, 23 4/7 weeks, 23 5/7 weeks, 23 6/7 weeks, 24 weeks, 24 1/7 weeks, 24 2/7 weeks, 24 3/7 weeks, 24 4/7 weeks, 24 5/7 weeks, 24 6/7 weeks, 25 weeks, 25 1/7 weeks, 25 2/7 weeks, 25 3/7 weeks, 25 4/7 weeks, 25 5/7 weeks, 25 6/7 weeks, 26 weeks, 26 1/7 weeks, 26 2/7 weeks, 26 3/7 weeks, 26 4/7 weeks, 26 5/7 weeks, 26 6/7 weeks, 27 weeks, 27 1/7 weeks, 27 2/7 weeks, 27 3/7 weeks, 27 4/7 weeks, 27 5/7 weeks, 27 6/7 weeks, 28 weeks, 28 1/7 weeks, 28 2/7 weeks, 28 3/7 weeks, 28 4/7 weeks, 28 5/7 weeks, 28 6/7 weeks, 29 weeks, 29 1/7 weeks, 29 2/7 weeks, 29 3/7 weeks, 29 4/7 weeks, 29 5/7 weeks, 29 6/7 weeks, 30 weeks, 30 1/7 weeks, 30 2/7 weeks, 30 3/7 weeks, 30 4/7 weeks, 30 5/7 weeks, 30 6/7 weeks, 31 weeks, 31 1/7 weeks, 31 2/7 weeks, 31 3/7 weeks, 31 4/7 weeks, 31 5/7 weeks, 31 6/7 weeks, 32 weeks, 32 1/7 weeks, 32 2/7 weeks, 32 3/7 weeks, 32 4/7 weeks, 32 5/7 weeks, 32 6/7 weeks, 33 weeks, 33 1/7 weeks, 33 2/7 weeks, 33 3/7 weeks, 33 4/7 weeks, 33 5/7 weeks, 33 6/7 weeks, 34 weeks, 34 1/7 weeks, 34 2/7 weeks, 34 3/7 weeks, 34 4/7 weeks, 34 5/7 weeks, 34 6/7 weeks, 35 weeks, 35 1/7 weeks, 35 2/7 weeks, 35 3/7 weeks, 35 4/7 weeks, 35 5/7 weeks, 35 6/7 weeks, 36 weeks, 36 1/7 weeks, 36 2/7 weeks, 36 3/7 weeks, 36 4/7 weeks, 36 5/7 weeks, 36 6/7 weeks, 37 weeks, 37 1/7 weeks, 37 2/7 weeks, 37 3/7 weeks, 37 4/7 weeks, 37 5/7 weeks, 37 6/7 weeks, 38 weeks, 38 1/7 weeks, 38 2/7 weeks, 38 3/7 weeks, 38 4/7 weeks, 38 5/7 weeks, 38 6/7 weeks, 39 weeks, 39 1/7 weeks, 39 2/7 weeks, 39 3/7 weeks, 39 4/7 weeks, 39 5/7 weeks, 39 6/7 weeks, 40 weeks, 40 1/7 weeks, 40 2/7 weeks, 40 3/7 weeks, 40 4/7 weeks, 40 5/7 weeks, 40 6/7 weeks, 41 weeks, 41 1/7 weeks, 41 2/7 weeks, 41 3/7 weeks, 41 4/7 weeks, 41 5/7 weeks, 41 6/7 weeks, 42 weeks, 42 1/7 weeks, 42 2/7 weeks, 42 3/7 weeks, 42 4/7 weeks, 42 5/7 weeks, 42 6/7 weeks, 43 weeks, 43 1/7 weeks, 43 2/7 weeks, 43 3/7 weeks, 43 4/7 weeks, 43 5/7 weeks, 43 6/7 weeks, 44 weeks, 44 1/7 weeks, 44 2/7 weeks, 44 3/7 weeks, 44 4/7 weeks, 44 5/7 weeks, 44 6/7 weeks, 45 weeks, 45 1/7 weeks, 45 2/7 weeks, 45 3/7 weeks, 45 4/7 weeks, 45 5/7 weeks, 45 6/7 weeks, 46 weeks, 46 1/7 weeks, 46 2/7 weeks, 46 3/7 weeks, 46 4/7 weeks, 46 5/7 weeks, 46 6/7 weeks, 47 weeks, 47 1/7 weeks, 47 2/7 weeks, 47 3/7 weeks, 47 4/7 weeks, 47 5/7 weeks, 47 6/7 weeks, or 48 weeks).

In some embodiments, the first treatment period has a duration of from about four weeks to about 44 weeks, a duration of from about four weeks to about 36 weeks, a duration of from about four weeks to about 24 weeks, a duration of from about five weeks to about 20 weeks, a duration of from about six weeks to about 18 weeks, a duration of from about eight weeks to about 16 weeks, or a duration of from about 10 weeks to about 14 weeks (e.g., a duration of about 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, 6 weeks, 6 1/7 weeks, 6 2/7 weeks, 6 3/7 weeks, 6 4/7 weeks, 6 5/7 weeks, 6 6/7 weeks, 7 weeks, 7 1/7 weeks, 7 2/7 weeks, 7 3/7 weeks, 7 4/7 weeks, 7 5/7 weeks, 7 6/7 weeks, 8 weeks, 8 1/7 weeks, 8 2/7 weeks, 8 3/7 weeks, 8 4/7 weeks, 8 5/7 weeks, 8 6/7 weeks, 9 weeks, 9 1/7 weeks, 9 2/7 weeks, 9 3/7 weeks, 9 4/7 weeks, 9 5/7 weeks, 9 6/7 weeks, 10 weeks, 10 1/7 weeks, 10 2/7 weeks, 10 3/7 weeks, 10 4/7 weeks, 10 5/7 weeks, 10 6/7 weeks, 11 weeks, 11 1/7 weeks, 11 2/7 weeks, 11 3/7 weeks, 11 4/7 weeks, 11 5/7 weeks, 11 6/7 weeks, 12 weeks, 12 1/7 weeks, 12 2/7 weeks, 12 3/7 weeks, 12 4/7 weeks, 12 5/7 weeks, 12 6/7 weeks, 13 weeks, 13 1/7 weeks, 13 2/7 weeks, 13 3/7 weeks, 13 4/7 weeks, 13 5/7 weeks, 13 6/7 weeks, 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, 16 weeks, 16 1/7 weeks, 16 2/7 weeks, 16 3/7 weeks, 16 4/7 weeks, 16 5/7 weeks, 16 6/7 weeks, 17 weeks, 17 1/7 weeks, 17 2/7 weeks, 17 3/7 weeks, 17 4/7 weeks, 17 5/7 weeks, 17 6/7 weeks, 18 weeks, 18 1/7 weeks, 18 2/7 weeks, 18 3/7 weeks, 18 4/7 weeks, 18 5/7 weeks, 18 6/7 weeks, 19 weeks, 19 1/7 weeks, 19 2/7 weeks, 19 3/7 weeks, 19 4/7 weeks, 19 5/7 weeks, 19 6/7 weeks, 20 weeks, 20 1/7 weeks, 20 2/7 weeks, 20 3/7 weeks, 20 4/7 weeks, 20 5/7 weeks, 20 6/7 weeks, 21 weeks, 21 1/7 weeks, 21 2/7 weeks, 21 3/7 weeks, 21 4/7 weeks, 21 5/7 weeks, 21 6/7 weeks, 22 weeks, 22 1/7 weeks, 22 2/7 weeks, 22 3/7 weeks, 22 4/7 weeks, 22 5/7 weeks, 22 6/7 weeks, 23 weeks, 23 1/7 weeks, 23 2/7 weeks, 23 3/7 weeks, 23 4/7 weeks, 23 5/7 weeks, 23 6/7 weeks, 24 weeks, 24 1/7 weeks, 24 2/7 weeks, 24 3/7 weeks, 24 4/7 weeks, 24 5/7 weeks, 24 6/7 weeks, 25 weeks, 25 1/7 weeks, 25 2/7 weeks, 25 3/7 weeks, 25 4/7 weeks, 25 5/7 weeks, 25 6/7 weeks, 26 weeks, 26 1/7 weeks, 26 2/7 weeks, 26 3/7 weeks, 26 4/7 weeks, 26 5/7 weeks, 26 6/7 weeks, 27 weeks, 27 1/7 weeks, 27 2/7 weeks, 27 3/7 weeks, 27 4/7 weeks, 27 5/7 weeks, 27 6/7 weeks, 28 weeks, 28 1/7 weeks, 28 2/7 weeks, 28 3/7 weeks, 28 4/7 weeks, 28 5/7 weeks, 28 6/7 weeks, 29 weeks, 29 1/7 weeks, 29 2/7 weeks, 29 3/7 weeks, 29 4/7 weeks, 29 5/7 weeks, 29 6/7 weeks, 30 weeks, 30 1/7 weeks, 30 2/7 weeks, 30 3/7 weeks, 30 4/7 weeks, 30 5/7 weeks, 30 6/7 weeks, 31 weeks, 31 1/7 weeks, 31 2/7 weeks, 31 3/7 weeks, 31 4/7 weeks, 31 5/7 weeks, 31 6/7 weeks, 32 weeks, 32 1/7 weeks, 32 2/7 weeks, 32 3/7 weeks, 32 4/7 weeks, 32 5/7 weeks, 32 6/7 weeks, 33 weeks, 33 1/7 weeks, 33 2/7 weeks, 33 3/7 weeks, 33 4/7 weeks, 33 5/7 weeks, 33 6/7 weeks, 34 weeks, 34 1/7 weeks, 34 2/7 weeks, 34 3/7 weeks, 34 4/7 weeks, 34 5/7 weeks, 34 6/7 weeks, 35 weeks, 35 1/7 weeks, 35 2/7 weeks, 35 3/7 weeks, 35 4/7 weeks, 35 5/7 weeks, 35 6/7 weeks, 36 weeks, 36 1/7 weeks, 36 2/7 weeks, 36 3/7 weeks, 36 4/7 weeks, 36 5/7 weeks, 36 6/7 weeks, 37 weeks, 37 1/7 weeks, 37 2/7 weeks, 37 3/7 weeks, 37 4/7 weeks, 37 5/7 weeks, 37 6/7 weeks, 38 weeks, 38 1/7 weeks, 38 2/7 weeks, 38 3/7 weeks, 38 4/7 weeks, 38 5/7 weeks, 38 6/7 weeks, 39 weeks, 39 1/7 weeks, 39 2/7 weeks, 39 3/7 weeks, 39 4/7 weeks, 39 5/7 weeks, 39 6/7 weeks, 40 weeks, 40 1/7 weeks, 40 2/7 weeks, 40 3/7 weeks, 40 4/7 weeks, 40 5/7 weeks, 40 6/7 weeks, 41 weeks, 41 1/7 weeks, 41 2/7 weeks, 41 3/7 weeks, 41 4/7 weeks, 41 5/7 weeks, 41 6/7 weeks, 42 weeks, 42 1/7 weeks, 42 2/7 weeks, 42 3/7 weeks, 42 4/7 weeks, 42 5/7 weeks, 42 6/7 weeks, 43 weeks, 43 1/7 weeks, 43 2/7 weeks, 43 3/7 weeks, 43 4/7 weeks, 43 5/7 weeks, 43 6/7 weeks, or 44 weeks). In some embodiments, the first treatment period has a duration of about 12 weeks.

In some embodiments, the first treatment period has a duration of from about 14 weeks to about 40 weeks, a duration of from about 16 weeks to about 32 weeks, a duration of from about 18 weeks to about 30 weeks, a duration of from about 20 weeks to about 28 weeks, or a duration of from about 22 weeks to about 26 weeks (e.g., a duration of about 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, 16 weeks, 16 1/7 weeks, 16 2/7 weeks, 16 3/7 weeks, 16 4/7 weeks, 16 5/7 weeks, 16 6/7 weeks, 17 weeks, 17 1/7 weeks, 17 2/7 weeks, 17 3/7 weeks, 17 4/7 weeks, 17 5/7 weeks, 17 6/7 weeks, 18 weeks, 18 1/7 weeks, 18 2/7 weeks, 18 3/7 weeks, 18 4/7 weeks, 18 5/7 weeks, 18 6/7 weeks, 19 weeks, 19 1/7 weeks, 19 2/7 weeks, 19 3/7 weeks, 19 4/7 weeks, 19 5/7 weeks, 19 6/7 weeks, 20 weeks, 20 1/7 weeks, 20 2/7 weeks, 20 3/7 weeks, 20 4/7 weeks, 20 5/7 weeks, 20 6/7 weeks, 21 weeks, 21 1/7 weeks, 21 2/7 weeks, 21 3/7 weeks, 21 4/7 weeks, 21 5/7 weeks, 21 6/7 weeks, 22 weeks, 22 1/7 weeks, 22 2/7 weeks, 22 3/7 weeks, 22 4/7 weeks, 22 5/7 weeks, 22 6/7 weeks, 23 weeks, 23 1/7 weeks, 23 2/7 weeks, 23 3/7 weeks, 23 4/7 weeks, 23 5/7 weeks, 23 6/7 weeks, 24 weeks, 24 1/7 weeks, 24 2/7 weeks, 24 3/7 weeks, 24 4/7 weeks, 24 5/7 weeks, 24 6/7 weeks, 25 weeks, 25 1/7 weeks, 25 2/7 weeks, 25 3/7 weeks, 25 4/7 weeks, 25 5/7 weeks, 25 6/7 weeks, 26 weeks, 26 1/7 weeks, 26 2/7 weeks, 26 3/7 weeks, 26 4/7 weeks, 26 5/7 weeks, 26 6/7 weeks, 27 weeks, 27 1/7 weeks, 27 2/7 weeks, 27 3/7 weeks, 27 4/7 weeks, 27 5/7 weeks, 27 6/7 weeks, 28 weeks, 28 1/7 weeks, 28 2/7 weeks, 28 3/7 weeks, 28 4/7 weeks, 28 5/7 weeks, 28 6/7 weeks, 29 weeks, 29 1/7 weeks, 29 2/7 weeks, 29 3/7 weeks, 29 4/7 weeks, 29 5/7 weeks, 29 6/7 weeks, 30 weeks, 30 1/7 weeks, 30 2/7 weeks, 30 3/7 weeks, 30 4/7 weeks, 30 5/7 weeks, 30 6/7 weeks, 31 weeks, 31 1/7 weeks, 31 2/7 weeks, 31 3/7 weeks, 31 4/7 weeks, 31 5/7 weeks, 31 6/7 weeks, 32 weeks, 32 1/7 weeks, 32 2/7 weeks, 32 3/7 weeks, 32 4/7 weeks, 32 5/7 weeks, 32 6/7 weeks, 33 weeks, 33 1/7 weeks, 33 2/7 weeks, 33 3/7 weeks, 33 4/7 weeks, 33 5/7 weeks, 33 6/7 weeks, 34 weeks, 34 1/7 weeks, 34 2/7 weeks, 34 3/7 weeks, 34 4/7 weeks, 34 5/7 weeks, 34 6/7 weeks, 35 weeks, 35 1/7 weeks, 35 2/7 weeks, 35 3/7 weeks, 35 4/7 weeks, 35 5/7 weeks, 35 6/7 weeks, 36 weeks, 36 1/7 weeks, 36 2/7 weeks, 36 3/7 weeks, 36 4/7 weeks, 36 5/7 weeks, 36 6/7 weeks, 37 weeks, 37 1/7 weeks, 37 2/7 weeks, 37 3/7 weeks, 37 4/7 weeks, 37 5/7 weeks, 37 6/7 weeks, 38 weeks, 38 1/7 weeks, 38 2/7 weeks, 38 3/7 weeks, 38 4/7 weeks, 38 5/7 weeks, 38 6/7 weeks, 39 weeks, 39 1/7 weeks, 39 2/7 weeks, 39 3/7 weeks, 39 4/7 weeks, 39 5/7 weeks, 39 6/7 weeks, or 40 weeks). In some embodiments, the first treatment period has a duration of about 24 weeks.

In some embodiments of the disclosure, the second treatment period has a duration of at least four weeks (e.g., a duration of about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). For example, the second treatment period may have a duration of at least eight weeks (e.g., a duration of about 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). In some embodiments, the second treatment period has a duration of at least 10 weeks (e.g., a duration of about 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). In some embodiments, the second treatment period has a duration of at least 12 weeks (e.g., a duration of about 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). In some embodiments, the second treatment period has a duration of at least 24 weeks (e.g., a duration of about 24 weeks).

In some embodiments, the second treatment period has a duration of from about four weeks to about 12 months (e.g., a duration of about 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, 6 weeks, 6 1/7 weeks, 6 2/7 weeks, 6 3/7 weeks, 6 4/7 weeks, 6 5/7 weeks, 6 6/7 weeks, 7 weeks, 7 1/7 weeks, 7 2/7 weeks, 7 3/7 weeks, 7 4/7 weeks, 7 5/7 weeks, 7 6/7 weeks, 8 weeks, 8 1/7 weeks, 8 2/7 weeks, 8 3/7 weeks, 8 4/7 weeks, 8 5/7 weeks, 8 6/7 weeks, 9 weeks, 9 1/7 weeks, 9 2/7 weeks, 9 3/7 weeks, 9 4/7 weeks, 9 5/7 weeks, 9 6/7 weeks, 10 weeks, 10 1/7 weeks, 10 2/7 weeks, 10 3/7 weeks, 10 4/7 weeks, 10 5/7 weeks, 10 6/7 weeks, 11 weeks, 11 1/7 weeks, 11 2/7 weeks, 11 3/7 weeks, 11 4/7 weeks, 11 5/7 weeks, 11 6/7 weeks, 12 weeks, 12 1/7 weeks, 12 2/7 weeks, 12 3/7 weeks, 12 4/7 weeks, 12 5/7 weeks, 12 6/7 weeks, 13 weeks, 13 1/7 weeks, 13 2/7 weeks, 13 3/7 weeks, 13 4/7 weeks, 13 5/7 weeks, 13 6/7 weeks, 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, 16 weeks, 16 1/7 weeks, 16 2/7 weeks, 16 3/7 weeks, 16 4/7 weeks, 16 5/7 weeks, 16 6/7 weeks, 17 weeks, 17 1/7 weeks, 17 2/7 weeks, 17 3/7 weeks, 17 4/7 weeks, 17 5/7 weeks, 17 6/7 weeks, 18 weeks, 18 1/7 weeks, 18 2/7 weeks, 18 3/7 weeks, 18 4/7 weeks, 18 5/7 weeks, 18 6/7 weeks, 19 weeks, 19 1/7 weeks, 19 2/7 weeks, 19 3/7 weeks, 19 4/7 weeks, 19 5/7 weeks, 19 6/7 weeks, 20 weeks, 20 1/7 weeks, 20 2/7 weeks, 20 3/7 weeks, 20 4/7 weeks, 20 5/7 weeks, 20 6/7 weeks, 21 weeks, 21 1/7 weeks, 21 2/7 weeks, 21 3/7 weeks, 21 4/7 weeks, 21 5/7 weeks, 21 6/7 weeks, 22 weeks, 22 1/7 weeks, 22 2/7 weeks, 22 3/7 weeks, 22 4/7 weeks, 22 5/7 weeks, 22 6/7 weeks, 23 weeks, 23 1/7 weeks, 23 2/7 weeks, 23 3/7 weeks, 23 4/7 weeks, 23 5/7 weeks, 23 6/7 weeks, 24 weeks, 24 1/7 weeks, 24 2/7 weeks, 24 3/7 weeks, 24 4/7 weeks, 24 5/7 weeks, 24 6/7 weeks, 25 weeks, 25 1/7 weeks, 25 2/7 weeks, 25 3/7 weeks, 25 4/7 weeks, 25 5/7 weeks, 25 6/7 weeks, 26 weeks, 26 1/7 weeks, 26 2/7 weeks, 26 3/7 weeks, 26 4/7 weeks, 26 5/7 weeks, 26 6/7 weeks, 27 weeks, 27 1/7 weeks, 27 2/7 weeks, 27 3/7 weeks, 27 4/7 weeks, 27 5/7 weeks, 27 6/7 weeks, 28 weeks, 28 1/7 weeks, 28 2/7 weeks, 28 3/7 weeks, 28 4/7 weeks, 28 5/7 weeks, 28 6/7 weeks, 29 weeks, 29 1/7 weeks, 29 2/7 weeks, 29 3/7 weeks, 29 4/7 weeks, 29 5/7 weeks, 29 6/7 weeks, 30 weeks, 30 1/7 weeks, 30 2/7 weeks, 30 3/7 weeks, 30 4/7 weeks, 30 5/7 weeks, 30 6/7 weeks, 31 weeks, 31 1/7 weeks, 31 2/7 weeks, 31 3/7 weeks, 31 4/7 weeks, 31 5/7 weeks, 31 6/7 weeks, 32 weeks, 32 1/7 weeks, 32 2/7 weeks, 32 3/7 weeks, 32 4/7 weeks, 32 5/7 weeks, 32 6/7 weeks, 33 weeks, 33 1/7 weeks, 33 2/7 weeks, 33 3/7 weeks, 33 4/7 weeks, 33 5/7 weeks, 33 6/7 weeks, 34 weeks, 34 1/7 weeks, 34 2/7 weeks, 34 3/7 weeks, 34 4/7 weeks, 34 5/7 weeks, 34 6/7 weeks, 35 weeks, 35 1/7 weeks, 35 2/7 weeks, 35 3/7 weeks, 35 4/7 weeks, 35 5/7 weeks, 35 6/7 weeks, 36 weeks, 36 1/7 weeks, 36 2/7 weeks, 36 3/7 weeks, 36 4/7 weeks, 36 5/7 weeks, 36 6/7 weeks, 37 weeks, 37 1/7 weeks, 37 2/7 weeks, 37 3/7 weeks, 37 4/7 weeks, 37 5/7 weeks, 37 6/7 weeks, 38 weeks, 38 1/7 weeks, 38 2/7 weeks, 38 3/7 weeks, 38 4/7 weeks, 38 5/7 weeks, 38 6/7 weeks, 39 weeks, 39 1/7 weeks, 39 2/7 weeks, 39 3/7 weeks, 39 4/7 weeks, 39 5/7 weeks, 39 6/7 weeks, 40 weeks, 40 1/7 weeks, 40 2/7 weeks, 40 3/7 weeks, 40 4/7 weeks, 40 5/7 weeks, 40 6/7 weeks, 41 weeks, 41 1/7 weeks, 41 2/7 weeks, 41 3/7 weeks, 41 4/7 weeks, 41 5/7 weeks, 41 6/7 weeks, 42 weeks, 42 1/7 weeks, 42 2/7 weeks, 42 3/7 weeks, 42 4/7 weeks, 42 5/7 weeks, 42 6/7 weeks, 43 weeks, 43 1/7 weeks, 43 2/7 weeks, 43 3/7 weeks, 43 4/7 weeks, 43 5/7 weeks, 43 6/7 weeks, 44 weeks, 44 1/7 weeks, 44 2/7 weeks, 44 3/7 weeks, 44 4/7 weeks, 44 5/7 weeks, 44 6/7 weeks, 45 weeks, 45 1/7 weeks, 45 2/7 weeks, 45 3/7 weeks, 45 4/7 weeks, 45 5/7 weeks, 45 6/7 weeks, 46 weeks, 46 1/7 weeks, 46 2/7 weeks, 46 3/7 weeks, 46 4/7 weeks, 46 5/7 weeks, 46 6/7 weeks, 47 weeks, 47 1/7 weeks, 47 2/7 weeks, 47 3/7 weeks, 47 4/7 weeks, 47 5/7 weeks, 47 6/7 weeks, or 48 weeks).

In some embodiments, the second treatment period has a duration of from about four weeks to about 44 weeks, a duration of from about four weeks to about 36 weeks, a duration of from about four weeks to about 24 weeks, a duration of from about five weeks to about 20 weeks, a duration of from about six weeks to about 18 weeks, a duration of from about eight weeks to about 16 weeks, or a duration of from about 10 weeks to about 14 weeks (e.g., a duration of about 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, 6 weeks, 6 1/7 weeks, 6 2/7 weeks, 6 3/7 weeks, 6 4/7 weeks, 6 5/7 weeks, 6 6/7 weeks, 7 weeks, 7 1/7 weeks, 7 2/7 weeks, 7 3/7 weeks, 7 4/7 weeks, 7 5/7 weeks, 7 6/7 weeks, 8 weeks, 8 1/7 weeks, 8 2/7 weeks, 8 3/7 weeks, 8 4/7 weeks, 8 5/7 weeks, 8 6/7 weeks, 9 weeks, 9 1/7 weeks, 9 2/7 weeks, 9 3/7 weeks, 9 4/7 weeks, 9 5/7 weeks, 9 6/7 weeks, 10 weeks, 10 1/7 weeks, 10 2/7 weeks, 10 3/7 weeks, 10 4/7 weeks, 10 5/7 weeks, 10 6/7 weeks, 11 weeks, 11 1/7 weeks, 11 2/7 weeks, 11 3/7 weeks, 11 4/7 weeks, 11 5/7 weeks, 11 6/7 weeks, 12 weeks, 12 1/7 weeks, 12 2/7 weeks, 12 3/7 weeks, 12 4/7 weeks, 12 5/7 weeks, 12 6/7 weeks, 13 weeks, 13 1/7 weeks, 13 2/7 weeks, 13 3/7 weeks, 13 4/7 weeks, 13 5/7 weeks, 13 6/7 weeks, 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, 16 weeks, 16 1/7 weeks, 16 2/7 weeks, 16 3/7 weeks, 16 4/7 weeks, 16 5/7 weeks, 16 6/7 weeks, 17 weeks, 17 1/7 weeks, 17 2/7 weeks, 17 3/7 weeks, 17 4/7 weeks, 17 5/7 weeks, 17 6/7 weeks, 18 weeks, 18 1/7 weeks, 18 2/7 weeks, 18 3/7 weeks, 18 4/7 weeks, 18 5/7 weeks, 18 6/7 weeks, 19 weeks, 19 1/7 weeks, 19 2/7 weeks, 19 3/7 weeks, 19 4/7 weeks, 19 5/7 weeks, 19 6/7 weeks, 20 weeks, 20 1/7 weeks, 20 2/7 weeks, 20 3/7 weeks, 20 4/7 weeks, 20 5/7 weeks, 20 6/7 weeks, 21 weeks, 21 1/7 weeks, 21 2/7 weeks, 21 3/7 weeks, 21 4/7 weeks, 21 5/7 weeks, 21 6/7 weeks, 22 weeks, 22 1/7 weeks, 22 2/7 weeks, 22 3/7 weeks, 22 4/7 weeks, 22 5/7 weeks, 22 6/7 weeks, 23 weeks, 23 1/7 weeks, 23 2/7 weeks, 23 3/7 weeks, 23 4/7 weeks, 23 5/7 weeks, 23 6/7 weeks, 24 weeks, 24 1/7 weeks, 24 2/7 weeks, 24 3/7 weeks, 24 4/7 weeks, 24 5/7 weeks, 24 6/7 weeks, 25 weeks, 25 1/7 weeks, 25 2/7 weeks, 25 3/7 weeks, 25 4/7 weeks, 25 5/7 weeks, 25 6/7 weeks, 26 weeks, 26 1/7 weeks, 26 2/7 weeks, 26 3/7 weeks, 26 4/7 weeks, 26 5/7 weeks, 26 6/7 weeks, 27 weeks, 27 1/7 weeks, 27 2/7 weeks, 27 3/7 weeks, 27 4/7 weeks, 27 5/7 weeks, 27 6/7 weeks, 28 weeks, 28 1/7 weeks, 28 2/7 weeks, 28 3/7 weeks, 28 4/7 weeks, 28 5/7 weeks, 28 6/7 weeks, 29 weeks, 29 1/7 weeks, 29 2/7 weeks, 29 3/7 weeks, 29 4/7 weeks, 29 5/7 weeks, 29 6/7 weeks, 30 weeks, 30 1/7 weeks, 30 2/7 weeks, 30 3/7 weeks, 30 4/7 weeks, 30 5/7 weeks, 30 6/7 weeks, 31 weeks, 31 1/7 weeks, 31 2/7 weeks, 31 3/7 weeks, 31 4/7 weeks, 31 5/7 weeks, 31 6/7 weeks, 32 weeks, 32 1/7 weeks, 32 2/7 weeks, 32 3/7 weeks, 32 4/7 weeks, 32 5/7 weeks, 32 6/7 weeks, 33 weeks, 33 1/7 weeks, 33 2/7 weeks, 33 3/7 weeks, 33 4/7 weeks, 33 5/7 weeks, 33 6/7 weeks, 34 weeks, 34 1/7 weeks, 34 2/7 weeks, 34 3/7 weeks, 34 4/7 weeks, 34 5/7 weeks, 34 6/7 weeks, 35 weeks, 35 1/7 weeks, 35 2/7 weeks, 35 3/7 weeks, 35 4/7 weeks, 35 5/7 weeks, 35 6/7 weeks, 36 weeks, 36 1/7 weeks, 36 2/7 weeks, 36 3/7 weeks, 36 4/7 weeks, 36 5/7 weeks, 36 6/7 weeks, 37 weeks, 37 1/7 weeks, 37 2/7 weeks, 37 3/7 weeks, 37 4/7 weeks, 37 5/7 weeks, 37 6/7 weeks, 38 weeks, 38 1/7 weeks, 38 2/7 weeks, 38 3/7 weeks, 38 4/7 weeks, 38 5/7 weeks, 38 6/7 weeks, 39 weeks, 39 1/7 weeks, 39 2/7 weeks, 39 3/7 weeks, 39 4/7 weeks, 39 5/7 weeks, 39 6/7 weeks, 40 weeks, 40 1/7 weeks, 40 2/7 weeks, 40 3/7 weeks, 40 4/7 weeks, 40 5/7 weeks, 40 6/7 weeks, 41 weeks, 41 1/7 weeks, 41 2/7 weeks, 41 3/7 weeks, 41 4/7 weeks, 41 5/7 weeks, 41 6/7 weeks, 42 weeks, 42 1/7 weeks, 42 2/7 weeks, 42 3/7 weeks, 42 4/7 weeks, 42 5/7 weeks, 42 6/7 weeks, 43 weeks, 43 1/7 weeks, 43 2/7 weeks, 43 3/7 weeks, 43 4/7 weeks, 43 5/7 weeks, 43 6/7 weeks, or 44 weeks). In some embodiments, the second treatment period has a duration of about 12 weeks.

In some embodiments, the second treatment period has a duration of from about 14 weeks to about 40 weeks, a duration of from about 16 weeks to about 32 weeks, a duration of from about 18 weeks to about 30 weeks, a duration of from about 20 weeks to about 28 weeks, or a duration of from about 22 weeks to about 26 weeks (e.g., a duration of about 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, 16 weeks, 16 1/7 weeks, 16 2/7 weeks, 16 3/7 weeks, 16 4/7 weeks, 16 5/7 weeks, 16 6/7 weeks, 17 weeks, 17 1/7 weeks, 17 2/7 weeks, 17 3/7 weeks, 17 4/7 weeks, 17 5/7 weeks, 17 6/7 weeks, 18 weeks, 18 1/7 weeks, 18 2/7 weeks, 18 3/7 weeks, 18 4/7 weeks, 18 5/7 weeks, 18 6/7 weeks, 19 weeks, 19 1/7 weeks, 19 2/7 weeks, 19 3/7 weeks, 19 4/7 weeks, 19 5/7 weeks, 19 6/7 weeks, 20 weeks, 20 1/7 weeks, 20 2/7 weeks, 20 3/7 weeks, 20 4/7 weeks, 20 5/7 weeks, 20 6/7 weeks, 21 weeks, 21 1/7 weeks, 21 2/7 weeks, 21 3/7 weeks, 21 4/7 weeks, 21 5/7 weeks, 21 6/7 weeks, 22 weeks, 22 1/7 weeks, 22 2/7 weeks, 22 3/7 weeks, 22 4/7 weeks, 22 5/7 weeks, 22 6/7 weeks, 23 weeks, 23 1/7 weeks, 23 2/7 weeks, 23 3/7 weeks, 23 4/7 weeks, 23 5/7 weeks, 23 6/7 weeks, 24 weeks, 24 1/7 weeks, 24 2/7 weeks, 24 3/7 weeks, 24 4/7 weeks, 24 5/7 weeks, 24 6/7 weeks, 25 weeks, 25 1/7 weeks, 25 2/7 weeks, 25 3/7 weeks, 25 4/7 weeks, 25 5/7 weeks, 25 6/7 weeks, 26 weeks, 26 1/7 weeks, 26 2/7 weeks, 26 3/7 weeks, 26 4/7 weeks, 26 5/7 weeks, 26 6/7 weeks, 27 weeks, 27 1/7 weeks, 27 2/7 weeks, 27 3/7 weeks, 27 4/7 weeks, 27 5/7 weeks, 27 6/7 weeks, 28 weeks, 28 1/7 weeks, 28 2/7 weeks, 28 3/7 weeks, 28 4/7 weeks, 28 5/7 weeks, 28 6/7 weeks, 29 weeks, 29 1/7 weeks, 29 2/7 weeks, 29 3/7 weeks, 29 4/7 weeks, 29 5/7 weeks, 29 6/7 weeks, 30 weeks, 30 1/7 weeks, 30 2/7 weeks, 30 3/7 weeks, 30 4/7 weeks, 30 5/7 weeks, 30 6/7 weeks, 31 weeks, 31 1/7 weeks, 31 2/7 weeks, 31 3/7 weeks, 31 4/7 weeks, 31 5/7 weeks, 31 6/7 weeks, 32 weeks, 32 1/7 weeks, 32 2/7 weeks, 32 3/7 weeks, 32 4/7 weeks, 32 5/7 weeks, 32 6/7 weeks, 33 weeks, 33 1/7 weeks, 33 2/7 weeks, 33 3/7 weeks, 33 4/7 weeks, 33 5/7 weeks, 33 6/7 weeks, 34 weeks, 34 1/7 weeks, 34 2/7 weeks, 34 3/7 weeks, 34 4/7 weeks, 34 5/7 weeks, 34 6/7 weeks, 35 weeks, 35 1/7 weeks, 35 2/7 weeks, 35 3/7 weeks, 35 4/7 weeks, 35 5/7 weeks, 35 6/7 weeks, 36 weeks, 36 1/7 weeks, 36 2/7 weeks, 36 3/7 weeks, 36 4/7 weeks, 36 5/7 weeks, 36 6/7 weeks, 37 weeks, 37 1/7 weeks, 37 2/7 weeks, 37 3/7 weeks, 37 4/7 weeks, 37 5/7 weeks, 37 6/7 weeks, 38 weeks, 38 1/7 weeks, 38 2/7 weeks, 38 3/7 weeks, 38 4/7 weeks, 38 5/7 weeks, 38 6/7 weeks, 39 weeks, 39 1/7 weeks, 39 2/7 weeks, 39 3/7 weeks, 39 4/7 weeks, 39 5/7 weeks, 39 6/7 weeks, or 40 weeks). In some embodiments, the second treatment period has a duration of about 24 weeks.

In some embodiments of the disclosure, the time elapsed between the end of the first treatment period and commencement of the second treatment period is at least about two weeks (e.g., about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more).

In some embodiments, the time elapsed between the end of the first treatment period and commencement of the second treatment period is at least about four weeks (e.g., about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more).

In some embodiments, the time elapsed between the end of the first treatment period and commencement of the second treatment period is at least about eight weeks (e.g., about 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more).

In some embodiments, the time elapsed between the end of the first treatment period and commencement of the second treatment period is at least about 12 weeks (e.g., about 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more).

In some embodiments, the time elapsed between the end of the first treatment period and commencement of the second treatment period is at least about 24 weeks (e.g., about 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more).

In some embodiments, the time elapsed between the end of the first treatment period and commencement of the second treatment period is from about one week to about 12 weeks, from about one week to about six weeks, or from about one week to about four weeks (e.g., about 1 week, 1 1/7 weeks, 1 2/7 weeks, 1 3/7 weeks, 1 4/7 weeks, 1 5/7 weeks, 1 6/7 weeks, 2 weeks, 2 1/7 weeks, 2 2/7 weeks, 2 3/7 weeks, 2 4/7 weeks, 2 5/7 weeks, 2 6/7 weeks, 3 weeks, 3 1/7 weeks, 3 2/7 weeks, 3 3/7 weeks, 3 4/7 weeks, 3 5/7 weeks, 3 6/7 weeks, 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, 6 weeks, 6 1/7 weeks, 6 2/7 weeks, 6 3/7 weeks, 6 4/7 weeks, 6 5/7 weeks, 6 6/7 weeks, 7 weeks, 7 1/7 weeks, 7 2/7 weeks, 7 3/7 weeks, 7 4/7 weeks, 7 5/7 weeks, 7 6/7 weeks, 8 weeks, 8 1/7 weeks, 8 2/7 weeks, 8 3/7 weeks, 8 4/7 weeks, 8 5/7 weeks, 8 6/7 weeks, 9 weeks, 9 1/7 weeks, 9 2/7 weeks, 9 3/7 weeks, 9 4/7 weeks, 9 5/7 weeks, 9 6/7 weeks, 10 weeks, 10 1/7 weeks, 10 2/7 weeks, 10 3/7 weeks, 10 4/7 weeks, 10 5/7 weeks, 10 6/7 weeks, 11 weeks, 11 1/7 weeks, 11 2/7 weeks, 11 3/7 weeks, 11 4/7 weeks, 11 5/7 weeks, 11 6/7 weeks, 12 weeks).

In some embodiments, the time elapsed between the end of the first treatment period and commencement of the second treatment period is about one week, about two weeks, about three weeks, about four weeks, about five weeks, or about six weeks. In some embodiments, the time elapsed between the end of the first treatment period and commencement of the second treatment period is about 1 week, 1 1/7 weeks, 1 2/7 weeks, 1 3/7 weeks, 1 4/7 weeks, 1 5/7 weeks, 1 6/7 weeks, 2 weeks, 2 1/7 weeks, 2 2/7 weeks, 2 3/7 weeks, 2 4/7 weeks, 2 5/7 weeks, 2 6/7 weeks, 3 weeks, 3 1/7 weeks, 3 2/7 weeks, 3 3/7 weeks, 3 4/7 weeks, 3 5/7 weeks, 3 6/7 weeks, 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, or 6 weeks.

In some embodiments, the time elapsed between the end of the first treatment period and commencement of the second treatment period is from about one month to about eight months, from about one month to about 6 months, or from about one month to about four months. In some embodiments, the time elapsed between the end of the first treatment period and commencement of the second treatment period is about 4 weeks, 4 1/7 weeks, 4 2/7 weeks, 4 3/7 weeks, 4 4/7 weeks, 4 5/7 weeks, 4 6/7 weeks, 5 weeks, 5 1/7 weeks, 5 2/7 weeks, 5 3/7 weeks, 5 4/7 weeks, 5 5/7 weeks, 5 6/7 weeks, 6 weeks, 6 1/7 weeks, 6 2/7 weeks, 6 3/7 weeks, 6 4/7 weeks, 6 5/7 weeks, 6 6/7 weeks, 7 weeks, 7 1/7 weeks, 7 2/7 weeks, 7 3/7 weeks, 7 4/7 weeks, 7 5/7 weeks, 7 6/7 weeks, 8 weeks, 8 1/7 weeks, 8 2/7 weeks, 8 3/7 weeks, 8 4/7 weeks, 8 5/7 weeks, 8 6/7 weeks, 9 weeks, 9 1/7 weeks, 9 2/7 weeks, 9 3/7 weeks, 9 4/7 weeks, 9 5/7 weeks, 9 6/7 weeks, 10 weeks, 10 1/7 weeks, 10 2/7 weeks, 10 3/7 weeks, 10 4/7 weeks, 10 5/7 weeks, 10 6/7 weeks, 11 weeks, 11 1/7 weeks, 11 2/7 weeks, 11 3/7 weeks, 11 4/7 weeks, 11 5/7 weeks, 11 6/7 weeks, 12 weeks, 12 1/7 weeks, 12 2/7 weeks, 12 3/7 weeks, 12 4/7 weeks, 12 5/7 weeks, 12 6/7 weeks, 13 weeks, 13 1/7 weeks, 13 2/7 weeks, 13 3/7 weeks, 13 4/7 weeks, 13 5/7 weeks, 13 6/7 weeks, 14 weeks, 14 1/7 weeks, 14 2/7 weeks, 14 3/7 weeks, 14 4/7 weeks, 14 5/7 weeks, 14 6/7 weeks, 15 weeks, 15 1/7 weeks, 15 2/7 weeks, 15 3/7 weeks, 15 4/7 weeks, 15 5/7 weeks, 15 6/7 weeks, or 16 weeks.

In some embodiments, the time elapsed between the end of the first treatment period and commencement of the second treatment period is about one month.

In some embodiments, the time elapsed between the end of the first treatment period and commencement of the second treatment period is about two months.

In some embodiments, the time elapsed between the end of the first treatment period and commencement of the second treatment period is about three months.

In some embodiments, the time elapsed between the end of the first treatment period and commencement of the second treatment period is about four months.

In some embodiments, the time elapsed between the end of the first treatment period and commencement of the second treatment period is about five months.

In some embodiments, the time elapsed between the end of the first treatment period and commencement of the second treatment period is about six months.

In some embodiments of the disclosure, add-back therapy is administered (e.g., periodically administered) to the patient during the first and/or second treatment period.

In some embodiments of the disclosure, the add-back therapy is not administered to the patient during the time elapsed between the end of the first treatment period and commencement of the second treatment period.

In some embodiments, the add-back therapy is administered to the patient concurrently with the GnRH antagonist, prior to administration of the GnRH antagonist, or following administration of the GnRH antagonist. In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of β17-estradiol, ethinyl estradiol, or a conjugated estrogen, such as a conjugated equine estrogen) and/or a progestin (e.g., norethindrone or a compound that is metabolized in vivo to produce norethindrone, such as an ester of norethindrone that is de-esterified in vivo to produce norethindrone, for instance, norethindrone acetate (also referred to herein as “NETA”), among other agents, such as progesterone, norgestimate, medroxyprogesterone, and drospirenone) in the form of a single pharmaceutical composition, such as a single tablet, capsule, gel cap, powder, liquid solution, or liquid suspension. In some embodiments, the add-back therapy is administered orally, transdermally, or intravaginally.

In some embodiments, the add-back therapy is administered to the patient in one or more doses per day, week, month, or year, such as daily, for example, from 1 to 10 times daily, or more (e.g., 1, 2, 3, 4, 5, 6, 7, 8, 9, 10, or more, times daily). In some embodiments, the add-back therapy is administered to the patient once daily, for example, concurrently with the GnRH antagonist. For example, the GnRH antagonist may be administered to the patient orally, and concurrently with oral administration of the GnRH antagonist, the add-back therapy may be administered to the patient orally, transdermally, or intravaginally. In some embodiments, the add-back therapy is administered to the patient in the form of a pharmaceutical composition that further includes the GnRH antagonist, such as a single tablet, capsule, gel cap, powder, liquid solution, or liquid suspension, for instance, as described above and herein.

In some embodiments, the add-back therapy is administered to the patient once daily, following administration of the GnRH antagonist. For example, the GnRH antagonist may be administered to the patient orally, and following oral administration of the GnRH antagonist, the add-back therapy may be administered to the patient orally, transdermally, or intravaginally.

In some embodiments, the add-back therapy is administered to the patient once daily, prior to administration of the GnRH antagonist. For example, the GnRH antagonist may be administered to the patient orally, and prior to oral administration of the GnRH antagonist, the add-back therapy may be administered to the patient orally, transdermally, or intravaginally.

In some embodiments, the add-back therapy includes an estrogen. In some embodiments, the estrogen is selected from the group consisting of β17-estradiol, ethinyl estradiol, and conjugated estrogens, such as conjugated equine estrogens.

In some embodiments, the estrogen is β17-estradiol. The β17-estradiol may be administered to the patient, for example, at a dose of from about 0.1 mg to about 2.5 mg, such as at a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, or 2.5 mg, for instance, by oral administration. In some embodiments, the β17-estradiol is administered to the patient at a dose of 1.0 mg, for instance, by oral administration. In some embodiments, the β17-estradiol is administered to the patient at a dose of 0.5 mg, for instance, by oral administration.

The β17-estradiol may be administered to the patient one or more times per day, week, or month. The β17-estradiol may be administered to the patient, for example, in an amount of from about 0.1 mg/day to about 2.5 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, or 2.5 mg/day, for instance, by oral administration. In some embodiments, the β17-estradiol is administered to the patient in an amount of 1.0 mg/day, for instance, by oral administration. In some embodiments, the β17-estradiol is administered to the patient in an amount of 0.5 mg/day, for instance, by oral administration.

In some embodiments, the estrogen is ethinyl estradiol. The ethinyl estradiol may be administered to the patient, for example, at a dose of from about 1.0 μg to about 6.0 μg, such as at a dose of about 1.0 μg, 1.1 μg, 1.2 μg, 1.3 μg, 1.4 μg, 1.5 μg, 1.6 μg, 1.7 μg, 1.8 μg, 1.9 μg, 2.0 μg, 2.1 μg, 2.2 μg, 2.3 μg, 2.4 μg, 2.5 μg, 2.6 μg, 2.7 μg, 2.8 μg, 2.9 μg, 3.0 μg, 3.1 μg, 3.2 μg, 3.3 μg, 3.4 μg, 3.5 μg, 3.6 μg, 3.7 μg, 3.8 μg, 3.9 μg, 4.0 μg, 4.1 μg, 4.2 μg, 4.2 μg, 4.3 μg, 4.4 μg, 4.5 μg, 4.6 μg, 4.7 μg, 4.8 μg, 4.9 μg, 5.0 μg, 5.1 μg, 5.2 μg, 5.3 μg, 5.4 μg, 5.5 μg, 5.6 μg, 5.7 μg, 5.8 μg, 5.9 μg, or 6.0 μg, for instance, by oral administration. In some embodiments, the ethinyl estradiol is administered to the patient at a dose of 5.0 μg, for instance, by oral administration. In some embodiments, the ethinyl estradiol is administered to the patient at a dose of 2.5 μg, for instance, by oral administration.

The ethinyl estradiol may be administered to the patient one or more times per day, week, or month. The ethinyl estradiol may be administered to the patient, for example, in an amount of about 1.0 μg/day to about 6.0 μg/day, such as in an amount of about 1.0 μg/day, 1.1 μg/day, 1.2 μg/day, 1.3 μg/day, 1.4 μg/day, 1.5 μg/day, 1.6 μg/day, 1.7 μg/day, 1.8 μg/day, 1.9 μg/day, 2.0 μg/day, 2.1 μg/day, 2.2 μg/day, 2.3 μg/day, 2.4 μg/day, 2.5 μg/day, 2.6 μg/day, 2.7 μg/day, 2.8 μg/day, 2.9 μg/day, 3.0 μg/day, 3.1 μg/day, 3.2 μg/day, 3.3 μg/day, 3.4 μg/day, 3.5 μg/day, 3.6 μg/day, 3.7 μg/day, 3.8 μg/day, 3.9 μg/day, 4.0 μg/day, 4.1 μg/day, 4.2 μg/day, 4.2 μg/day, 4.3 μg/day, 4.4 μg/day, 4.5 μg/day, 4.6 μg/day, 4.7 μg/day, 4.8 μg/day, 4.9 μg/day, 5.0 μg/day, 5.1 μg/day, 5.2 μg/day, 5.3 μg/day, 5.4 μg/day, 5.5 μg/day, 5.6 μg/day, 5.7 μg/day, 5.8 μg/day, 5.9 μg/day, or 6.0 μg/day, for instance, by oral administration. In some embodiments, the ethinyl estradiol is administered to the patient in an amount of 5.0 μg/day, for instance, by oral administration. In some embodiments, the ethinyl estradiol is administered to the patient in an amount of 2.5 μg/day, for instance, by oral administration.

In some embodiments, the estrogen is a conjugated estrogen, such as a conjugated equine estrogen. The conjugated estrogen may be administered to the patient, for example, at a dose of from about 0.1 mg to about 2.0 mg, such as at a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg, for instance, by oral administration. In some embodiments, the conjugated estrogen is administered to the patient at a dose of 0.625 mg, for instance, by oral administration. In some embodiments, the conjugated estrogen is administered to the patient at a dose of 0.45 mg, for instance, by oral administration. In some embodiments, the conjugated estrogen is administered to the patient at a dose of 0.3 mg, for instance, by oral administration.

The conjugated estrogen may be administered to the patient one or more times per day, week, or month. The conjugated estrogen may be administered to the patient, for example, in an amount of from about 0.1 mg/day to about 2.0 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, or 2.0 mg/day, for instance, by oral administration. In some embodiments, the conjugated estrogen is administered to the patient in an amount of 0.625 mg/day, for instance, by oral administration. In some embodiments, the conjugated estrogen is administered to the patient in an amount of 0.45 mg/day, for instance, by oral administration. In some embodiments, the conjugated estrogen is administered to the patient in an amount of 0.3 mg/day, for instance, by oral administration.

In some embodiments, the add-back therapy includes a progestin. In some embodiments, the progestin is selected from the group consisting of norethindrone or an ester thereof, such as norethindrone acetate, or another agent such as progesterone, norgestimate, medroxyprogesterone, or drospirenone.

In some embodiments, the progestin is norethindrone or a compound that is metabolized in vivo to produce norethindrone, such as an ester of norethindrone that is de-esterified in vivo to produce norethindrone, for instance, norethindrone acetate.

In some embodiments, the progestin is norethindrone. The norethindrone may be administered to the patient, for example, at a dose of from about 0.05 mg to about 5.0 mg, such as at a dose of about 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, or 5.0 mg, for instance, by oral administration. In some embodiments, the norethindrone is administered to the patient at a dose of 1.0 mg, for instance, by oral administration. In some embodiments, the norethindrone is administered to the patient at a dose of 0.5 mg, for instance, by oral administration. In some embodiments, the norethindrone is administered to the patient at a dose of 0.1 mg, for instance, by oral administration.

The norethindrone may be administered to the patient one or more times per day, week, or month. The norethindrone may be administered to the patient, for example, in an amount of from about 0.05 mg/day to about 5.0 mg/day, such as in an amount of about 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, or 5.0 mg/day, for instance, by oral administration. In some embodiments, the norethindrone is administered to the patient in an amount of 1.0 mg/day, for instance, by oral administration. In some embodiments, the norethindrone is administered to the patient in an amount of 0.5 mg/day, for instance, by oral administration. In some embodiments, the norethindrone is administered to the patient in an amount of 0.1 mg/day, for instance, by oral administration.

In some embodiments, the progestin is norethindrone acetate. The norethindrone acetate may be administered to the patient, for example, at a dose of from about 0.05 mg to about 5.0 mg, such as at a dose of about 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, or 5.0 mg, for instance, by oral administration. In some embodiments, the norethindrone acetate is administered to the patient at a dose of 1.0 mg, for instance, by oral administration. In some embodiments, the norethindrone acetate is administered to the patient at a dose of 0.5 mg, for instance, by oral administration. In some embodiments, the norethindrone acetate is administered to the patient at a dose of 0.1 mg, for instance, by oral administration.

The norethindrone acetate may be administered to the patient one or more times per day, week, or month. The norethindrone acetate may be administered to the patient, for example, in an amount of from about 0.05 mg/day to about 5.0 mg/day, such as in an amount of about 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, or 5.0 mg/day, for instance, by oral administration. In some embodiments, the norethindrone acetate is administered to the patient in an amount of 1.0 mg/day, for instance, by oral administration. In some embodiments, the norethindrone acetate is administered to the patient in an amount of 0.5 mg/day, for instance, by oral administration. In some embodiments, the norethindrone acetate is administered to the patient in an amount of 0.1 mg/day, for instance, by oral administration.

In some embodiments, the progestin is progesterone. The progesterone may be administered to the patient, for example, at a dose of from about 50 mg to about 250 mg, such as a dose of about 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 250 mg, for instance, by oral administration. In some embodiments, the progesterone is administered to the patient at a dose of 200 mg, for instance, by oral administration. In some embodiments, the progesterone is administered to the patient at a dose of 100 mg, for instance, by oral administration.

The progesterone may be administered to the patient one or more times per day, week, or month. The progesterone may be administered to the patient, for example, in an amount of from about 50 mg/day to about 250 mg/day, such as a dose of about 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 150 mg/day, 155 mg/day, 160 mg/day, 165 mg/day, 170 mg/day, 175 mg/day, 180 mg/day, 185 mg/day, 190 mg/day, 195 mg/day, 200 mg/day, 205 mg/day, 210 mg/day, 215 mg/day, 220 mg/day, 225 mg/day, 230 mg/day, 235 mg/day, 240 mg/day, 245 mg/day, or 250 mg/day, for instance, by oral administration. In some embodiments, the progesterone is administered to the patient in an amount of 200 mg/day, for instance, by oral administration. In some embodiments, the progesterone is administered to the patient in an amount of 100 mg/day, for instance, by oral administration.

In some embodiments, the progestin is norgestimate. The norgestimate may be administered to the patient, for example, at a dose of from about 0.01 mg to about 2.0 mg, such as at a dose of about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg, for instance, by oral administration. In some embodiments, the norgestimate is administered to the patient at a dose of 0.09 mg, for instance, by oral administration.

The norgestimate may be administered to the patient one or more times per day, week, or month. The norgestimate may be administered to the patient, for example, in an amount of from about 0.01 mg/day to about 2.0 mg/day, such as in an amount of about 0.01 mg/day, 0.02 mg/day, 0.03 mg/day, 0.04 mg/day, 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, or 2.0 mg/day, for instance, by oral administration. In some embodiments, the norgestimate is administered to the patient in an amount of 0.09 mg/day, for instance, by oral administration.

In some embodiments, the progestin is medroxyprogesterone. The medroxyprogesterone may be administered to the patient, for example, at a dose of from about 0.5 mg to about 10.0 mg, such as at a dose of about 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, or 10.0 mg, for instance, by oral administration. In some embodiments, the medroxyprogesterone is administered to the patient at a dose of 5.0 mg, for instance, by oral administration. In some embodiments, the medroxyprogesterone is administered to the patient at a dose of 2.5 mg, for instance, by oral administration. In some embodiments, the medroxyprogesterone is administered to the patient at a dose of 1.5 mg, for instance, by oral administration.

The medroxyprogesterone may be administered to the patient one or more times per day, week, or month. The medroxyprogesterone may be administered to the patient, for example, in an amount of from about 0.5 mg/day to about 10.0 mg/day, such as in an amount of about 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, 5.0 mg/day, 5.1 mg/day, 5.2 mg/day, 5.3 mg/day, 5.4 mg/day, 5.5 mg/day, 5.6 mg/day, 5.7 mg/day, 5.8 mg/day, 5.9 mg/day, 6.0 mg/day, 6.1 mg/day, 6.2 mg/day, 6.3 mg/day, 6.4 mg/day, 6.5 mg/day, 6.6 mg/day, 6.7 mg/day, 6.8 mg/day, 6.9 mg/day, 7.0 mg/day, 7.1 mg/day, 7.2 mg/day, 7.3 mg/day, 7.4 mg/day, 7.5 mg/day, 7.6 mg/day, 7.7 mg/day, 7.8 mg/day, 7.9 mg/day, 8.0 mg/day, 8.1 mg/day, 8.2 mg/day, 8.3 mg/day, 8.4 mg/day, 8.5 mg/day, 8.6 mg/day, 8.7 mg/day, 8.8 mg/day, 8.9 mg/day, 9.0 mg/day, 9.1 mg/day, 9.2 mg/day, 9.3 mg/day, 9.4 mg/day, 9.5 mg/day, 9.6 mg/day, 9.7 mg/day, 9.8 mg/day, 9.9 mg/day, or 10.0 mg/day, for instance, by oral administration. In some embodiments, the medroxyprogesterone is administered to the patient in an amount of 5.0 mg/day, for instance, by oral administration. In some embodiments, the medroxyprogesterone is administered to the patient in an amount of 2.5 mg/day, for instance, by oral administration. In some embodiments, the medroxyprogesterone is administered to the patient in an amount of 1.5 mg/day, for instance, by oral administration.

In some embodiments, the progestin is drospirenone. The drospirenone may be administered to the patient, for example, at a dose of from about 0.1 mg to about 1.0 mg, such as at a dose of about 0.1 mg, 0.2 mg, 0.3 mg, 0.4 mg, 0.5 mg, 0.6 mg, 0.7 mg, 0.8 mg, 0.9 mg, or 1.0 mg, for instance, by oral administration. In some embodiments, the drospirenone is administered to the patient at a dose of 0.5 mg, for instance, by oral administration. In some embodiments, the drospirenone is administered to the patient at a dose of 0.25 mg, for instance, by oral administration.

The drospirenone may be administered to the patient one or more times per day, week, or month. The drospirenone may be administered to the patient, for example, in an amount of from about 0.1 mg/day to about 1.0 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, or 1.0 mg/day, for instance, by oral administration. In some embodiments, the drospirenone is administered to the patient in an amount of 0.5 mg/day, for instance, by oral administration. In some embodiments, the drospirenone is administered to the patient in an amount of 0.25 mg/day, for instance, by oral administration.

In some embodiments, the add-back therapy includes an estrogen and a progestin. In some embodiments, the add-back therapy includes β17-estradiol and norethindrone or a compound that is metabolized in vivo to produce norethindrone, such as an ester of norethindrone that is de-esterified in vivo to produce norethindrone, for instance, norethindrone acetate.

In some embodiments, the add-back therapy includes from about 0.75 mg to about 1.25 mg of β17-estradiol, e.g., administered orally, and from about 0.25 mg to about 0.75 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 1.0 mg of β17-estradiol, e.g., administered orally, and 0.5 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 1.0 mg of β17-estradiol, e.g., administered orally, and, in the same pharmaceutical composition, 0.5 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 1.0 mg of β17-estradiol, e.g., administered orally, and, in a separate pharmaceutical composition, 0.5 mg of norethindrone or a compound that is metabolized in vivo to produce norethindrone, such as an ester of norethindrone that is de-esterified in vivo to produce norethindrone, for instance, norethindrone acetate, e.g., administered orally.

In some embodiments, the GnRH antagonist is administered to the patient in a fixed-dose composition that contains the GnRH antagonist (e.g., in an amount of about 100 mg or about 200 mg of the compound of any one of formulas (I)-(VI)), from about 0.75 mg to about 1.25 mg of β17-estradiol, and from about 0.25 mg to about 0.75 mg of norethindrone or a compound that is metabolized in vivo to produce norethindrone, such as an ester of norethindrone that is de-esterified in vivo to produce norethindrone, for instance, norethindrone acetate. In some embodiments, the GnRH antagonist is administered to the patient in a fixed-dose composition that contains the GnRH antagonist (e.g., in an amount of about 100 mg or about 200 mg of the compound of any one of formulas (I)-(VI)), about 1.0 mg of β17-estradiol (e.g., 1.0 mg of β17-estradiol), and about 0.5 mg of norethindrone or a compound that is metabolized in vivo to produce norethindrone, such as an ester of norethindrone that is de-esterified in vivo to produce norethindrone, for instance, norethindrone acetate (e.g., 0.5 mg of norethindrone or a compound that is metabolized in vivo to produce norethindrone, such as an ester of norethindrone that is de-esterified in vivo to produce norethindrone, for instance, norethindrone acetate). In some embodiments, the GnRH antagonist is administered to the patient in a fixed-dose composition that contains the GnRH antagonist (e.g., in an amount of about 100 mg or about 200 mg of the compound of any one of formulas (I)-(VI)), 1.0 mg of β17-estradiol, and 0.5 mg of norethindrone acetate.

In some embodiments, the above fixed-dose composition is administered to the patient in one or more doses per 12 hours, 24 hours, 48 hours, 72 hours, week, month, or year, such as in from 1 to 10 doses per 12 hours (e.g., 1 dose every 12 hours, 2 doses every 12 hours, 3 doses every 12 hours, 4 doses every 12 hours, 5 doses every 12 hours, 6 doses every 12 hours, 7 doses every 12 hours, 8 doses every 12 hours, 9 doses every 12 hours, or 10 doses every 12 hours), from 1 to 10 doses per 24 hours (e.g., 1 dose every 24 hours, 2 doses every 24 hours, 3 doses every 24 hours, 4 doses every 24 hours, 5 doses every 24 hours, 6 doses every 24 hours, 7 doses every 24 hours, 8 doses every 24 hours, 9 doses every 24 hours, or 10 doses every 24 hours), from 1 to 10 doses per 48 hours (e.g., 1 dose every 48 hours, 2 doses every 48 hours, 3 doses every 48 hours, 4 doses every 48 hours, 5 doses every 48 hours, 6 doses every 48 hours, 7 doses every 48 hours, 8 doses every 48 hours, 9 doses every 48 hours, or 10 doses every 48 hours), from 1 to 10 doses per 72 hours (e.g., 1 dose every 72 hours, 2 doses every 72 hours, 3 doses every 72 hours, 4 doses every 72 hours, 5 doses every 72 hours, 6 doses every 72 hours, 7 doses every 72 hours, 8 doses every 72 hours, 9 doses every 72 hours, or 10 doses every 72 hours), from 1 to 10 doses per week (e.g., 1 dose every week, 2 doses every week, 3 doses every week, 4 doses every week, 5 doses every week, 6 doses every week, 7 doses every week, 8 doses every week, 9 doses every week, or 10 doses every week), or from 1 to 60 doses per month (e.g., from 30-60 doses per month, such as 1 time daily, 2 times daily, 3 times daily, 4 times daily, 5 times daily, 6 times daily, 7 times daily, 8 times daily, 9 times daily, 10 times daily, 7 times weekly, 8 times weekly, 9 times weekly, 10 times weekly, 11 times weekly, 12 times weekly, 13 times weekly, 14 times weekly, or more), among others. In some embodiments, the above fixed-dose composition is administered to the patient once daily.

In some embodiments, the add-back therapy includes from about 0.25 mg to about 0.75 mg of β17-estradiol, e.g., administered orally, and from about 0.05 mg to about 0.2 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 0.5 mg of β17-estradiol, e.g., administered orally, and 0.1 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 0.5 mg of β17-estradiol, e.g., administered orally, and, in the same pharmaceutical composition, 0.1 mg of norethindrone or a compound that is metabolized in vivo to produce norethindrone, such as an ester of norethindrone that is de-esterified in vivo to produce norethindrone, for instance, norethindrone acetate, e.g., administered orally. In some embodiments, the add-back therapy includes 0.5 mg of β17-estradiol, e.g., administered orally, and, in a separate pharmaceutical composition, 0.1 mg of norethindrone or a compound that is metabolized in vivo to produce norethindrone, such as an ester of norethindrone that is de-esterified in vivo to produce norethindrone, for instance, norethindrone acetate, e.g., administered orally.

In some embodiments, the GnRH antagonist is administered to the patient in a fixed-dose composition that contains the GnRH antagonist (e.g., in an amount of about 100 mg or about 200 mg of the compound of any one of formulas (I)-(VI)), from about 0.25 mg to about 0.75 mg of β17-estradiol, and from about 0.05 mg to about 0.2 mg of norethindrone or a compound that is metabolized in vivo to produce norethindrone, such as an ester of norethindrone that is de-esterified in vivo to produce norethindrone, for instance, norethindrone acetate. In some embodiments, the GnRH antagonist is administered to the patient in a fixed-dose composition that contains the GnRH antagonist (e.g., in an amount of about 100 mg or about 200 mg of the compound of any one of formulas (I)-(VI)), about 0.5 mg of β17-estradiol (e.g., 0.5 mg of β17-estradiol), and about 0.1 mg of norethindrone or a compound that is metabolized in vivo to produce norethindrone, such as an ester of norethindrone that is de-esterified in vivo to produce norethindrone, for instance, norethindrone acetate (e.g., 0.1 mg of norethindrone or a compound that is metabolized in vivo to produce norethindrone, such as an ester of norethindrone that is de-esterified in vivo to produce norethindrone, for instance, norethindrone acetate). In some embodiments, the GnRH antagonist is administered to the patient in a fixed-dose composition that contains the GnRH antagonist (e.g., in an amount of about 100 mg or about 200 mg of the compound of any one of formulas (I)-(VI)), 0.5 mg of β17-estradiol, and 0.1 mg of norethindrone acetate.

In some embodiments of any of the foregoing aspects of the disclosure, the patient is a pre-menopausal female of from about 18 to about 48 years of age, such as a patient of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 years of age.

In some embodiments, the patient has been determined to exhibit a serum concentration of FSH of about 20 IU/L or less prior to commencement of the first and/or second treatment period, such as a serum concentration of FSH of from about 5 IU/L to about 20 IU/L (e.g., a serum concentration of FSH of about 5 IU/L, 6 IU/L, 7 IU/L, 8 IU/L, 9 IU/L, 10 IU/L, 11 IU/L, 12 IU/L, 13 IU/L, 14 IU/L, 15 IU/L, 16 IU/L, 17 IU/L, 18 IU/L, 19 IU/L, or 20 IU/L.

In some embodiments, the patient has been determined to exhibit a rectal (type II) and/or vaginal (type III) endometriosis node of at least 2 cm prior to commencement of the first and/or second treatment period. The length of the type II and/or type III endometriosis node may be assessed, for example, by way of magnetic resonance imaging (MRI).

In some embodiments, the patient has been determined to exhibit a junctional-zone width of about 12 mm or more prior to commencement of the first and/or second treatment period, such as a junctional zone width of from about 12 mm to about 20 mm, or more (e.g., a junctional zone width of from about 12 mm to about 20 mm, from about 12 mm to about 19 mm, from about 12 mm to about 18 mm, from about 12 mm to about 17 mm, from about 12 mm to about 16 mm, from about 12 mm to about 15 mm, from about 12 mm to about 14 mm, or more) prior to commencement of the first and/or second treatment period. The junctional zone width may be assessed, for example, by way of MRI.

In some embodiments of any of the foregoing aspects of the disclosure, prior to commencing treatment with the GnRH antagonist, the patient has been administered (e.g., and failed to respond to) a selective progesterone receptor modulatory (SPRM), such as ulipristal acetate (UPA). In some embodiments, prior to commencing treatment with the GnRH antagonist, the patient has been periodically administered the SPRM, such as UPA, in an amount of from about 1 mg to about 10 mg per dose (e.g., in an amount of about 1 mg, 2 mg, 3 mg, 4 mg, 5 mg, 6 mg, 7 mg, 8 mg, 9 mg, or 10 mg per dose) over the course of a treatment period having a duration of, for example, from about 1 week to about 6 months (e.g., over the course of a treatment period having a duration of about 1 week, 2 weeks, 3 weeks, 4 weeks, 1 month, 2 months, 3 months, 4 months, 5 months, or 6 months). In some embodiments, prior to commencing treatment with the GnRH antagonist, the patient has been administered the SPRM, such as UPA, in one or more doses per day (e.g., in a single dose per day) totaling from about 1 mg to about 10 mg per day (e.g., totaling about 1 mg, 2 mg, 3 mg, 4 mg, 5 mg, 6 mg, 7 mg, 8 mg, 9 mg, or 10 mg per day). In some embodiments, the patient failed to respond to treatment with the SPRM.

In some embodiments, the patient exhibits a reduction in serum concentration of FSH, LH, and/or E2 following administration of the GnRH antagonist to the patient. The reduction in serum concentration of LH, FSH, and/or E2 may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient exhibits a reduction in uterine bleeding following administration of the GnRH antagonist to the patient. The reduction in dyspareunia may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in uterine bleeding may be assessed by way of an alkaline hematin method, for example, as described herein.

In some embodiments, the patient exhibits amenorrhea following administration of the GnRH antagonist to the patient. The amenorrhea may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient exhibits a reduction in the volume of one or more rectovaginal endometriosis nodes following administration of the GnRH antagonist to the patient. The reduction in the volume of the one or more rectovaginal endometriosis nodes may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in the volume of the one or more rectovaginal endometriosis nodes may be assessed, for example, by way of MRI and/or TVUS.

In some embodiments, the patient exhibits a reduction in bowel involvement of one or more type III endometriosis nodes following administration of the GnRH antagonist to the patient. The reduction in bowel involvement of one or more type III endometriosis nodes may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in bowel involvement of one or more type III endometriosis nodes may be assessed, for example, by way of MRI.

In some embodiments, the patient exhibits a reduction in pelvic pain following administration of the GnRH antagonist to the patient. The reduction in pelvic pain may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in pelvic pain may be assessed by way of a modified Biberoglu & Behrman (mB&B) score, Numerical Rating Scale (NRS) score, or Verbal Rating Scale (VRS) score.

In some embodiments, the patient exhibits a reduction in dysmenorrhea following administration of the GnRH antagonist to the patient. The reduction in dysmenorrhea may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in dysmenorrhea may be assessed by way of an mB&B score, NRS score, or VRS score.

In some embodiments, the patient exhibits a reduction in dyspareunia following administration of the GnRH antagonist to the patient. The reduction in dyspareunia may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in dyspareunia may be assessed by way of an mB&B score, NRS score, or VRS score.

In some embodiments, the patient exhibits a reduction in dyschezia following administration of the GnRH antagonist to the patient. The reduction in dyschezia may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in dyschezia may be assessed byway of an mB&B score, NRS score, or VRS score.

In some embodiments, the patient exhibits a reduction in uterine volume following administration of the GnRH antagonist to the patient. The reduction in uterine volume may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period). The reduction in uterine volume may be assessed, for example, by way of MRI or transvaginal ultrasound (TVUS).

In some embodiments, the patient exhibits a reduction in the thickness of the anterior and/or posterior region of the uterine myometrium following administration of the GnRH antagonist to the patient. The reduction in the thickness of the anterior and/or posterior region of the uterine myometrium may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient exhibits a reduction in uterine tenderness following administration of the GnRH antagonist to the patient. The reduction in uterine tenderness may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient exhibits a reduction in the diameter of a junctional zone of adenomyosis following administration of the GnRH antagonist to the patient. The reduction in the diameter of a junctional zone of adenomyosis may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient exhibits an improvement in Endometriosis Health Profile questionnaire (EHP-30) score following administration of the GnRH antagonist to the patient. The improvement in the EHP-30 score may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient exhibits a positive Patient Global Impression of Change (PGIC) score following administration of the GnRH antagonist to the patient. The positive PGIC score may be effectuated within from about one day to about 36 weeks of commencement of the second treatment period, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of commencement of the second treatment period (e.g., within from about 12 weeks to about 24 weeks of commencement of the second treatment period, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of commencement of the second treatment period).

In some embodiments, the patient does not exhibit a reduction in bone mineral density of greater than 5% at the end of the second treatment period relative to a measurement of the patient's bone mineral density obtained prior to, or during, the first treatment period. In some embodiments, the patient does not exhibit a reduction in BMD of greater than 4% at the end of the second treatment period relative to a measurement of the patient's bone mineral density obtained prior to, or during, the first treatment period. In some embodiments, the patient does not exhibit a reduction in BMD of greater than 3% at the end of the second treatment period relative to a measurement of the patient's bone mineral density obtained prior to, or during, the first treatment period. In some embodiments, the patient does not exhibit a reduction in BMD of greater than 2% at the end of the second treatment period relative to a measurement of the patient's bone mineral density obtained prior to, or during, the first treatment period.

In some embodiments, the patient does not exhibit a reduction in BMD of greater than 1% at the end of the second treatment period relative to a measurement of the patient's bone mineral density obtained prior to, or during, the first treatment period.

Techniques for assessing BMD that may be used in conjunction with the methods of the present disclosure include, for example, dual energy X-ray absorptiometry, such as in the spine and/or femur of the patient. In some embodiments, BMD is assessed by comparing the concentration of bone specific alkaline phosphatase (BAP) in a sample isolated from the patient following the administration to the concentration of BAP in a sample isolated from the patient prior to administration of the GnRH antagonist. In some embodiments, the BMD is assessed by comparing the concentration of deoxypyridinoline (DPD) in a sample isolated from the patient following the administration to the concentration of DPD in a sample isolated from the patient prior to administration of the GnRH antagonist. In some embodiments, the BMD is assessed by comparing the concentration of type I collagen C-terminal telopeptide (CTX) in a sample isolated from the patient following the administration to the concentration of CTX in a sample isolated from the patient prior to administration of the GnRH antagonist. In some embodiments, the BMD is assessed by comparing the concentration of procollagen 1 N-terminal peptide (P1NP) in a sample isolated from the patient following the administration to the concentration of P1NP in a sample isolated from the patient prior to administration of the GnRH antagonist.

In another aspect, the disclosure features a kit containing a GnRH antagonist, such as a GnRH antagonist of any of the above aspects or embodiments of the disclosure. The kit may further contain a package insert, such as a package insert instructing a user of the kit to administer the GnRH antagonist to a patient in accordance with the method of any one of the foregoing aspects or embodiments of the disclosure. In some embodiments, the GnRH antagonist contained within the kit is a compound represented by formula (I)

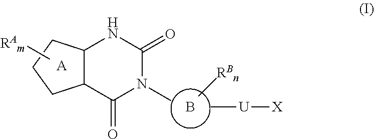

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

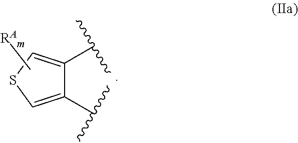

In some embodiments of formula (I) or (IIa), m is 1.

In some embodiments of formula (I) or (IIa), the ring A is an optionally substituted thiophene ring represented by formula (IIb)

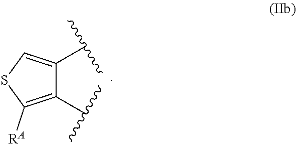

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

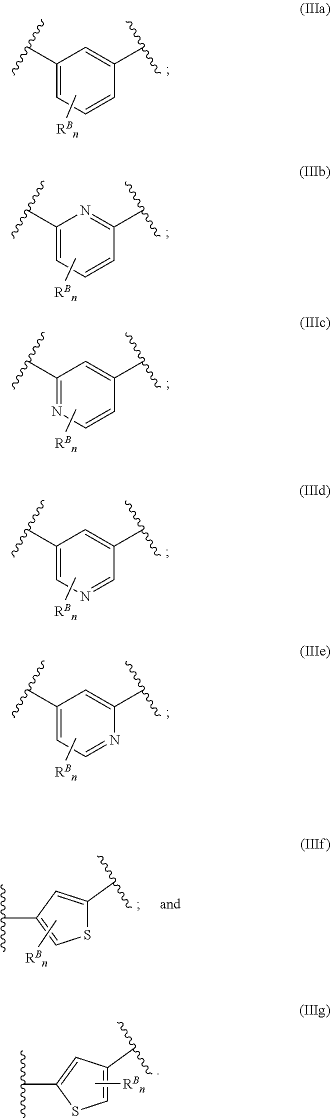

In some embodiments of formula (I), (IIa), (IIb), or any one of (IIIa)-(IIIg), n is 2.

In some embodiments of formula (I), (IIa), (IIb), or any one of (IIIa)-(IIIg), the ring B is represented by a formula selected from the group consisting of:

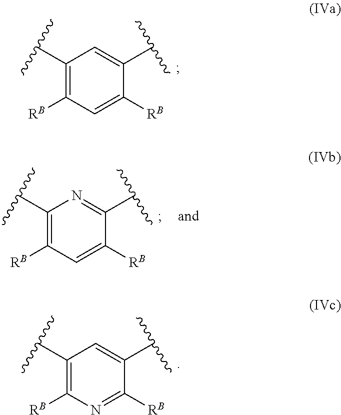

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), U is a single bond.

In some embodiments of formula (I), (IIa), (IIb), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIIa)-(IIIg), or any one of (IVa)-(IVc), Y is an optionally substituted benzene ring represented by formula (V)

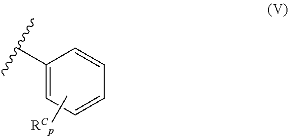

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), Y is a substituted benzene ring represented by formula (Va)

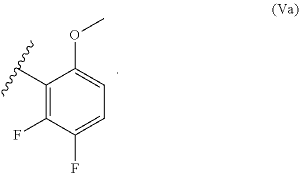 [SMILES: *c1c(OC)ccc(F)c1F]

In some embodiments, the compound is represented by formula (Ia)

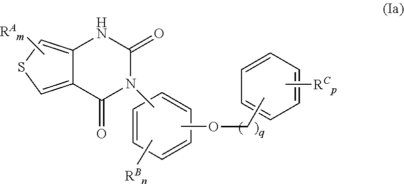

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

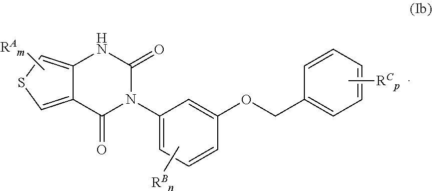

In some embodiments, the compound is represented by formula (Ic)

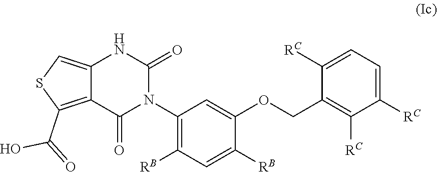

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, represented by formula (VI)

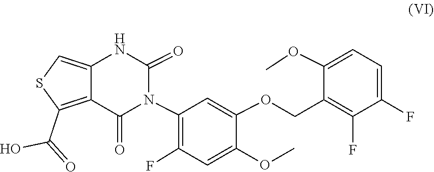 [SMILES: COc1cc(F)c(-n2c(=O)[nH]c3csc(C(=O)O)c3c2=O)cc1OCc1c(OC)ccc(F)c1F]

or a pharmaceutically acceptable salt thereof.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹⁹F solid-state NMR peaks centered at about −151.8 ppm, −145.2 ppm, and −131.6 ppm.

In some embodiments, the GnRH antagonist contained within the kit is a compound represented by formula (VII)

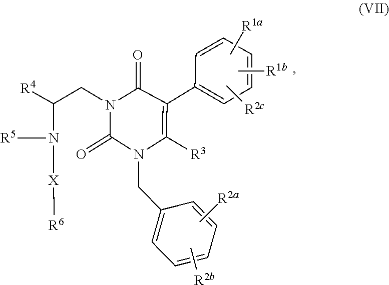

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist is a compound represented by formula (VIII)

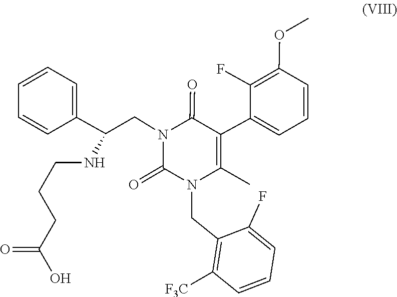 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

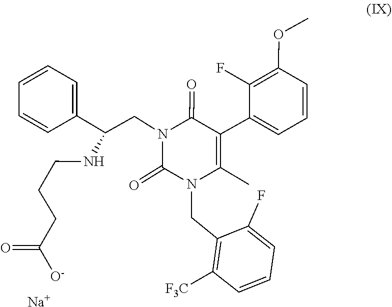 [SMILES: COc1cccc(-c2c(C)n(Cc3c(F)cccc3C(F)(F)F)c(=O)n(C[C@H](NCCCC(=O)[O-])c3ccccc3)c2=O)c1F ; [Na+]]

In some embodiments, the GnRH antagonist contained within the kit is a compound represented by formula (X)

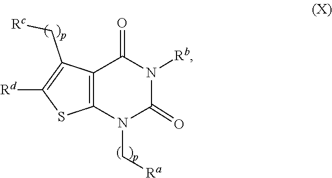

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a compound represented by formula (XI)

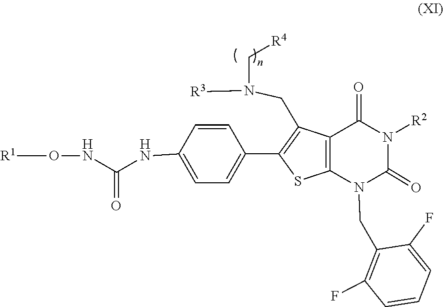

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1) a halogen, (2′) a hydroxy group, (3′) a C₁₋₄alkyl and (4′) a C₁₋₄alkoxy, (4) a phenyl which may have a substituent selected from the group consisting of (1) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist is a compound represented by formula (XII), below.

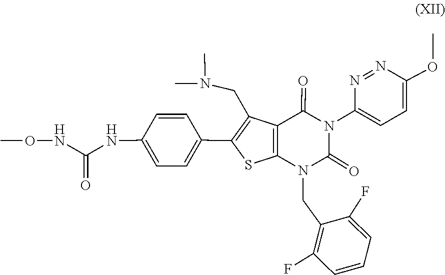

In some embodiments, the GnRH antagonist contained within the kit is a compound represented by formula (XIII)

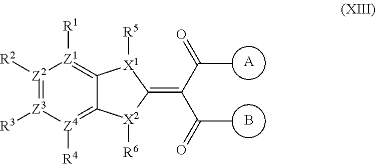

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁- (wherein n₁₀₁ is an integer of 0 to 2), H—S(O)n₁₀₁-, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist is a compound represented by formula (XIV), below.

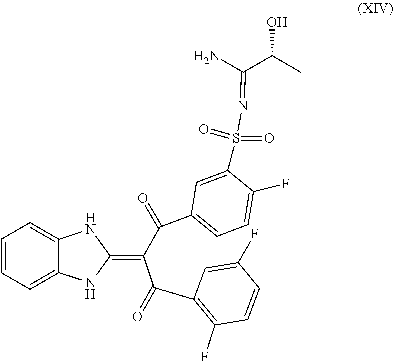 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the GnRH antagonist contained within the kit is SK12670 or BAY-784, or a variant or derivative thereof.

### Definitions

As used herein, the term “about” refers to a value that is within 10% above or below the value being described. For instance, a value of “about 5 mg” refers to a quantity that is from 4.5 mg to 5.5 mg.

As used herein, the term “abnormal uterine bleeding” refers to uterine blood loss that occurs either at an inappropriate time during a patient's menstrual cycle or in an amount that exceeds typical menstrual blood loss, such as “heavy menstrual blood loss” and “menorrhagia,” which refer to menstrual blood loss of 80 ml or more (e.g., 80 ml, 90 ml, 100 ml, 110 ml, 120 ml, 130 ml, 140 ml, 150 ml, 160 ml, 170 ml, 180 ml, 190 ml, 200 ml, or more) per menstrual cycle (The Menorrhagia Research Group. Quantification of menstrual blood loss. The Obstetrician & Gynaecologist 6:88-92 (2004)).

As used herein, the term “add-back therapy” refers to the administration of estrogen during a treatment regimen, such as treatment with a GnRH antagonist (e.g., 3-[2-fluoro-5-(2,3-difluoro-6-methoxybenzyloxy)-4-methoxyphenyl]-2,4-dioxo-1,2,3,4-tetrahydrothieno [3,4d]pyrimidine-5-carboxylic acid or a pharmaceutically acceptable salt thereof, such as the choline salt thereof, as described herein), so as to counteract side effects that may otherwise be associated with excessive suppression of estradiol. Such side effects may include, for example, a reduction in bone mineral density (BMD). A patient's BMD may be assessed by dual energy X-ray absorptiometry, for instance, in the spine or femur of the patient. Add-back therapy may be administered to a patient according to the methods described herein so as to mitigate a reduction in BMD caused by the administration of a GnRH antagonist. For instance, add-back therapy may be administered to a patient undergoing GnRH antagonist therapy such that the patient does not exhibit a reduction in BMD of greater than 5% (e.g., no greater than 5%, 4%, 3%, 2%, 1%, 0.9%, 0.8%, 0.7%, 0.6%, 0.5%, 0.4%, 0.3%, 0.2%, 0.1%, or less). Add-back therapy may include estrogen in the form of β17-estradiol, ethinyl estrogen, or a conjugated estrogen, such as a conjugated equine estrogen, and may further include one or more additional agents, such as a progestin (e.g., norethindrone or a compound that is metabolized in vivo to produce norethindrone, such as an ester of norethindrone that is de-esterified in vivo to produce norethindrone, for instance, norethindrone acetate, among other progestins such as progesterone, norgestimate, medroxyprogesterone, and drospirenone). Add-back therapy may be formulated for oral administration, such as in the form of a tablet, capsule, gel cap, powder, liquid solution, or liquid suspension. Add-back therapy may feature a co-formulation containing estrogen (e.g., in the form of β17-estradiol) and an additional agent such as a progestin (e.g., norethindrone or a compound that is metabolized in vivo to produce norethindrone, such as an ester of norethindrone that is de-esterified in vivo to produce norethindrone, for instance, norethindrone acetate). For instance, add-back therapy may be administered to a patient in the form of a single tablet, capsule, gel cap, powder, liquid solution, or liquid suspension that contains both estrogen (e.g., in the form of β17-estradiol) and a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate) in the form of a single pharmaceutical composition, such as a single tablet, capsule, gel cap, powder, liquid solution, or liquid suspension.

As used herein, a compound, such as a GnRH antagonist, estrogen, or progestin, among others, that is “administered” to a patient, such as a patient having an estrogen-dependent disease described herein, may be administered in an electrostatically neutral and/or nonionized form (e.g., in the form of a neutral carboxylic acid, a neutral amine, and the like) and/or in the form of a pharmaceutically acceptable salt, particularly if the compound contains a substituent that readily ionizes at physiological pH. For example, a compound containing a carboxylic acid substituent may be administered to a patient (e.g., a patient suffering from an estrogen-dependent disease described herein) in the form of the neutral, uncharged carboxylic acid and/or in the form of a carboxylate salt containing a pharmaceutically acceptable cation. Similarly, a compound containing an amine substituent may be administered to the patient in the form of the neutral, uncharged amine and/or in the form of an ammonium salt containing a pharmaceutically acceptable anion.

For example, as used herein, a GnRH antagonist of the disclosure, such as an optionally substituted thieno[3,4d]pyrimidine compound containing a carboxylic acid substituent, may be “administered” to a patient in the form of the neutral, uncharged carboxylic acid and/or in the form of a pharmaceutically acceptable salt (e.g., a salt containing the corresponding carboxylate anion and a pharmaceutically acceptable cation). Accordingly, as used herein, a GnRH antagonist of the formula 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid may be “administered” to a patient in the form of the neutral, uncharged carboxylic acid and/or in the form of a pharmaceutically acceptable salt (e.g., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a pharmaceutically acceptable cation). For example, a GnRH antagonist of the formula 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid may be “administered” to a patient in the form of the neutral, uncharged carboxylic acid and/or in the form of a choline salt (i.e., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a choline cation).

For example, as used herein, a GnRH antagonist of the disclosure, such as an optionally substituted 3-aminoalkyl pyrimidine-2,4(1H,3H)-dione compound containing a carboxylic acid substituent, may be “administered” to a patient in the form of the neutral, uncharged carboxylic acid and/or in the form of a pharmaceutically acceptable salt (e.g., a salt containing the corresponding carboxylate anion and a pharmaceutically acceptable cation). Accordingly, as used herein, a GnRH antagonist of the formula 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid may be “administered” to a patient in the form of the neutral, uncharged carboxylic acid and/or in the form of a pharmaceutically acceptable salt (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation). For example, a GnRH antagonist of the formula 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid may be “administered” to a patient in the form of the neutral, uncharged carboxylic acid and/or in the form of a sodium salt (i.e., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a sodium cation).

For example, as used herein, a GnRH antagonist of the disclosure, such as an optionally substituted thieno[2,3d]pyrimidine compound containing an amine substituent, may be “administered” to a patient in the form of the neutral, uncharged amine and/or in the form of a pharmaceutically acceptable salt (e.g., a salt containing the corresponding ammonium cation and a pharmaceutically acceptable anion). Accordingly, as used herein, a GnRH antagonist of the formula N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea may be “administered” to a patient in the form of the neutral, uncharged amine and/or in the form of a pharmaceutically acceptable salt (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion). For example, a GnRH antagonist of the formula N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea may be “administered” to a patient in the form of the neutral, uncharged amine and/or in the form of a chloride salt (i.e., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a chloride anion).

As used herein, a compound, such as a GnRH antagonist, estrogen, or progestin, among others, that is “administered” to a patient (e.g., a patient having an estrogen-dependent disease described herein) in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. As used herein, an amount of a pharmaceutically acceptable salt of a compound that is “equivalent” to a recited amount of the compound is an amount of the pharmaceutically acceptable salt that contains the same molar quantity of the compound as that contained by the recited amount of the compound. One can readily calculate the amount of a pharmaceutically acceptable salt of a compound that is “equivalent” to a recited amount of the compound using standard stoichiometry calculations known in the art.

Accordingly, as used herein, a GnRH antagonist of the disclosure, such as an optionally substituted thieno[3,4d]pyrimidine compound containing a carboxylic acid substituent, that is “administered” to a patient in a recited amount (e.g., per dose, per day, per week, per month, etc.) may be administered to the patient in the recited amount of an electrostatically neutral and/or nonionized of the compound or in an equivalent amount of a pharmaceutically acceptable salt of the compound. For example, as an optionally substituted thieno[3,4d]pyrimidine compound containing a carboxylic acid substituent, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, that is “administered” to a patient in a recited amount may be administered to the patient in the recited amount of the neutral, uncharged form of the compound or in an equivalent amount of a pharmaceutically acceptable salt of the compound (e.g., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a pharmaceutically acceptable cation, such as a choline cation). Accordingly, an optionally substituted thieno[3,4d]pyrimidine compound containing a carboxylic acid substituent, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, that is “administered” to a patient in a recited amount, such as a recited amount of from 25 mg to 400 mg (e.g., 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg) may be administered to the patient in the recited amount of the neutral, uncharged form of the compound or in an equivalent amount of a pharmaceutically acceptable salt of the compound (e.g., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a pharmaceutically acceptable cation, such as a choline cation.

Accordingly, as used herein, a GnRH antagonist of the disclosure, such as an optionally substituted 3-aminoalkyl pyrimidine-2,4(1H,3H)-dione compound containing a carboxylic acid substituent, that is “administered” to a patient in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. For example, as an optionally substituted 3-aminoalkyl pyrimidine-2,4(1H,3H)-dione compound containing a carboxylic acid substituent, such as 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid, that is “administered” to a patient in a recited amount may be administered to the patient in the recited amount of the neutral, uncharged form of the compound or in an equivalent amount of a pharmaceutically acceptable salt of the compound (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation, such as a sodium cation). Accordingly, an optionally substituted 3-aminoalkyl pyrimidine-2,4(1H,3H)-dione compound containing a carboxylic acid substituent, such as 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid, that is “administered” to a patient in a recited amount, such as a recited amount of from 50 mg to 650 mg (e.g., 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg) may be administered to the patient in the recited amount of the neutral, uncharged form of the compound or in an equivalent amount of a pharmaceutically acceptable salt of the compound (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation, such as a sodium cation.

Accordingly, as used herein, a GnRH antagonist of the disclosure, such as an optionally substituted thieno[2,3d]pyrimidine compound containing an amine substituent, that is “administered” to a patient in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. For example, as an optionally substituted thieno[2,3d]pyrimidine compound containing an amine substituent, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, that is “administered” to a patient in a recited amount may be administered to the patient in the recited amount of the neutral, uncharged form of the compound or in an equivalent amount of a pharmaceutically acceptable salt of the compound (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion, such as a chloride anion). Accordingly, an optionally substituted thieno[2,3d]pyrimidine compound containing an amine substituent, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, that is “administered” to a patient in a recited amount, such as a recited amount of from 10 mg to 60 mg (e.g., 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg) may be administered to the patient in the recited amount of the neutral, uncharged form of the compound or in an equivalent amount of a pharmaceutically acceptable salt of the compound (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion, such as a chloride anion.

As used herein, the term “affinity” refers to the strength of a binding interaction between two molecules, such as a ligand and a receptor. The term “Kᵢ”, as used herein, is intended to refer to the inhibition constant of an antagonist for a particular molecule of interest, and can be expressed as a molar concentration (M). Kᵢ values for antagonist-target interactions can be determined, e.g., using methods established in the art. Methods that can be used to determine the Kᵢ of an antagonist for a molecular target include competitive binding experiments, e.g., as described in U.S. Pat. No. 9,040,693. The term “K_{d}”, as used herein, is intended to refer to the dissociation constant, which can be obtained, e.g., from the ratio of the rate constant for the dissociation of the two molecules (k_{d}) to the rate constant for the association of the two molecules (kₐ) and is expressed as a molar concentration (M). K_{d} values for receptor-ligand interactions can be determined, e.g., using methods established in the art. Methods that can be used to determine the K_{d} of a receptor-ligand interaction include surface plasmon resonance, e.g., through the use of a biosensor system such as a BIACORE® system.

As used herein, the term “amenorrhea” refers to the absence or near absence of uterine blood loss in a female patient, such as a female human patient undergoing GnRH antagonist treatment according to a dosing regimen described herein. As such, amenorrhea is a clinical indicator of reduced menstrual blood loss, such as reduced menstrual blood loss in a patient suffering from an estrogen-dependent disease (e.g., uterine fibroids, endometriosis (such as rectovaginal endometriosis), and adenomyosis, among others described herein) and undergoing GnRH antagonist treatment according to a dosing regimen described herein.

As used herein, the terms “benefit” and “response” are used interchangeably in the context of a subject undergoing therapy for the treatment of an estrogen-dependent disease described herein. These terms refers to any clinical improvement in the subject's condition. For example, clinical benefits in the context of a subject administered a gonadotropin-releasing hormone (GnRH) antagonist for the treatment of uterine fibroids, one of the estrogen-dependent diseases described herein, include, without limitation, (i) a reduction in serum concentration of FSH, LH, and/or E2 following administration of the GnRH antagonist to the patient, (ii) a reduction in uterine bleeding following administration of the GnRH antagonist to the patient; (iii) achievement of amenorrhea following administration of the GnRH antagonist to the patient; (iv) a reduction in the volume of one or more uterine fibroids following administration of the GnRH antagonist to the patient, (v) a reduction in pelvic pain following administration of the GnRH antagonist to the patient; (vi) a reduction in dysmenorrhea following administration of the GnRH antagonist to the patient; (vii) a reduction in dyspareunia following administration of the GnRH antagonist to the patient; (viii) a reduction in dyschezia following administration of the GnRH antagonist to the patient; and (ix) an improvement in the patient's overall well-being as determined, for example, by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

Similarly, exemplary clinical benefits in the context of a subject administered a GnRH antagonist for the treatment of endometriosis (e.g., rectovaginal endometriosis) include, without limitation, (i) a reduction in serum concentration of FSH, LH, and/or E2 following administration of the GnRH antagonist to the patient, (ii) a reduction in the volume of one or more endometriosis nodes (e.g., one or more rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and (x) an improvement in the patient's overall well-being as determined by an improvement in the patient's EHP-30 score following administration of the GnRH antagonist to the patient and/or by way of a positive PGIC score following administration of the GnRH antagonist to the patient.

Exemplary clinical benefits in the context of a subject administered a GnRH antagonist for the treatment of adenomyosis, another estrogen-dependent disease described herein, include, without limitation, (i) a reduction in serum concentration of FSH, LH, and/or E2 following administration of the GnRH antagonist to the patient, (ii) a reduction in uterine volume following administration of the GnRH antagonist to the patient, (iii) a reduction in the thickness of the anterior and/or posterior region of the uterine myometrium following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine tenderness following administration of the GnRH antagonist to the patient; (ix) a reduction in uterine bleeding following administration of the GnRH antagonist to the patient; (x) achievement of amenorrhea following administration of the GnRH antagonist to the patient; (xi) a reduction in the diameter of a junctional zone of adenomyosis following administration of the GnRH antagonist to the patient; and (xii) an improvement in the patient's overall well-being as determined by an improvement in the patient's EHP-30 score following administration of the GnRH antagonist to the patient and/or by way of a positive PGIC score following administration of the GnRH antagonist to the patient.

As used herein, the term “Biberoglu and Behrman scale” or “B&B scale” or a modification thereof, such as a “modified Biberoglu and Behrman scale” refers to a multi-point scale that can be used to indicate the severity of one or more symptoms experienced by patient suffering from an estrogen-dependent disease, such as endometriosis, among others. A B&B score can be assessed by verbally prompting the patient to indicate the degree of function or quality of life being experienced. A B&B score can be used, e.g., to assess the severity of such symptoms as dysmenorrhea, dyspareunia, chronic pelvic pain, pelvic tenderness, and induration, among others. Methods of determining a B&B score are described in detail, e.g., in Biberoglu and Behrman, Am. J. Obstet. Gynecol. 139:645 (1981).

As used herein, the term “crystalline” or “crystalline form” means having a physical state that is a regular three-dimensional array of atoms, ions, molecules or molecular assemblies. Crystalline forms have lattice arrays of building blocks called asymmetric units that are arranged according to well-defined symmetries into unit cells that are repeated in three-dimensions. In contrast, the term “amorphous” or “amorphous form” refers to an unorganized (no orderly) structure. The physical state of a therapeutic compound may be determined by exemplary techniques such as x-ray diffraction, polarized light microscopy and/or differential scanning calorimetry.

As used herein, the term “dose” refers to the quantity of a therapeutic agent, such as a GnRH antagonist described herein, that is administered to a subject at a particular instant for the treatment of a disorder or condition, such as to treat or ameliorate one or more symptoms of an estrogen-dependent disease described herein (e.g., uterine fibroids, endometriosis, such as rectovaginal endometriosis, and/or uterine fibroids). A therapeutic agent as described herein may be administered in a single dose or in multiple doses over the course of a treatment period, as defined herein. In each case, the therapeutic agent may be administered using one or more “unit dosage forms” of the therapeutic agent, a term that refers to a one or more discrete compositions containing a therapeutic agent that collectively constitute a single dose of the agent. For instance, a single dose of 200 mg of a therapeutic agent may be administered using, e.g., two 100 mg unit dosage forms of the therapeutic agent. The unit dosage forms may be, for example, solid unit dosage forms, such as tablets or capsules, among others.

As used herein, the term “dual energy X-ray absorptiometry” (DEXA) refers to a spectroscopic method of measuring bone mineral density in a patient (e.g., a human patient) in which X-ray radiation of two distinct frequencies are transmitted towards a target bone of the patient. The absorption of the transmitted radiation can subsequently be correlated with a measure of the bone mineral density within the target bone. Methods of determining bone mineral density using DEXA are described in detail, e.g., in Mazess et al., American Journal of Clinical Nutrition 51:1106-1112 (1990).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “Endometriosis Health Profile-30” or “EHP-30” refers to a questionnaire that can be used to evaluate quality of life in patient suffering from an estrogen-dependent disease, such as endometriosis, among others. A score obtained from this questionnaire (i.e., an “EHP-30 score”) may provide an indication of the patient's degree of pain, feeling of control and powerlessness, emotional well-being, social support, and/or self-image. Exemplary methods that can be used to perform an EHP-30 questionnaire and procedures for interpreting the scores obtained therefrom are known in the art are described, e.g., in Renouvel et al., Journal de Gynécologie Obstétrique et Biologie de la Reproduction 38:404-410 (2009), the disclosure of which is incorporated herein by reference as it pertains to methods for conducting and evaluating an EHP-30 questionnaire.

As used herein, the term “estrogen-dependent disease” refers to a disease or condition that is exacerbated or caused by excessive, inappropriate, or unregulated estrogen (e.g., β17-estradiol) production and/or an aberrant physiological response to estrogen. Estrogen-dependent diseases include those exacerbated or caused by circulating β17-estradiol levels in excess of, for example, about 60 pg/ml. Examples of such diseases include uterine fibroids, endometriosis (e.g., rectovaginal endometriosis), and adenomyosis. Additional examples of estrogen-dependent diseases include, without limitation, benign prostatic hypertrophy, precocious puberty, premenstrual syndrome, polycystic ovary syndrome, hirsutism, short stature, sleep disorders, acne, baldness, and irritable bowel syndrome, among others.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “gonadotropin-releasing hormone antagonist” or “GnRH antagonist” refers to a compound that specifically binds the GnRH receptor and is capable of inhibiting receptor signalling, e.g., such that release of one or more gonadotropins (such as follicle-stimulating hormone and luteinizing hormone) is inhibited. GnRH antagonists for use with the compositions and methods described herein include thieno[3,4d]pyrimidine derivatives and variants, such as those described in U.S. Pat. No. 9,040,693, the disclosure of which is incorporated herein by reference in its entirety. Exemplary GnRH antagonists include 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof, such as the choline salt thereof, e.g., as described in U.S. Pat. No. 9,169,266, the disclosure of which is incorporated herein by reference in its entirety. Additional examples of GnRH antagonists that may be used in conjunction with the compositions and methods described herein include optionally substituted 3-aminoalkyl pyrimidine-2,4(1H,3H)-dione derivatives, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof, and related compounds described in U.S. Pat. No. 7,056,927, the disclosure of which is incorporated herein by reference in its entirety. Further examples of GnRH antagonists that may be used in conjunction with the compositions and methods described herein include optionally substituted thieno[2,3d]pyrimidine derivatives, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof, and related compounds described in U.S. Pat. No. 7,300,935, the disclosure of which is incorporated herein by reference in its entirety. Additional examples of GnRH antagonists that may be used in conjunction with the compositions and methods described herein include optionally substituted propane-1,3-dione derivatives, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707, and related compounds described in U.S. Pat. No. 6,960,591, the disclosure of which is incorporated herein by reference in its entirety.

As used herein, the term “IC₅₀” refers to the concentration of a substance (antagonist) that reduces the efficacy of a reference agonist or the constitutive activity of a biological target by 50%, e.g., as measured in a competitive ligand binding assay. Exemplary competitive ligand binding assays include competitive radioligand binding assays, competitive enzyme-linked immunosorbant assays (ELISA), and fluorescence anisotropy-based assays, among others known in the art.

As used herein in the context of a GnRH antagonist and add-back therapy, the term “in combination with” refers to administration of the GnRH antagonist and add-back therapy agent(s) such that the later-administered of these substances is provided to the patient at a time when there is still a detectable concentration in the patient's blood of the earlier-administered of these substances. The GnRH antagonist and add-back therapy need not be administered at the exact same moment for these substances to be administered “in combination with” one another.

As used herein, the term “menstrual cycle” refers to a recurring cycle of physiological changes in females, such as human females, that is associated with reproductive fertility. While the cycle length may vary from woman to woman, 28 days is generally taken as representative of the average ovulatory cycle in human females.

As used herein, the term “Numerical Rating Score” (NRS) refers to a score within an 11-point numerical scale of 0-10 that indicates the degree of pain experienced by a patient, such as a patient having an estrogen-dependent disease described herein. For instance, a score of 0 may indicate the patient is experiencing no pain, while scores from 1-3 may indicate that the patient is experiencing mild pain. A score of from 4-6 may indicate that the patient is experiencing moderate pain, and a score of from 7-10 may indicate that the patient is experiencing severe pain. Typically, to determine a NRS score, the patient is asked to indicate the level of pain currently being experienced, as well as the pain experienced at its most intense and least intense occurrences. Methods for determining a NRS are described in detail, e.g., in McCaffery et al., Pain: Clinical Manual for Nursing Practice. Baltimore (1993), the disclosure of which is incorporated herein by reference as it pertains to methods for obtaining and evaluating an NRS.

As used herein, the term “pharmaceutical composition” means a mixture containing a therapeutic compound to be administered to a patient, such as a mammal, e.g., a human, in order to prevent, treat or control a particular disease or condition affecting the mammal, such as preterm labor or dysmenorrhea.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein in the context of administration of a therapeutic agent, the term “periodically” refers to administration of the agent two or more times over the course of a treatment period (e.g., two or more times daily, weekly, monthly, or yearly).

As used herein, a “reduced bone mineral density” at the end of a first treatment period relative to a measurement of a patient's bone mineral density obtained during, or prior to commencement of, the first treatment period refers to a reduction in bone mineral density of at least 0.1% (e.g., a reduction in bone mineral density of from 0.1% to 5%, such as a reduction in bone mineral density of 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5%) at the end of the first treatment period relative to a measurement of a patient's bone mineral density obtained during, or prior to commencement of, the first treatment period. In some embodiments of the disclosure, the reduction in bone mineral density at the end of the first treatment period relative to a measurement of a patient's bone mineral density obtained during, or prior to commencement of, the first treatment period is a reduction of at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, or at least 5%.

In some embodiments, the reduction in bone mineral density at the end of the first treatment period relative to a measurement of a patient's bone mineral density obtained during, or prior to commencement of, the first treatment period is a reduction of 0.1% to 5%, 0.1% to 4.5%, 0.1% to 4%, 0.1% to 3.5%, 0.1% to 3%, 0.1% to 2.5%, 0.1% to 2%, 0.1% to 1.5%, 0.1% to 1%, or 0.1% to 0.5%.

In some embodiments, the reduction in bone mineral density at the end of the first treatment period relative to a measurement of a patient's bone mineral density obtained during, or prior to commencement of, the first treatment period is a reduction of 0.5% to 5%, 0.5% to 4.5%, 0.5% to 4%, 0.5% to 3.5%, 0.5% to 3%, 0.5% to 2.5%, 0.5% to 2%, 0.5% to 1.5%, or 0.5% to 1%.

In some embodiments, the reduction in bone mineral density at the end of the first treatment period relative to a measurement of a patient's bone mineral density obtained during, or prior to commencement of, the first treatment period is a reduction of 1% to 5%, 1% to 4.5%, 1% to 4%, 1% to 3.5%, 1% to 3%, 1% to 2.5%, 1% to 2%, or 1% to 1.5%.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a patient.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein, e.g., with a K_{D} of less than 100 nM. For example, a ligand that specifically binds to a protein may bind to the protein with a K_{D} of up to 100 nM (e.g., between 1 pM and 100 nM). A ligand that does not exhibit specific binding to a protein or a domain thereof may exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject’ and “patient” are used interchangeably and refer to an organism, such as a mammal (e.g., a human) that receives treatment for an estrogen-dependent disease described herein, such as uterine fibroids, endometriosis (e.g., rectovaginal endometriosis) and/or adenomyosis using the compositions and methods described herein and/or that is diagnosed as having one or more of these diseases in accordance with the methods described herein. Examples of patients include pre-menopausal female human patients. For instance, uterine fibroids patients in need of treatment in accordance with the compositions and methods described herein include those experiencing heavy menstrual bleeding (i.e., blood loss in excess of 80 ml per menstrual cycle). Examples of adenomyosis patients in need of treatment using a GnRH antagonist according to the methods described herein include, e.g., adenomyosis patients diagnosed as having a junctional-zone width of about 12 mm or more prior to administration of the GnRH antagonist to the patient (e.g., a junctional zone width of from about 12 mm to about 20 mm, from about 12 mm to about 19 mm, from about 12 mm to about 18 mm, from about 12 mm to about 17 mm, from about 12 mm to about 16 mm, from about 12 mm to about 15 mm, from about 12 mm to about 14 mm, or more, prior to administration of the GnRH antagonist to the patient). Examples of endometriosis patients (e.g., rectovaginal endometriosis patients) in need of treatment using a GnRH antagonist according to the methods described herein include, e.g., rectovaginal endometriosis patients exhibiting a rectal (type II) and/or vaginal (type Ill) endometriosis node of at least 2 cm prior to administration of the GnRH antagonist to the patient, such as a rectal (type II) and/or vaginal (type III) endometriosis node of from about 2 cm to about 10 cm, or more (e.g., a rectal (type II) and/or vaginal (type III) endometriosis node of from about 2 cm to about 9 cm, from about 2 cm to about 8 cm, from about 2 cm to about 7 cm, from about 2 cm to about 6 cm, from about 2 cm to about 5 cm, or from about 2 cm to about 4 cm, or more) prior to administration of the GnRH antagonist to the patient.

As used herein in the context of a GnRH antagonist, the term “therapeutically effective amount” refers to the quantity of a GnRH antagonist that, when administered to a patient (e.g., a patient suffering from an estrogen-dependent disease), is capable of promoting a reduction in endogenous β17-estradiol levels to concentrations that are less likely to trigger the onset of, or sustain the progression of, an estrogen-dependent disease, such as a concentration of less than about 60 μg/ml in circulating blood. Exemplary therapeutically effective amounts of a GnRH antagonist include, e.g., from about 50 mg to about 200 mg of a compound represented by any one of formulas (I)-(VIa), among other dosage quantities described herein.

As used herein, the terms “treat” or “treatment” refer to therapeutic treatment, in which the object is to prevent or slow down (lessen) an undesired physiological change or disorder in a human patient, such as the progression of an estrogen-dependent disease described herein, including uterine fibroids, endometriosis (e.g., rectovaginal endometriosis) and adenomyosis, among others. Beneficial or desired clinical results include, but are not limited to, alleviation of symptoms, such as a reduction in pelvic pain, a reduction in dysmenorrhea, a reduction in dyspareunia, a reduction in dyschezia, and a reduction in uterine bleeding, among other desired benefits described herein. As a non-limiting example, a patient, such as a female human patient, suffering from uterine fibroids may considered to be treated using a GnRH antagonist described herein if the patient exhibits (i) a reduction in uterine blood loss (e.g., elimination of heavy menstrual blood loss) following administration of the GnRH antagonist to the patient; and/or (ii) induction of amenorrhea following administration of the GnRH antagonist to the patient.

Similarly, clinical indicators of successful treatment of a patient having endometriosis (e.g., rectovaginal endometriosis) using a GnRH antagonist described herein include (i) a reduction in the volume of one or more endometriosis nodes (e.g., rectovaginal endometriosis nodes) following administration of the GnRH antagonist to the patient; (ii) a reduction in bowel involvement of one or more type III endometriosis nodes following administration of the GnRH antagonist to the patient; (iii) a reduction in pelvic pain following administration of the GnRH antagonist to the patient; (iv) a reduction in dysmenorrhea following administration of the GnRH antagonist to the patient; (v) a reduction in dyspareunia following administration of the GnRH antagonist to the patient; (vi) a reduction in dyschezia following administration of the GnRH antagonist to the patient; (vii) a reduction in uterine tenderness following administration of the GnRH antagonist to the patient; (viii) a reduction in uterine bleeding following administration of the GnRH antagonist to the patient; (ix) achievement of amenorrhea following administration of the GnRH antagonist to the patient; and/or (x) an improvement in the patient's overall well-being as determined by an improvement in the patient's EHP-30 score following administration of the GnRH antagonist to the patient and/or the observation of a positive PGIC score following administration of the GnRH antagonist to the patient.

Clinical indicators of successful treatment of a patient having adenomyosis, another estrogen-dependent disease described herein, include, without limitation, (i) a reduction in uterine volume following administration of the GnRH antagonist to the patient; (ii) a reduction in the thickness of the anterior and/or posterior region of the uterine myometrium following administration of the GnRH antagonist to the patient; (iii) a reduction in pelvic pain following administration of the GnRH antagonist to the patient; (iv) a reduction in dysmenorrhea following administration of the GnRH antagonist to the patient; (v) a reduction in dyspareunia following administration of the GnRH antagonist to the patient; (vi) a reduction in dyschezia following administration of the GnRH antagonist to the patient; (vii) a reduction in uterine tenderness following administration of the GnRH antagonist to the patient; (viii) a reduction in uterine bleeding following administration of the GnRH antagonist to the patient; (ix) achievement of amenorrhea following administration of the GnRH antagonist to the patient; (x) a reduction in the diameter of a junctional zone of adenomyosis following administration of the GnRH antagonist to the patient; and/or (xi) an improvement in the patient's overall well-being as determined by an improvement in the patient's EHP-30 score following administration of the GnRH antagonist to the patient and/or the observation of a positive PGIC score following administration of the GnRH antagonist to the patient.

As used herein, the term “treatment period” refers to a duration of time over which a patient may be periodically administered a therapeutic agent, such as a GnRH antagonist described herein. Treatment periods as described herein may have a duration of, for example, several days, weeks, or months. For instance, a treatment period for administration of a thieno[3,4d]pyrimidine derivative, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof, such as the choline salt thereof, may last for from about four weeks to about six months (e.g., from about 28 days to about 180 days, about 30 days to about 175 days, about 35 days to about 170 days, about 40 days to about 165 days, about 45 days to about 160 days, about 50 days to about 155 days, about 55 days to about 150 days, about 60 days to about 145 days, about 65 days to about 140 days, about 70 days to about 135 days, about 75 days, to about 130 days, about 80 days to about 125 days, about 85 days, to about 120 days, or about 90 days to about 115 days). In some embodiments, the GnRH antagonist is periodically administered to the patient over a treatment period of from about eight weeks to about sixteen weeks (e.g., from about 60 days to about 110 days, about 65 days to about 105 days, about 70 days to about 100 days, about 75 days to about 95 days, or about 80 days, to about 90 days). In some embodiments, the GnRH antagonist is periodically administered to the patient over a treatment period of about twelve weeks. In some embodiments, the GnRH antagonist is periodically administered to the patient over a treatment period of from about 20 weeks to about 30 weeks (e.g., from about 140 days to about 210 days, about 150 days to about 100 days, about 60 days to about 90 days, about 65 days to about 85 days, or about 68 days). In some embodiments, the GnRH antagonist is periodically administered to the patient over a treatment period of about 24 weeks.

As used herein, the terms “first treatment period,” “second treatment period,” and the like refer to the chronological order in which the treatment periods occur. For the avoidance of doubt, it is to be understood that a “second treatment period,” when described in the context of a “first treatment period,” is subsequent to the first treatment period.

As used herein, the term “Verbal Rating Score” (VRS) refers to a subjective multi-point scale used to indicate the level of pain being experienced by a patient undergoing therapy or that has previously undergone therapy for a disease or condition, such as an estrogen-dependent disease described herein. The VRS may be a five-point scale and can be assessed by prompting the patient with one or more questions in order to determine the level of pain currently being experienced by the patient. Methods for assessing a VRS are described in detail, e.g., in Jensen et al., Journal of Pain and Symptom Management 41:1073-1093 (2011), the disclosure of which is incorporated herein by reference as it pertains to methods for obtaining and evaluating a VRS.

As used herein, the term “aryl” refers to an unsaturated aromatic carbocyclic group of from 6 to 14 carbon atoms having a single ring (e.g., optionally substituted phenyl) or multiple condensed rings (e.g., optionally substituted naphthyl). Exemplary aryl groups include phenyl, naphthyl, phenanthrenyl, and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “halogen atom” refers to a fluorine atom, a chlorine atom, a bromine atom, or an iodine atom.

As used herein, the term “heteroaryl” refers to a monocyclic heteroaromatic, or a bicyclic or a tricyclic fused-ring heteroaromatic group. Exemplary heteroaryl groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heterocycloalkyl” refers to a 3 to 8-membered heterocycloalkyl group having 1 or more heteroatoms, such as a nitrogen atom, an oxygen atom, a sulfur atom, and the like, and optionally having 1 or 2 oxo groups such as pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the terms “lower alkyl” and “C₁₋₆ alkyl” refer to an optionally branched alkyl moiety having from 1 to 6 carbon atoms, such as methyl, ethyl, propyl, isopropyl, butyl, isobutyl, sec-butyl, tert-butyl, pentyl, isopentyl, neopentyl, tert-pentyl, hexyl, and the like.

As used herein, the term “lower alkylene” refers to an optionally branched alkylene group having from 1 to 6 carbon atoms, such as methylene, ethylene, methylmethylene, trimethylene, dimethylmethylene, ethylmethylene, methylethylene, propylmethylene, isopropylmethylene, dimethylethylene, butylmethylene, ethylmethylmethylene, pentamethylene, diethylmethylene, dimethyltrimethylene, hexamethylene, diethylethylene and the like.

As used herein, the term “lower alkenyl” refers to an optionally branched alkenyl moiety having from 2 to 6 carbon atoms, such as vinyl, allyl, 1-propenyl, isopropenyl, 1-butenyl, 2-butenyl, 2-methylallyl, and the like.

As used herein, the term “lower alkynyl” refers to an optionally branched alkynyl moiety having from 2 to 6 carbon atoms, such as ethynyl, 2-propynyl, and the like.

As used herein, the term “optionally fused” refers to a cyclic chemical group that may be fused with a ring system, such as cycloalkyl, heterocycloalkyl, aryl, or heteroaryl. Exemplary ring systems that may be fused to an optionally fused chemical group include, e.g., indolyl, isoindolyl, benzofuranyl, isobenzofuranyl, benzothiophenyl, benzoxazolyl, benzothiazolyl, benzoisoxazolyl, benzoisothiazolyl, indazolyl, benzimidazolyl, quinolinyl, isoquinolinyl, phthalazinyl, quinoxalinyl, quinazolinyl, cinnolinyl, indolizinyl, naphthyridinyl, pteridinyl, indanyl, naphtyl, 1,2,3,4-tetrahydronaphthyl, indolinyl, isoindolinyl, 2,3,4,5-tetrahydrobenzo[b]oxepinyl, 6,7,8,9-tetrahydro-5H-benzocycloheptenyl, chromanyl, and the like.

As used herein, the term “optionally substituted” refers to a chemical moiety that may have one or more (e.g., 1, 2, 3, 4, 5, 6, 7, 8, 9, 10, or more chemical substituents, such as lower alkyl, lower alkenyl, lower alkynyl, cycloalkyl, heterocyclolalkyl, aryl, alkylaryl, heteroaryl, alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

As used herein, the term “sulfinyl” refers to the chemical moiety “—S(O)—R” in which R represents, e.g., hydrogen, aryl, heteroaryl, optionally substituted alkyl, optionally substituted alkenyl, or optionally substituted alkynyl.

As used herein, the term “sulfonyl” refers to the chemical moiety “—SO₂—R” in which R represents, e.g., hydrogen, aryl, heteroaryl, optionally substituted alkyl, optionally substituted alkenyl, or optionally substituted alkynyl.

One of skill in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure is to be understood as representing any such isomeric or isotopic form, individually or in combination.

### BRIEF DESCRIPTION OF THE FIGURES

FIG. 1 is a graph showing the median serum concentration of β17-estradiol (E2) in a series of female human patients suffering from endometriosis that were treated with varying amounts of a GnRH antagonist represented by formula (VI). As described in Example 1, below, patients were administered the indicated amount of the GnRH antagonist once daily over the course of 24 weeks. After 24 weeks, treatment with the GnRH antagonist was discontinued. Values along the x-axis denote the time at which the patients' E2 level was assessed relative to the onset of GnRH antagonist administration. Values along the y-axis represent the concentration of E2 in a serum sample obtained from the patient, in pg/ml. Patients were treated with the GnRH antagonist represented by formula (VI) in an amount of 50 mg/day, 75 mg/day, 100 mg/day, or 200 mg/day. Patients initially treated with placebo (plc) were administered the GnRH antagonist in an amount of 100 mg/day after 12 weeks. Also, after 12 weeks, patients that were initially administered 75 mg/day were provided a higher (100 mg/day) or lower (50 mg/day) dosage of the GnRH antagonist based on the patients' E2 levels at the 12-week point. The E2 levels for this group of patients is represented by “75 mg (TD).” Patients that were administered 75 mg through the end of the treatment period are represented by “75 mg (FD).”

FIG. 2 a graph showing the dyspareunia score in a series of female human patients suffering from endometriosis that were treated with varying amounts of a GnRH antagonist represented by formula (VI). As described in Example 1, below, patients were administered the indicated amount of the GnRH antagonist once daily over the course of 24 weeks. After 24 weeks, treatment with the GnRH antagonist was discontinued. Values along the x-axis denote the time at which patients' dyspareunia score was assessed using the Verbal Rating Score (VRS). Values along the y-axis represent the average change in the patients' VRS score relative to a baseline measurement of the patients' VRS score obtained prior to the onset of GnRH antagonist treatment (CFB). Patients were treated with the GnRH antagonist represented by formula (VI) in an amount of 50 mg/day, 75 mg/day, 100 mg/day, or 200 mg/day. Patients initially treated with placebo (plc) were administered the GnRH antagonist in an amount of 100 mg/day after 12 weeks. Also, after 12 weeks, patients that were initially administered 75 mg/day were provided a higher (100 mg/day) or lower (50 mg/day) dosage of the GnRH antagonist based on the patients' E2 levels at the 12-week point. The E2 levels for this group of patients is represented by “75 mg (TD).” Patients that were administered 75 mg through the end of the treatment period are represented by “75 mg (FD).”

FIG. 3 a graph showing the dyschezia score in a series of female human patients suffering from endometriosis that were treated with varying amounts of a GnRH antagonist represented by formula (VI). As described in Example 1, below, patients were administered the indicated amount of the GnRH antagonist once daily over the course of 24 weeks. After 24 weeks, treatment with the GnRH antagonist was discontinued. Values along the x-axis denote the time at which patients' dyschezia score was assessed using the Numerical Rating Score (NRS). Values along the y-axis represent the average change in the patients' NRS score relative to a baseline measurement of the patients' NRS score obtained prior to the onset of GnRH antagonist treatment (CFB). Patients were treated with the GnRH antagonist represented by formula (VI) in an amount of 50 mg/day, 75 mg/day, 100 mg/day, or 200 mg/day. Patients initially treated with placebo (plc) were administered the GnRH antagonist in an amount of 100 mg/day after 12 weeks. Also, after 12 weeks, patients that were initially administered 75 mg/day were provided a higher (100 mg/day) or lower (50 mg/day) dosage of the GnRH antagonist based on the patients' E2 levels at the 12-week point. The E2 levels for this group of patients is represented by “75 mg (TD).” Patients that were administered 75 mg through the end of the treatment period are represented by “75 mg (FD).”

FIGS. 4A-4D are magnetic resonance imaging (MRI) results showing the uterine adenomyotic condition of a patient prior to, and during the course of her treatment with, a GnRH antagonist represented by formula (VI). As described in Example 3, below, a cohort of n=6 female human patients suffering from uterine adenomyosis was treated with a GnRH antagonist represented by formula (VI). One of these patients in particular was extensively monitored prior to commencement of GnRH antagonist therapy as part of a longitudinal case study. Before undergoing treatment with the GnRH antagonist represented by formula (VI), this patient was first administered a selective progesterone receptor modulator (SPRM), ulipristal acetate (UPA), in an effort to treat the adenomyosis. After failing to respond to treatment with UPA, the patient was re-evaluated and administered compound (VI). FIGS. 4A-4D are MRI scans of this patient's uterus throughout the duration of this longitudinal case study. FIG. 4A is an MRI scan showing the condition of the patient's uterus prior to the commencement of treatment with UPA. FIG. 4A shows an enlarged uterus with diffuse and disseminated adenomyosis. FIG. 4B is an MRI scan obtained after the conclusion of 3 months of treatment with UPA, which was administered in an amount of 5 mg/day. FIG. 4B shows a worsened disease state, as evidenced by numerous spots typical of adenomyosis. FIG. 4B also shows an asymmetric, heterogeneous myometrium with multiple myometrial cysts, which is evidence of dilated islets of ectopic endometrium. FIG. 4C is an MRI scan obtained over one year after treatment with UPA discontinued. FIG. 4C shows a very large uterus, with clear indications typical of severe, full-thickness adenomyosis. FIG. 4D is an MRI scan obtained from the patient after 12 weeks of treatment with the GnRH antagonist represented by formula (VI), which was administered in a once-daily dose of 200 mg/day. As FIG. 4D shows, after 12 weeks of this treatment modality, the patient exhibited a significant reduction in uterine size and adenomyotic lesions.

FIG. 5 is a graph showing the effect of compound (VI) on menstrual bleeding patterns in uterine fibroids patients treated with compound (VI) for 24 weeks in accordance with the procedure described in Example 4, below. Values along the y axis represent the proportion of patients exhibiting menstrual blood loss of less than ≤80 mL, as assessed by way of the alkaline hematin method, and a 50% reduction in menstrual blood loss relative to baseline. Error bars represent 95% confidence intervals.

FIG. 6 is a graph showing the effect of compound (VI) on menstrual bleeding patterns in uterine fibroids patients treated with compound (VI) for 52 weeks in accordance with the procedure described in Example 4, below. Values along the y axis represent the proportion of patients exhibiting menstrual blood loss of less than ≤80 mL, as assessed by way of the alkaline hematin method, and a 50% reduction in menstrual blood loss relative to baseline. Error bars represent 95% confidence intervals.

FIGS. 7A and 7B are graphs showing the effect of compound (VI) on menstrual bleeding patterns in uterine fibroids patients treated with compound (VI) for 52 weeks in accordance with the procedure described in Example 4, below. Particularly, FIGS. 7A and 7B show the effect of compound (VI) on menstrual blood loss as assessed at various timepoints during the PRIMROSE 1 and PRIMROSE 2 trials described in Example 4, below. Values along the y axis represent the average change from baseline (CFB) in menstrual blood loss. Error bars represent 95% confidence intervals.

FIGS. 8A and 8B are graphs showing the effect of compound (VI) on pain levels in uterine fibroids patients treated with compound (VI) for 24 weeks in accordance with the procedure described in Example 4, below. Particularly, FIGS. 8A and 8B show the effect of compound (VI) on pain as assessed at various timepoints during the PRIMROSE 1 and PRIMROSE 2 trials by way of a Verbal Rating Score (VRS). Values along the y axis represent the average change from baseline (CFB) in pain score (FIG. 8A) and the proportion of patients with a score of 1 or less after 24 weeks of treatment (FIG. 8B).

FIG. 9 is a graph showing the effect of compound (VI) on bone mineral density levels in uterine fibroids patients treated with compound (VI) for 24 weeks and 52 weeks in accordance with the procedure described in Example 4, below. Bone mineral density was assessed in the lumbar spine. Values along the y axis represent the average change from baseline (CFB) in bone mineral density throughout the PRIMROSE 1 and PRIMROSE 2 studies, descried in Example 4.

### DETAILED DESCRIPTION

The compositions and methods of the present disclosure can be used to treat a variety of estrogen-dependent disorders, including uterine fibroids and endometriosis, among others. For example, using the compositions and methods described herein, a patient, such as a female human patient, may be administered a gonadotropin-releasing hormone (GnRH) antagonist in order to not only treat the cause of the estrogen-dependent pathology, but also to alleviate one or more symptoms associated these conditions. Exemplary disease that are induced by elevated estrogen production, and that can be treated using the compositions and methods described herein, include, without limitation, uterine fibroids, endometriosis (e.g., rectovaginal endometriosis), and adenomyosis, among others.

GnRH antagonists that may be used for the treatment of an estrogen-dependent disorder as described herein include optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof. Other GnRH antagonists useful in conjunction with the compositions and methods of the disclosure include optionally substituted 3-aminoalkyl pyrimidine-2,4(1H,3H)-dione derivatives, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof. In some embodiments, the GnRH antagonist is an optionally substituted thieno[2,3d]pyrimidine derivative, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof. In some embodiments, the GnRH antagonist is an optionally substituted propane-1,3-dione derivative, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Additional GnRH antagonists that may be used in conjunction with the compositions and methods described herein include SK12670 and BAY-784, as well as derivatives and variants thereof, among others.

Estrogen-dependent diseases, such as uterine fibroids, endometriosis (e.g., rectovaginal endometriosis), and adenomyosis, among others described herein, may be effectuated by circulating concentrations of β17-estradiol (E2) in excess of about 60 pg/ml. Using the compositions and methods described herein, a GnRH antagonist may be administered to the patient so as to suppress E2 production to healthy levels, such as a concentration of from about 10 pg/ml to about 60 pg/ml, in order to correct the underlying physiological cause of the disease and ameliorate one or more symptoms associated therewith. However, excessive depletion of endogenous E2 (for example, to levels of less than about 10 pg/ml) may lead to undesirable side effects, such as a reduction in bone mineral density.

The present disclosure is based, in part, on the surprising observation that administration of a GnRH antagonist, such as an optionally substituted thieno[3,4d]pyrimidine derivative (e.g., 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof), effectuates a sustained reduction in estrogen-dependent disease symptomology, even after administration of the GnRH antagonist is halted. This enables administration of the GnRH antagonist to be temporarily paused so as to allow a patient's serum E2 concentration to be replenished, for example, in the event of a reduction in bone mineral density, while maintaining an alleviating effect on estrogen-dependent disease symptoms. In some embodiments, once the patient's bone mineral density has increased, administration of the GnRH antagonist is re-initiated.

In some embodiments, in addition (or as an alternative) to temporarily pausing GnRH antagonist treatment, a patient suffering from an estrogen-dependent disease may be administered a reduced dosage of a GnRH antagonist if a reduction in bone mineral density occurs. The disclosure also provides dosing regimens in which a patient is administered a GnRH antagonist that is not an optionally substituted thieno[3,4d]pyrimidine derivative (e.g., 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof) and is subsequently treated with such a compound, in view, for example, of such compound's beneficial ability to sustain a reduction in disease symptoms even after its administration has been discontinued.

The sections that follow provide a detailed description of GnRH antagonists and other agents that may be used in conjunction with the compositions and methods of the disclosure, as well as a description of various estrogen-dependent diseases that may be treated using these therapeutics.

### GnRH Antagonists

### Thieno[3,4d]pyrimidines

GnRH antagonists for use with the compositions and methods described herein include thieno[3,4d]pyrimidine derivatives and variants, such as those described in U.S. Pat. No. 9,040,693, the disclosure of which is incorporated herein by reference in its entirety. Exemplary GnRH antagonists include those represented by formula (I)

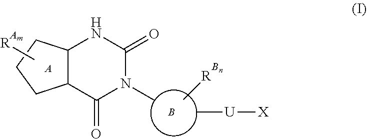

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group;

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments, the ring A is a thiophene ring represented by formula (IIa)

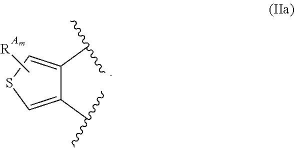

In some embodiments, m is 1 or 2. In some embodiments, m is 1. For instance, the ring A may be an optionally substituted thiophene ring represented by formula (IIb)

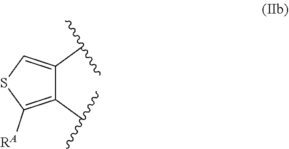

Each R^{A} may independently be, for example, a halogen atom (e.g., fluorine, chlorine, bromine, or iodine), an optionally substituted lower alkyl group, COOW¹, or CONW²W³, wherein W² to W³ independently are a hydrogen atom or an optionally substituted lower alkyl group, or W² and W³ may bind together with the neighboring nitrogen atom to form an optionally substituted cyclic amino group. In some embodiments, each R^{A} is COOH or pharmaceutically acceptable salt thereof.

In some embodiments, ring B is an optionally substituted benzene ring, pyridine ring, or thiophene ring. For instance, ring B may be represented by a formula selected from the group consisting of:

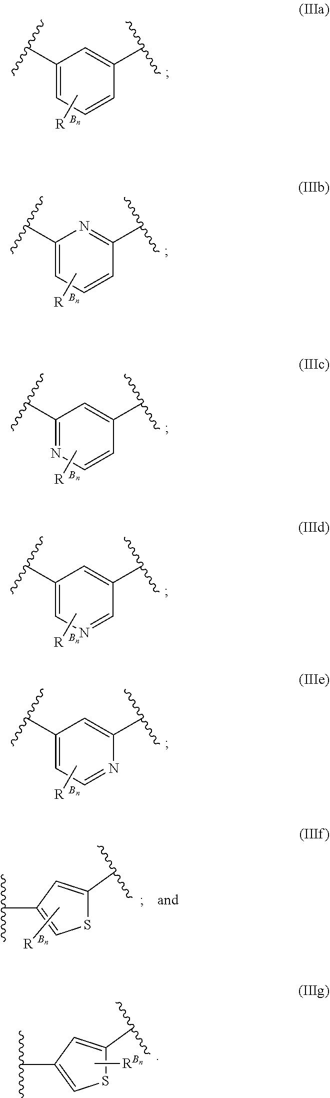

In some embodiments, n is 1 or 2. For instance, in some embodiments, n is 1. Ring B may be, for example, represented by a formula selected from the group consisting of:

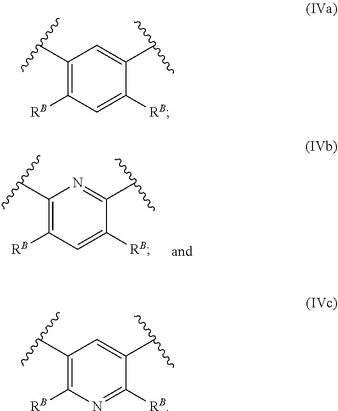

In some embodiments, each R^{B} is independently a halogen atom, an optionally substituted lower alkyl group, or OW⁴, wherein each W⁴ is independently a hydrogen atom or an optionally substituted lower alkyl group. For instance, each R^{B} may be independently a fluorine atom, chlorine atom, bromine atom, methyl group, or methoxy group.

In some embodiments, U is a single bond. X may be, for example, a group represented by —O-L-Y. L may be, for example, a methylene group. In some embodiments, Y is an optionally substituted benzene ring represented by formula (V)

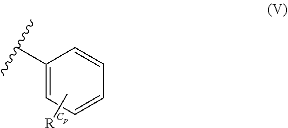

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments, Y is a substituted benzene ring represented by formula (Va)

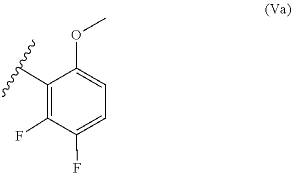 [SMILES: *c1c(OC)ccc(F)c1F]

For example, GnRH antagonists that may be used for the treatment of the endometrial growth disorders described herein include those thieno[3,4d]pyrimidine compounds described in Table 1, below. The synthesis and characterization of these compounds is reported, for instance, in U.S. Pat. No. 9,040,693, incorporated herein by reference.

TABLE 1
Exemplary Thieno[3,4d]pyrimidine GnRH Antagonists Useful for the Treatment of Estrogen-Dependent Diseases

| No. | Compound | Reported ¹H NMR spectral properties |
|---|---|---|
| 1 | 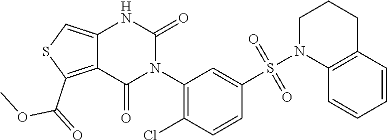 [SMILES: COC(=O)c1scc2[nH]c(=O)n(-c3cc(S(=O)(=O)N4CCCc5ccccc54)ccc3Cl)c(=O)c12] | (CDCl₃) 1.6-1.75 (2H, m), 2.45-2.6 (2H, m), 3.7-3.85 (2H, m), 3.94 (3H, s), 6.82 (1H, s), 7.0-7.15 (2H, m), 7.15-7.25 (1H, m), 7.49 (1H, dd, J = 8.4 Hz, 2.0 Hz), 7.54 (1H, d, J = 8.4 Hz), 7.6 (1H, d, J = 2.0 Hz), 7.7-7.8 (1H, m), 9.47 (1H, s) |
| 2 | 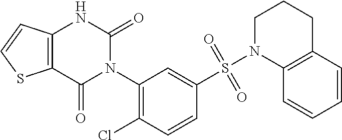 [SMILES: O=c1[nH]c2ccsc2c(=O)n1-c1cc(S(=O)(=O)N2CCCc3ccccc32)ccc1Cl] | (CDCl₃) 1.6-1.75 (2H, m), 2.4-2.6 (2H, m), 3.7-3.8 (1H, m), 3.8-3.9 (1H, m), 6.82 (1H, d, J = 5.2 Hz), 7.0-7.15 (2H, m), 7.15-7.25 (1H, m), 7.46 (1H, dd, J = 8.3 Hz, 2.1 Hz), 7.54 (1H, d, J = 8.3 Hz), 7.65 (1H, d, J = 2.1 Hz), 7.7-7.8 (2H, m), 9.65 (1H, s) |
| 3 | 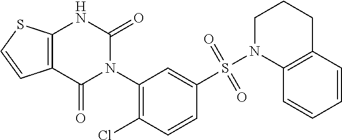 [SMILES: O=c1[nH]c2sccc2c(=O)n1-c1cc(S(=O)(=O)N2CCCc3ccccc32)ccc1Cl] | (CDCl₃) 1.6-1.75 (2H, m), 2.4-2.6 (2H, m), 3.7-3.9 (2H, m), 6.89 (1H, d, J = 5.8 Hz), 7.0- 7.15 (2H, m), 7.15-7.25 (1H, m), 7.32 (1H, d, J = 5.8 Hz), 7.46 (1H, dd, J = 8.4 Hz, 2.1 Hz), 7.55 (1H, d, J = 8.4 Hz), 7.64 (1H, d, J = 2.1 Hz), 7.75- 7.8 (1H, m), 9.54 (1H, brs) |
| 4 | 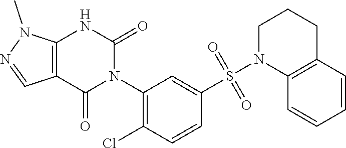 [SMILES: Cn1ncc2c(=O)n(-c3cc(S(=O)(=O)N4CCCc5ccccc54)ccc3Cl)c(=O)[nH]c21] | (CDCl₃) 1.6-1.8 (2H, m), 2.4-2.6 (2H, m), 3.65-3.8 (4H, m), 3.8-3.9 (1H, m), 7.0-7.15 (2H, m), 7.15-7.25 (1H, m), 7.49 (1H, dd, J = 8.4 Hz, 1.9 Hz), 7.55 (1H, d, J = 8.4 Hz), 7.59 (1H, d, J = 1.9 Hz), 7.75 (1H, d, J = 7.5 Hz), 7.91 (1H, s) |
| 5 | 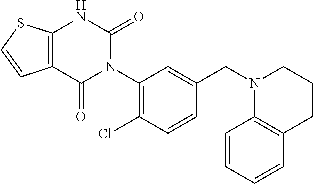 [SMILES: O=c1[nH]c2sccc2c(=O)n1-c1cc(CN2CCCc3ccccc32)ccc1Cl] | (CDCl₃) 1.95-2.05 (2H, m), 2.75-2.85 (2H, m), 3.3-3.35 (2H, m), 4.48 (2H, s), 6.5 (1H, d, J = 8.4 Hz), 6.55-6.65 (1H, m), 6.81 (1H, d, J = 5.6 Hz), 6.9-7.0 (2H m), 7.2-7.35 (3H, m), 7.51 (1H, d, J = 8.4 Hz), 9.94 (1H, brs) |
| 6 | 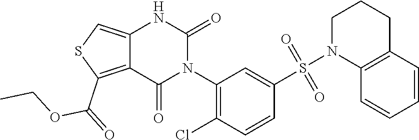 [SMILES: CCOC(=O)c1scc2[nH]c(=O)n(-c3cc(S(=O)(=O)N4CCCc5ccccc54)ccc3Cl)c(=O)c12] | (CDCl₃) 1.38 (3H, t, J = 7.1 Hz), 1.6- 1.75 (2H, m), 2.4-2.6 (2H, m), 3.7-3.85 (2H, m), 4.41 (2H, q, J = 7.1 Hz), 6.82 (1H, s), 7.0-7.15 (2H, m), 7.15-7.25 (1H, m), 7.47 (1H, dd, J = 8.5 Hz, 2.2 Hz), 7.53 (1H, d, J = 8.5 Hz), 7.6 (1H, d, J = 2.2 Hz), 7.7-7.8 (1H, m), 9.11 (1H, s) |
| 7 | 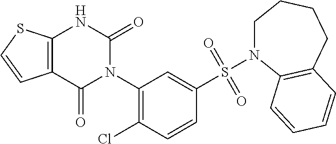 [SMILES: O=c1[nH]c2sccc2c(=O)n1-c1cc(S(=O)(=O)N2CCCCc3ccccc32)ccc1Cl] | (CDCl₃) 1.2-1.35 (2H, m), 1.75-1.9 (2H, m), 2.4-2.55 (2H, m), 3.2-4.3 (2H, br), 6.87 (1H, d, J = 5.3 Hz), 7.1-7.2 (3H, m), 7.25-7.35 (2H, m), 7.63 (1H, d, J = 8.5 Hz), 7.69 (1H, dd, J = 8.5 Hz, 1.9 Hz), 7.78 (1H, d, J = 1.9 Hz), 9.5-10.2 (1H, br) |
| 8 | 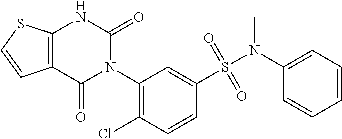 [SMILES: CN(c1ccccc1)S(=O)(=O)c1ccc(Cl)c(-n2c(=O)[nH]c3sccc3c2=O)c1] | (CDCl₃) 3.2 (3H, s), 6.87 (1H, d, J = 5.4 Hz), 7.1-7.15 (2H, m), 7.2-7.35 (4H, m), 7.54 (1H, dd, J = 8.4 Hz, 2.0 Hz), 7.6 (1H, d, J = 2.0 Hz), 7.64 (1H, d, J = 8.4 Hz), 9.5-11.0 (1H, br) |
| 9 | 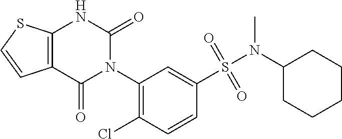 [SMILES: CN(C1CCCCC1)S(=O)(=O)c1ccc(Cl)c(-n2c(=O)[nH]c3sccc3c2=O)c1] | (CDCl₃) 0.9-1.8 (10H, m), 2.77 (3H, s), 3.7-3.8 (1H, m), 6.87 (1H, d, J = 5.7 Hz), 7.3 (1H, d, J = 5.7 Hz), 7.69 (1H, d, J = 8.1 Hz), 7.8-7.9 (2H, m), 9.5-10.5 (1H, br) |
| 10 | 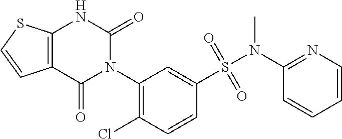 [SMILES: CN(c1ccccn1)S(=O)(=O)c1ccc(Cl)c(-n2c(=O)[nH]c3sccc3c2=O)c1] | (DMSO-d₆) 3.26 (3H, s), 7.19 (1H, d, J = 5.7 Hz), 7.22 (1H, d, J = 5.7 Hz), 7.25- 7.3 (1H, m), 7.45-7.55 (1H, m), 7.62 (1H, dd, J = 8.5 Hz, 2.1 Hz), 7.8-7.9 (2H, m), 8.04 (1H, d, J = 2.1 Hz), 8.3-8.4 (1H, m), 12.54 (1H, s) |
| 11 | 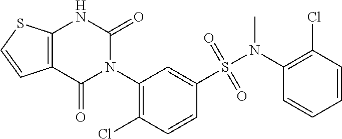 [SMILES: CN(c1ccccc1Cl)S(=O)(=O)c1ccc(Cl)c(-n2c(=O)[nH]c3sccc3c2=O)c1] | (DMSO-d₆) 3.16 (3H, s), 7.05 (1H, d, J = 7.8 Hz), 7.15-7.45 (4H, m), 7.5-7.6 (1H, m), 7.75-8.1 (3H, m), 12.55 (1H, s) |
| 12 | 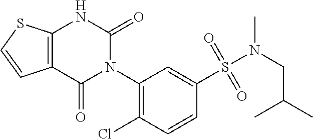 [SMILES: CC(C)CN(C)S(=O)(=O)c1ccc(Cl)c(-n2c(=O)[nH]c3sccc3c2=O)c1] | (DMSO-d₆) 0.8-0.9 (6H, m), 1.75-1.95 (1H, m), 2.6-2.8 (5H, m), 7.15-7.25 (2H, m), 7.8- 7.95 (2H, m), 8.0-8.1 (1H, m), 12.53 (1H, s) |
| 13 | 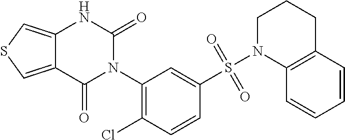 [SMILES: O=c1[nH]c2cscc2c(=O)n1-c1cc(S(=O)(=O)N2CCCc3ccccc32)ccc1Cl] | (DMSO-d₆) 1.6-1.75 (2H, m), 2.4-2.55 (2H, m), 3.7-3.85 (2H, m), 6.95 (1H, d, J = 3.1 Hz), 7.05-7.25 (3H, m), 7.52 (1H, dd, J = 8.5 Hz, 2.0 Hz), 7.56 (1H, d, J = 8.1 Hz), 7.78 (1H, d, J = 8.5 Hz), 8.04 (1H, d, J = 2.0 Hz), 8.52 (1H, d, J = 3.1 Hz), 11.53 (1H, s) |
| 14 | 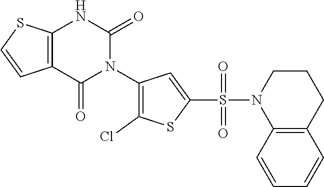 [SMILES: O=c1[nH]c2sccc2c(=O)n1-c1cc(S(=O)(=O)N2CCCc3ccccc32)sc1Cl] | (CDCl₃) 1.75-1.9 (2H, m), 2.55-2.7 (2H, m), 3.75-3.95 (2H, m), 6.9 (1H, d, J = 5.5 Hz), 7.05-7.25 (4H, m), 7.29 (1H, d, J = 5.5 Hz), 7.74 (1H, d, J = 8.2 Hz), 10.17 (1H, s) |
| 15 | 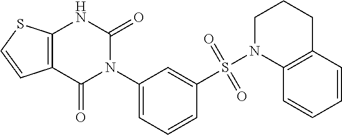 [SMILES: O=c1[nH]c2sccc2c(=O)n1-c1cccc(S(=O)(=O)N2CCCc3ccccc32)c1] | (DMSO-d₆) 1.6-1.7 (2H, m), 2.4-2.5 (2H, m), 3.7-3.8 (2H, m), 7.05-7.1 (2H, m), 7.1-7.25 (3H, m), 7.45-7.65 (4H, m), 7.74 (1H, d, J = 1.0 Hz), 12.37 (1H, s) |
| 16 | 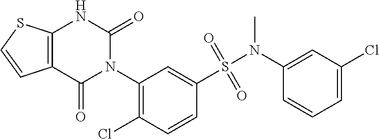 [SMILES: CN(c1cccc(Cl)c1)S(=O)(=O)c1ccc(Cl)c(-n2c(=O)[nH]c3sccc3c2=O)c1] | (DMSO-d₆) 3.18 (3H, s), 7.0-7.1 (1H, m), 7.15- 7.25 (3H, m), 7.35-7.4 (2H, m), 7.6 (1H, dd, J = 8.5 Hz, 2.3 Hz), 7.87 (1H, d, J = 8.5 Hz), 7.96 (1H, d, J = 2.3 Hz), 12.54 (1H, s) |
| 17 | 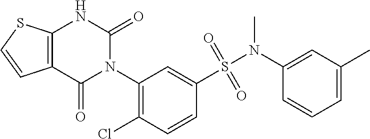 [SMILES: Cc1cccc(N(C)S(=O)(=O)c2ccc(Cl)c(-n3c(=O)[nH]c4sccc4c3=O)c2)c1] | (DMSO-d₆) 2.26 (3H, s), 3.15 (3H, s), 6.8-6.95 (2H, m), 7.05-7.25 (4H, m), 7.6 (1H, dd, J = 8.4 Hz, 2.3 Hz), 7.8-7.9 (2H, m), 12.54 (1H, s) |
| 18 | 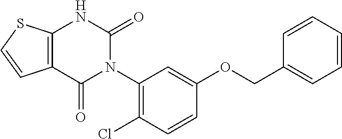 [SMILES: O=c1[nH]c2sccc2c(=O)n1-c1cc(OCc2ccccc2)ccc1Cl] | (DMSO-d₆) 5.1 (2H, s), 7.14 (1H, dd, J = 9.0 Hz, 2.9 Hz), 7.18 (1H, d, J = 5.6 Hz), 7.22 (1H, d, J = 5.6 Hz), 7.25 (1H, d, J = 2.9 Hz), 7.3-7.5 (5H, m), 7.53 (1H, d, J = 9.0 Hz), 12.48 (1H, s) |
| 19 | 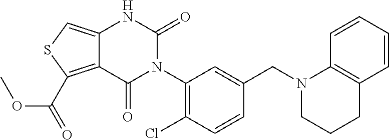 [SMILES: COC(=O)c1scc2[nH]c(=O)n(-c3cc(CN4CCCc5ccccc54)ccc3Cl)c(=O)c12] | (DMSO-d₆) 1.85-2.0 (2H, m), 2.7-2.8 (2H, m), 3.3-3.4 (2H, m), 3.81 (3H, s), 4.5 (2H, s), 6.4-6.5 (2H, m), 6.85-6.95 (2H, m), 7.2 (1H, s), 7.31 (1H, dd, J = 8.2 Hz, 2.1 Hz), 7.4 (1H, d, J = 2.1 Hz), 7.56 (1H, d, J = 8.2 Hz), 11.61 (1H, s) |
| 20 | 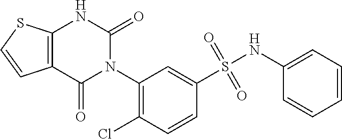 [SMILES: O=c1[nH]c2sccc2c(=O)n1-c1cc(S(=O)(=O)Nc2ccccc2)ccc1Cl] | (DMSO-d₆) 7.05-7.3 (7H, m), 7.75-7.9 (2H, m), 7.95-8.05 (1H, m), 10.48 (1H, s), 12.56 (1H, s) |
| 21 | 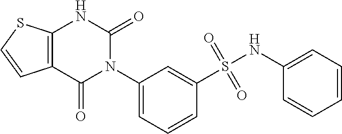 [SMILES: O=c1[nH]c2sccc2c(=O)n1-c1cccc(S(=O)(=O)Nc2ccccc2)c1] | (DMSO-d₆) 7.0-7.3 (7H, m), 7.55-7.7 (2H, m), 7.75-7.85 (2H, m), 10.4 (1H, s), 12.4 (1H, s) |
| 22 | 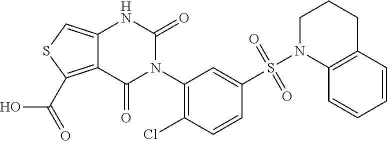 [SMILES: O=C(O)c1scc2[nH]c(=O)n(-c3cc(S(=O)(=O)N4CCCc5ccccc54)ccc3Cl)c(=O)c12] | (DMSO-d₆) 1.6-1.7 (2H, m), 2.4-2.55 (2H, m), 3.7-3.8 (2H, m), 7.05-7.25 (3H, m), 7.39 (1H, s), 7.55 (1H, d, J = 8.2 Hz), 7.6 (1H, dd, J = 8.5 Hz, 2.4 Hz), 7.83 (1H, d, J = 8.5 Hz), 8.09 (1H, d, J = 2.4 Hz), 12.03 (1H, s), 14.23 (1H, s) |
| 23 | 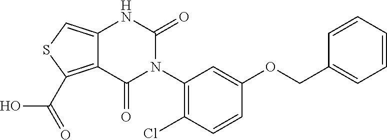 [SMILES: O=C(O)c1scc2[nH]c(=O)n(-c3cc(OCc4ccccc4)ccc3Cl)c(=O)c12] | (DMSO-d₆) 5.11 (2H, s), 7.19 (1H, dd, J = 9.0 Hz, 2.9 Hz), 7.3-7.45 (5H, m), 7.45-7.5 (2H, m), 7.57 (1H, d, J = 9.0 Hz), 12.04 (1H, s), 14.45 (1H, brs) |
| 24 | 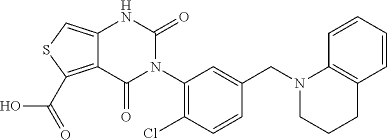 [SMILES: O=C(O)c1scc2[nH]c(=O)n(-c3cc(CN4CCCc5ccccc54)ccc3Cl)c(=O)c12] | (DMSO-d₆) 1.85-2.0 (2H, m), 2.7-2.8 (2H, m), 3.3-3.4 (2H, m), 4.52 (2H, s), 6.44 (1H, d, J = 8.2 Hz), 6.45-6.5 (1H, m), 6.85-6.95 (2H, m), 7.36 (1H, d, J = 8.2 Hz), 7.38 (1H, s), 7.48 (1H, d, J = 2.1 Hz), 7.61 (1H, d, J = 8.2 Hz), 12.0 (1H, s), 14.45 (1H, brs) |
| 25 | 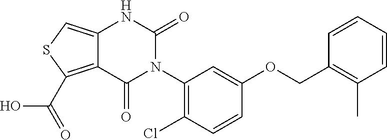 [SMILES: Cc1ccccc1COc1ccc(Cl)c(-n2c(=O)[nH]c3csc(C(=O)O)c3c2=O)c1] | (DMSO-d₆) 2.33 (3H, s), 5.09 (2H, s), 7.15-7.3 (4H, m), 7.35 (1H, d, J = 2.7 Hz), 7.41 (1H, s), 7.43 (1H, d, J = 7.7 Hz), 7.58 (1H, d, J = 9.0 Hz), 12.04 (1H, s), 14.44 (1H, s) |
| 26 | 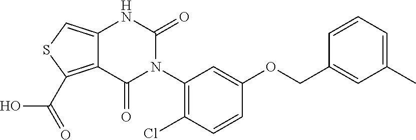 [SMILES: Cc1cccc(COc2ccc(Cl)c(-n3c(=O)[nH]c4csc(C(=O)O)c4c3=O)c2)c1] | (DMSO-d₆) 2.32 (3H, s), 5.07 (2H, s), 7.1-7.35 (6H, m), 7.41 (1H, s), 7.57 (1H, d, J = 8.7 Hz), 12.04 (1H, s), 14.45 (1H, brs) |
| 27 | 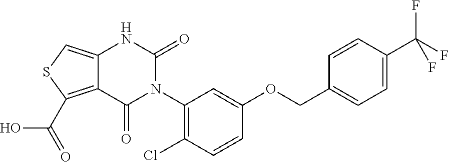 [SMILES: O=C(O)c1scc2[nH]c(=O)n(-c3cc(OCc4ccc(C(F)(F)F)cc4)ccc3Cl)c(=O)c12] | (DMSO-d₆) 5.24 (2H, s), 7.2 (1H, dd, J = 8.8 Hz, 3.0 Hz), 7.35 (1H, d, J = 3.0 Hz), 7.41 (1H, s), 7.59 (1H, d, J = 8.8 Hz), 7.69 (2H, d, J = 8.2 Hz), 7.78 (2H, d, J = 8.2 Hz), 12.04 (1H, s), 14.43 (1H, s) |
| 28 | 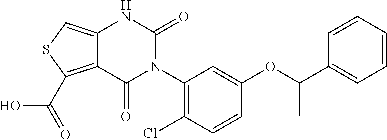 [SMILES: CC(Oc1ccc(Cl)c(-n2c(=O)[nH]c3csc(C(=O)O)c3c2=O)c1)c1ccccc1] | (DMSO-d₆) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 29 | 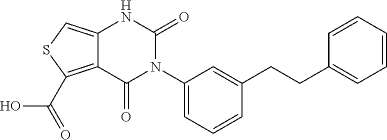 [SMILES: O=C(O)c1scc2[nH]c(=O)n(-c3cccc(CCc4ccccc4)c3)c(=O)c12] | (DMSO-d₆) 2.85-3.0 (4H, m), 7.15-7.35 (8H, m), 7.38 (1H, s), 7.41 (1H, t, J = 7.7 Hz), 11.92 (1H, s), 14.94 (1H, brs) |
| 30 | 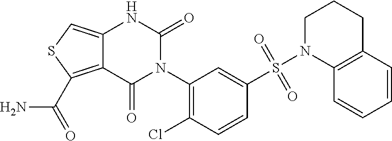 [SMILES: NC(=O)c1scc2[nH]c(=O)n(-c3cc(S(=O)(=O)N4CCCc5ccccc54)ccc3Cl)c(=O)c12] | (DMSO-d₆) 1.6-1.7 (2H, m), 2.4-2.55 (2H, m), 3.7-3.85 (2H, m), 7.0-7.2 (3H, m), 7.24 (1H, s), 7.56 (1H, d, J = 8.3 Hz), 7.59 (1H, dd, J = 8.5 Hz, 2.1 Hz), 7.81 (1H, d, J = 8.5 Hz), 8.05-8.15 (2H, m), 9.55 (1H, s), 11.81 (1H, s) |
| 31 | 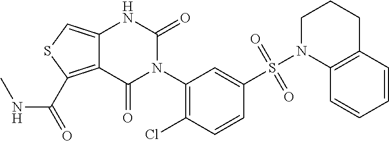 [SMILES: CNC(=O)c1scc2[nH]c(=O)n(-c3cc(S(=O)(=O)N4CCCc5ccccc54)ccc3Cl)c(=O)c12] | (CDCl₃) 1.65-1.75 (2H, m), 2.4-2.6 (2H, m), 2.99 (3H, d, J = 4.7 Hz), 3.7-3.9 (2H, m), 6.91 (1H, s), 6.95-7.15 (2H, m), 7.15-7.25 (1H, m), 7.5-7.65 (3H, m), 7.7-7.8 (1H, m), 8.91 (1H, s), 10.05- 10.15 (1H, m) |
| 32 | 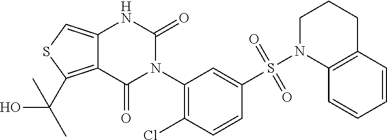 [SMILES: CC(C)(O)c1scc2[nH]c(=O)n(-c3cc(S(=O)(=O)N4CCCc5ccccc54)ccc3Cl)c(=O)c12] | (CDCl₃) 1.55 (6H, s), 1.6-1.75 (2H, m), 2.4-2.55 (2H, m), 3.7-3.9 (2H, m), 6.04 (1H, s), 6.49 (1H, s), 7.0-7.15 (2H, m), 7.15-7.25 (2H, m), 7.5-7.55 (1H, m), 7.55-7.6 (2H, m), 7.76 (1H, d, J = 8.3 Hz), 8.41 (1H, s) |
| 33 | 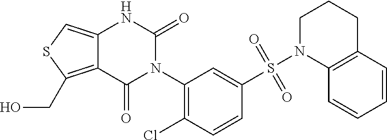 [SMILES: O=c1[nH]c2csc(CO)c2c(=O)n1-c1cc(S(=O)(=O)N2CCCc3ccccc32)ccc1Cl] | (DMSO-d₆) 1.6-1.75 (2H, m), 2.45-2.55 (2H, m), 3.7-3.85 (2H, m), 4.95-5.05 (2H, m), 5.99 (1H, t, J = 5.5 Hz), 6.73 (1H, s), 7.05-7.15 (2H, m), 7.15-7.25 (1H, m), 7.5-7.6 (2H, m), 7.78 (1H, d, J = 8.5 Hz), 7.97 (1H, d, J = 2.3 Hz), 11.41 (1H, s) |
| 34 | 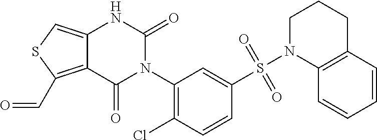 [SMILES: O=Cc1scc2[nH]c(=O)n(-c3cc(S(=O)(=O)N4CCCc5ccccc54)ccc3Cl)c(=O)c12] | (DMSO-d₆) 1.6-1.75 (2H, m), 2.45-2.55 (2H, m), 3.7-3.8 (2H, m), 7.05-7.15 (2H, m), 7.15-7.25 (1H, m), 7.5-7.6 (3H, m), 7.82 (1H, d, J = 8.5 Hz), 8.09 (1H, d, J = 2.1 Hz), 10.5-10.55 (1H,m), 11.88 (1H, s) |
| 35 | 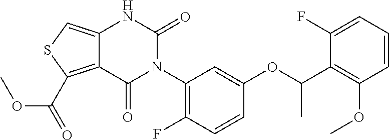 [SMILES: COC(=O)c1scc2[nH]c(=O)n(-c3cc(OC(C)c4c(F)cccc4OC)ccc3F)c(=O)c12] | (DMSO-d₆) 1.64 (3H, d, J = 6.5 Hz), 3.8- 3.9 (6H, m), 5.79 (1H, q, J = 6.5 Hz), 6.7-6.8 (1H, m), 6.85-6.95 (2H, m), 6.95- 7.05 (1H, m), 7.15-7.25 (2H, m), 7.25- 7.35 (1H, m), 11.63 (1H, s) |
| 36 | 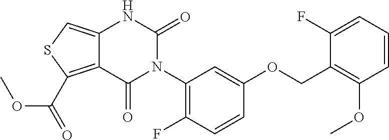 [SMILES: COC(=O)c1scc2[nH]c(=O)n(-c3cc(OCc4c(F)cccc4OC)ccc3F)c(=O)c12] | (DMSO-d₆) 3.83 (3H, s), 3.85 (3H, s), 4.99 (2H, s), 6.85-7.0 (2H, m), 7.1-7.2 (2H, m), 7.21 (1H, s), 7.25-7.35 (1H, m), 7.4-7.5 (1H, m), 11.68 (1H, s) |
| 37 | 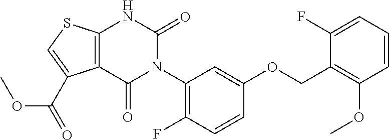 [SMILES: COC(=O)c1csc2[nH]c(=O)n(-c3cc(OCc4c(F)cccc4OC)ccc3F)c(=O)c12] | (DMSO-d₆) 3.75 (3H, s), 3.85 (3H, s), 4.99 (2H, s), 6.85-7.0 (2H, m), 7.1-7.2 (2H, m), 7.25-7.35 (1H, m), 7.4-7.5 (1H, m), 7.65 (1H, s), 12.64 (1H, s) |
| 38 | 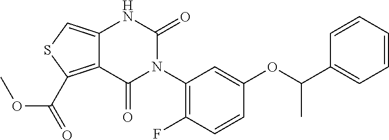 [SMILES: COC(=O)c1scc2[nH]c(=O)n(-c3cc(OC(C)c4ccccc4)ccc3F)c(=O)c12] | (DMSO-d₆) 1.54 (3H, d, J = 6.3 Hz), 3.8- 3.85 (3H, m), 5.46 (1H, q, J = 6.3 Hz), 6.9-7.0 (1H, m), 7.05-7.1 (1H, m), 7.15- 7.45 (7H, m), 11.63 (1H, s) |
| 39 | 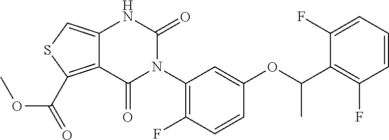 [SMILES: COC(=O)c1scc2[nH]c(=O)n(-c3cc(OC(C)c4c(F)cccc4F)ccc3F)c(=O)c12] | (DMSO-d₆) 1.7 (3H, d, J = 6.8 Hz), 3.8- 3.85 (3H, m), 5.7-5.8 (1H, m), 6.9-7.0 (1H, m), 7.05-7.15 (3H, m), 7.15-7.3 (2H, m), 7.35-7.5 (1H, m), 11.63 (1H, s) |
| 40 | 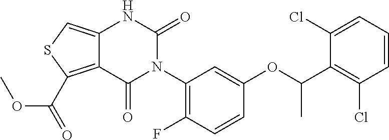 [SMILES: COC(=O)c1scc2[nH]c(=O)n(-c3cc(OC(C)c4c(Cl)cccc4Cl)ccc3F)c(=O)c12] | (DMSO-d₆) 1.71 (3H, d, J = 6.6 Hz), 3.82 (3H, s), 5.95-6.05 (1H, m), 6.8-6.9 (1H, m), 7.0-7.05 (1H, m), 7.15-7.3 (2H, m), 7.3-7.4 (1H, m), 7.45-7.5 (2H, m), 11.63 (1H, s) |
| 41 | 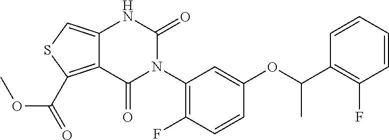 [SMILES: COC(=O)c1scc2[nH]c(=O)n(-c3cc(OC(C)c4ccccc4F)ccc3F)c(=O)c12] | (DMSO-d₆) 1.58 (3H, d, J = 6.3 Hz), 3.8- 3.85 (3H, m), 5.66 (1H, q, J = 6.3 Hz), 6.9-7.0 (1H, m), 7.05-7.15 (1H, m), 7.15-7.3 (4H, m), 7.3-7.4 (1H, m), 7.45- 7.55 (1H, m), 11.63 (1H, s) |
| 42 | 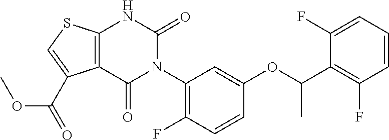 [SMILES: COC(=O)c1csc2[nH]c(=O)n(-c3cc(OC(C)c4c(F)cccc4F)ccc3F)c(=O)c12] | (DMSO-d₆) 1.69 (3H, d, J = 6.4 Hz), 3.7- 3.75 (3H, m), 5.7-5.8 (1H, m), 6.9-7.0 (1H, m), 7.05-7.15 (3H, m), 7.2-7.3 (1H, m), 7.35-7.5 (1H, m), 7.6-7.65 (1H, m), 12.59 (1H, s) |
| 43 | 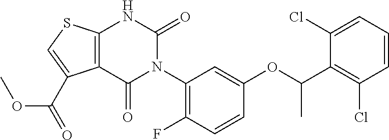 [SMILES: COC(=O)c1csc2[nH]c(=O)n(-c3cc(OC(C)c4c(Cl)cccc4Cl)ccc3F)c(=O)c12] | (DMSO-d₆) 1.71 (3H, d, J = 6.6 Hz), 3.74 (3H, s), 5.95-6.05 (1H, m), 6.8-6.9 (1H, m), 6.95-7.05 (1H, m), 7.2-7.3 (1H, m), 7.3-7.4 (1H, m), 7.4-7.5 (2H, m), 7.6-7.65 (1H, m), 12.59 (1H, s) |
| 44 | 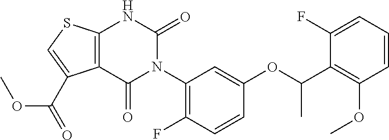 [SMILES: COC(=O)c1csc2[nH]c(=O)n(-c3cc(OC(C)c4c(F)cccc4OC)ccc3F)c(=O)c12] | (DMSO-d₆) 1.64 (3H, d, J = 6.7 Hz), 3.7- 3.8 (3H, m), 3.8-3.9 (3H, m), 5.75-5.85 (1H, m), 6.7-6.8 (1H, m), 6.85-6.95 (2H, m), 6.95-7.05 (1H, m), 7.15-7.25 (1H, m), 7.25-7.35 (1H, m), 7.63 (1H, s), 12.58 (1H, s) |
| 45 | 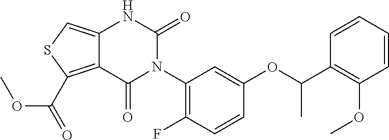 [SMILES: COC(=O)c1scc2[nH]c(=O)n(-c3cc(OC(C)c4ccccc4OC)ccc3F)c(=O)c12] | (DMSO-d₆) 1.51 (3H, d, J = 6.3 Hz), 3.8- 3.9 (6H, m), 5.6-5.7 (1H, m), 6.8-6.9 (1H, m), 6.9-7.0 (1H, m), 7.0-7.1 (2H, m), 7.15-7.3 (3H, m), 7.3-7.4 (1H, m), 11.6 (1H, s) |
| 46 | 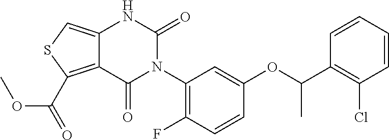 [SMILES: COC(=O)c1scc2[nH]c(=O)n(-c3cc(OC(C)c4ccccc4Cl)ccc3F)c(=O)c12] | (DMSO-d₆) 1.57 (3H, d, J = 6.3 Hz), 3.8- 3.85 3H, m), 5.6-5.7 (1H, m), 6.8-6.9 (1H, m), 7.05-7.1 (1H, m), 7.15-7.3 (2H, m), 7.3-7.4 m), 7.45-7.5 (1H, m), 7.5-7.6 (1H, m), 11.61 (1H, s) |
| 47 | 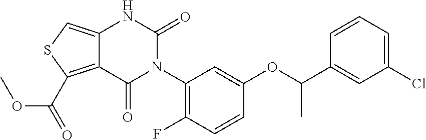 [SMILES: COC(=O)c1scc2[nH]c(=O)n(-c3cc(OC(C)c4cccc(Cl)c4)ccc3F)c(=O)c12] | (DMSO-d₆) 1.54 (3H, d, J = 6.2 Hz), 3.75-3.85 (3H, m), 5.45-5.55 (1H, m), 6.95-7.05 (1H, m), 7.05-7.15 (1H, m), 7.15-7.25 (2H, m), 7.3-7.45 (3H, m), 7.49 (1H, s), 11.61 (1H, s) |
| 48 | 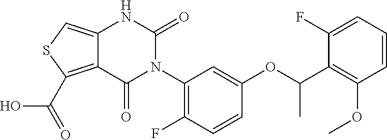 [SMILES: COc1cccc(F)c1C(C)Oc1ccc(F)c(-n2c(=O)[nH]c3csc(C(=O)O)c3c2=O)c1] | (DMSO-d₆) 1.65 (3H, d, J = 6.5 Hz), 3.8- 3.9 3H, m), 5.75-5.85 (1H, m), 6.7-6.8 (1H, m), 6.85-7.0 (2H, m), 7.05-7.1 (1H, m), 7.2-7.35 (2H, m), 7.37 (1H, d, J = 3.6 Hz), 12.01 (1H, s), 14.43 (1H, s) |
| 49 | 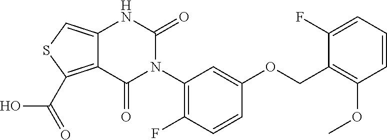 [SMILES: COc1cccc(F)c1COc1ccc(F)c(-n2c(=O)[nH]c3csc(C(=O)O)c3c2=O)c1] | (DMSO-d₆) 3.85 (3H, s), 5.0 (2H, s), 6.88 (1H, t, J = 8.7 Hz), 6.95 (1H, d, J = 8.7 Hz), 7.15-7.25 (2H, m), 7.3-7.5 (3H, m), 12.06 (1H, s), 14.43 (1H, s) |
| 50 | 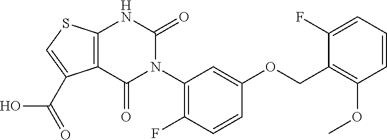 [SMILES: COc1cccc(F)c1COc1ccc(F)c(-n2c(=O)[nH]c3scc(C(=O)O)c3c2=O)c1] | (DMSO-d₆) 3.85 (3H, s), 5.0 (2H, s), 6.88 (1H, t, J = 8.6 Hz), 6.95 (1H, d, J = 8.6 Hz), 7.1-7.2 (1H, m), 7.2-7.25 (1H, m), 7.3-7.4 (1H, m), 7.4-7.5 (1H, m), 7.94 (1H, s), 13.04 (1H, s), 13.93 (1H, s) |
| 51 | 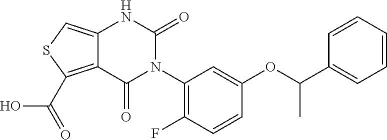 [SMILES: CC(Oc1ccc(F)c(-n2c(=O)[nH]c3csc(C(=O)O)c3c2=O)c1)c1ccccc1] | (DMSO-d₆) 1.5-1.6 (3H, m), 5.4-5.5 (1H, m), 6.95-7.05 (1H, m), 7.1-7.2 (1H, m), 7.2-7.4 (5H, m), 7.4-7.45 (2H, m), 11.95-12.05 (1H, m), 14.42 (1H, s) |
| 52 | 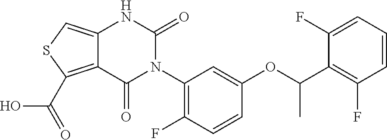 [SMILES: CC(Oc1ccc(F)c(-n2c(=O)[nH]c3csc(C(=O)O)c3c2=O)c1)c1c(F)cccc1F] | (DMSO-d₆) 1.7 (3H, d, J = 6.6 Hz), 5.76 (1H, q, J = 6.6 Hz), 7.0-7.2 (4H, m), 7.25-7.35 (1H, m), 7.35-7.5 (2H, m), 11.95-12.05 (1H, m), 14.42 (1H, s) |
| 53 | 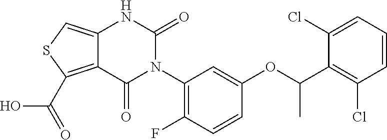 [SMILES: CC(Oc1ccc(F)c(-n2c(=O)[nH]c3csc(C(=O)O)c3c2=O)c1)c1c(Cl)cccc1Cl] | (DMSO-d₆) 1.65-1.75 (3H, m), 6.03 (1H, q, J = 6.6 Hz), 6.85-6.95 (1H, m), 7.05- 7.15 (1H, m), 7.25-7.4 (3H, m), 7.4-7.5 (2H, m), 11.95-12.05 (1H, m), 14.4 (1H, s) |
| 54 | 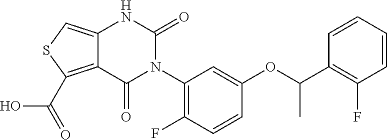 [SMILES: CC(Oc1ccc(F)c(-n2c(=O)[nH]c3csc(C(=O)O)c3c2=O)c1)c1ccccc1F] | (DMSO-d₆) 1.55-1.65 (3H, m), 5.65 (1H, q, J = 6.5 Hz), 7.0-7.05 (1H, m), 7.15- 7.25 (3H, m), 7.25-7.4 (3H, m), 7.45- 7.55 (1H, m), 11.95- 12.05 (1H, m), 14.42 (1H, s) |
| 55 | 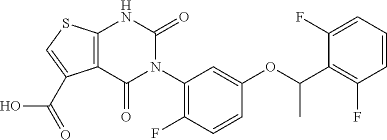 [SMILES: CC(Oc1ccc(F)c(-n2c(=O)[nH]c3scc(C(=O)O)c3c2=O)c1)c1c(F)cccc1F] | (DMSO-d₆) 1.65-1.75 (3H, m), 5.7-5.8 (1H, m), 6.95-7.2 (4H, m), 72-7.5 (2H, m), 7.93 (1H, d, J = 7.2 Hz), 12.98 (1H, s), 13.85-14.0 (1H, m) |
| 56 | 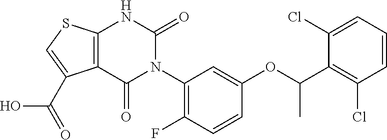 [SMILES: CC(Oc1ccc(F)c(-n2c(=O)[nH]c3scc(C(=O)O)c3c2=O)c1)c1c(Cl)cccc1Cl] | (DMSO-d₆) 1.72 (3H, d, J = 6.6 Hz), 6.0- 6.1 (1H, m), 6.85-6.95 (1H, m), 7.05-7.1 (1H, m), 7.25-7.4 (2H, m), 7.45-7.5 (2H, m), 7.92 (1H, d, J = 11.1 Hz), 12.98 (1H, brs), 13.85-14.0 (1H, m) |
| 57 | 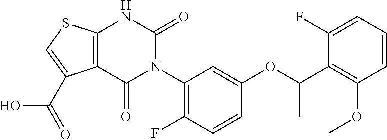 [SMILES: COc1cccc(F)c1C(C)Oc1ccc(F)c(-n2c(=O)[nH]c3scc(C(=O)O)c3c2=O)c1] | (DMSO-d₆) 1.65 (3H, d, J = 6.6 Hz), 3.8- 3.9 (3H, m), 5.79 (1H, q, J = 6.6 Hz), 6.7-6.8 (1H, m), 6.85-6.95 (2H, m), 7.0- 7.1 (1H, m), 7.2-7.35 (2H, m), 7.85-8.0 (1H, m), 12.98 (1H, brs), 13.85-14.05 (1H, m) |
| 58 | 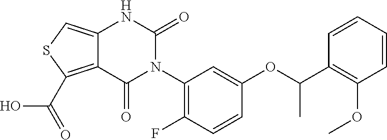 [SMILES: COc1ccccc1C(C)Oc1ccc(F)c(-n2c(=O)[nH]c3csc(C(=O)O)c3c2=O)c1] | (DMSO-d₆) 1.52 (3H, d, J = 6.3 Hz), 3.8- 3.9 (3H, m), 5.64 (1H, q, J = 6.3 Hz), 6.85-7.0 (2H, m), 7.0-7.15 (2H, m), 7.2- 7.4 (4H, m), 11.95-12.0 (1H, m), 14.41 (1H, s) |
| 59 | 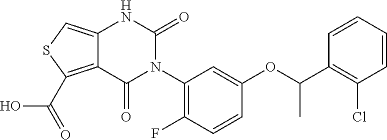 [SMILES: CC(Oc1ccc(F)c(-n2c(=O)[nH]c3csc(C(=O)O)c3c2=O)c1)c1ccccc1Cl] | (DMSO-d₆) 1.45-1.65 (3H, m), 5.55-5.8 (1H, m), 6.8-7.7 (8H, m), 11.98 (1H, s), 14.39 (1H, s) |
| 60 | 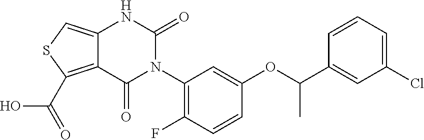 [SMILES: CC(Oc1ccc(F)c(-n2c(=O)[nH]c3csc(C(=O)O)c3c2=O)c1)c1cccc(Cl)c1] | (DMSO-d₆) 1.45-1.65 (3H, m), 5.4-5.6 (1H, m), 6.95-7.6 (8H, m), 11.99 (1H, s), 14.39 (1H, s) |
| 61 | 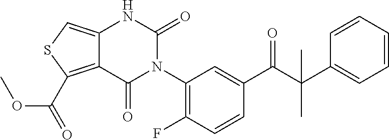 [SMILES: COC(=O)c1scc2[nH]c(=O)n(-c3cc(C(=O)C(C)(C)c4ccccc4)ccc3F)c(=O)c12] | (DMSO-d₆) 1.56 (3H, s), 1.57 (3H, s), 3.82 (3H, s), 7.15-7.45 (8H, m), 7.8-7.9 (1H, m), 11.68 (1H, s) |
| 62 | 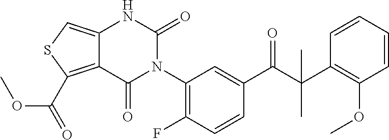 [SMILES: COC(=O)c1scc2[nH]c(=O)n(-c3cc(C(=O)C(C)(C)c4ccccc4OC)ccc3F)c(=O)c12] | (DMSO-d₆) 1.5-1.6 (6H, m), 3.34 (3H, s), 3.82 (3H, s), 6.84 (1H, d, J = 8.2 Hz), 7.0-7.1 (1H, m), 7.1-7.3 (3H, m), 7.3-7.4 (1H, m), 7.5-7.6 (1H, m), 7.8-7.9 (1H, m), 11.63 (1H, s) |
| 63 | 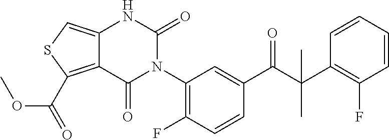 [SMILES: COC(=O)c1scc2[nH]c(=O)n(-c3cc(C(=O)C(C)(C)c4ccccc4F)ccc3F)c(=O)c12] | (DMSO-d₆) 1.59 (3H, s), 1.6 (3H, s), 3.82 (3H, s), 7.05-7.15 (1H, m), 7.19 (1H, s), 7.2-7.4 (3H, m), 7.4-7.5 (1H, m), 7.65-7.75 (1H, m), 7.9-8.0 (1H, m), 11.7 (1H, s) |
| 64 | 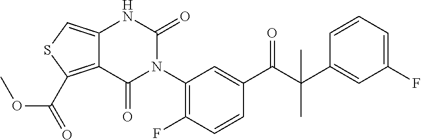 [SMILES: COC(=O)c1scc2[nH]c(=O)n(-c3cc(C(=O)C(C)(C)c4cccc(F)c4)ccc3F)c(=O)c12] | (DMSO-d₆) 1.57 (3H, s), 1.58 (3H, s), 3.82 (3H, s), 7.06 (1H, d, J = 8.4 Hz), 7.1-7.25 (3H, m), 7.25-7.35 (1H, m), 7.35-7.45 (2H, m), 7.8-7.9 (1H, m), 11.68 (1H, s) |
| 65 | 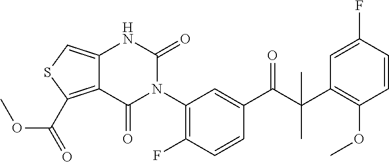 [SMILES: COC(=O)c1scc2[nH]c(=O)n(-c3cc(C(=O)C(C)(C)c4cc(F)ccc4OC)ccc3F)c(=O)c12] | (DMSO-d₆) 1.54 (6H, s), 3.31 (3H, s), 3.82 (3H, s), 6.8-6.9 (1H, m), 7.0-7.1 (1H, m), 7.15-7.25 (2H, m), 7.3-7.45 (2H, m), 7.8-7.9 (1H, m), 11.63 (1H, s) |
| 66 | 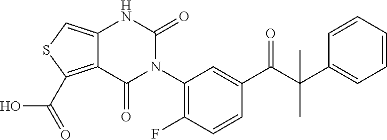 [SMILES: CC(C)(C(=O)c1ccc(F)c(-n2c(=O)[nH]c3csc(C(=O)O)c3c2=O)c1)c1ccccc1] | (DMSO-d₆) 1.55-1.6 (6H, m), 7.25-7.45 (8H, m), 7.9-8.0 (1H, m), 12.0 (1H, s), 14.29 (1H, s) |
| 67 | 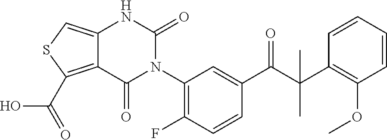 [SMILES: COc1ccccc1C(C)(C)C(=O)c1ccc(F)c(-n2c(=O)[nH]c3csc(C(=O)O)c3c2=O)c1] | (DMSO-d₆) 1.55 (3H, s), 1.56 (3H, s), 3.33 (3H, s), 6.84 (1H, d, J = 8.2 Hz), 7.0-7.1 (1H, m), 7.15-7.3 (2H, m), 7.37 (1H, s), 7.4-7.5 (1H, m), 7.5-7.55 (1H, m), 7.9-7.95 (1H, m), 11.99 (1H, s), 14.35 (1H, s) |
| 68 | 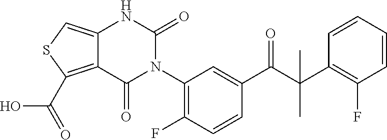 [SMILES: CC(C)(C(=O)c1ccc(F)c(-n2c(=O)[nH]c3csc(C(=O)O)c3c2=O)c1)c1ccccc1F] | (DMSO-d₆) 1.59 (3H, s), 1.62 (3H, s), 7.0-7.1 (1H, m), 7.25-7.4 (4H, m), 7.5- 7.6 (1H, m), 7.65-7.75 (1H, m), 7.95- 8.05 (1H, m), 12.01 (1H, s), 14.29 (1H, s) |
| 69 | 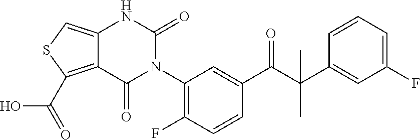 [SMILES: CC(C)(C(=O)c1ccc(F)c(-n2c(=O)[nH]c3csc(C(=O)O)c3c2=O)c1)c1cccc(F)c1] | (DMSO-d₆) 1.58 (3H, s), 1.59 (3H, s), 7.0-7.25 (3H, m), 7.3-7.45 (3H, m), 7.45-7.55 (1H, m), 7.9-7.95 (1H, m), 12.02 (1H, s), 14.29 (1H, s) |
| 70 | 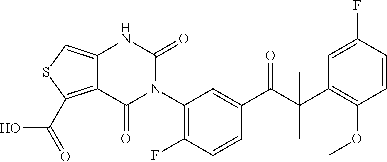 [SMILES: COc1ccc(F)cc1C(C)(C)C(=O)c1ccc(F)c(-n2c(=O)[nH]c3csc(C(=O)O)c3c2=O)c1] | (DMSO-d₆) 1.54 (3H, s), 1.56 (3H, s), 3.31 (3H, s), 6.8-6.9 (1H, m), 7.0-7.1 (1H, m), 7.26 (1H, t, J = 9.2 Mz), 7.3-7.4 (2H, m), 7.5-7.6 (1H, m), 7.85-7.95 (1H, m), 11.99 (1H, s), 14.36 (1H, s) |
| 71 | 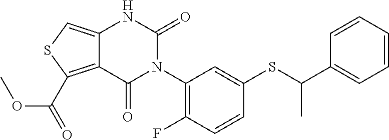 [SMILES: COC(=O)c1scc2[nH]c(=O)n(-c3cc(SC(C)c4ccccc4)ccc3F)c(=O)c12] | (DMSO-d₆) 1.55-1.6 (3H, m), 3.82 (3H, s), 4.6-4.7 (1H, m), 7.1-7.2 (2H, m), 7.2- 7.45 (8H, m), 11.49 (1H, s) |
| 72 | 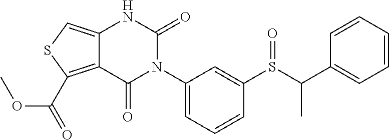 [SMILES: COC(=O)c1scc2[nH]c(=O)n(-c3cccc(S(=O)C(C)c4ccccc4)c3)c(=O)c12] | (DMSO-d₆) 1.36 (3H, d, J = 7.2 Hz), 3.82 (3H, s), 4.05-4.15 (1H, m), 7.15- 7.65 (10H, m), 11.54 (1H, s) |
| 73 | 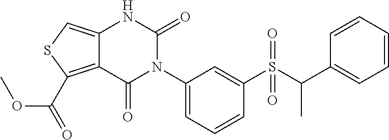 [SMILES: COC(=O)c1scc2[nH]c(=O)n(-c3cccc(S(=O)(=O)C(C)c4ccccc4)c3)c(=O)c12] | (DMSO-d₆) 1.56 (3H, d, J = 7.1 Hz), 3.83 (3H, s), 4.71 (1H, q, J = 7.1 Hz), 7.18 (1H, s), 7.2-7.35 (5H, m), 7.6-7.7 (3H, m), 7.75-7.8 (1H, m), 11.56 (1H, s) |
| 74 | 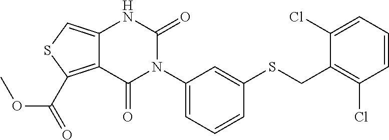 [SMILES: COC(=O)c1scc2[nH]c(=O)n(-c3cccc(SCc4c(Cl)cccc4Cl)c3)c(=O)c12] | (DMSO-d₆) 3.82 (3H, s), 4.44 (2H, s), 7.1-7.25 (2H, m), 7.3-7.4 (1H, m), 7.4- 7.45 (3H, m), 7.45-7.55 (2H, m), 11.5 (1H, s) |
| 75 | 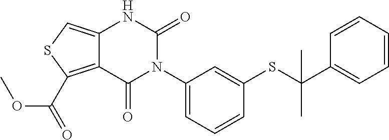 [SMILES: COC(=O)c1scc2[nH]c(=O)n(-c3cccc(SC(C)(C)c4ccccc4)c3)c(=O)c12] | (DMSO-d₆) 1.64 (6H, s), 3.82 (3H, s), 7.05-7.25 (4H, m), 7.25-7.4 (4H, m), 7.4-7.5 (2H, m), 11.45 (1H, s) |
| 76 | 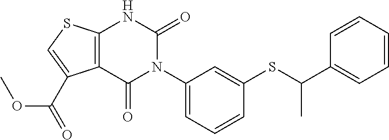 [SMILES: COC(=O)c1csc2[nH]c(=O)n(-c3cccc(SC(C)c4ccccc4)c3)c(=O)c12] | (DMSO-d₆) 1.57 (3H, d, J = 6.9 Hz), 3.74 (3H, s), 4.66 (1H, q, J = 6.9 Hz), 7.1-7.15 (1H, m), 7.2-7.45 (8H, m), 7.59 (1H, s), 12.44 (1H, s) |
| 77 | 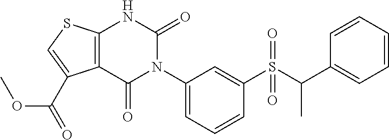 [SMILES: COC(=O)c1csc2[nH]c(=O)n(-c3cccc(S(=O)(=O)C(C)c4ccccc4)c3)c(=O)c12] | (DMSO-d₆) 1.56 (3H, d, J = 6.8 Hz), 3.76 (3H, s), 4.65-4.75 (1H, m), 7.2-7.35 (5H, m), 7.6-7.75 (4H, m), 7.78 (1H, s), 12.52 (1H, s) |
| 78 | 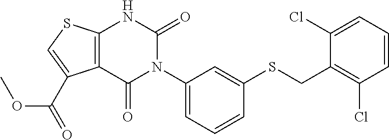 [SMILES: COC(=O)c1csc2[nH]c(=O)n(-c3cccc(SCc4c(Cl)cccc4Cl)c3)c(=O)c12] | (DMSO-d₆) 3.74 (3H, s), 4.44 (2H, s), 7.15-7.25 (1H, m), 7.3-7.55 (6H, m), 7.59 (1H, s), 12.45 (1H, s) |
| 79 | 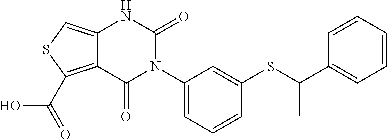 [SMILES: CC(Sc1cccc(-n2c(=O)[nH]c3csc(C(=O)O)c3c2=O)c1)c1ccccc1] | (DMSO-d₆) 1.58 (3H, d, J = 6.9 Hz), 4.65 (1H, q, J = 6.9 Hz), 7.2-7.5 (10H, m), 11.93 (1H, s), 14.87 (1H, s) |
| 80 | 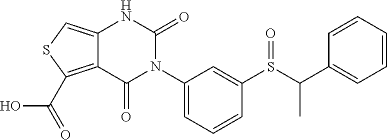 [SMILES: CC(c1ccccc1)S(=O)c1cccc(-n2c(=O)[nH]c3csc(C(=O)O)c3c2=O)c1] | (DMSO-d₆) 1.39 (3H, d, J = 7.3 Hz), 4.05-4.15 (1H, m), 7.2-7.8 (10H, m), 11.95 (1H, s), 14.8 (1H, s) |
| 81 | 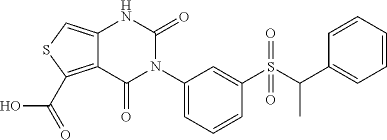 [SMILES: CC(c1ccccc1)S(=O)(=O)c1cccc(-n2c(=O)[nH]c3csc(C(=O)O)c3c2=O)c1] | (DMSO-d₆) 1.59 (3H, d, J = 7.7 Hz), 4.72 (1H, q, J = 7.7 Hz), 7.2-7.35 (5H, m), 7.39 (1H, s), 7.65-7.9 (4H, m), 11.96 (1H, s), 14.73 (1H, s) |
| 82 | 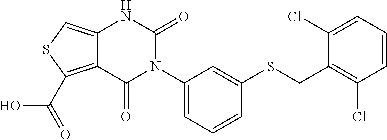 [SMILES: O=C(O)c1scc2[nH]c(=O)n(-c3cccc(SCc4c(Cl)cccc4Cl)c3)c(=O)c12] | (DMSO-d₆) 4.44 (2H, s), 7.25-7.45 (3H, m), 7.45-7.55 (5H, m), 11.93 (1H, s), 14.87 (1H, s) |
| 83 | 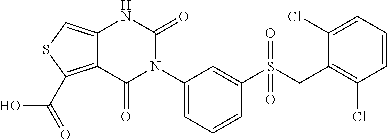 [SMILES: O=C(O)c1scc2[nH]c(=O)n(-c3cccc(S(=O)(=O)Cc4c(Cl)cccc4Cl)c3)c(=O)c12] | (DMSO-d₆) 4.94 (2H, s), 7.35-7.55 (4H, m), 7.75-7.85 (3H, m), 7.95-8.0 (1H, m), 11.96 (1H, s), 14.75 (1H, s) |
| 84 | 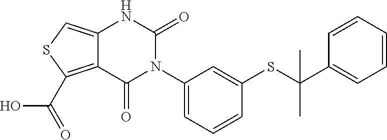 [SMILES: CC(C)(Sc1cccc(-n2c(=O)[nH]c3csc(C(=O)O)c3c2=O)c1)c1ccccc1] | (DMSO-d₆) 1.65 (6H, s), 7.1-7.5 (10H, m), 11.88 (1H, s), 14.84 (1H, s) |
| 85 | 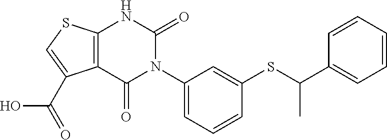 [SMILES: CC(Sc1cccc(-n2c(=O)[nH]c3scc(C(=O)O)c3c2=O)c1)c1ccccc1] | (DMSO-d₆) 1.58 (3H, d, J = 7.0 Hz), 4.66 (1H, q, J = 7.0 Hz), 7.15-7.45 (9H, m), 8.0 (1H, s), 12.94 (1H, s), 14.43 (1H, s) |
| 86 | 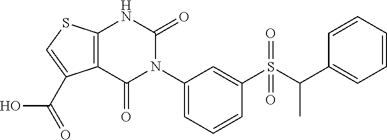 [SMILES: CC(c1ccccc1)S(=O)(=O)c1cccc(-n2c(=O)[nH]c3scc(C(=O)O)c3c2=O)c1] | (DMSO-d₆) 1.58 (3H, d, J = 7.0 Hz), 4.72 (1H, q, J = 7.0 Hz), 7.2-7.35 (5H, m), 7.65-7.8 (3H, m), 7.84 (1H, s), 7.98 (1H, s), 12.96 (1H, s), 14.26 (1H, s) |
| 87 | 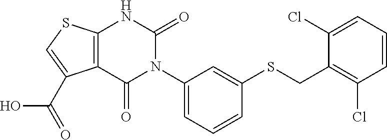 [SMILES: O=C(O)c1csc2[nH]c(=O)n(-c3cccc(SCc4c(Cl)cccc4Cl)c3)c(=O)c12] | (DMSO-d₆) 4.45 (2H, s), 7.25-7.3 (1H, m), 7.3-7.4 (1H, m), 7.45-7.55 (5H, m), 8.0 (1H, s), 12.94 (1H, s), 14.42 (1H, s) |
| 88 | 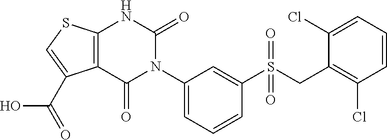 [SMILES: O=C(O)c1csc2[nH]c(=O)n(-c3cccc(S(=O)(=O)Cc4c(Cl)cccc4Cl)c3)c(=O)c12] | (DMSO-d₆) 4.94 (2H, s), 7.35-7.45 (1H, m), 7.45-7.55 (2H, m), 7.7-7.85 (3H, m), 7.95-8.0 (2H, m), 12.96 (1H, s), 14.3 (1H, s) |
| 89 | 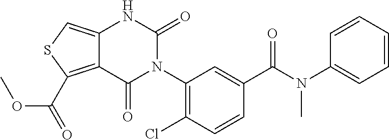 [SMILES: COC(=O)c1scc2[nH]c(=O)n(-c3cc(C(=O)N(C)c4ccccc4)ccc3Cl)c(=O)c12] | (DMSO-d₆) 3.37 (3H, s), 3.83 (3H, s), 7.15-7.25 (5H, m), 7.25-7.35 (2H, m), 7.44 (1H, d, J = 8.1 Hz), 7.56 (1H, d, J = 1.9 Hz), 11.63 (1H, s) |
| 90 | 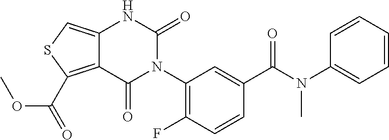 [SMILES: COC(=O)c1scc2[nH]c(=O)n(-c3cc(C(=O)N(C)c4ccccc4)ccc3F)c(=O)c12] | (DMSO-d₆) 3.37 (3H, s), 3.83 (3H, s), 7.15-7.35 (8H, m), 7.53 (1H, dd, J = 7.3 Hz, 1.9 Hz), 11.63 (1H, s) |
| 91 | 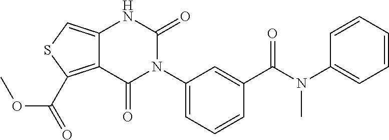 [SMILES: COC(=O)c1scc2[nH]c(=O)n(-c3cccc(C(=O)N(C)c4ccccc4)c3)c(=O)c12] | (DMSO-d₆) 3.37 (3H, s), 3.82 (3H, s), 7.1-7.4 10H, m), 11.46 (1H, s) |
| 92 | 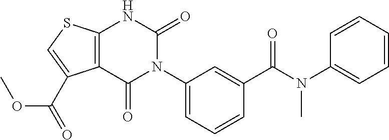 [SMILES: COC(=O)c1csc2[nH]c(=O)n(-c3cccc(C(=O)N(C)c4ccccc4)c3)c(=O)c12] | (DMSO-d₆) 3.37 (3H, s), 3.74 (3H, s), 7.1-7.4 (9H, m), 7.58 (1H, s), 12.42 (1H, s) |
| 93 | 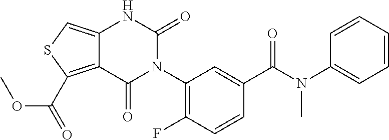 [SMILES: COC(=O)c1scc2[nH]c(=O)n(-c3cc(C(=O)N(C)c4ccccc4)ccc3F)c(=O)c12] | (DMSO-d₆) 3.38 (3H, s), 7.15-7.4 (8H, m), 7.5-7.6 (1H, m), 11.96 (1H, s), 14.34 (1H, s) |
| 94 | 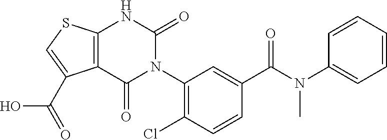 [SMILES: CN(C(=O)c1ccc(Cl)c(-n2c(=O)[nH]c3scc(C(=O)O)c3c2=O)c1)c1ccccc1] | (DMSO-d₆) 3.37 (3H, s), 7.15-7.4 (7H, m), 7.44 (1H, d, J = 8.7 Hz), 7.72 (1H, s) |
| 95 | 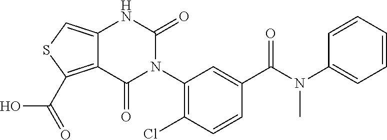 [SMILES: CN(C(=O)c1ccc(Cl)c(-n2c(=O)[nH]c3csc(C(=O)O)c3c2=O)c1)c1ccccc1] | (DMSO-d₆) 3.38 (3H, s), 7.15-7.25 (3H, m), 7.25-7.35 (3H, m), 7.38 (1H, s), 7.5 (1H, d, J = 7.5 Hz), 7.58 (1H, d, J = 1.9 Hz), 11.98 (1H, s), 14.33 (1H, s) |
| 96 | 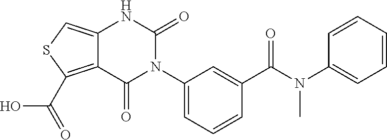 [SMILES: CN(C(=O)c1cccc(-n2c(=O)[nH]c3csc(C(=O)O)c3c2=O)c1)c1ccccc1] | (DMSO-d₆) 3.37 (3H, s), 7.15-7.4 (10H, m), 11.89 (1H, s), 14.81 (1H, s) |
| 97 | 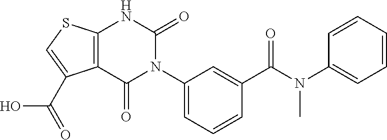 [SMILES: CN(C(=O)c1cccc(-n2c(=O)[nH]c3scc(C(=O)O)c3c2=O)c1)c1ccccc1] | (DMSO-d₆) 3.37 (3H, s), 7.15-7.4 (9H, m), 7.99 (1H, s), 12.9 (1H, s), 14.37 (1H, s) |
| 98 | 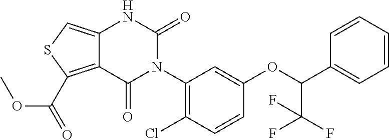 [SMILES: COC(=O)c1scc2[nH]c(=O)n(-c3cc(OC(c4ccccc4)C(F)(F)F)ccc3Cl)c(=O)c12] | (DMSO-d₆) 3.75-3.85 (3H, m), 6.3-6.4 (1H, m), 7.05-7.15 (1H, m), 7.2 (1H, s), 7.32 (1H, dd, J = 7.6 Hz, 2.9 Hz), 7.45- 7.55 (4H, m), 7.55-7.65 (2H, m), 11.66 (1H, s) |
| 99 | 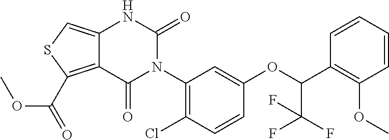 | (DMSO-d₆) 3.75-3.95 (6H, m), 6.2-6.35 (1H, m), 6.9-7.0 (1H, m), 7.0-7.1 (1H, m), 7.17 (1H, d, J = 8.0 Hz), 7.21 (1H, s), 7.31 (1H, dd, J = 6.4 Hz, 3.0 Hz), 7.4-7.55 (3H, m), 11.64 (1H, s) |
| 100 | 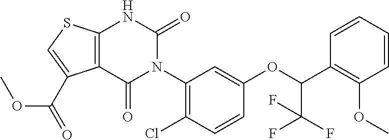 | (DMSO-d₆) 3.7-3.75 (3H, m), 3.85-3.95 (3H, m), 6.2-6.35 (1H, m), 6.9-7.0 (1H, m), 7.04 (1H, t, J = 7.6 Hz), 7.15-7.2 (1H, m), 7.25-7.35 (1H, m), 7.4-7.55 (3H, m), 7.64 (1H, s), 12.6 (1H, s) |
| 101 | 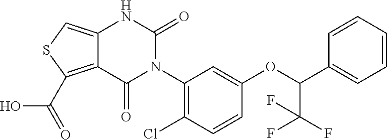 [SMILES: O=C(O)c1scc2[nH]c(=O)n(-c3cc(OC(c4ccccc4)C(F)(F)F)ccc3Cl)c(=O)c12] | (DMSO-d₆) 6.3-6.4 (1H, m), 7.1-7.2 (1H, m), 7.35-7.4 (2H, m), 7.45-7.55 (3H, m), 7.55-7.65 (3H, m), 11.95-12.1 (1H, m), 14.32 (1H, s) |
| 102 | 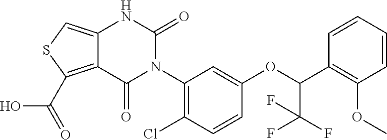 [SMILES: COc1ccccc1C(Oc1ccc(Cl)c(-n2c(=O)[nH]c3csc(C(=O)O)c3c2=O)c1)C(F)(F)F] | (DMSO-d₆) 3.85-3.95 (3H, m), 6.25-6.35 (1H, m), 6.95-7.1 (2H, m), 7.15-7.2 (1H, m), 7.35-7.5 (4H, m), 7.56 (1H, d, J = 8.9 Hz), 12.0-12.1 (1H, m), 14.34 (1H, s) |
| 103 | 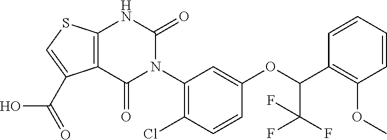 [SMILES: COc1ccccc1C(Oc1ccc(Cl)c(-n2c(=O)[nH]c3scc(C(=O)O)c3c2=O)c1)C(F)(F)F] | (DMSO-d₆) 3.85-3.95 (3H, m), 6.25-6.35 (1H, m), 6.95-7.1 (2H, m), 7.15-7.2 (1H, m), 7.3-7.4 (1H, m), 7.4-7.5 (2H, m), 7.55 (1H, d, J = 9.0 Hz), 7.92 (1H, d, J = 4.5 Hz), 13.0 (1H, brs), 13.8-13.95 (1H, m) |
| 104 | 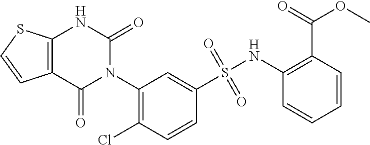 [SMILES: COC(=O)c1ccccc1NS(=O)(=O)c1ccc(Cl)c(-n2c(=O)[nH]c3sccc3c2=O)c1] | (CDCl3) 3.89 (3H, s), 6.84 (1H, d, J = 5.8 Hz), 7.05-7.1 (1H, m), 7.28 (1H, d, J = 5.8 Hz), 7.45-7.55 (1H, m), 7.59 (1H, d, J = 8.5 Hz), 7.65-7.75 (1H, m), 7.8- 7.85 (1H, m), 7.85-8.0 (2H, m), 10.06 (1H, s), 10.75 (1H, s) |
| 105 | 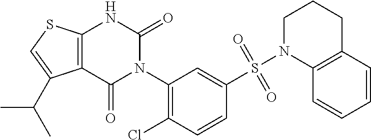 [SMILES: CC(C)c1csc2[nH]c(=O)n(-c3cc(S(=O)(=O)N4CCCc5ccccc54)ccc3Cl)c(=O)c12] | (CDCl3) 1.6-1.75 (2H, m), 2.45-2.55 (2H, m), 3.45-3.6 (1H, m), 3.7-3.85 (2H, m), 6.45 (1H, d, J = 0.5 Hz), 7.01 (1H, d, J = 7.0 Hz), 7.05-7.25 (2H, m), 7.5-7.6 (3H, m), 7.76 (1H, d, J = 7.7 Hz), 10.68 (1H, s) |
| 106 | 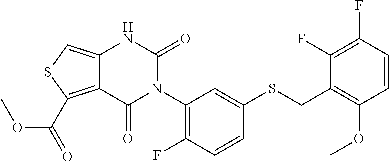 [SMILES: COC(=O)c1scc2[nH]c(=O)n(-c3cc(SCc4c(OC)ccc(F)c4F)ccc3F)c(=O)c12] | (DMSO-d6) 3.74 (3H, s), 3.83 (3H, s), 4.15-4.2 (2H, m), 6.75-6.85 (1H, m), 7.21 (1H, s), 7.25-7.4 (2H, m), 7.4-7.5 (1H, m), 7.5-7.55 (1H, m), 11.65 (1H, s) |
| 107 | 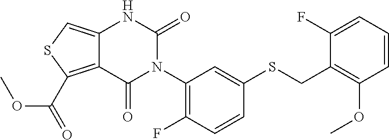 [SMILES: COC(=O)c1scc2[nH]c(=O)n(-c3cc(SCc4c(F)cccc4OC)ccc3F)c(=O)c12] | (DMSO-d6) 3.77 (3H, s), 3.83 (3H, s), 4.15 (2H, s), 6.75-6.9 (2H, m), 7.15-7.55 (5H, m), 11.65 (1H, s) |
| 108 | 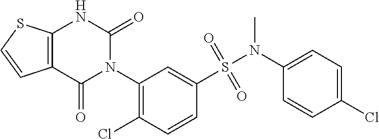 [SMILES: CN(c1ccc(Cl)cc1)S(=O)(=O)c1ccc(Cl)c(-n2c(=O)[nH]c3sccc3c2=O)c1] | (CDCl3) 3.16 (3H, s), 6.88 (1H, d, J = 5.8 Hz), 7.0-7.1 (2H, m), 7.25-7.35 (3H, m), 7.5-7.6 (2H, m), 7.6-7.7 (1H, m), 9.5-11.0 (1H, br) |
| 109 | 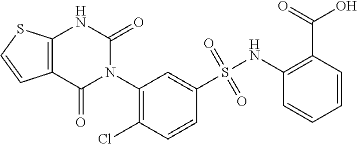 [SMILES: O=C(O)c1ccccc1NS(=O)(=O)c1ccc(Cl)c(-n2c(=O)[nH]c3sccc3c2=O)c1] | (DMSO-d6) 7.1-7.25 (3H, m), 7.44 (1H, d, J = 8.5 Hz), 7.5-7.6 (1H, m), 7.87 (1H, d, J = 8.5 Hz), 7.9-8.0 (2H, m), 8.18 (1H, d, J = 2.2 Hz), 11.0-12.0 (1H, br), 12.52 (1H, s) |
| 110 | 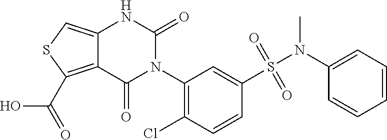 [SMILES: CN(c1ccccc1)S(=O)(=O)c1ccc(Cl)c(-n2c(=O)[nH]c3csc(C(=O)O)c3c2=O)c1] | (CDCl3) 3.22 (3H, s), 7.05-7.15 (3H, m), 7.25-7.35 (3H, m), 7.45-7.55 (1H, m), 7.71 (1H, s), 9.22 (1H, s), 14.14 (1H, s) |
| 111 | 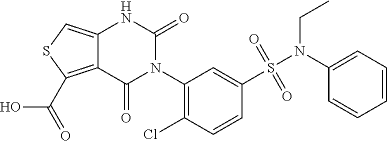 [SMILES: CCN(c1ccccc1)S(=O)(=O)c1ccc(Cl)c(-n2c(=O)[nH]c3csc(C(=O)O)c3c2=O)c1] | (CDCl3) 1.11 (3H, t, J = 7.1 Hz), 3.5-3.6 (1H, m), 3.65-3.8 (1H, m), 7.05-7.1 (2H, m), 7.13 (1H, s), 7.25-7.35 (3H, m), 7.55 (1H, d, J = 2.2 Hz), 7.7 (1H, d, J = 8.4 Hz), 7.77 (1H, dd, J = 8.4 Hz, 2.2 Hz), 9.22 (1H, s), 14.17 (1H, s) |
| 112 | 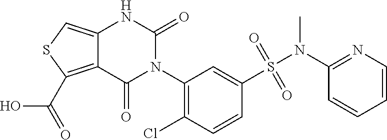 [SMILES: CN(c1ccccn1)S(=O)(=O)c1ccc(Cl)c(-n2c(=O)[nH]c3csc(C(=O)O)c3c2=O)c1] | (CDCl3) 3.33 (3H, s), 7.1 (1H, s), 7.15- 7.2 (1H, m), 7.57 (1H, d, J = 8.2 Hz), 7.6-7.75 (4H, m), 8.3-8.4 (1H, m), 9.05 (1H, s), 14.09 (1H, s) |
| 113 | 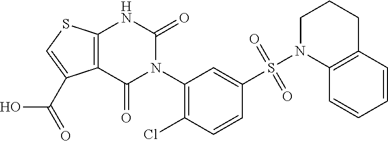 [SMILES: O=C(O)c1csc2[nH]c(=O)n(-c3cc(S(=O)(=O)N4CCCc5ccccc54)ccc3Cl)c(=O)c12] | (DMSO-d6) 1.6-1.75 (2H, m), 2.45-2.55 (2H, m), 3.7-3.85 (2H, m), 6.9-7.0 (1H, m), 7.05-7.25 (3H, m), 7.5-7.6 (2H, m), 7.78 (1H, d, J = 8.5 Hz), 8.0-8.05 (1H, m), 8.5-8.55 (1H, m), 11.52 (1H, s) |
| 114 | 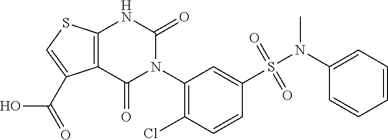 [SMILES: CN(c1ccccc1)S(=O)(=O)c1ccc(Cl)c(-n2c(=O)[nH]c3scc(C(=O)O)c3c2=O)c1] | (DMSO-d6) 3.17 (3H, s), 7.05-7.15 (2H, m), 7.25-7.4 (3H, m), 7.63 (1H, dd, J = 8.6 Hz, 2.2 Hz), 7.85-7.95 (3H, m), 12.5- 13.5 (1H, br), 13.79 (1H, s) |
| 115 | 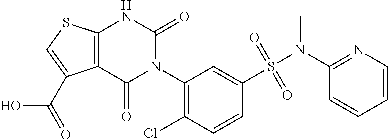 [SMILES: CN(c1ccccn1)S(=O)(=O)c1ccc(Cl)c(-n2c(=O)[nH]c3scc(C(=O)O)c3c2=O)c1] | (DMSO-d6) 3.26 (3H, s), 7.25-7.3 (1H, m), 7.45-7.55 (1H, m), 7.65-7.75 (1H, m), 7.8-7.95 (3H, m), 8.05-8.1 (1H, m), 8.3-8.4 (1H, m) |
| 116 | 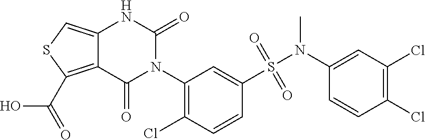 [SMILES: CN(c1ccc(Cl)c(Cl)c1)S(=O)(=O)c1ccc(Cl)c(-n2c(=O)[nH]c3csc(C(=O)O)c3c2=O)c1] | (CDCl3) 3.17 (3H, s), 6.9-7.0 (1H, m), 7.13 (1H, s), 7.29 (1H, d, J = 2.5 Hz), 7.39 (1H, d, J = 8.4 Hz), 7.51 (1H, d, J = 1.2 Hz), 7.7-7.8 (2H, m), 9.12 (1H, s), 14.05 (1H, s) |
| 117 | 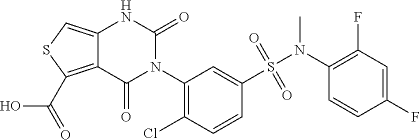 [SMILES: CN(c1ccc(F)cc1F)S(=O)(=O)c1ccc(Cl)c(-n2c(=O)[nH]c3csc(C(=O)O)c3c2=O)c1] | (CDCl3) 3.25 (3H, s), 6.75-6.9 (2H, m), 7.13 (1H, s), 7.2-7.3 (1H, m), 7.63 (1H, d, J = 2.1 Hz), 7.75 (1H, d, J = 8.6 Hz), 7.8-7.85 (1H, m), 9.03 (1H, s), 14.11 (1H, s) |
| 118 | 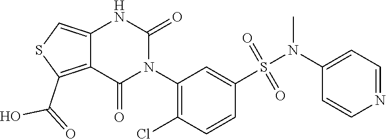 [SMILES: CN(c1ccncc1)S(=O)(=O)c1ccc(Cl)c(-n2c(=O)[nH]c3csc(C(=O)O)c3c2=O)c1] | (CDCl3) 3.22 (3H, s), 7.08 (1H, s), 7.15- 7.25 (2H, m), 7.38 (1H, d, J = 1.7 Hz), 7.7-7.8 (2H, m), 8.55-8.65 (2H, m) |
| 119 | 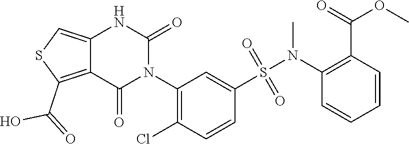 [SMILES: COC(=O)c1ccccc1N(C)S(=O)(=O)c1ccc(Cl)c(-n2c(=O)[nH]c3csc(C(=O)O)c3c2=O)c1] | (DMSO-d6) 3.23 (3H, s), 3.8 (3H, s), 6.9-6.95 (1H, m), 7.35-7.55 (3H, m), 7.7-7.85 (2H, m), 7.9-8.0 (2H, m), 12.03 (1H, s), 14.29 (1H, s) |
| 120 | 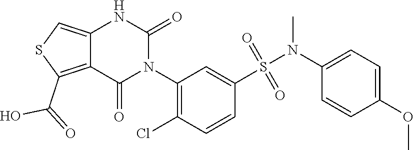 [SMILES: COc1ccc(N(C)S(=O)(=O)c2ccc(Cl)c(-n3c(=O)[nH]c4csc(C(=O)O)c4c3=O)c2)cc1] | (DMSO-d6) 3.12 (3H, s), 3.73 (3H, s), 6.85-6.9 (2H, m), 6.95-7.0 (2H, m), 7.39 (1H, s), 7.68 (1H, dd, J = 8.4 Hz, 2.3 Hz), 7.85-7.95 (2H, m), 12.03 (1H, s), 14.3 (1H, s) |
| 121 | 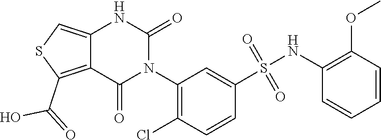 [SMILES: COc1ccccc1NS(=O)(=O)c1ccc(Cl)c(-n2c(=O)[nH]c3csc(C(=O)O)c3c2=O)c1] | (DMSO-d6) 3.51 (3H, s), 6.85-6.95 (2H, m), 7.15-7.25 (2H, m), 7.38 (1H, s), 7.7- 7.8 (1H, m), 7.85-7.95 (1H, m), 7.95-8.0 (1H, m), 9.74 (1H, s), 12.02 (1H, s), 13.5-15.0 (1H, br) |
| 122 | 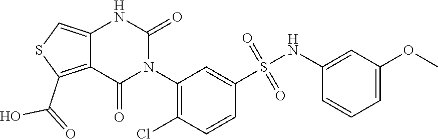 [SMILES: COc1cccc(NS(=O)(=O)c2ccc(Cl)c(-n3c(=O)[nH]c4csc(C(=O)O)c4c3=O)c2)c1] | (DMSO-d6) 3.67 (3H, s), 6.6-6.75 (3H, m), 7.1-7.2 (1H, m), 7.38 (1H, s), 7.84 (1H, dd, J = 8.5 Hz, 2.2 Hz), 7.89 (1H, d, J = 8.5 Hz), 8.13 (1H, d, J = 2.2 Hz), 10.49 (1H, s), 12.04 (1H, s), 14.22 (1H, s) |
| 123 | 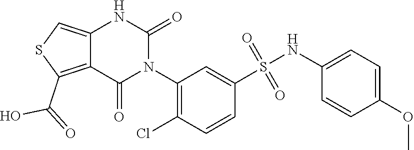 [SMILES: COc1ccc(NS(=O)(=O)c2ccc(Cl)c(-n3c(=O)[nH]c4csc(C(=O)O)c4c3=O)c2)cc1] | (DMSO-d6) 3.69 (3H, s), 6.83 (2H, d, J = 8.9 Hz), 6.99 (2H, d, J = 8.9 Hz), 7.38 (1H, s), 7.75 (1H, dd, J = 8.5 Hz, 2.0 Hz), 7.88 (1H, d, J = 8.5 Hz), 7.99 (1H, d, J = 2.0 Hz), 10.08 (1H, s), 12.03 (1H, s), 14.24 (1H, s) |
| 124 | 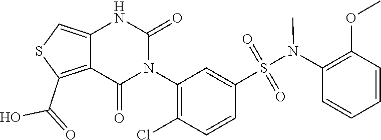 [SMILES: COc1ccccc1N(C)S(=O)(=O)c1ccc(Cl)c(-n2c(=O)[nH]c3csc(C(=O)O)c3c2=O)c1] | (DMSO-d6) 3.17 (3H, s), 3.47 (3H, s), 6.9-7.0 (2H, m), 7.15-7.2 (1H, m), 7.25- 7.35 (1H, m), 7.39 (1H, s), 7.71 (1H, dd, J = 8.6 Hz, 2.2 Hz), 7.9 (1H, d, J = 8.6 Hz), 8.02 (1H, d, J = 2.2 Hz), 12.02 (1H, s), 14.31 (1H, s) |
| 125 | 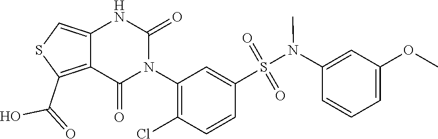 [SMILES: COc1cccc(N(C)S(=O)(=O)c2ccc(Cl)c(-n3c(=O)[nH]c4csc(C(=O)O)c4c3=O)c2)c1] | (DMSO-d6) 3.18 (3H, s), 3.69 (3H, s), 6.55-6.7 (2H, m), 6.8-6.9 (1H, m), 7.2- 7.3 (1H, m), 7.35-7.4 (1H, m), 7.6-7.7 (1H, m), 7.85-8.05 (2H, m), 12.04 (1H, s), 14.26 (1H, s) |
| 126 | 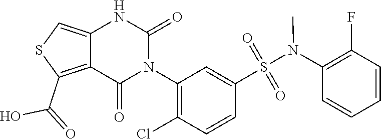 [SMILES: CN(c1ccccc1F)S(=O)(=O)c1ccc(Cl)c(-n2c(=O)[nH]c3csc(C(=O)O)c3c2=O)c1] | (DMSO-d6) 3.18 (3H, s), 7.1-7.35 (3H, m), 7.35-7.45 (2H, m), 7.81 (1H, d, J = 2.3 Hz), 7.95 (1H, d, J = 8.5 Hz), 8.03 (1H, d, J = 2.3 Hz), 12.03 (1H, s), 14.29 (1H, s) |
| 127 | 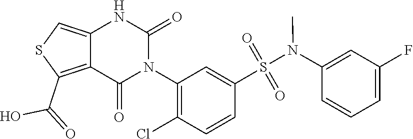 [SMILES: CN(c1cccc(F)c1)S(=O)(=O)c1ccc(Cl)c(-n2c(=O)[nH]c3csc(C(=O)O)c3c2=O)c1] | (DMSO-d6) 3.19 (3H, s), 6.95-7.05 (2H, m), 7.1-7.2 (1H, m), 7.35-7.45 (2H, m), 7.7-7.75 (1H, m), 7.9-8.0 (2H, m), 12.04 (1H, s), 14.27 (1H, s) |
| 128 | 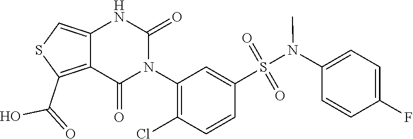 [SMILES: CN(c1ccc(F)cc1)S(=O)(=O)c1ccc(Cl)c(-n2c(=O)[nH]c3csc(C(=O)O)c3c2=O)c1] | (DMSO-d6) 3.15 (3H, s), 7.05-7.25 (4H, m), 7.38 (1H, s), 7.69 (1H, dd, J = 8.5 Hz, 2.5 Hz), 7.9 (1H. d, J = 2.5 Hz), 7.93 (1H, d, J = 8.5 Hz), 12.05 (1H, s), 14.29 (1H, s) |
| 129 | 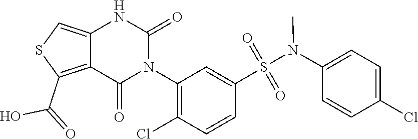 [SMILES: CN(c1ccc(Cl)cc1)S(=O)(=O)c1ccc(Cl)c(-n2c(=O)[nH]c3csc(C(=O)O)c3c2=O)c1] | (DMSO-d6) 3.15 (3H, s), 7.1-7.15 (2H, m), 7.35-7.45 (3H, m), 7.7 (1H, dd, J = 8.6 Hz, 2.1 Hz), 7.9-7.95 (2H, m), 12.05 (1H, s), 14.28 (1H, s) |
| 130 | 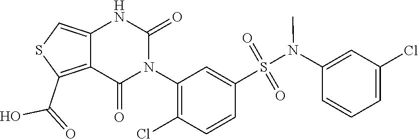 [SMILES: CN(c1cccc(Cl)c1)S(=O)(=O)c1ccc(Cl)c(-n2c(=O)[nH]c3csc(C(=O)O)c3c2=O)c1] | (DMSO-d6) 3.19 (3H, s), 7.05-7.1 (1H, m), 7.2-7.25 (1H, m), 7.35-7.45 (3H, m), 7.72 (1H, dd, J = 8.5 Hz, 2.1 Hz), 7.9- 8.0 (2H, m), 12.06 (1H, s), 14.3 (1H, s) |
| 131 | 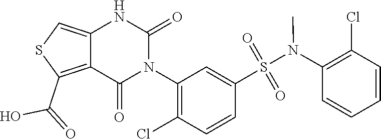 [SMILES: CN(c1ccccc1Cl)S(=O)(=O)c1ccc(Cl)c(-n2c(=O)[nH]c3csc(C(=O)O)c3c2=O)c1] | (DMSO-d6) 3.16 (3H, s), 7.0-7.1 (1H, m), 7.25-7.45 (3H, m), 7.55-7.6 (1H, m), 7.85-7.9 (1H, m), 7.97 (1H, d, J = 8.5 Hz), 8.1 (1H, s), 12.06 (1H, s), 14.31 (1H, s) |
| 132 | 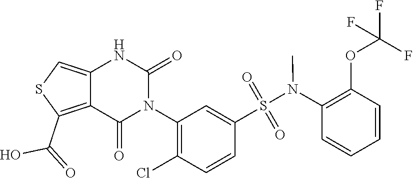 [SMILES: CN(c1ccccc1OC(F)(F)F)S(=O)(=O)c1ccc(Cl)c(-n2c(=O)[nH]c3csc(C(=O)O)c3c2=O)c1] | (DMSO-d6) 3.13 (3H, s), 7.0-7.1 (1H, m), 7.3-7.4 (2H, m), 7.45-7.55 (2H, m), 7.86 (1H, dd, J = 8.5 Hz, 2.4 Hz), 7.98 (1H, d, J = 8.5 Hz), 8.02 (1H, d, J = 2.4 Hz), 12.03 (1H, s), 14.3 (1H, s) |
| 133 | 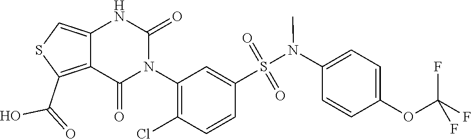 [SMILES: CN(c1ccc(OC(F)(F)F)cc1)S(=O)(=O)c1ccc(Cl)c(-n2c(=O)[nH]c3csc(C(=O)O)c3c2=O)c1] | (DMSO-d6) 3.17 (3H, s), 7.2-7.3 (2H, m), 7.3-7.4 (3H, m), 7.71 (1H, dd, J = 8.5 Hz, 2.1 Hz), 7.9 (1H, d, J = 2.1 Hz), 7.93 (1H, d, J = 8.5 Hz), 12.0 (1H, s), 14.25 (1H, s) |
| 134 | 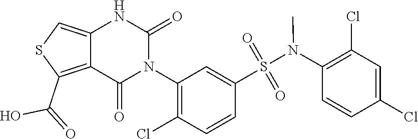 [SMILES: CN(c1ccc(Cl)cc1Cl)S(=O)(=O)c1ccc(Cl)c(-n2c(=O)[nH]c3csc(C(=O)O)c3c2=O)c1] | (DMSO-d6) 3.15 (3H, s), 7.06 (1H, d, J = 8.5 Hz), 7.35-7.45 (2H, m), 7.76 (1H, d, J = 2.4 Hz), 7.85-7.9 (1H, m), 7.97 (1H, d, J = 8.54 Hz), 8.07 (1H, d, J = 2.2 Hz), 12.03 (1H, s), 14.27 (1H, s) |
| 135 | 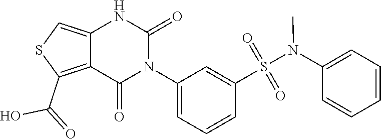 [SMILES: CN(c1ccccc1)S(=O)(=O)c1cccc(-n2c(=O)[nH]c3csc(C(=O)O)c3c2=O)c1] | (DMSO-d6) 3.16 (3H, s), 7.05-7.1 (2H, m), 7.25-7.4 (4H, m), 7.55-7.65 (1H, m), 7.7-7.8 (3H, m), 11.95 (1H, s), 14.77 (1H, s) |
| 136 | 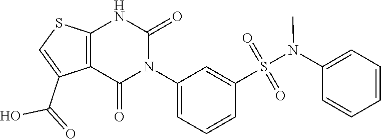 [SMILES: CN(c1ccccc1)S(=O)(=O)c1cccc(-n2c(=O)[nH]c3scc(C(=O)O)c3c2=O)c1] | (DMSO-d6) 3.16 (3H, s), 7.05-7.1 (2H, m), 7.25-7.4 (3H, m), 7.55-7.65 (1H, m), 7.7-7.8 (3H, m), 7.98 (1H, s), 12.95 (1H, s), 14.31 (1H, s) |
| 137 | 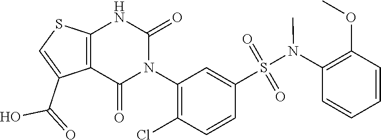 [SMILES: COc1ccccc1N(C)S(=O)(=O)c1ccc(Cl)c(-n2c(=O)[nH]c3scc(C(=O)O)c3c2=O)c1] | (DMSO-d6) 3.16 (3H, s), 3.47 (3H, s), 6.85-7.0 (2H, m), 7.1-7.2 (1H, m), 7.25- 7.35 (1H, m), 7.69 (1H, dd, J = 8.4 Hz, 2.1 Hz), 7.85-7.95 (2H, m), 7.99 (1H, d, J = 2.1 Hz), 12.5-13.5 (1H, br), 13.83 (1H, brs) |
| 138 | 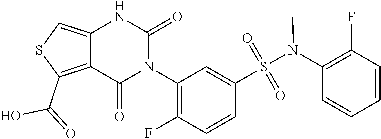 [SMILES: CN(c1ccccc1F)S(=O)(=O)c1ccc(F)c(-n2c(=O)[nH]c3csc(C(=O)O)c3c2=O)c1] | (DMSO-d6) 3.17 (3H, s), 7.1-7.25 (2H, m), 7.25-7.35 (1H, m), 7.35-7.45 (2H, m), 7.65-7.75 (1H, m), 7.8-7.9 (1H, m), 8.02 (1H, dd, J = 6.6 Hz, 2.3 Hz), 12.03 (1H, s), 14.31 (1H, s) |
| 139 | 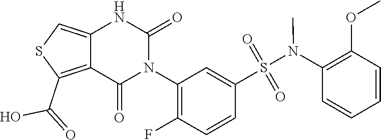 [SMILES: COc1ccccc1N(C)S(=O)(=O)c1ccc(F)c(-n2c(=O)[nH]c3csc(C(=O)O)c3c2=O)c1] | (DMSO-d6) 3.16 (3H, s), 3.47 (3H, s), 6.9-7.0 (2H, m), 7.19 (1H, dd, J = 8.0 Hz, 1.5 Hz), 7.3-7.35 (1H, m), 7.37 (1H, s), 7.6-7.7 (1H, m), 7.7-7.8 (1H, m), 8.0 (1H, dd, J = 6.6 Hz, 2.5 Hz), 12.02 (1H, s), 14.32 (1H, s) |
| 140 | 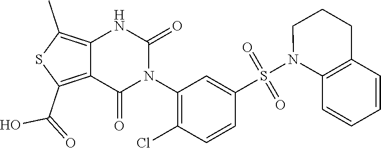 [SMILES: Cc1sc(C(=O)O)c2c(=O)n(-c3cc(S(=O)(=O)N4CCCc5ccccc54)ccc3Cl)c(=O)[nH]c12] | (DMSO-d6) 1.6-1.7 (2H, m), 2.45-2.55 (5H, m), 3.7-3.8 (2H, m), 7.05-7.25 (3H, m), 7.56 (1H, d, J = 8.5 Hz), 7.6-7.65 (1H, m), 7.84 (1H, d, J = 8.5 Hz), 8.1 (1H, d, J = 2.4 Hz), 11.94 (1H, s), 14.22 (1H, s) |
| 141 | 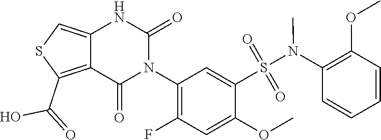 [SMILES: COc1ccccc1N(C)S(=O)(=O)c1cc(-n2c(=O)[nH]c3csc(C(=O)O)c3c2=O)c(F)cc1OC] | (CD3OD) 3.36 (3H, s), 3.54 (3H, s), 4.01 (3H, s), 6.85-6.95 (2H, m), 7.15- 7.35 (4H, m), 7.69 (1H, d, J = 8.4 Hz) |
| 142 | 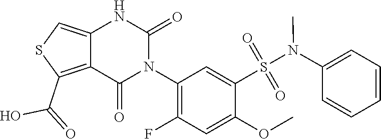 [SMILES: COc1cc(F)c(-n2c(=O)[nH]c3csc(C(=O)O)c3c2=O)cc1S(=O)(=O)N(C)c1ccccc1] | (DMSO-d6) 3.3 (3H, s), 3.87 (3H, s), 7.15-7.4 (6H, m), 7.42 (1H, d, J = 11.7 Hz), 7.92 (1H, d, J = 8.3 Hz), 11.95 (1H, s), 14.39 (1H, s) |
| 143 | 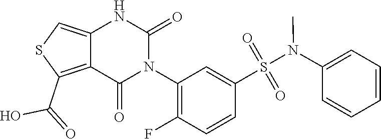 [SMILES: CN(c1ccccc1)S(=O)(=O)c1ccc(F)c(-n2c(=O)[nH]c3csc(C(=O)O)c3c2=O)c1] | (DMSO-d6) 3.17 (3H, s), 7.05-7.15 (2H, m), 7.25-7.4 (4H, m), 7.6-7.75 (2H, m), 7.9-8.0 (1H, m), 12.02 (1H, s), 14.29 (1H, s) |
| 144 | 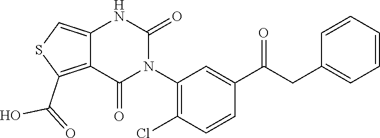 [SMILES: O=C(Cc1ccccc1)c1ccc(Cl)c(-n2c(=O)[nH]c3csc(C(=O)O)c3c2=O)c1] | (DMSO-d6) 4.42 (2H, s), 7.2-7.45 (6H, m), 7.86 (1H, d, J = 8.5 Hz), 8.2 (1H, dd, J = 8.5 Hz, 2.2 Hz), 8.29 (1H, d, J = 2.2 Hz), 12.06 (1H, brs), 14.32 (1H, brs) |
| 145 | 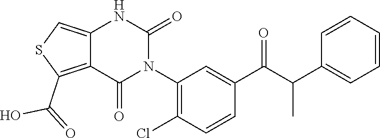 [SMILES: CC(C(=O)c1ccc(Cl)c(-n2c(=O)[nH]c3csc(C(=O)O)c3c2=O)c1)c1ccccc1] | (DMSO-d6) 1.4-1.5 (3H, m), 4.9-5.0 (1H, m), 7.15-7.45 (6H, m), 7.7-7.8 (1H, m), 8.1-8.15 (1H, m), 8.2-8.3 (1H, m), 12.0-12.1 (1H, m), 14.2-14.35 (1H, m) |
| 146 | 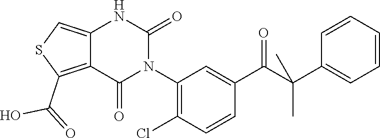 [SMILES: CC(C)(C(=O)c1ccc(Cl)c(-n2c(=O)[nH]c3csc(C(=O)O)c3c2=O)c1)c1ccccc1] | (DMSO-d6) 1.57 (6H, s), 7.25-7.45 (7H, m), 7.5-7.6 (1H, m), 7.9-8.0 (1H, m), 11.9-12.1 (1H, br), 14.2-14.4 (1H, br) |
| 147 | 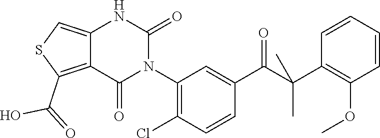 [SMILES: COc1ccccc1C(C)(C)C(=O)c1ccc(Cl)c(-n2c(=O)[nH]c3csc(C(=O)O)c3c2=O)c1] | (DMSO-d6) 1.55 (3H, s), 1.57 (3H, s), 3.33 (3H, s), 6.8-6.85 (1H, m), 7.0-7.1 (1H, m), 7.2-7.3 (1H, m), 7.3-7.5 (3H, m), 7.5-7.55 (1H, m), 7.94 (1H, d, J = 2.4 Hz), 12.0 (1H, s), 14.37 (1H, brs) |
| 148 | 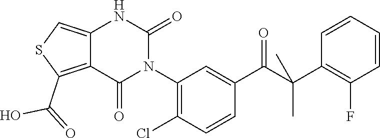 [SMILES: CC(C)(C(=O)c1ccc(Cl)c(-n2c(=O)[nH]c3csc(C(=O)O)c3c2=O)c1)c1ccccc1F] | (DMSO-d6) 1.6 (3H, s), 1.62 (3H, s), 7.0-7.15 (1H, m), 7.25-7.4 (3H, m), 7.47 (1H, dd, J = 8.7 Hz, 2.0 Hz), 7.57 (1H, d, J = 8.7 Hz), 7.65-7.75 (1H, m), 8.01 (1H, d, J = 2.0 Hz), 12.03 (1H, s), 14.3 (1H, brs) |
| 149 | 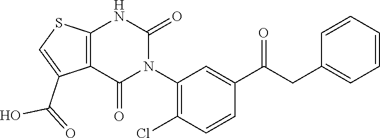 [SMILES: O=C(Cc1ccccc1)c1ccc(Cl)c(-n2c(=O)[nH]c3scc(C(=O)O)c3c2=O)c1] | (DMSO-d6) 4.42 (2H, s), 7.2-7.35 (5H, m), 7.86 (1H, d, J = 8.5 Hz), 7.94 (1H, s), 8.15-8.3 (2H, m), 13.84 (1H, s) |
| 150 | 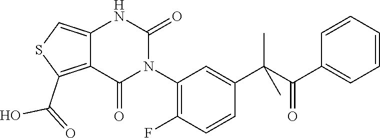 [SMILES: CC(C)(C(=O)c1ccccc1)c1ccc(F)c(-n2c(=O)[nH]c3csc(C(=O)O)c3c2=O)c1] | (DMSO-d6) 1.55 (6H, s), 7.25-7.55 (8H, m), 7.6-7.65 (1H, m), 11.99 (1H, s), 14.46 (1H, brs) |
| 151 | 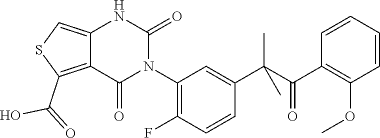 [SMILES: COc1ccccc1C(=O)C(C)(C)c1ccc(F)c(-n2c(=O)[nH]c3csc(C(=O)O)c3c2=O)c1] | (DMSO-d6) 1.49 (3H, s), 1.5 (3H, s), 3.66 (3H, s), 6.55-6.65 (1H, m), 6.75- 6.85 (1H, m), 6.95-7.05 (1H, m), 7.25- 7.5 (4H, m), 7.5-7.6 (1H, m), 11.99 (1H, s), 14.5 (1H, s) |
| 152 | 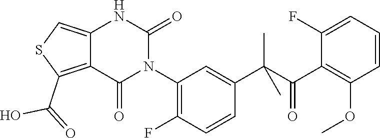 [SMILES: COc1cccc(F)c1C(=O)C(C)(C)c1ccc(F)c(-n2c(=O)[nH]c3csc(C(=O)O)c3c2=O)c1] | (DMSO-d6) 1.536 (3H, s), 1.543 (3H, s), 3.61 (3H, s), 6.75-6.85 (2H, m), 7.3-7.4 (3H, m), 7.45-7.6 (2H, m), 11.98 (1H, s), 14.5 (1H, s) |
| 153 | 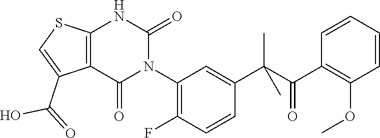 [SMILES: COc1ccccc1C(=O)C(C)(C)c1ccc(F)c(-n2c(=O)[nH]c3scc(C(=O)O)c3c2=O)c1] | (DMSO-d6) 1.49 (3H, s), 1.5 (3H, s), 3.66 (3H, s), 6.55-6.65 (1H, m), 6.75- 6.85 (1H, m), 6.95-7.05 (1H, m), 7.25- 7.55 (4H, m), 7.94 (1H, s), 12.8-13.1 (1H, br), 14.01 (1H, s) |
| 154 | 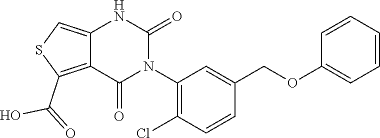 [SMILES: O=C(O)c1scc2[nH]c(=O)n(-c3cc(COc4ccccc4)ccc3Cl)c(=O)c12] | (DMSO-d6) 5.15 (2H, s), 6.9-7.1 (3H, m), 7.25-7.35 (2H, m), 7.4 (1H, s), 7.55- 7.65 (1H, m), 7.65-7.75 (2H, m), 12.05 (1H, s), 14.42 (1H, s) |
| 155 | 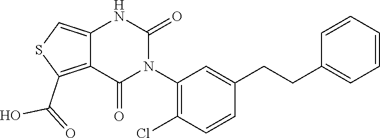 [SMILES: O=C(O)c1scc2[nH]c(=O)n(-c3cc(CCc4ccccc4)ccc3Cl)c(=O)c12] | (DMSO-d6) 2.85-2.95 (4H, m), 7.1-7.6 (9H, m), 12.04 (1H, s), 14.46 (1H, s) |
| 156 | 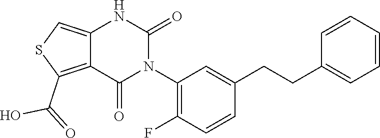 [SMILES: O=C(O)c1scc2[nH]c(=O)n(-c3cc(CCc4ccccc4)ccc3F)c(=O)c12] | (DMSO-d6) 2.8-3.0 (4H, m), 7.15-7.45 (9H, m), 12.03 (1H, s), 14.46 (1H, brs) |
| 157 | 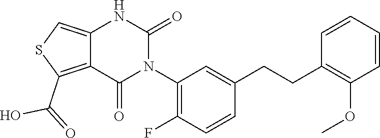 [SMILES: COc1ccccc1CCc1ccc(F)c(-n2c(=O)[nH]c3csc(C(=O)O)c3c2=O)c1] | (DMSO-d6) 2.8-2.9 (4H, m), 3.79 (3H, s), 6.8-6.9 (1H, m), 6.9-7.0 (1H, m), 7.1-7.25 (2H, m), 7.25-7.45 (4H, m), 12.02 (1H, s), 14.48 (1H, brs) |
| 158 | 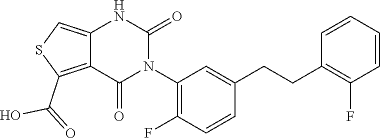 [SMILES: O=C(O)c1scc2[nH]c(=O)n(-c3cc(CCc4ccccc4F)ccc3F)c(=O)c12] | (DMSO-d6) 2.85-3.0 (4H, m), 7.05-7.2 (2H. m), 7.2-7.45 (6H, m), 12.03 (1H, s). 14.46 (1H, brs) |
| 159 | 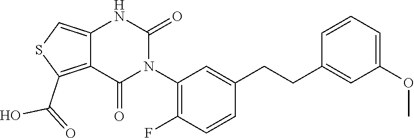 [SMILES: COc1cccc(CCc2ccc(F)c(-n3c(=O)[nH]c4csc(C(=O)O)c4c3=O)c2)c1] | (DMSO-d6) 2.8-2.95 (4H, m), 3.72 (3H, s), 6.7-6.85 (3H, m), 7.15-7.25 (1H, m), 7.25-7.45 (4H, m), 11.95-12.1 (1H, br), 14.35-14.55 (1H, br) |
| 160 | 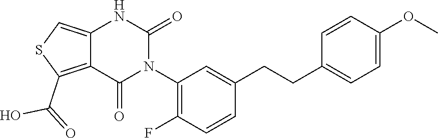 [SMILES: COc1ccc(CCc2ccc(F)c(-n3c(=O)[nH]c4csc(C(=O)O)c4c3=O)c2)cc1] | (DMSO-d6) 2.75-2.95 (4H, m), 3.71 (3H, s), 6.8-6.9 (2H, m), 7.1-7.2 (2H, m), 7.25-7.45 (4H, m), 12.01 (1H, brs), 14.4- 14.55 (1H, br) |
| 161 | 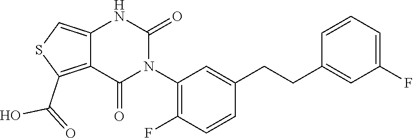 [SMILES: O=C(O)c1scc2[nH]c(=O)n(-c3cc(CCc4cccc(F)c4)ccc3F)c(=O)c12] | (DMSO-d6) 2.85-3.0 (4H, m), 6.95-7.05 (1H, m), 7.05-7.15 (2H, m), 7.25-7.45 (5H, m), 12.03 (1H, brs), 14.3-14.6 (1H, br) |
| 162 | 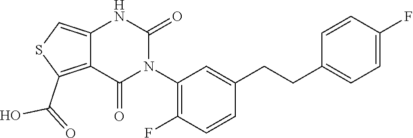 [SMILES: O=C(O)c1scc2[nH]c(=O)n(-c3cc(CCc4ccc(F)cc4)ccc3F)c(=O)c12] | (DMSO-d6) 2.8-2.95 (4H, m), 7.05-7.15 (2H, m), 7.2-7.45 (6H, m), 12.03 (1H, brs), 14.3-14.6 (1H, br) |
| 163 | 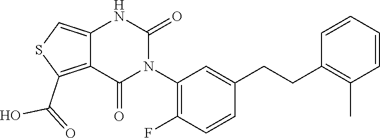 [SMILES: Cc1ccccc1CCc1ccc(F)c(-n2c(=O)[nH]c3csc(C(=O)O)c3c2=O)c1] | (DMSO-d6) 2.28 (3H, s), 2.8-2.9 (4H, m), 7.05-7.25 (4H, m), 7.25-7.5 (4H, m), 12.04 (1H, brs), 14.47 (1H, brs) |
| 164 | 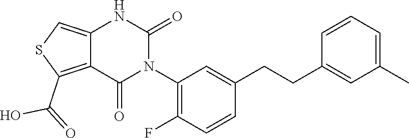 [SMILES: Cc1cccc(CCc2ccc(F)c(-n3c(=O)[nH]c4csc(C(=O)O)c4c3=O)c2)c1] | (DMSO-d6) 2.27 (3H, s), 2.8-2.95 (4H, m), 6.95-7.1 (3H, m), 7.1-7.2 (1H, m), 7.25-7.45 (4H, m), 12.03 (1H, brs), 14.47 (1H, brs) |
| 165 | 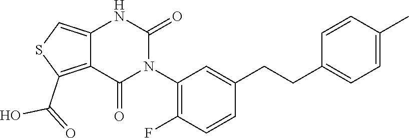 [SMILES: Cc1ccc(CCc2ccc(F)c(-n3c(=O)[nH]c4csc(C(=O)O)c4c3=O)c2)cc1] | (DMSO-d6) 2.25 (3H, s), 2.8-2.95 (4H, m), 7.05-7.15 (4H, m), 7.25-7.45 (4H, m), 12.03 (1H, brs), 14.35-14.6 (1H, br) |
| 166 | 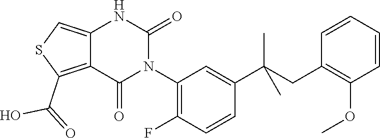 [SMILES: COc1ccccc1CC(C)(C)c1ccc(F)c(-n2c(=O)[nH]c3csc(C(=O)O)c3c2=O)c1] | (DMSO-d6) 1.23 (3H, s), 1.25 (3H, s), 2.85 (2H, s), 3.67 (3H, s), 6.7-6.8 (2H, m), 6.85- 6.95 (1H, m), 7.1-7.2 (1H, m), 7.25-7.35 (1H, m), 7.35-7.45 (2H, m), 7.5-7.6 (1H, m), 12.0 (1H, s), 14.55 (1H, s) |
| 167 | 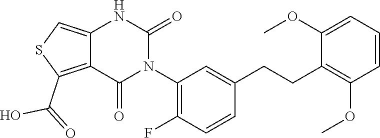 [SMILES: COc1cccc(OC)c1CCc1ccc(F)c(-n2c(=O)[nH]c3csc(C(=O)O)c3c2=O)c1] | (DMSO-d6) 2.65-2.75 (2H, m), 2.8-2.9 (2H, m), 3.75 (6H, s), 6.55-6.7 (2H, m), 7.1-7.2 (1H, m), 7.25-7.45 (4H, m), 12.0 (1H, s), 14.48 (1H, brs) |
| 168 | 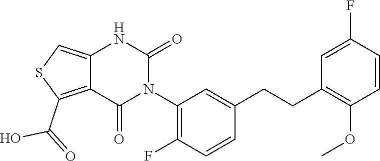 [SMILES: COc1ccc(F)cc1CCc1ccc(F)c(-n2c(=O)[nH]c3csc(C(=O)O)c3c2=O)c1] | (DMSO-d6) 2.8-2.95 (4H, m), 3.77 (3H, s), 6.9-7.1 (3H, m), 7.25-7.45 (4H, m), 12.01 (1H, s), 14.46 (1H, brs) |
| 169 | 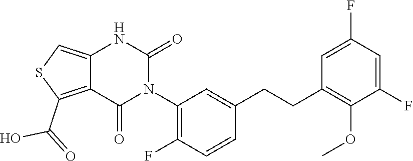 [SMILES: COc1c(F)cc(F)cc1CCc1ccc(F)c(-n2c(=O)[nH]c3csc(C(=O)O)c3c2=O)c1] | (DMSO-d6) 2.85-2.95 (4H, m), 3.75 (3H, s), 6.9-7.05 (1H, m), 7.05-7.2 (1H, m), 7.3-7.45 (4H, m), 12.01 (1H, s), 14.45 (1H, brs) |
| 170 | 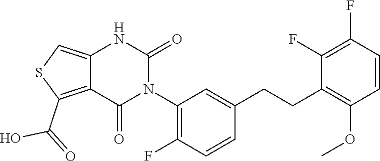 [SMILES: COc1ccc(F)c(F)c1CCc1ccc(F)c(-n2c(=O)[nH]c3csc(C(=O)O)c3c2=O)c1] | (DMSO-d6) 2.75-2.85 (2H, m), 2.85- 2.95 (2H, m), 3.77 (3H, s), 6.75-6.85 (1H, m), 7.15-7.35 (4H, m), 7.37 (1H, s), 11.99 (1H, s), 14.46 (1H, brs) |
| 171 | 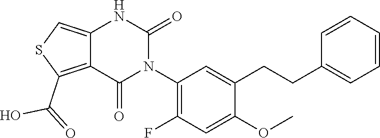 [SMILES: COc1cc(F)c(-n2c(=O)[nH]c3csc(C(=O)O)c3c2=O)cc1CCc1ccccc1] | (DMSO-d6) 2.75-2.9 (4H, m), 3.86 (3H, s), 7.08 (1H, d, J = 12.2 Hz), 7.15-7.35 (6H, m), 7.38 (1H, s), 11.97 (1H, s), 14.55 (1H, brs) |
| 172 | 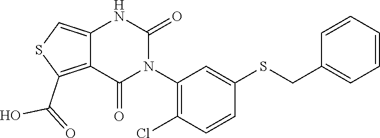 [SMILES: O=C(O)c1scc2[nH]c(=O)n(-c3cc(SCc4ccccc4)ccc3Cl)c(=O)c12] | (DMSO-d6) 4.28 (2H, s), 7.2-7.35 (3H, m), 7.35-7.5 (4H, m), 7.58 (1H, d, J = 8.5 Hz), 7.66 (1H, d, J = 2.2 Hz), 12.06 (1H, s), 14.41 (1H, s) |
| 173 | 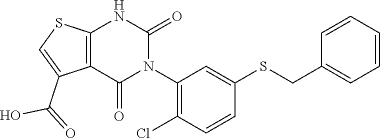 [SMILES: O=C(O)c1csc2[nH]c(=O)n(-c3cc(SCc4ccccc4)ccc3Cl)c(=O)c12] | (DMSO-d6) 4.29 (2H, s), 7.2-7.45 (6H, m), 7.57 (1H, d, J = 8.5 Hz), 7.64 (1H, d, J = 2.3 Hz), 7.94 (1H, s), 13.03 (1H, s), 13.94 (1H, s) |
| 174 | 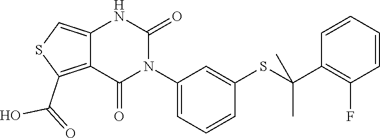 [SMILES: CC(C)(Sc1cccc(-n2c(=O)[nH]c3csc(C(=O)O)c3c2=O)c1)c1ccccc1F] | (DMSO-d6) 1.69 (6H, s), 7.0-7.2 (4H, m), 7.25-7.4 (5H, m), 11.89 (1H, s), 14.86 (1H, s) |
| 175 | 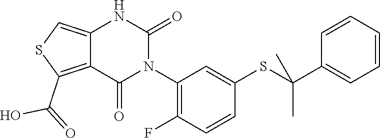 [SMILES: CC(C)(Sc1ccc(F)c(-n2c(=O)[nH]c3csc(C(=O)O)c3c2=O)c1)c1ccccc1] | (DMSO-d6) 4.24 (2H, s), 7.2-7.5 (8H, m), 7.62 (1H, dd, J = 6.7 Hz, 2.2 Hz), 12.05 (1H, s), 14.41 (1H, s) |
| 176 | 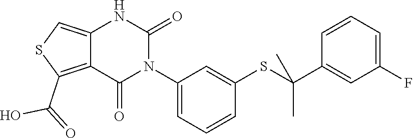 [SMILES: CC(C)(Sc1cccc(-n2c(=O)[nH]c3csc(C(=O)O)c3c2=O)c1)c1cccc(F)c1] | (DMSO-d6) 1.65 (6H, s), 7.0-7.1 (1H, m), 7.1-7.45 (8H, m), 11.88 (1H, s), 14.83 (1H, s) |
| 177 | 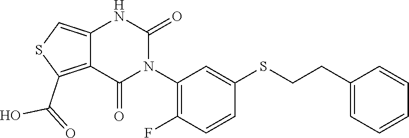 [SMILES: O=C(O)c1scc2[nH]c(=O)n(-c3cc(SCCc4ccccc4)ccc3F)c(=O)c12] | (DMSO-d6) 2.8-2.9 (2H, m), 3.15-3.25 (2H, m), 7.15-7.55 (8H, m), 7.58 (1H, dd, J = 6.8 Hz, 2.3 Hz) |
| 178 | 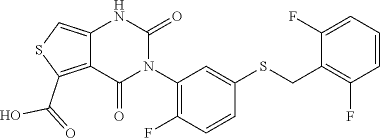 [SMILES: O=C(O)c1scc2[nH]c(=O)n(-c3cc(SCc4c(F)cccc4F)ccc3F)c(=O)c12] | (DMSO-d6) 4.21 (2H, s), 7.0-7.15 (2H, m), 7.3-7.45 (3H, m), 7.5-7.65 (2H, m), 12.0 (1H, s), 14.37 (1H, s) |
| 179 | 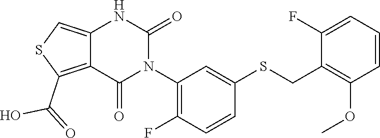 [SMILES: COc1cccc(F)c1CSc1ccc(F)c(-n2c(=O)[nH]c3csc(C(=O)O)c3c2=O)c1] | (DMSO-d6) 3.76 (3H, s), 4.15 (2H, s), 6.75-6.9 (2H, m), 7.25-7.35 (2H, m), 7.35-7.45 (1H, m), 7.45-7.6 (2H, m) |
| 180 | 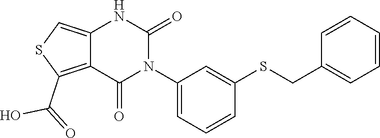 [SMILES: O=C(O)c1scc2[nH]c(=O)n(-c3cccc(SCc4ccccc4)c3)c(=O)c12] | (DMSO-d6) 4.27 (2H, s), 7.15-7.5 (10H, m), 11.93 (1H, s), 14.88 (1H, s) |
| 181 | 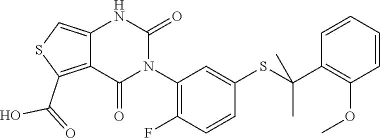 [SMILES: COc1ccccc1C(C)(C)Sc1ccc(F)c(-n2c(=O)[nH]c3csc(C(=O)O)c3c2=O)c1] | (DMSO-d6) 1.65-1.7 (6H, m), 3.86 (3H, s) 6.75-6.85 (1H, m), 7.0-7.15 (3H, m), 7.2-7.3 (2H, m), 7.37 (1H, s), 7.42 (1H, dd, J = 6.9 Hz, 2.2 Hz), 11.98 (1H, s), 14.38 (1H, s) |
| 182 | 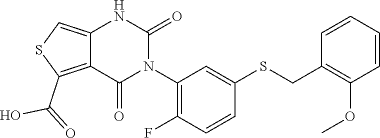 [SMILES: COc1ccccc1CSc1ccc(F)c(-n2c(=O)[nH]c3csc(C(=O)O)c3c2=O)c1] | (DMSO-d6) 3.79 (3H, s), 4.16 (2H, s), 6.8-6.9 (1H, m), 6.95-7.05 (1H, m), 7.2- 7.3 (2H, m), 7.3-7.5 (3H, m), 7.59 (1H, dd, J = 7.0 hz, 2.2 Hz), 12.02 (1H, s), 14.39 (1H, s) |
| 183 | 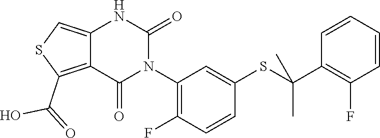 [SMILES: CC(C)(Sc1ccc(F)c(-n2c(=O)[nH]c3csc(C(=O)O)c3c2=O)c1)c1ccccc1F] | (DMSO-d6) 1.69 (6H, s), 7.0-7.35 (6H, m), 7.37 (1H, s), 7.46 (1H, dd, J = 6.7 Hz, 2.2 Hz), 11.98 (1H, s), 14.37 (1H, brs) |
| 184 | 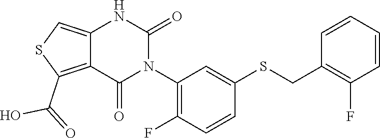 [SMILES: O=C(O)c1scc2[nH]c(=O)n(-c3cc(SCc4ccccc4F)ccc3F)c(=O)c12] | (DMSO-d6) 4.24 (2H, s), 7.05-7.25 (2H, m), 7.25-7.45 (4H, m), 7.45-7.55 (1H, m), 7.63 (1H, dd, J = 6.8 Hz, 2.5 Hz), 12.03 (1H, s), 14.38 (1H, s) |
| 185 | 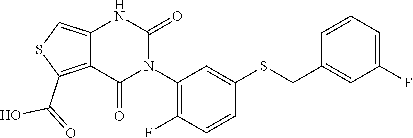 [SMILES: O=C(O)c1scc2[nH]c(=O)n(-c3cc(SCc4cccc(F)c4)ccc3F)c(=O)c12] | (DMSO-d6) 4.25 (2H, s), 7.0-7.1 (1H, m), 7.15-7.2 (2H, m), 7.25-7.5 (4H, m), 7.62 (1H, dd, J = 6.7 Hz, 2.3 Hz), 12.03 (1H, s), 14.37 (1H, s) |
| 186 | 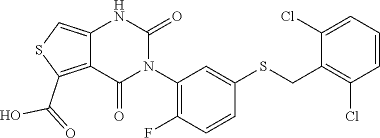 [SMILES: O=C(O)c1scc2[nH]c(=O)n(-c3cc(SCc4c(Cl)cccc4Cl)ccc3F)c(=O)c12] | (DMSO-d6) 4.4 (2H, s), 7.3-7.6 (6H, m), 7.65-7.75 (1H, m), 12.01 (1H, s), 14.37 (1H, s) |
| 187 | 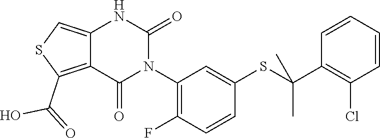 [SMILES: CC(C)(Sc1ccc(F)c(-n2c(=O)[nH]c3csc(C(=O)O)c3c2=O)c1)c1ccccc1Cl] | (DMSO-d6) 1.75-1.8 (6H, m), 7.05-7.15 (1H, m), 7.15-7.3 (4H, m), 7.37 (1H, s), 7.4-7.5 (2H, m), 11.97 (1H, s), 14.37 (1H, s) |
| 188 | 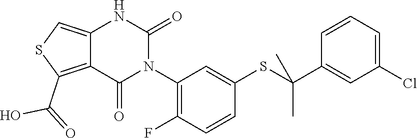 [SMILES: CC(C)(Sc1ccc(F)c(-n2c(=O)[nH]c3csc(C(=O)O)c3c2=O)c1)c1cccc(Cl)c1] | (DMSO-d6) 1.65 (6H, s), 7.15-7.25 (1H, m), 7.25-7.45 (6H, m), 7.5-7.6 (1H, m), 12.0 (1H, s), 14.38 (1H, s) |
| 189 | 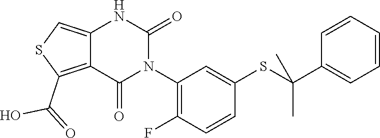 [SMILES: CC(C)(Sc1ccc(F)c(-n2c(=O)[nH]c3csc(C(=O)O)c3c2=O)c1)c1ccccc1] | (DMSO-d6) 1.64 (6H, s), 7.15-7.35 (5H, m), 7.37 (1H, s), 7.4-7.45 (2H, m), 7.52 (1H, dd, J = 7.3 Hz, 2.3 Hz), 11.99 (1H, s), 14.37 (1H, brs) |
| 190 | 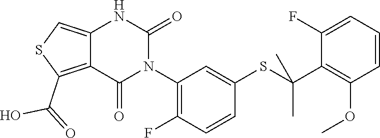 [SMILES: COc1cccc(F)c1C(C)(C)Sc1ccc(F)c(-n2c(=O)[nH]c3csc(C(=O)O)c3c2=O)c1] | (DMSO-d6) 1.75-1.85 (6H, m), 3.77 (3H, s), 6.6-6.7 (1H, m), 6.85 (1H, d, J = 8.1 Hz), 7.05-7.15 (1H, m), 7.2-7.35 (2H, m), 7.37 (1H, s), 7.4-7.45 (1H, m), 11.97 (1H, s), 14.4 (1H, brs) |
| 191 | 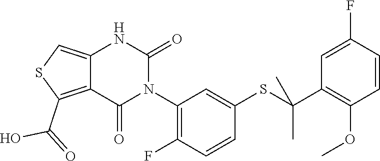 [SMILES: COc1ccc(F)cc1C(C)(C)Sc1ccc(F)c(-n2c(=O)[nH]c3csc(C(=O)O)c3c2=O)c1] | (DMSO-d6) 1.65-1.7 (6H, m), 3.85 (3H, s), 6.75-6.8 (1H, m), 7.0-7.1 (2H, m), 7.1-7.2 (1H, m), 7.25-7.35 (1H, m), 7.37 (1H, s), 7.4 (1H, dd, J = 7.3 Hz, 2.2 hz), 11.98 (1H, s), 14.39 (1H, brs) |
| 192 | 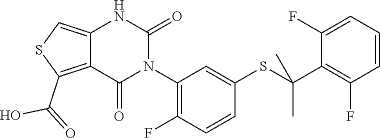 [SMILES: CC(C)(Sc1ccc(F)c(-n2c(=O)[nH]c3csc(C(=O)O)c3c2=O)c1)c1c(F)cccc1F] | (DMSO-d6) 1.81 (6H, s), 6.9-7.05 (2H, m), 7.1-7.2 (1H, m), 7.25-7.4 (3H, m), 7.45-7.5 (1H, m), 11.97 (1H, s), 14.38 (1H, brs) |
| 193 | 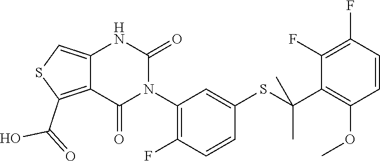 [SMILES: COc1ccc(F)c(F)c1C(C)(C)Sc1ccc(F)c(-n2c(=O)[nH]c3csc(C(=O)O)c3c2=O)c1] | (DMSO-d6) 1.8-1.85 (6H, m), 3.76 (3H, s), 6.75-6.85 (1H, m), 7.15-7.45 (5H, m), 11.98 (1H, s), 14.38 (1H, brs) |
| 194 | 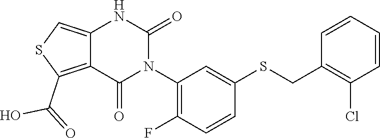 [SMILES: O=C(O)c1scc2[nH]c(=O)n(-c3cc(SCc4ccccc4Cl)ccc3F)c(=O)c12] | (DMSO-d6) 4.29 (2H, s), 7.2-7.35 (2H, m), 7.35-7.55 (5H, m), 7.6-7.7 (1H, m), 12.02 (1H, s), 14.38 (1H, brs) |
| 195 | 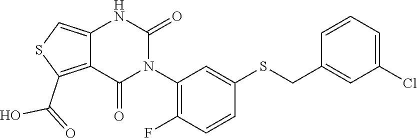 [SMILES: O=C(O)c1scc2[nH]c(=O)n(-c3cc(SCc4cccc(Cl)c4)ccc3F)c(=O)c12] | (DMSO-d6) 4.24 (2H, s), 7.25-7.5 (7H, m), 7.6-7.65 (1H, m), 12.03 (1H, s), 14.38 (1H, brs) |
| 196 | 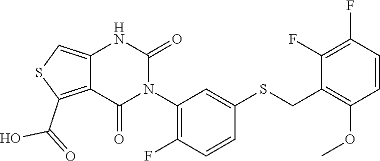 [SMILES: COc1ccc(F)c(F)c1CSc1ccc(F)c(-n2c(=O)[nH]c3csc(C(=O)O)c3c2=O)c1] | (DMSO-d6) 3.73 (3H, s), 4.16 (2H, s), 6.75-6.85 (1H, m), 7.1-7.6 (5H, m) |
| 197 | 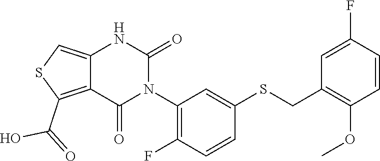 [SMILES: COc1ccc(F)cc1CSc1ccc(F)c(-n2c(=O)[nH]c3csc(C(=O)O)c3c2=O)c1] | (DMSO-d6) 3.77 (3H, s), 4.15 (2H, s), 6.95-7.15 (3H, m), 7.35-7.5 (3H, m), 7.6-7.65 (1H, m), 12.06 (1H, s), 14.41 (1H, s) |
| 198 | 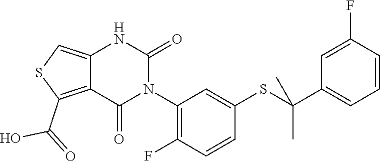 [SMILES: CC(C)(Sc1ccc(F)c(-n2c(=O)[nH]c3csc(C(=O)O)c3c2=O)c1)c1cccc(F)c1] | (DMSO-d6) 1.6-1.7 (6H, m), 7.0-7.1 (1H, m), 7.15-7.25 (3H, m), 7.3-7.4 (3H, m), 7.5-7.55 (1H, m), 12.01 (1H, s), 14.4 (1H, s) |
| 199 | 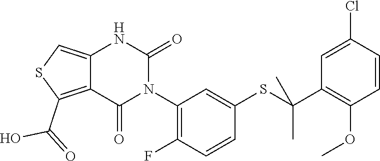 [SMILES: COc1ccc(Cl)cc1C(C)(C)Sc1ccc(F)c(-n2c(=O)[nH]c3csc(C(=O)O)c3c2=O)c1] | (DMSO-d6) 1.66 (3H, s), 1.67 (3H, s), 3.86 (3H, s), 7.0 (1H, d, J = 2.5 Hz), 7.06 (1H, d, J = 8.8 Hz), 7.1-7.2 (1H, m), 7.25-7.35 (2H, m), 7.38 (1H, s), 7.4-7.5 (1H, m), 12.01 (1H, s), 14.41 (1H, brs) |
| 200 | 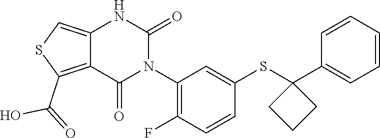 [SMILES: O=C(O)c1scc2[nH]c(=O)n(-c3cc(SC4(c5ccccc5)CCC4)ccc3F)c(=O)c12] | (DMSO-d6) 1.75-1.9 (1H, m), 2.15-2.3 (1H, m), 2.4-2.65 (4H, m), 7.0-7.2 (4H, m), 7.2-7.35 (3H, m), 7.37 (1H, s), 7.45- 7.5 (1H, m), 11.98 (1H, s), 14.41 (1H, brs) |
| 201 | 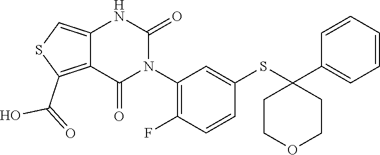 [SMILES: O=C(O)c1scc2[nH]c(=O)n(-c3cc(SC4(c5ccccc5)CCOCC4)ccc3F)c(=O)c12] | (DMSO-d6) 2.05-2.25 (4H, m), 3.5-3.65 (2H, m), 3.85-3.95 (2H, m). 6.85-6.95 (1H, m), 7.15-7.4 (8H, m), 11.98 (1H, s), 14.38 (1H, brs) |
| 202 | 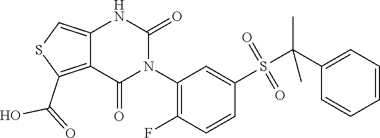 [SMILES: CC(C)(c1ccccc1)S(=O)(=O)c1ccc(F)c(-n2c(=O)[nH]c3csc(C(=O)O)c3c2=O)c1] | (DMSO-d6) 1.73 (6H, s), 7.3-7.4 (7H, m), 7.5-7.6 (1H, m), 7.95 (1H, dd, J = 6.7 Hz, 2.2 Hz), 12.01 (1H, s), 14.25 (1H, s) |
| 203 | 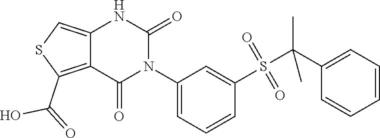 [SMILES: CC(C)(c1ccccc1)S(=O)(=O)c1cccc(-n2c(=O)[nH]c3csc(C(=O)O)c3c2=O)c1] | (DMSO-d6) 1.7 (6H, s), 6.89 (1H, s), 7.2-7.4 (6H, m), 7.5-7.7 (3H, m) |
| 204 | 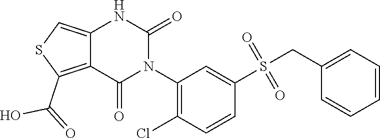 [SMILES: O=C(O)c1scc2[nH]c(=O)n(-c3cc(S(=O)(=O)Cc4ccccc4)ccc3Cl)c(=O)c12] | (DMSO-d6) 4.77 (2H, s), 7.15-7.25 (2H, m), 7.25-7.35 (3H, m), 7.39 (1H, s), 7.87 (1H, dd, J = 8.6 Hz, 2.2 Hz), 7.95 (1H, d, J = 8.6 Hz), 8.09 (1H, d, J = 2.2 Hz), 12.08 (1H, s), 14.26 (1H, s) |
| 205 | 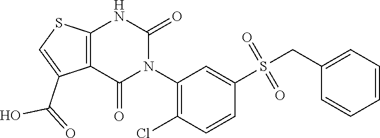 [SMILES: O=C(O)c1csc2[nH]c(=O)n(-c3cc(S(=O)(=O)Cc4ccccc4)ccc3Cl)c(=O)c12] | (DMSO-d6) 4.77 (2H, s), 7.15-7.25 (2H, m), 7.25-7.35 (3H, m), 7.8-8.0 (3H, m), 8.08 (1H, d, J = 2.3 Hz), 13.78 (1H, s) |
| 206 | 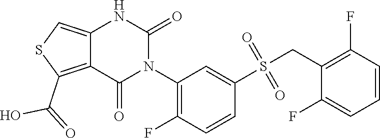 [SMILES: O=C(O)c1scc2[nH]c(=O)n(-c3cc(S(=O)(=O)Cc4c(F)cccc4F)ccc3F)c(=O)c12] | (DMSO-d6) 4.76 (2H, s), 7.05-7.15 (2H, m), 7.36 (1H, s), 7.4-7.55 (1H, m), 7.7- 7.8 (1H, m), 7.95-8.1 (2H, m), 12.02 (1H, s), 14.26 (1H, s) |
| 207 | 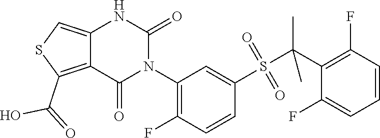 [SMILES: CC(C)(c1c(F)cccc1F)S(=O)(=O)c1ccc(F)c(-n2c(=O)[nH]c3csc(C(=O)O)c3c2=O)c1] | (DMSO-d6) 1.89 (6H, s), 7.0-7.1 (2H, m), 7.36 (1H, s), 7.4-7.5 (1H, m), 7.6-7.7 (2H, m), 8.0-8.1 (1H, m), 12.0 (1H, s), 14.28 (1H, s) |
| 208 | 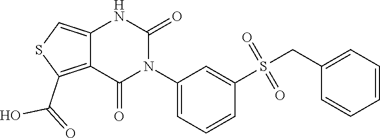 [SMILES: O=C(O)c1scc2[nH]c(=O)n(-c3cccc(S(=O)(=O)Cc4ccccc4)c3)c(=O)c12] | (DMSO-d6) 4.72 (2H, s), 7.15-7.25 (2H, m), 7.25-7.35 (3H, m), 7.39 (1H, s), 7.7- 7.95 (4H, m), 11.97 (1H, s), 14.72 (1H, s) |
| 209 | 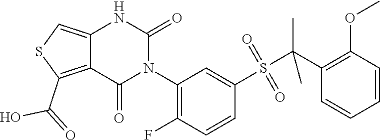 [SMILES: COc1ccccc1C(C)(C)S(=O)(=O)c1ccc(F)c(-n2c(=O)[nH]c3csc(C(=O)O)c3c2=O)c1] | (DMSO-d6) 1.86 (6H, s), 3.35 (3H, s), 6.83 (1H, d, J = 8.6 Hz), 6.9-7.0 (1H, m), 7.25-7.35 (2H, m), 7.37 (1H, s), 7.4-7.45 (1H, m), 7.5-7.6 (1H, m), 7.85-7.95 (1H, m), 12.0 (1H, s), 14.29 (1H, s) |
| 210 | 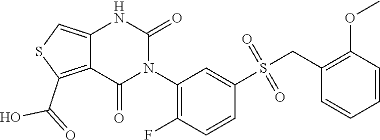 [SMILES: COc1ccccc1CS(=O)(=O)c1ccc(F)c(-n2c(=O)[nH]c3csc(C(=O)O)c3c2=O)c1] | (DMSO-d6) 3.46 (3H, s), 4.63 (2H, s), 6.85-6.95 (2H, m), 7.2-7.35 (2H, m), 7.37 (1H, s), 7.6-7.75 (2H, m), 7.9-8.0 (1H, m), 12.02 (1H, s), 14.26 (1H, s) |
| 211 | 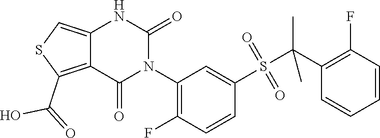 [SMILES: CC(C)(c1ccccc1F)S(=O)(=O)c1ccc(F)c(-n2c(=O)[nH]c3csc(C(=O)O)c3c2=O)c1] | (DMSO-d6) 1.81 (6H, s), 7.05-7.2 (2H, m), 7.3-7.45 (3H, m), 7.5-7.65 (2H, m), 7.98 (1H, dd, J = 6.7 Hz, 2.3 Hz), 12.01 (1H, s), 14.27 (1H, s) |
| 212 | 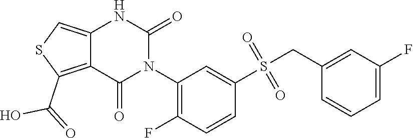 [SMILES: O=C(O)c1scc2[nH]c(=O)n(-c3cc(S(=O)(=O)Cc4cccc(F)c4)ccc3F)c(=O)c12] | (DMSO-d6) 4.81 (2H, s), 6.95-7.05 (2H, m), 7.1-7.2 (1H, m), 7.3-7.4 (2H, m), 7.7-7.75 (1H, m), 7.9-8.0 (1H, m), 8.04 (1H, dd, J = 6.8 Hz, 2.6 Hz), 12.05 (1H, s), 14.24 (1H, s) |
| 213 | 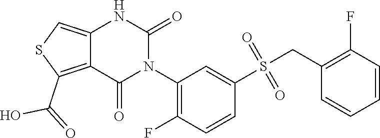 [SMILES: O=C(O)c1scc2[nH]c(=O)n(-c3cc(S(=O)(=O)Cc4ccccc4F)ccc3F)c(=O)c12] | (DMSO-d6) 4.7-4.8 (2H, m), 7.1-7.3 (3H, m), 7.35-7.45 (2H, m), 7.65-7.75 (1H, m), 7.9-8.0 (1H, m), 8.05 (1H, dd, J = 6.8 Hz, 2.5 Hz), 12.03 (1H, s), 14.25 (1H, s) |
| 214 | 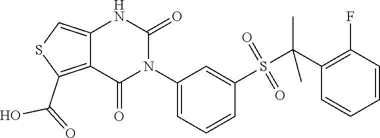 [SMILES: CC(C)(c1ccccc1F)S(=O)(=O)c1cccc(-n2c(=O)[nH]c3csc(C(=O)O)c3c2=O)c1] | (DMSO-d6) 1.75-1.85 (6H, m), 7.05-7.2 (2H, m), 7.3-7.5 (4H, m), 7.6-7.8 (3H, m), 11.91 (1H, s), 14.72 (1H, s) |
| 215 | 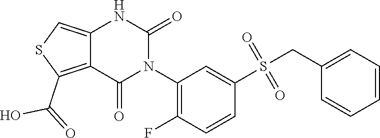 [SMILES: O=C(O)c1scc2[nH]c(=O)n(-c3cc(S(=O)(=O)Cc4ccccc4)ccc3F)c(=O)c12] | (DMSO-d6) 4.74 (2H, s), 7.15-7.2 (2H, m), 7.25-7.35 (3H, m), 7.37 (1H, s), 7.65-7.75 (1H, m), 7.85-7.95 (1H, m), 8.07 (1H, dd, J = 6.6 Hz, 2.5 Hz), 12.04 (1H, s), 14.24 (1H, s) |
| 216 | 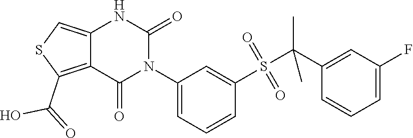 [SMILES: CC(C)(c1cccc(F)c1)S(=O)(=O)c1cccc(-n2c(=O)[nH]c3csc(C(=O)O)c3c2=O)c1] | (DMSO-d6) 1.72 (6H, s), 7.1-7.25 (3H, m), 7.3-7.45 (3H, m), 7.6-7.7 (1H, m), 7.7-7.8 (2H, m), 11.92 (1H, s), 14.7 (1H, s) |
| 217 | 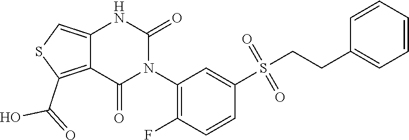 [SMILES: O=C(O)c1scc2[nH]c(=O)n(-c3cc(S(=O)(=O)CCc4ccccc4)ccc3F)c(=O)c12] | (DMSO-d6) 2.85-2.95 (2H, m), 3.65- 3.75 (2H, m), 7.15-7.3 (5H, m), 7.37 (1H, s), 7.7-7.8 (1H, m), 8.1-8.15 (1H, m), 8.27 (1H, dd, J = 6.6 Hz, 2.6 Hz), 12.05 (1H, s), 14.23 (1H, brs) |
| 218 | 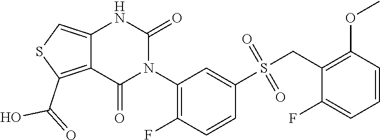 [SMILES: COc1cccc(F)c1CS(=O)(=O)c1ccc(F)c(-n2c(=O)[nH]c3csc(C(=O)O)c3c2=O)c1] | (DMSO-d6) 3.47 (3H, s), 4.62 (2H, s), 6.75 (1H, d, J = 8.4 Hz), 6.8-6.9 (1H, m), 7.3-7.4 (2H, m), 7.65-7.75 (1H, m), 7.8- 7.9 (1H, m), 7.95-8.05 (1H, m), 12.0 (1H, s), 14.27 (1H, brs) |
| 219 | 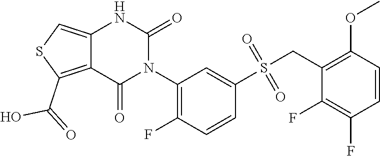 [SMILES: COc1ccc(F)c(F)c1CS(=O)(=O)c1ccc(F)c(-n2c(=O)[nH]c3csc(C(=O)O)c3c2=O)c1] | (DMSO-d6) 3.45 (3H, s), 4.68 (2H, s), 6.7-6.75 (1H, m), 7.35-7.45 (2H, m), 7.65-7.75 (1H, m), 7.85-8.0 (2H, m), 12.01 (1H, s), 14.25 (1H, brs) |
| 220 | 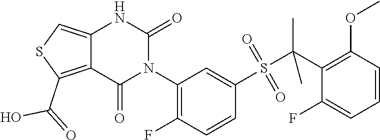 [SMILES: COc1cccc(F)c1C(C)(C)S(=O)(=O)c1ccc(F)c(-n2c(=O)[nH]c3csc(C(=O)O)c3c2=O)c1] | (DMSO-d6) 1.9-2.0 (6H, m), 3.38 (3H, s), 6.65-6.8 (2H, m), 7.3-7.4 (2H, m), 7.4-7.5 (1H, m), 7.55-7.6 (1H, m), 7.95- 8.0 (1H, m), 12.0 (1H, s), 14.28 (1H, brs) |
| 221 | 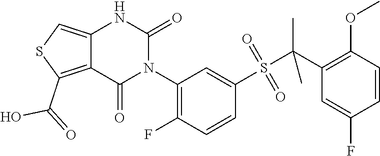 [SMILES: COc1ccc(F)cc1C(C)(C)S(=O)(=O)c1ccc(F)c(-n2c(=O)[nH]c3csc(C(=O)O)c3c2=O)c1] | (DMSO-d6) 1.85 (6H, s), 3.34 (3H, s), 6.84 (1H, dd, J = 9.1 Hz, 5.2 Hz), 7.1- 7.25 (2H, m), 7.37 (1H, s), 7.4-7.45 (1H, m), 7.55-7.65 (1H, m), 7.9-8.0 (1H, m), 12.0 (1H, s), 14.29 (1H, brs) |
| 222 | 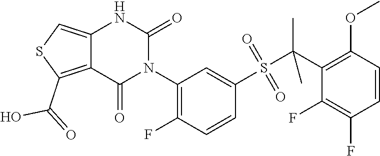 [SMILES: COc1ccc(F)c(F)c1C(C)(C)S(=O)(=O)c1ccc(F)c(-n2c(=O)[nH]c3csc(C(=O)O)c3c2=O)c1] | (DMSO-d6) 1.9-2.0 (6H, m), 3.36 (3H, s), 6.65-6.7 (1H, m), 7.35-7.45 (2H, m), 7.5-7.65 (2H, m), 7.95-8.0 (1H, m), 12.0 (1H, s), 14.28 (1H, brs) |
| 223 | 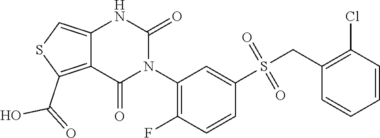 [SMILES: O=C(O)c1scc2[nH]c(=O)n(-c3cc(S(=O)(=O)Cc4ccccc4Cl)ccc3F)c(=O)c12] | (DMSO-d6) 4.85 (2H, s), 7.3-7.45 (5H, m), 7.65-7.75 (1H, m), 7.8-7.9 (1H, m), 8.05-8.1 (1H, m), 12.03 (1H, s), 14.25 (1H, brs) |
| 224 | 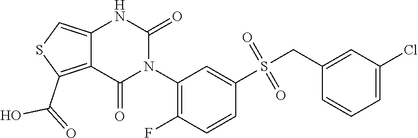 [SMILES: O=C(O)c1scc2[nH]c(=O)n(-c3cc(S(=O)(=O)Cc4cccc(Cl)c4)ccc3F)c(=O)c12] | (DMSO-d6) 4.81 (2H, s), 7.05-7.15 (1H, m), 7.25-7.45 (4H, m), 7.7-7.8 (1H, m), 7.9-8.0 (1H, m), 8.05-8.1 (1H, m), 12.05 (1H, s), 14.24 (1H, brs) |
| 225 | 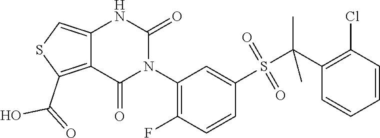 [SMILES: CC(C)(c1ccccc1Cl)S(=O)(=O)c1ccc(F)c(-n2c(=O)[nH]c3csc(C(=O)O)c3c2=O)c1] | (DMSO-d6) 1.95 (6H, s), 7.25-7.45 (5H, m), 7.5-7.65 (2H, m), 8.03 (1H, dd, J = 6.8 Hz, 2.4 Hz), 12.0 (1H, s), 14.28 (1H, s) |
| 226 | 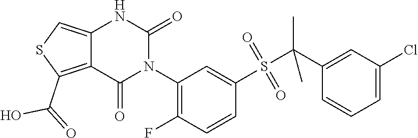 [SMILES: CC(C)(c1cccc(Cl)c1)S(=O)(=O)c1ccc(F)c(-n2c(=O)[nH]c3csc(C(=O)O)c3c2=O)c1] | (DMSO-d6) 1.72 (6H, s), 7.3-7.5 (6H, m), 7.55-7.65 (1H, m), 8.01 (1H, dd, J = 6.7 Hz, 2.3 Hz), 12.02 (1H, s), 14.25 (1H, s) |
| 227 | 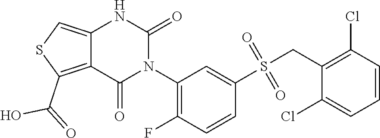 [SMILES: O=C(O)c1scc2[nH]c(=O)n(-c3cc(S(=O)(=O)Cc4c(Cl)cccc4Cl)ccc3F)c(=O)c12] | (DMSO-d6) 4.96 (2H, s), 7.35-7.45 (2H, m), 7.45-7.55 (2H, m), 7.65-7.75 (1H, m), 7.85-7.95 (1H, m), 8.15-8.2 (1H, m), 12.05 (1H, s), 14.28 (1H, s) |
| 228 | 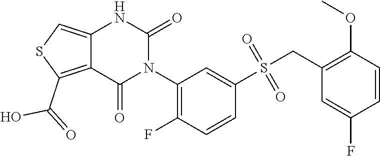 [SMILES: COc1ccc(F)cc1CS(=O)(=O)c1ccc(F)c(-n2c(=O)[nH]c3csc(C(=O)O)c3c2=O)c1] | (DMSO-d6) 3.43 (3H, s), 4.6-4.7 (2H, m), 6.85-6.9 (1H, m), 7.1-7.2 (2H, m), 7.37 (1H, s), 7.65-7.7 (1H, m), 7.75-7.85 (1H, m), 7.9-8.0 (1H, m), 12.04 (1H, s), 14.29 (1H, s) |
| 229 | 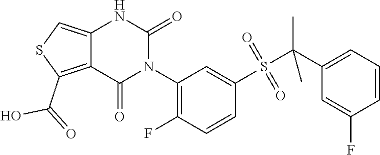 [SMILES: CC(C)(c1cccc(F)c1)S(=O)(=O)c1ccc(F)c(-n2c(=O)[nH]c3csc(C(=O)O)c3c2=O)c1] | (DMSO-d6) 1.72 (3H, s), 1.73 (3H, s), 7.15-7.25 (3H, m), 7.35-7.45 (2H, m), 7.45-7.55 (1H, m), 7.6 (1H, t, J = 9.1 Hz), 7.9-8.0 (1H, m), 12.04 (1H, s), 14.28 (1H, brs) |
| 230 | 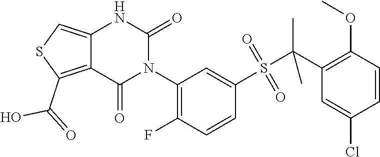 [SMILES: COc1ccc(Cl)cc1C(C)(C)S(=O)(=O)c1ccc(F)c(-n2c(=O)[nH]c3csc(C(=O)O)c3c2=O)c1] | (DMSO-d6) 1.86 (6H, s), 6.87 (1H, d, J = 8.8 Hz), 7.35-7.45 (4H, m), 7.55-7.65 (1H, m), 7.95-8.0 (1H, m), 12.03 (1H, s), 14.3 (1H, brs) |
| 231 | 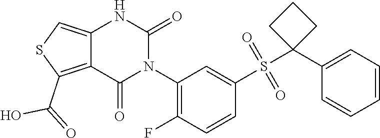 [SMILES: O=C(O)c1scc2[nH]c(=O)n(-c3cc(S(=O)(=O)C4(c5ccccc5)CCC4)ccc3F)c(=O)c12] | (DMSO-d6) 1.8-1.95 (1H, m), 2.0-2.15 (1H, m), 2.55-2.7 (2H, m), 3.0-3.15 (2H, m), 6.95-7.05 (2H, m), 7.2-7.35 (4H, m), 7.37 (1H, s), 7.45-7.55 (1H, m), 7.9- 8.0 (1H, m), 12.0 (1H, s), 14.28 (1H, brs) |
| 232 | 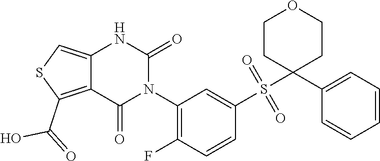 [SMILES: O=C(O)c1scc2[nH]c(=O)n(-c3cc(S(=O)(=O)C4(c5ccccc5)CCOCC4)ccc3F)c(=O)c12] | (DMSO-d6) 2.2-2.35 (2H, m), 3.05-3.2 (2H, m), 3.8-3.9 (2H, m), 7.2-7.4 (7H, m), 7.45-7.55 (1H, m), 7.8-7.9 (1H, m), 12.02 (1H. s), 14.25 (1H, brs) |
| 233 | 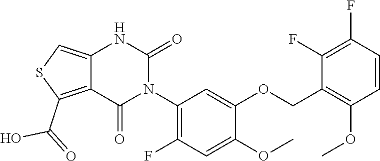 [SMILES: COc1cc(F)c(-n2c(=O)[nH]c3csc(C(=O)O)c3c2=O)cc1OCc1c(OC)ccc(F)c1F] | (DMSO-d6) 3.75-3.85 (6H, m), 4.96 (2H, s), 6.85-6.95 (1H, m), 7.13 (1H, d, J = 11.3 Hz), 7.26 (1H, d, J = 7.2 Hz), 7.39 (1H, s), 7.4-7.55 (1H, m), 12.0 (1H, s), 14.53 (1H, s) |
| 234 | 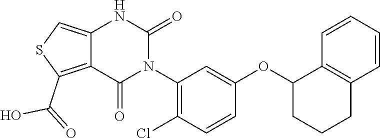 [SMILES: O=C(O)c1scc2[nH]c(=O)n(-c3cc(OC4CCCc5ccccc54)ccc3Cl)c(=O)c12] | (DMSO-d6) 1.7-2.1 (4H, m), 2.65-2.9 (2H, m), 5.45-5.5 (1H, m), 7.1-7.45 (7H, m), 7.57 (1H, d, J = 9.0 Hz), 12.0-12.1 (1H, m), 14.45 (1H, s) |
| 235 | 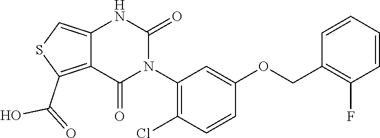 [SMILES: O=C(O)c1scc2[nH]c(=O)n(-c3cc(OCc4ccccc4F)ccc3Cl)c(=O)c12] | (DMSO-d6) 5.14 (2H, s), 7.15-7.5 (6H, m), 7.55-7.65 (2H, m), 12.05 (1H, s), 14.43 (1H, s) |
| 236 | 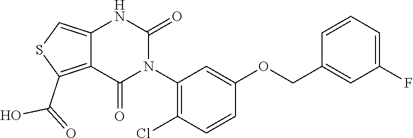 [SMILES: O=C(O)c1scc2[nH]c(=O)n(-c3cc(OCc4cccc(F)c4)ccc3Cl)c(=O)c12] | (DMSO-d6) 5.14 (2H, s), 7.15-7.25 (2H, m), 7.25-7.5 (5H, m), 7.58 (1H, d, J = 9.1 Hz), 12.05 (1H, s), 14.43 (1H, s) |
| 237 | 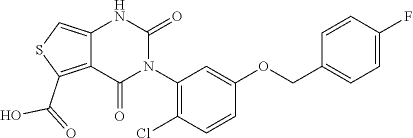 [SMILES: O=C(O)c1scc2[nH]c(=O)n(-c3cc(OCc4ccc(F)cc4)ccc3Cl)c(=O)c12] | (DMSO-d6) 5.09 (2H, s), 7.15-7.3 (3H, m), 7.32 (1H, d, J = 2.9 Hz), 7.41 (1H, s), 7.5-7.6 (3H, m), 12.04 (1H, s), 14.44 (1H, s) |
| 238 | 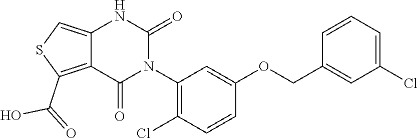 [SMILES: O=C(O)c1scc2[nH]c(=O)n(-c3cc(OCc4cccc(Cl)c4)ccc3Cl)c(=O)c12] | (DMSO-d6) 5.13 (2H, s), 7.19 (1H, dd, J = 9.0 Hz, 3.0 Hz), 7.3-7.5 (5H, m), 7.5- 7.6 (2H, m), 12.05 (1H, s), 14.44 (1H, s) |
| 239 | 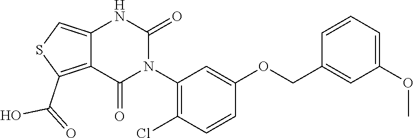 [SMILES: COc1cccc(COc2ccc(Cl)c(-n3c(=O)[nH]c4csc(C(=O)O)c4c3=O)c2)c1] | (DMSO-d6) 3.76 (3H, s), 5.08 (2H, s), 6.85-6.95 (1H, m), 7.0-7.05 (2H, m), 7.18 (1H, dd, J = 9.1 Hz, 3.1 Hz), 7.25- 7.35 (2H, m), 7.41 (1H, s), 7.57 (1H, d, J = 9.1 Hz), 12.04 (1H, s), 14.44 (1H, s) |
| 240 | 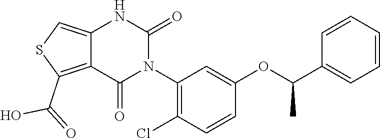 [SMILES: C[C@@H](Oc1ccc(Cl)c(-n2c(=O)[nH]c3csc(C(=O)O)c3c2=O)c1)c1ccccc1] | (DMSO-d6) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 241 | 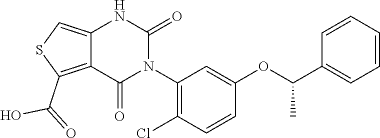 [SMILES: C[C@H](Oc1ccc(Cl)c(-n2c(=O)[nH]c3csc(C(=O)O)c3c2=O)c1)c1ccccc1] | (DMSO-d6) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 242 | 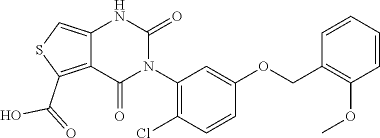 [SMILES: COc1ccccc1COc1ccc(Cl)c(-n2c(=O)[nH]c3csc(C(=O)O)c3c2=O)c1] | (DMSO-d6) 3.82 (3H, s), 5.04 (2H, s), 6.95-7.0 (1H, m), 7.06 (1H, d, J = 7.9 Hz), 7.17 (1H, dd, J = 9.1 Hz, 2.9 Hz), 7.3-7.45 (4H, m), 7.56 (1H, d, J = 9.1 Hz), 12.04 (1H, s), 14.46 (1H, s) |
| 243 | 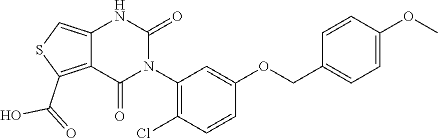 [SMILES: COc1ccc(COc2ccc(Cl)c(-n3c(=O)[nH]c4csc(C(=O)O)c4c3=O)c2)cc1] | (DMSO-d6) 3.76 (3H, s), 5.02 (2H, s), 6.9-7.0 (2H, m), 7.15-7.2 (1H, m), 7.3 (1H, d, J = 3.3 Hz), 7.35-7.45 (3H, m), 7.56 (1H, d, J = 9.0 Hz), 12.04 (1H, s), 14.46 (1H, s) |
| 244 | 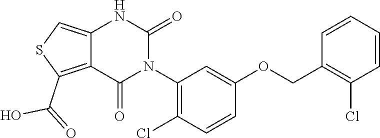 [SMILES: O=C(O)c1scc2[nH]c(=O)n(-c3cc(OCc4ccccc4Cl)ccc3Cl)c(=O)c12] | (DMSO-d6) 5.18 (2H, s), 7.02 (1H, s), 7.16 (1H, dd, J = 9.0 Hz, 2.7 Hz), 7.25- 7.4 (2H, m), 7.5-7.6 (2H, m), 7.8-7.9 (1H, m), 8.58 (1H, d, J = 4.5 Hz), 11.0- 12.5 (1H, br) |
| 245 | 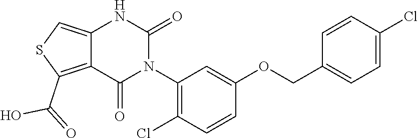 [SMILES: O=C(O)c1scc2[nH]c(=O)n(-c3cc(OCc4ccc(Cl)cc4)ccc3Cl)c(=O)c12] | (DMSO-d6) 5.11 (2H, s), 7.15-7.2 (1H, m), 7.32 (1H, d, J = 2.9 Hz), 7.41 (1H, s), 7.45-7.55 (4H, m), 7.57 (1H, d, J = 8.6 Hz), 12.06 (1H, s), 14.43 (1H, s) |
| 246 | 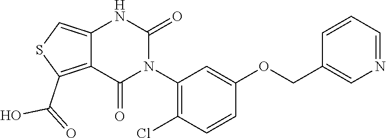 [SMILES: O=C(O)c1scc2[nH]c(=O)n(-c3cc(OCc4cccnc4)ccc3Cl)c(=O)c12] | (DMSO-d6) 5.16 (2H, s), 7.1-7.25 (2H, m), 7.31 (1H, d, J = 2.9 Hz), 7.4-7.5 (1H, m), 7.56 (1H, d, J = 9.0 Hz), 7.85-7.95 (1H, m), 8.5-8.6 (1H, m), 8.69 (1H, s), 11.0-13.0 (1H, br) |
| 247 | 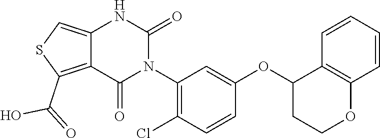 [SMILES: O=C(O)c1scc2[nH]c(=O)n(-c3cc(OC4CCOc5ccccc54)ccc3Cl)c(=O)c12] | (DMSO-d6) 2.1-2.25 (2H, m), 4.1-4.2 (1H, m), 4.25-4.35 (1H, m), 5.45-5.55 (1H, m), 6.8-6.95 (2H, m), 7.2-7.35 (3H, m), 7.35-7.45 (2H, m), 7.59 (1H, d, J = 8.7 Hz), 12.0-12.05 (1H, m), 14.42 (1H, s) |
| 248 | 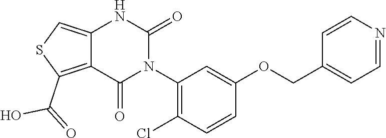 [SMILES: O=C(O)c1scc2[nH]c(=O)n(-c3cc(OCc4ccncc4)ccc3Cl)c(=O)c12] | (DMSO-d6) 5.2 (2H, s), 7.1-7.2 (2H, m), 7.3 (1H, d, J = 2.9 Hz), 7.4-7.5 (2H, m), 7.56 (1H, d, J = 8.8 Hz), 8.55-8.65 (2H, m), 11.0-13.0 (1H, br) |
| 249 | 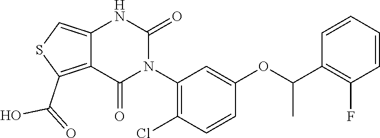 [SMILES: CC(Oc1ccc(Cl)c(-n2c(=O)[nH]c3csc(C(=O)O)c3c2=O)c1)c1ccccc1F] | (DMSO-d6) 1.6 (3H, d, J = 6.3 Hz), 5.65-5.75 (1H, m), 7.0-7.1 (1H, m), 7.15-7.3 (3H, m), 7.3-7.45 (2H, m), 7.45-7.55 (2H, m), 11.95-12.1 (1H, m) 14.42 (1H, s) |
| 250 | 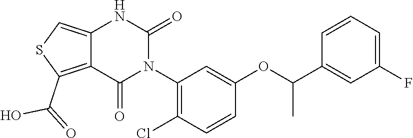 [SMILES: CC(Oc1ccc(Cl)c(-n2c(=O)[nH]c3csc(C(=O)O)c3c2=O)c1)c1cccc(F)c1] | (DMSO-d6) 1.5-1.6 (3H, m), 5.5-5.6 (1H, m), 7.0-7.15 (2H, m), 7.2-7.3 (3H, m), 7.35-7.55 (3H, m), 11.95-12.0 (1H, m), 14.41 (1H, s) |
| 251 | 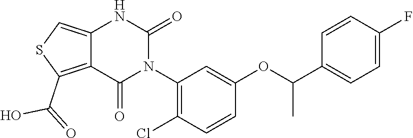 [SMILES: CC(Oc1ccc(Cl)c(-n2c(=O)[nH]c3csc(C(=O)O)c3c2=O)c1)c1ccc(F)cc1] | (DMSO-d6) 1.5-1.6 (3H, m), 5.45-5.6 (1H, m), 7.0-7.1 (1H, m), 7.15-7.25 (3H, m), 7.4 (1H, d, J = 4.1 Hz), 7.45-7.55 (3H, m), 11.95-12.05 (1H, m), 14.42 (1H, s) |
| 252 | 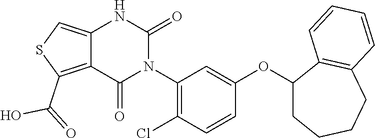 [SMILES: O=C(O)c1scc2[nH]c(=O)n(-c3cc(OC4CCCCc5ccccc54)ccc3Cl)c(=O)c12] | (DMSO-d6) 1.35-1.5 (1H, m), 1.65-2.05 (5H, m), 2.8-3.0 (2H, m), 5.45-5.6 (1H, m), 7.05-7.2 (4H, m), 7.25-7.35 (2H, m), 7.39 (1H, d, J = 3.8 Hz), 7.52 (1H, d, J = 8.8 Hz), 12.02 (1H, s), 14.43 (1H, s) |
| 253 | 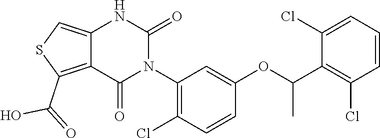 [SMILES: CC(Oc1ccc(Cl)c(-n2c(=O)[nH]c3csc(C(=O)O)c3c2=O)c1)c1c(Cl)cccc1Cl] | (DMSO-d6) 1.73 (3H, d, J = 6.6 Hz), 6.07 (1H, q, J = 6.6 Hz), 6.89 (1H, dd, J = 9.0 Hz, 3.0 Hz), 7.15-7.2 (1H, m), 7.3-7.4 (2H, m), 7.45-7.55 (3H, m), 12.0 (1H, s), 14.4 (1H, s) |
| 254 | 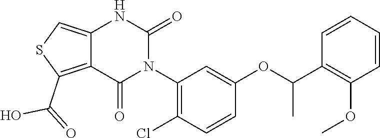 [SMILES: COc1ccccc1C(C)Oc1ccc(Cl)c(-n2c(=O)[nH]c3csc(C(=O)O)c3c2=O)c1] | (DMSO-d6) 1.53 (3H, d, J = 6.2 Hz), 5.68 (1H, q, J = 6.2 Hz), 6.85-7.0 (2H, m), 7.05 (1H, d, J = 8.4 Hz), 7.2 (1H, t, J = 3.2 Hz), 7.25-7.4 (3H, m), 7.47 (1H, d, J = 9.3 Hz), 12.01 (1H, S), 14.45 (1H, s) |
| 255 | 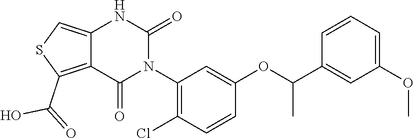 [SMILES: COc1cccc(C(C)Oc2ccc(Cl)c(-n3c(=O)[nH]c4csc(C(=O)O)c4c3=O)c2)c1] | (DMSO-d6) 1.5-1.6 (3H, m), 3.7-3.75 (3H, m), 5.4-5.5 (1H, m), 6.8-6.9 (1H, m), 6.95-7.1 (3H, m), 7.2-7.3 (2H, m), 7.4 (1H, d, J = 3.4 Hz), 7.47 (1H, dd, J = 9.0 Hz, 1.6 Hz), 11.95-12.05 (1H, m), 14.43 (1H, s) |
| 256 | 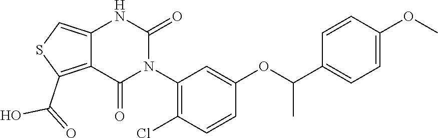 [SMILES: COc1ccc(C(C)Oc2ccc(Cl)c(-n3c(=O)[nH]c4csc(C(=O)O)c4c3=O)c2)cc1] | (DMSO-d6) 1.5-1.6 (3H, m), 3.73 (3H, s), 5.4-5.5 (1H, m), 6.85-6.95 (2H, m), 7.0-7.05 (1H, m), 7.15-7.25 (1H, m), 7.3-7.5 (4H, m), 11.95-12.05 (1H, m), 14.44 (1H, s) |
| 257 | 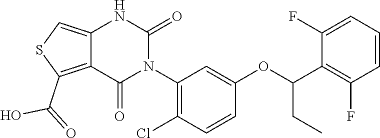 [SMILES: CCC(Oc1ccc(Cl)c(-n2c(=O)[nH]c3csc(C(=O)O)c3c2=O)c1)c1c(F)cccc1F] | (DMSO-d6) 0.93 (3H, t, J = 7.5 Hz), 1.9- 2.05 (1H, m), 2.1-2.25 (1H, m), 5.55 (1H, t, J = 7.3 Hz), 6.95-7.25 (4H, m), 7.35-7.5 (2H, m), 7.52 (1H, d, J = 8.7 Hz), 11.95-12.05 (1H, m), 14.4 (1H, s) |
| 258 | 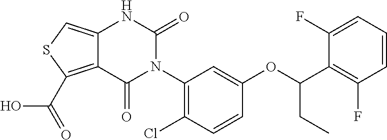 [SMILES: CCC(Oc1ccc(Cl)c(-n2c(=O)[nH]c3csc(C(=O)O)c3c2=O)c1)c1c(F)cccc1F] | (DMSO-d6) 0.93 (3H, t, J = 7.4 Hz), 1.9- 2.05 (1H, m), 2.1-2.25 (1H, m), 5.5-5.6 (1H, m), 6.95-7.25 (4H, m), 7.35-7.5 (1H, m), 7.52 (1H, d, J = 9.2 Hz), 7.95 (1H, d, J = 6.7 Hz), 12.99 (1H, s), 13.8- 14.0 (1H, m) |
| 259 | 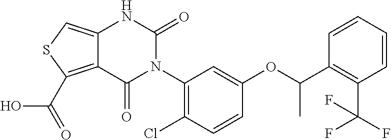 [SMILES: CC(Oc1ccc(Cl)c(-n2c(=O)[nH]c3csc(C(=O)O)c3c2=O)c1)c1ccccc1C(F)(F)F] | (DMSO-d6) 1.55-1.65 (3H, m), 5.65-5.8 (1H, m), 6.9-7.0 (1H, m), 7.2-7.3 (1H, m), 7.35-7.45 (1H, m), 7.45-7.6 (2H, m), 7.7-7.85 (3H, m), 12.03 (1H, s), 14.41 (1H, s) |
| 260 | 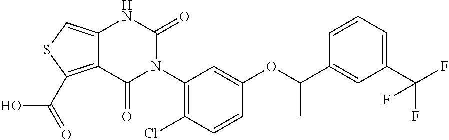 [SMILES: CC(Oc1ccc(Cl)c(-n2c(=O)[nH]c3csc(C(=O)O)c3c2=O)c1)c1cccc(C(F)(F)F)c1] | (DMSO-d6) 1.55-1.65 (3H, m), 5.6-5.7 (1H, m), 7.05-7.15 (1H, m), 7.25-7.3 (1H, m), 7.4 (1H, d, J = 5.6 Hz), 7.45- 7.55 (1H, m), 7.55-7.85 (4H, m), 11.95- 12.1 (1H, m), 14.4 (1H, s) |
| 261 | 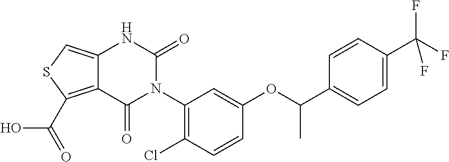 [SMILES: CC(Oc1ccc(Cl)c(-n2c(=O)[nH]c3csc(C(=O)O)c3c2=O)c1)c1ccc(C(F)(F)F)cc1] | (DMSO-d6) 1.55-1.65 (3H, m), 5.6-5.7 (1H, m), 7.0-7.1 (1H. m), 7.25(1H, dd, J = 6.6 Hz, 3.0 Hz), 7.4 (1H, d, J = 5.3 Hz), 7.45-7.55 (1H, m), 7.6-7.7 (2H, m), 7.7-7.8 (2H, m), 11.95-12.1 (1H, m), 14.4 (1H, s) |
| 262 | 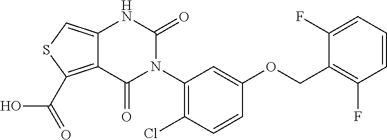 [SMILES: O=C(O)c1scc2[nH]c(=O)n(-c3cc(OCc4c(F)cccc4F)ccc3Cl)c(=O)c12] | (DMSO-d6) 5.13 (2H, s), 7.15-7.3 (3H, m), 7.34 (1H, d, J = 2.9 Hz), 7.4 (1H, s), 7.5-7.65 (2H, m), 12.03 (1H, s), 14.4 (1H, s) |
| 263 | 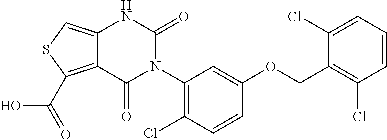 [SMILES: O=C(O)c1scc2[nH]c(=O)n(-c3cc(OCc4c(Cl)cccc4Cl)ccc3Cl)c(=O)c12] | (DMSO-d6) 5.24 (2H, s), 7.2-7.65 (7H, m), 12.02 (1H, s), 14.39 (1H, s) |
| 264 | 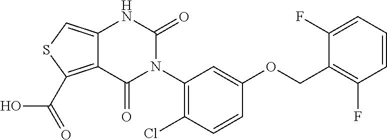 [SMILES: O=C(O)c1scc2[nH]c(=O)n(-c3cc(OCc4c(F)cccc4F)ccc3Cl)c(=O)c12] | (DMSO-d6) 5.12 (2H, s), 7.15-7.25 (3H, m), 7.32 (1H, d, J = 3.1 Hz), 7.5-7.65 (2H, m), 7.94 (1H, s), 12.8-13.2 (1H, br), 13.93 (1H, s) |
| 265 | 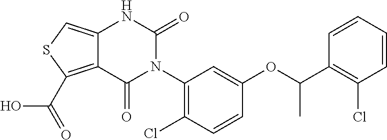 [SMILES: CC(Oc1ccc(Cl)c(-n2c(=O)[nH]c3csc(C(=O)O)c3c2=O)c1)c1ccccc1Cl] | (DMSO-d6) 1.59 (3H, d, J = 6.3 Hz), 5.71 (1H, q, J = 6.3 Hz), 6.85-6.95 (1H, m), 7.2-7.25 (1H, m), 7.3-7.45 (3H, m), 7.45-7.55 (3H, m), 11.95-12.05 (1H, m), 14.39 (1H, s) |
| 266 | 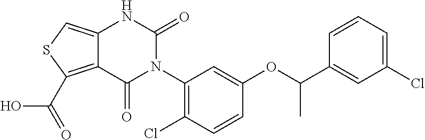 [SMILES: CC(Oc1ccc(Cl)c(-n2c(=O)[nH]c3csc(C(=O)O)c3c2=O)c1)c1cccc(Cl)c1] | (DMSO-d6) 1.56 (3H, d, J = 6.2 Hz), 5.45-5.6 (1H, m), 7.0-7.1 (1H, m), 7.2- 7.55 (7H, m), 11.95-12.05 (1H, m), 14.39 (1H, s) |
| 267 | 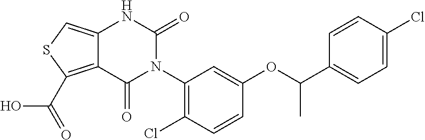 [SMILES: CC(Oc1ccc(Cl)c(-n2c(=O)[nH]c3csc(C(=O)O)c3c2=O)c1)c1ccc(Cl)cc1] | (DMSO-d6) 1.5-1.6 (3H, m), 5.45-5.6 (1H, m), 7.0-7.05 (1H, m), 7.22 (1H, dd, J = 8.9 Hz, 3.0 Hz), 7.35-7.5 (6H, m), 11.9-12.05 (1H, m), 14.39 (1H, s) |
| 268 | 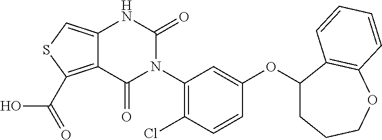 [SMILES: O=C(O)c1scc2[nH]c(=O)n(-c3cc(OC4CCCOc5ccccc54)ccc3Cl)c(=O)c12] | (DMSO-d6) 1.9-2.15 (4H, m), 3.75-3.85 (1H, m), 4.1-4.25 (1H, m), 5.45-5.55 (1H, m), 6.95-7.45 (7H, m), 7.53 (1H, d, J = 8.9 Hz), 11.95-12.05 (1H, m), 14.42 (1H, s) |
| 269 | 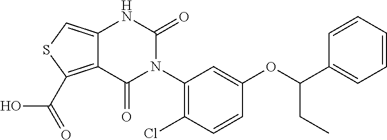 [SMILES: CCC(Oc1ccc(Cl)c(-n2c(=O)[nH]c3csc(C(=O)O)c3c2=O)c1)c1ccccc1] | (DMSO-d6) 0.91 (3H, t, J = 7.5 Hz), 1.75-2.0 (2H, m), 5.2-5.3 (1H, m), 6.95- 7.05 (1H, m), 7.15-7.5 (8H, m), 11.95- 12.05 (1H, m), 14.39 (1H, s) |
| 270 | 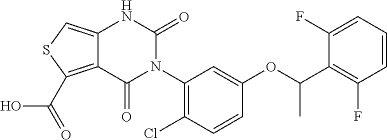 [SMILES: CC(Oc1ccc(Cl)c(-n2c(=O)[nH]c3csc(C(=O)O)c3c2=O)c1)c1c(F)cccc1F] | (DMSO-d6) 1.71 (3H, d, J = 6.5 Hz), 5.81 (1H, q, J = 6.5 Hz), 7.0-7.15 (3H, m), 7.2-7.25 (1H, m), 7.35-7.5 (2H, m), 7.53 (1H, d, J = 9.2 Hz), 11.95-12.05 (1H, m), 14.41 (1H, s) |
| 271 | 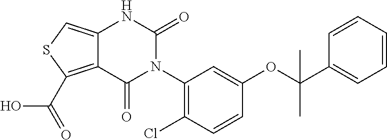 [SMILES: CC(C)(Oc1ccc(Cl)c(-n2c(=O)[nH]c3csc(C(=O)O)c3c2=O)c1)c1ccccc1] | (DMSO-d6) 1.65-1.75 (6H, m), 6.64 (1H, dd, J = 8.7 Hz, 2.8 Hz), 7.07 (1H, d, J = 2.8 Hz), 7.25-7.5 (7H, m), 11.97 (1H, s), 14.42 (1H, s) |
| 272 | 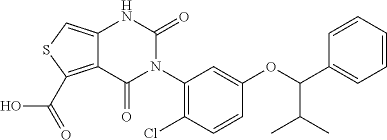 [SMILES: CC(C)C(Oc1ccc(Cl)c(-n2c(=O)[nH]c3csc(C(=O)O)c3c2=O)c1)c1ccccc1] | (DMSO-d6) 0.82 (3H, d, J = 6.4 Hz), 0.95-1.05 (3H, m), 2.05-2.15 (1H, m), 5.0-5.1 (1H, m), 6.95-7.05 (1H, m), 7.15-7.5 (8H, m), 11.95-12.05 (1H, m), 14.4 (1H, s) |
| 273 | 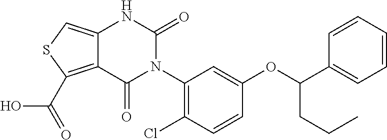 [SMILES: CCCC(Oc1ccc(Cl)c(-n2c(=O)[nH]c3csc(C(=O)O)c3c2=O)c1)c1ccccc1] | (DMSO-d6) 0.91 (3H, t, J = 7.4 Hz), 1.25-1.5 (2H, m), 1.7-1.8 (1H, m), 1.85- 2.0 (1H, m), 5.3-5.35 (1H, m), 6.95-7.05 (1H, m), 7.15-7.5 (8H, m), 11.95-12.05 (1H, m), 14.41 (1H, brs) |
| 274 | 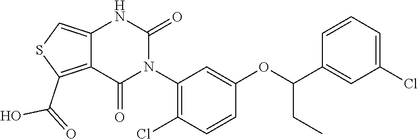 [SMILES: CCC(Oc1ccc(Cl)c(-n2c(=O)[nH]c3csc(C(=O)O)c3c2=O)c1)c1cccc(Cl)c1] | (DMSO-d6) 0.91 (3H, t, J = 7.4 Hz), 1.75-2.0 (2H, m), 5.25-5.35 (1H, m), 7.0-7.1 (1H, m), 7.2-7.25 (1H, m), 7.3- 7.55 (6H, m), 11.95-12.05 (1H, m), 14.4 (1H, s) |
| 275 | 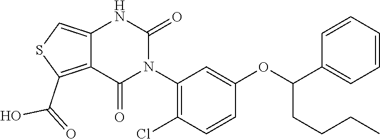 [SMILES: CCCCC(Oc1ccc(Cl)c(-n2c(=O)[nH]c3csc(C(=O)O)c3c2=O)c1)c1ccccc1] | (DMSO-d6) 0.85 (3H, t, J = 7.1 Hz), 1.2- 1.45 (4H, m), 1.7-1.85 (1H, m), 1.9-2.0 (1H, m), 5.25-5.35 (1H, m), 6.95-7.05 (1H, m), 7.15-7.5 (8H, m), 11.95-12.05 (1H, m), 14.41 (1H, s) |
| 276 | 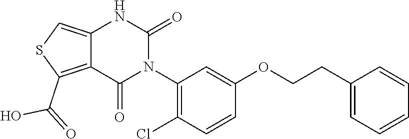 [SMILES: O=C(O)c1scc2[nH]c(=O)n(-c3cc(OCCc4ccccc4)ccc3Cl)c(=O)c12] | (DMSO-d6) 3.05 (2H, t, J = 7.0 Hz), 4.19 (2H, t, J = 7.0 Hz), 7.05-7.15 (1H, m), 7.2-7.35 (6H, m), 7.4 (1H, s), 7.54 (1H, d, J = 9.2 Hz), 12.03 (1H, s), 14.45 (1H, s) |
| 277 | 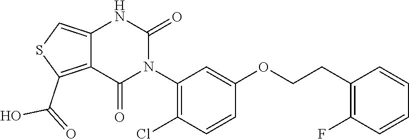 [SMILES: O=C(O)c1scc2[nH]c(=O)n(-c3cc(OCCc4ccccc4F)ccc3Cl)c(=O)c12] | (DMSO-d6) 3.09 (2H, t, J = 6.8 Hz), 4.19 (2H, t, J = 6.8 Hz), 7.05-7.35 (5H, m), 7.35-7.45 (2H, m), 7.54 (1H, d, J = 8.9 Hz), 12.03 (1H, s), 14.45 (1H, s) |
| 278 | 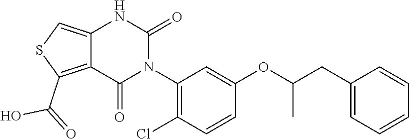 [SMILES: CC(Cc1ccccc1)Oc1ccc(Cl)c(-n2c(=O)[nH]c3csc(C(=O)O)c3c2=O)c1] | (DMSO-d6) 1.22 (3H, d, J = 6.0 Hz), 2.8-2.9 (1H, m), 2.95-3.05 (1H, m), 4.6- 4.75 (1H, m), 7.09 (1H, dd, J = 9.0 Hz, 3.0 Hz), 7.15-7.35 (6H, m), 7.35-7.45 (1H, m), 7.52 (1H, d, J = 9.2 Hz), 12.02 (1H, s), 14.46 (1H, s) |
| 279 | 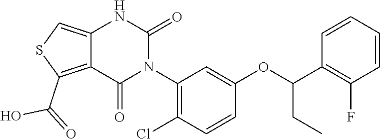 [SMILES: CCC(Oc1ccc(Cl)c(-n2c(=O)[nH]c3csc(C(=O)O)c3c2=O)c1)c1ccccc1F] | (DMSO-d6) 0.93 (3H, t, J = 7.5 Hz), 1.8- 2.05 (2H, m), 5.47 (1H, t, J = 6.1 Hz), 6.95-7.05 (1H, m), 7.15-7.25 (3H, m), 7.3-7.55 (4H, m), 11.95-12.05 (1H, m), 14.35-14.45 (1H, m) |
| 280 | 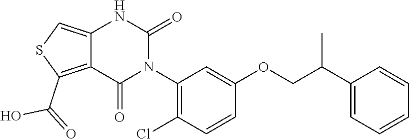 [SMILES: CC(COc1ccc(Cl)c(-n2c(=O)[nH]c3csc(C(=O)O)c3c2=O)c1)c1ccccc1] | (DMSO-d6) 1.32 (3H, d, J = 6.9 Hz), 3.15-3.3 (1H, m), 4.0-4.15 (2H, m), 7.09 (1H, dd, J = 8.9 Hz, 3.1 Hz), 7.15-7.45 (7H, m), 7.52 (1H, d, J = 8.9 Hz), 12.02 (1H, s), 14.45 (1H, s) |
| 281 | 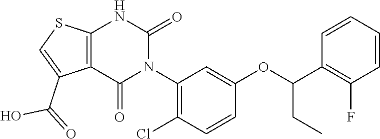 [SMILES: CCC(Oc1ccc(Cl)c(-n2c(=O)[nH]c3scc(C(=O)O)c3c2=O)c1)c1ccccc1F] | (DMSO-d6) 0.93 (3H, t, J = 7.5 Hz), 1.8- 2.05 (2H, m), 5.48 (1H, t, J = 6.5 Hz), 6.95-7.05 (1H, m), 7.15-7.25 (3H, m), 7.3-7.4 (1H, m), 7.4-7.55 (2H, m), 7.94 (1H, d, J = 3.4 Hz), 12.98 (1H, s), 13.93 (1H, s) |
| 282 | 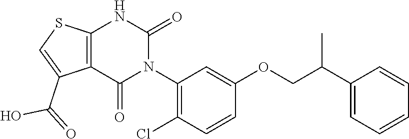 [SMILES: CC(COc1ccc(Cl)c(-n2c(=O)[nH]c3scc(C(=O)O)c3c2=O)c1)c1ccccc1] | (DMSO-d6) 1.32 (3H, d, J = 6.9 Hz), 3.15-3.3 (1H, m), 4.0-4.15 (2H, m), 7.05-7.1 (1H, m), 7.15-7.4 (6H, m), 7.52 (1H, d, J = 9.0 Hz), 7.95 (1H, s), 13.0 (1H, s), 13.98 (1H, s) |
| 283 | 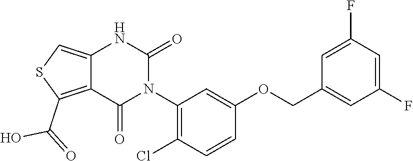 [SMILES: O=C(O)c1scc2[nH]c(=O)n(-c3cc(OCc4cc(F)cc(F)c4)ccc3Cl)c(=O)c12] | (DMSO-d6) 5.15 (2H, s), 7.15-7.3 (4H, m), 7.35 (1H, d, J = 2.8 Hz), 7.46 (1H, s), 7.59 (1H, d, J = 8.8 Hz), 12.19 (1H, s) |
| 284 | 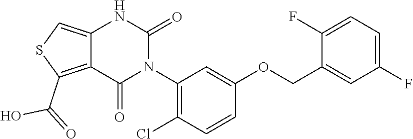 [SMILES: O=C(O)c1scc2[nH]c(=O)n(-c3cc(OCc4cc(F)ccc4F)ccc3Cl)c(=O)c12] | (DMSO-d6) 5.14 (2H, s), 7.2-7.5 (6H, m), 7.6 (1H, d, J = 8.7 Hz), 12.11 (1H, s), 14.44 (1H, s) |
| 285 | 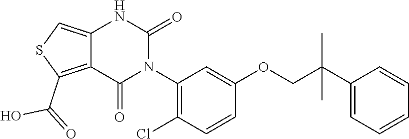 [SMILES: CC(C)(COc1ccc(Cl)c(-n2c(=O)[nH]c3csc(C(=O)O)c3c2=O)c1)c1ccccc1] | (DMSO-d6) 1.4 (6H, s), 4.01 (2H, s), 7.05-7.15 (1H, m), 7.15-7.25 (2H, m), 7.3-7.35 (2H, m), 7.39 (1H, s), 7.4-7.5 (2H, m), 7.52 (1H, d, J = 9.2 Hz), 12.0 (1H, s), 14.44 (1H, s) |
| 286 | 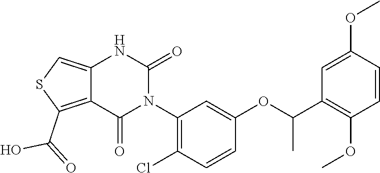 [SMILES: COc1ccc(OC)c(C(C)Oc2ccc(Cl)c(-n3c(=O)[nH]c4csc(C(=O)O)c4c3=O)c2)c1] | (DMSO-d6) 1.52 (3H, d, J = 6.3 Hz), 3.67 (3H, s), 3.75-3.85 (3H, m), 5.65 (1H, q, J = 6.3 Hz), 6.8-7.0 (4H, m), 7.15-7.25 (1H, m), 7.39 (1H, d, J = 1.9 Hz), 7.48 (1H, d, J = 8.9 Hz), 11.99 (1H, s), 14.42 (1H, s) |
| 287 | 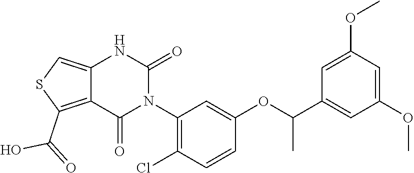 [SMILES: COc1cc(OC)cc(C(C)Oc2ccc(Cl)c(-n3c(=O)[nH]c4csc(C(=O)O)c4c3=O)c2)c1] | (DMSO-d6) 1.55 (3H, d, J = 6.3 Hz), 3.31 (3H, s), 3.7-3.75 (3H, m), 5.35-5.45 (1H, m), 6.35-6.45 (1H, m), 6.5-6.6 (2H, m), 7.0-7.1 (1H, m), 7.2-7.3 (1H, m), 7.39 (1H, d, J = 2.0 Hz), 7.45-7.5 (1H, m), 11.95-12.05 (1H, m), 14.41 (1H, s) |
| 288 | 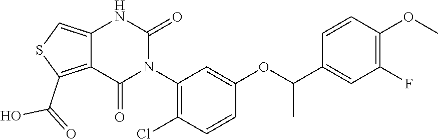 [SMILES: COc1ccc(C(C)Oc2ccc(Cl)c(-n3c(=O)[nH]c4csc(C(=O)O)c4c3=O)c2)cc1F] | (DMSO-d6) 1.5-1.6 (3H, m), 3.81 (3H, s), 5.4-5.5 (1H, m), 7.0-7.3 (5H, m), 7.4 (1H, d, J = 3.9 Hz), 7.48 (1H, dd, J = 8.9 Hz, 1.6 Hz), 11.95-12.05 (1H, m), 14.41 (1H, s) |
| 289 | 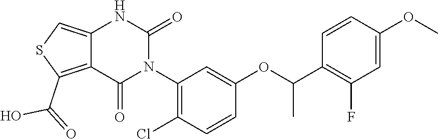 [SMILES: COc1ccc(C(C)Oc2ccc(Cl)c(-n3c(=O)[nH]c4csc(C(=O)O)c4c3=O)c2)c(F)c1] | (DMSO-d6) 1.58 (3H, d, J = 6.4 Hz), 3.75 (3H, s), 5.55-5.7 (1H, m), 6.75-6.9 (2H, m), 6.95-7.05 (1H, m), 7.15-7.55 (4H, m), 11.95-12.05 (1H, m), 14.42 (1H, s) |
| 290 | 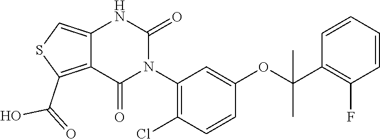 [SMILES: CC(C)(Oc1ccc(Cl)c(-n2c(=O)[nH]c3csc(C(=O)O)c3c2=O)c1)c1ccccc1F] | (DMSO-d6) 1.75 (6H, s), 6.78 (1H, dd, J = 9.0 Hz, 3.0 Hz), 7.0-7.55 (7H, m), 11.91 (1H, brs), 14.0-14.8 (1H, br) |
| 291 | 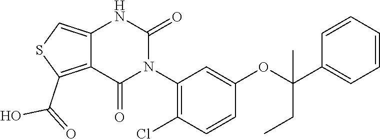 [SMILES: CCC(C)(Oc1ccc(Cl)c(-n2c(=O)[nH]c3csc(C(=O)O)c3c2=O)c1)c1ccccc1] | (DMSO-d6) 0.8 (3H, t, J = 7.3 Hz), 1.65- 1.7 (3H, m), 1.85-2.05 (2H, m), 6.55- 6.65 (1H, m), 7.08 (1H, dd, J = 6.6 Hz, 3.0 Hz), 7.25-7.45 (7H, m), 11.97 (1H, s), 14.43 (1H, s) |
| 292 | 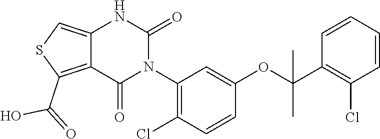 [SMILES: CC(C)(Oc1ccc(Cl)c(-n2c(=O)[nH]c3csc(C(=O)O)c3c2=O)c1)c1ccccc1Cl] | (DMSO-d6) 1.83 (6H, s), 6.69 (1H, dd, J = 9.1 Hz, 3.1 Hz), 7.06 (1H, d, J = 3.1 Hz), 7.3-7.5 (5H, m), 7.61 (1H, dd, J = 7.7 Hz, 1.8 Hz), 11.96 (1H, s), 14.42 (1H, s) |
| 293 | 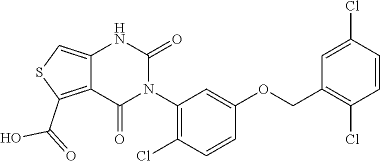 [SMILES: O=C(O)c1scc2[nH]c(=O)n(-c3cc(OCc4cc(Cl)ccc4Cl)ccc3Cl)c(=O)c12] | (DMSO-d6) 5.15 (2H, s), 7.15-7.75 (7H, m), 12.03 (1H, s), 14.41 (1H, s) |
| 294 | 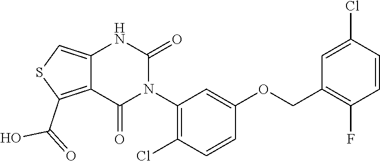 [SMILES: O=C(O)c1scc2[nH]c(=O)n(-c3cc(OCc4cc(Cl)ccc4F)ccc3Cl)c(=O)c12] | (DMSO-d6) 5.14 (2H, s), 7.23 (1H, dd, J = 8.8 Hz, 3.2 Hz), 7.3-7.45 (3H, m), 7.45-7.55 (1H, m), 7.59 (1H, d, J = 9.1 Hz), 7.65-7.7 (1H, m), 12.03 (1H, s), 14.41 (1H, s) |
| 295 | 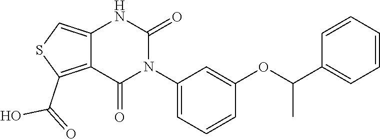 [SMILES: CC(Oc1cccc(-n2c(=O)[nH]c3csc(C(=O)O)c3c2=O)c1)c1ccccc1] | (DMSO-d6) 1.56 (3H, d, J = 6.3 Hz), 5.45-5.55 (1H, m), 6.85-6.9 (1H, m), 6.95-7.05 (2H, m), 7.25-7.45 (7H, m), 11.9 (1H, s) |
| 296 | 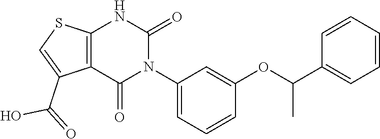 [SMILES: CC(Oc1cccc(-n2c(=O)[nH]c3scc(C(=O)O)c3c2=O)c1)c1ccccc1] | (DMSO-d6) 1.56 (3H, d, J = 6.4 Hz), 5.5 (1H, q, J = 6.4 Hz), 6.85-6.9 (1H, m), 6.9-7.05 (2H, m), 7.25-7.4 (4H, m), 7.4- 7.45 (2H, m), 7.99 (1H, s), 12.86 (1H, s), 14.43 (1H, s) |
| 297 | 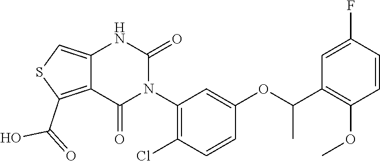 [SMILES: COc1ccc(F)cc1C(C)Oc1ccc(Cl)c(-n2c(=O)[nH]c3csc(C(=O)O)c3c2=O)c1] | (DMSO-d6) 1.52 (3H, d, J = 6.0 Hz), 3.84 (3H, s), 5.6-5.7 (1H, m), 6.85-7.55 (7H, m), 11.99 (1H, s), 14.42 (1H, s) |
| 298 | 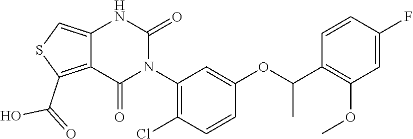 [SMILES: COc1cc(F)ccc1C(C)Oc1ccc(Cl)c(-n2c(=O)[nH]c3csc(C(=O)O)c3c2=O)c1] | (DMSO-d6) 1.52 (3H, d, J = 6.4 Hz), 3.85-3.9 (3H, m), 5.6-5.7 (1H, m), 6.75- 6.8 (1H, m), 6.85-7.0 (2H, m), 7.15-7.25 (1H, m), 7.3-7.45 (2H, m), 7.48 (1H, d, J = 9.2 Hz), 12.0 (1H, s), 14.44 (1H, s) |
| 299 | 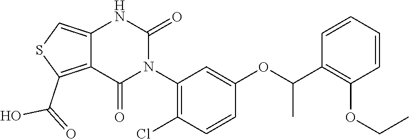 [SMILES: CCOc1ccccc1C(C)Oc1ccc(Cl)c(-n2c(=O)[nH]c3csc(C(=O)O)c3c2=O)c1] | (DMSO-d6) 1.3-1.4 (3H, m), 1.54 (3H, d, J = 6.3 Hz), 4.05-4.2 (2H, m), 5.65-5.75 (1H, m), 6.85-7.0 (2H, m), 7.0-7.05 (1H, m), 7.15-7.35 (3H, m), 7.39 (1H, d, J = 4.2 Hz), 7.48 (1H, d, J = 9.1 Hz), 11.95- 12.05 (1H, m), 14.42 (1H, s) |
| 300 | 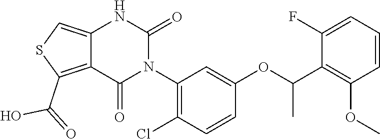 [SMILES: COc1cccc(F)c1C(C)Oc1ccc(Cl)c(-n2c(=O)[nH]c3csc(C(=O)O)c3c2=O)c1] | (DMSO-d6) 1.66 (3H, d, J = 6.6 Hz), 3.85-3.9 (3H, m), 5.8-5.9 (1H, m), 6.75- 6.85 (1H, m), 6.85-7.0 (2H, m), 7.17 (1H, d, J = 2.6 Hz), 7.25-7.4 (2H, m), 7.48 (1H, d, J = 8.6 Hz), 12.0 (1H, s), 14.42 (1H, s) |
| 301 | 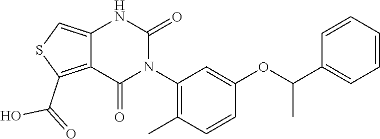 [SMILES: Cc1ccc(OC(C)c2ccccc2)cc1-n1c(=O)[nH]c2csc(C(=O)O)c2c1=O] | (DMSO-d6) 1.5-1.6 (3H, m), 1.95 (3H, s), 5.4-5.5 (1H, m), 6.85-6.95 (1H, m), 6.98 (1H, dd, J = 5.8 Hz, 2.6 Hz), 7.15- 7.45 (7H, m), 11.85-11.95 (1H, m), 14.76 (1H, s) |
| 302 | 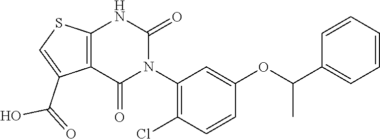 [SMILES: CC(Oc1ccc(Cl)c(-n2c(=O)[nH]c3scc(C(=O)O)c3c2=O)c1)c1ccccc1] | (DMSO-d6) 1.56 (3H, d, J = 6.3 Hz), 5.52 (1H, q, J = 6.3 Hz), 7.03 (1H, dd, J = 9.0 Hz, 3.0 Hz), 7.15-7.5 (7H, m), 7.9-8.0 (1H, m), 12.99 (1H, brs), 13.95 (1H, s) |
| 303 | 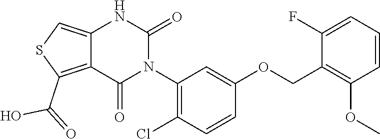 [SMILES: COc1cccc(F)c1COc1ccc(Cl)c(-n2c(=O)[nH]c3csc(C(=O)O)c3c2=O)c1] | (DMSO-d6) 3.85 (3H, s), 5.03 (2H, s), 6.85-7.0 (2H, m), 7.21 (1H, dd, J = 8.9 Hz, 3.1 Hz), 7.32 (1H, d, J = 3.1 Hz), 7.4-7.5 (2H, m), 7.58 (1H, d, J = 8.9 Hz), 12.06 (1H, s), 14.43 (1H, s) |
| 304 | 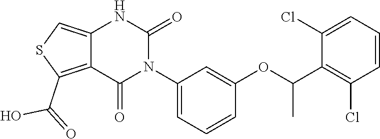 [SMILES: CC(Oc1cccc(-n2c(=O)[nH]c3csc(C(=O)O)c3c2=O)c1)c1c(Cl)cccc1Cl] | (DMSO-d6) 1.73 (3H, d, J = 6.7 Hz), 6.0-6.1 (1H, m), 6.8-6.85 (1H, m), 6.9- 7.0 (2H, m), 7.3-7.4 (3H, m), 7.45-7.5 (2H, m), 11.89 (1H, s), 14.86 (1H, s) |
| 305 | 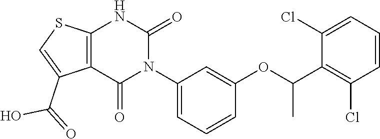 [SMILES: CC(Oc1cccc(-n2c(=O)[nH]c3scc(C(=O)O)c3c2=O)c1)c1c(Cl)cccc1Cl] | (DMSO-d6) 1.73 (3H, d, J = 6.7 Hz), 6.05 (1H, q, J = 6.7 Hz), 6.8-7.0 (3H, m), 7.3-7.4 (2H, m), 7.45-7.5 (2H, m), 7.99 (1H, s), 12.92 (1H, s), 14.43 (1H, s) |
| 306 | 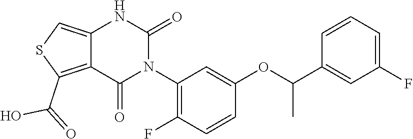 [SMILES: CC(Oc1ccc(F)c(-n2c(=O)[nH]c3csc(C(=O)O)c3c2=O)c1)c1cccc(F)c1] | (DMSO-d6) 1.56 (3H, d, J = 5.7 Hz), 5.45-5.55 (1H, m), 7.0-7.5 (8H, m), 11.95-12.05 (1H, m), 14.4 (1H, brs) |
| 307 | 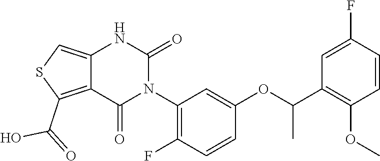 [SMILES: COc1ccc(F)cc1C(C)Oc1ccc(F)c(-n2c(=O)[nH]c3csc(C(=O)O)c3c2=O)c1] | (DMSO-d6) 1.52 (3H, d, J = 6.2 Hz), 3.84 (3H, s), 5.55-5.65 (1H, m), 6.9-7.0 (1H, m), 7.0-7.15 (4H, m), 7.2-7.3 (1H, m), 7.35-7.4 (1H, m), 11.98 (1H, s), 14.4 (1H, s) |
| 308 | 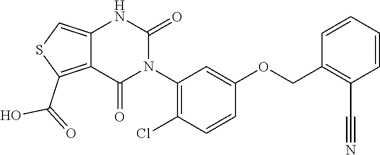 [SMILES: N#Cc1ccccc1COc1ccc(Cl)c(-n2c(=O)[nH]c3csc(C(=O)O)c3c2=O)c1] | (DMSO-d6) 5.2-5.35 (2H, m), 7.25 (1H, dd, J = 9.1 Hz, 3.0 Hz), 7.38 (1H, d, J = 3.0 Hz), 7.41 (1H, s), 7.55-7.65 (2H, m), 7.75-7.8 (2H, m), 7.94 (1H, d, J = 7.7 Hz), 12.05 (1H, s), 14.43 (1H, s) |
| 309 | 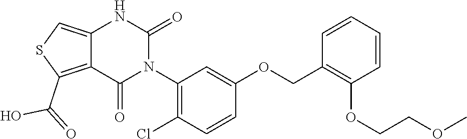 [SMILES: COCCOc1ccccc1COc1ccc(Cl)c(-n2c(=O)[nH]c3csc(C(=O)O)c3c2=O)c1] | (DMSO-d6) 3.26 (3H, s), 3.6-3.7 (2H, m), 4.1-4.2 (2H, m), 5.07 (2H, s), 6.95- 7.0 (1H, m), 7.07 (1H, d, J = 8.0 Hz), 7.15-7.2 (1H, m), 7.3-7.4 (2H, m), 7.4- 7.45 (2H, m), 7.55 (1H, d, J = 9.3 Hz), 12.04 (1H, s), 14.46 (1H, s) |
| 310 | 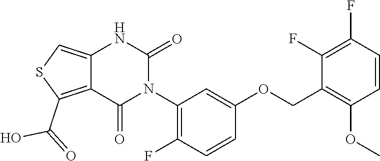 [SMILES: COc1ccc(F)c(F)c1COc1ccc(F)c(-n2c(=O)[nH]c3csc(C(=O)O)c3c2=O)c1] | (DMSO-d6) 3.83 (3H, s), 5.0-5.1 (2H, m), 6.9-6.95 (1H, m), 7.15-7.25 (2H, m), 7.3-7.4 (2H, m), 7.45-7.55 (1H, m), 12.02 (1H, s), 14.4 (1H, s) |
| 311 | 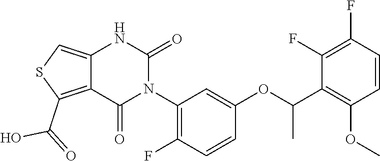 [SMILES: COc1ccc(F)c(F)c1C(C)Oc1ccc(F)c(-n2c(=O)[nH]c3csc(C(=O)O)c3c2=O)c1] | (DMSO-d6) 1.66 (3H, d, J = 6.5 Hz), 3.8-3.9 (3H, m), 5.75-5.85 (1H, m), 6.8- 7.0 (2H, m), 7.05-7.15 (1H, m), 7.2-7.4 (3H, m), 11.95-12.05 (1H, m), 14.39 (1H, s) |
| 312 | 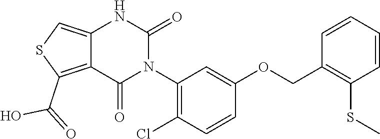 [SMILES: CSc1ccccc1COc1ccc(Cl)c(-n2c(=O)[nH]c3csc(C(=O)O)c3c2=O)c1] | (DMSO-d6) 2.48 (3H, s), 5.09 (2H, s), 7.15-7.25 (2H, m), 7.34 (1H, d, J = 2.9 Hz), 7.35-7.45 (3H, m), 7.49 (1H, d, J = 7.2 Hz), 7.58 (1H, d, J = 9.0 Hz), 12.04 (1H, s), 14.45 (1H, s) |
| 313 | 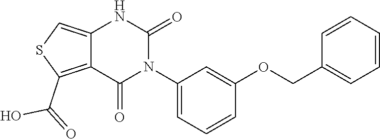 [SMILES: O=C(O)c1scc2[nH]c(=O)n(-c3cccc(OCc4ccccc4)c3)c(=O)c12] | (DMSO-d6) 5.1 (2H, s), 6.95-7.0 (1H, m), 7.05-7.15 (2H, m), 7.3-7.5 (7H, m), 11.88 (1H, s), 14.89 (1H, s) |
| 314 | 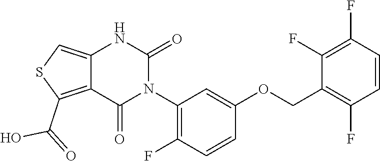 [SMILES: O=C(O)c1scc2[nH]c(=O)n(-c3cc(OCc4c(F)ccc(F)c4F)ccc3F)c(=O)c12] | (DMSO-d6) 5.14 (2H, s), 7.2-7.3 (3H, m), 7.35-7.45 (2H, m), 7.55-7.65 (1H, m), 12.02 (1H, s), 14.38 (1H, s) |
| 315 | 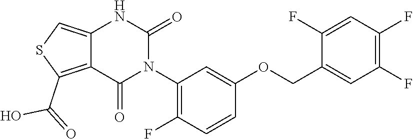 [SMILES: O=C(O)c1scc2[nH]c(=O)n(-c3cc(OCc4cc(F)c(F)cc4F)ccc3F)c(=O)c12] | (DMSO-d6) 5.08 (2H, s), 7.15-7.25 (1H, m), 7.25-7.3 (1H, m), 7.35-7.4 (2H, m), 7.55-7.7 (1H, m), 7.7-7.8 (1H, m), 12.0 (1H, s), 14.4 (1H, s) |
| 316 | 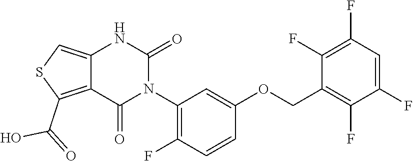 [SMILES: O=C(O)c1scc2[nH]c(=O)n(-c3cc(OCc4c(F)c(F)cc(F)c4F)ccc3F)c(=O)c12] | (DMSO-d6) 5.2 (2H, s), 7.2-7.3 (2H, m), 7.35-7.45 (2H, m), 7.95-8.05 (1H, m), 12.02 (1H, s), 14.38 (1H, s) |
| 317 | 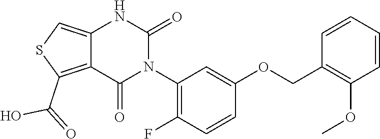 [SMILES: COc1ccccc1COc1ccc(F)c(-n2c(=O)[nH]c3csc(C(=O)O)c3c2=O)c1] | (DMSO-d6) 3.82 (3H, s), 5.02 (2H, s), 6.95-7.0 (1H, m), 7.06 (1H, d, J = 8.2 Hz), 7.1-7.2 (1H, m), 7.24 (1H, dd, J = 6.0 Hz, 3.2 Hz), 7.3-7.45 (4H, m), 12.01 (1H, s), 14.44 (1H, s) |
| 318 | 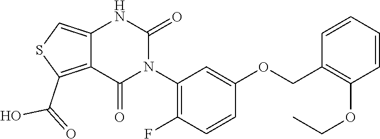 [SMILES: CCOc1ccccc1COc1ccc(F)c(-n2c(=O)[nH]c3csc(C(=O)O)c3c2=O)c1] | (DMSO-d6) 1.31 (3H, t, J = 6.9 Hz), 4.09 (2H, q, J = 6.9 Hz), 5.03 (2H, s), 6.9-7.0 (1H, m), 7.04 (1H, d, J = 8.2 Hz), 7.1-7.2 (1H, m), 7.2-7.25 (1H, m), 7.3- 7.45 (4H, m), 12.01 (1H, s), 14.43 (1H, s) |
| 319 | 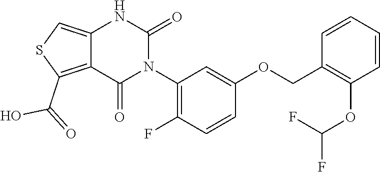 [SMILES: O=C(O)c1scc2[nH]c(=O)n(-c3cc(OCc4ccccc4OC(F)F)ccc3F)c(=O)c12] | (DMSO-d6) 5.08 (2H, s), 7.05-7.5 (7H, m), 7.55-7.65 (1H, m), 12.01 (1H, s), 14.42 (1H, s) |
| 320 | 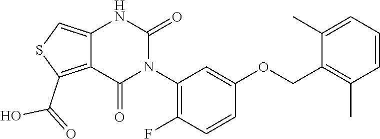 [SMILES: Cc1cccc(C)c1COc1ccc(F)c(-n2c(=O)[nH]c3csc(C(=O)O)c3c2=O)c1] | (DMSO-d6) 2.34 (6H, s), 5.03 (2H, s), 7.0-7.45 (7H, m), 12.0 (1H, s), 14.4 (1H, s) |
| 321 | 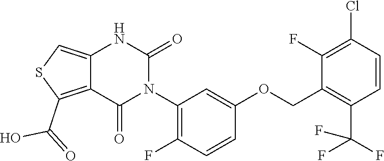 [SMILES: O=C(O)c1scc2[nH]c(=O)n(-c3cc(OCc4c(C(F)(F)F)ccc(Cl)c4F)ccc3F)c(=O)c12] | (DMSO-d6) 5.17 (2H, s), 7.2-7.3 (2H, m), 7.35-7.45 (2H, m), 7.74 (1H, d, J = 8.2 Hz), 7.9-8.0 (1H, m), 12.02 (1H, s), 14.38 (1H, s) |
| 322 | 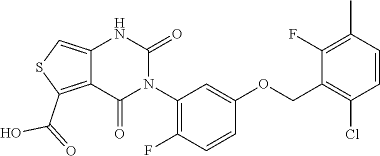 [SMILES: Cc1ccc(Cl)c(COc2ccc(F)c(-n3c(=O)[nH]c4csc(C(=O)O)c4c3=O)c2)c1F] | (DMSO-d6) 2.2-2.3 (3H, m), 5.05-5.15 (2H, m), 7.2-7.3 (2H, m), 7.32 (1H, d, J = 8.6 Hz), 7.35-7.45 (3H, m), 12.02 (1H, s), 14.39 (1H, s) |
| 323 | 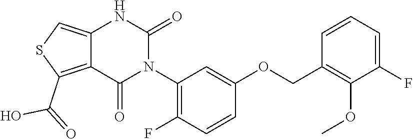 [SMILES: COc1c(F)cccc1COc1ccc(F)c(-n2c(=O)[nH]c3csc(C(=O)O)c3c2=O)c1] | (DMSO-d6) 3.85-3.95 (3H, m), 5.07 (2H, s), 7.1-7.4 (7H, m), 12.01 (1H, s), 14.42 (1H, s) |
| 324 | 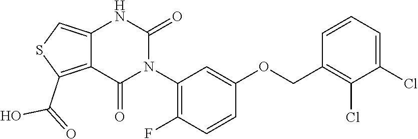 [SMILES: O=C(O)c1scc2[nH]c(=O)n(-c3cc(OCc4cccc(Cl)c4Cl)ccc3F)c(=O)c12] | (DMSO-d6) 5.18 (2H, s), 7.15-7.25 (1H, m), 7.25-7.35 (1H, m), 7.35-7.5 (3H, m), 7.55-7.7 (2H, m), 12.01 (1H, s), 14.41 (1H, s) |
| 325 | 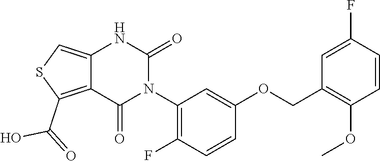 [SMILES: COc1ccc(F)cc1COc1ccc(F)c(-n2c(=O)[nH]c3csc(C(=O)O)c3c2=O)c1] | (DMSO-d6) 3.81 (3H, s), 5.02 (2H, s), 7.0-7.1 (1H, m), 7.1-7.2 (2H, m), 7.2- 7.4 (4H, m), 12.01 (1H, s), 14.42 (1H, s) |
| 326 | 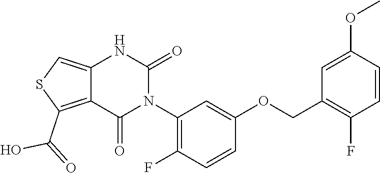 [SMILES: COc1ccc(F)c(COc2ccc(F)c(-n3c(=O)[nH]c4csc(C(=O)O)c4c3=O)c2)c1] | (DMSO-d6) 3.75 (3H, s), 5.08 (2H, s), 6.9-7.0 (1H, m), 7.1-7.25 (3H, m), 7.27 (1H, dd, J = 6.1 Hz, 3.2 Hz), 7.3-7.4 (2H, m), 12.02 (1H, s), 14.41 (1H, s) |
| 327 | 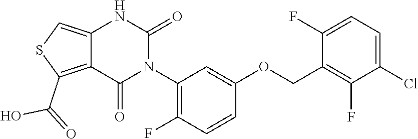 [SMILES: O=C(O)c1scc2[nH]c(=O)n(-c3cc(OCc4c(F)ccc(Cl)c4F)ccc3F)c(=O)c12] | (DMSO-d6) 5.14 (2H, s), 7.15-7.35 (3H, m), 7.35-7.45 (2H, m), 7.7-7.8 (1H, m), 12.02 (1H, s), 14.39 (1H, s) |
| 328 | 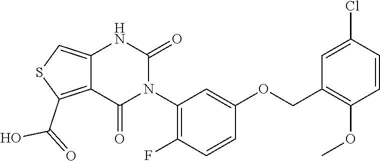 [SMILES: COc1ccc(Cl)cc1COc1ccc(F)c(-n2c(=O)[nH]c3csc(C(=O)O)c3c2=O)c1] | (DMSO-d6) 3.83 (3H, s), 5.01 (2H, s), 7.09 (1H, d, J = 8.8 Hz), 7.15-7.2 (1H, m), 7.25-7.35 (1H, m), 7.3-7.45 (3H, m), 7.48 (1H, d, J = 2.6 Hz), 12.02 (1H, s), 14.43 (1H, s) |
| 329 | 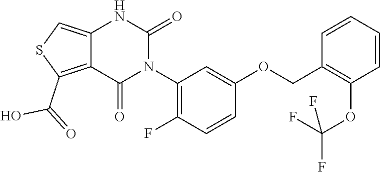 [SMILES: O=C(O)c1scc2[nH]c(=O)n(-c3cc(OCc4ccccc4OC(F)(F)F)ccc3F)c(=O)c12] | (DMSO-d6) 5.12 (2H, s), 7.15-7.25 (1H, m), 7.27 (1H, dd, J = 6.0 Hz, 3.2 Hz), 7.3-7.4 (2H, m), 7.4-7.5 (2H, m), 7.5-7.6 (1H, m), 7.7 (1H, dd, J = 7.4 Hz, 1.6 Hz), 12.01 (1H, s), 14.41 (1H, s) |
| 330 | 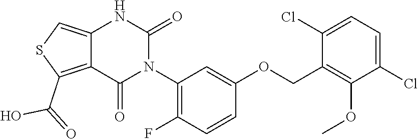 [SMILES: COc1c(Cl)ccc(Cl)c1COc1ccc(F)c(-n2c(=O)[nH]c3csc(C(=O)O)c3c2=O)c1] | (DMSO-d6) 3.85 (3H, s), 5.1 (2H, s), 7.2-7.35 (2H, m), 7.35-7.45 (3H, m), 7.63 (1H, d, J = 8.7 Hz), 12.01 (1H, s), 14.38 (1H, s) |
| 331 | 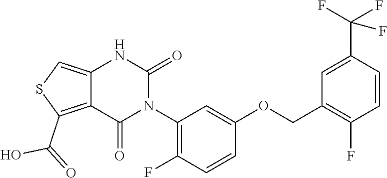 [SMILES: O=C(O)c1scc2[nH]c(=O)n(-c3cc(OCc4cc(C(F)(F)F)ccc4F)ccc3F)c(=O)c12] | (DMSO-d6) 5.21 (2H, s), 7.2-7.35 (2H, m), 7.35-7.45 (2H, m), 7.53 (1H, t, J = 9.2 Hz), 7.8-7.9 (1H, m), 8.02 (1H, d, J = 6.1 Hz), 12.02 (1H, s), 14.4 (1H, s) |
| 332 | 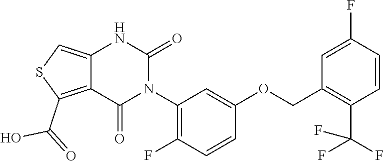 [SMILES: O=C(O)c1scc2[nH]c(=O)n(-c3cc(OCc4cc(F)ccc4C(F)(F)F)ccc3F)c(=O)c12] | (DMSO-d6) 5.21 (2H, s), 7.15-7.25 (1H, m), 7.3 (1H, dd, J = 5.8 Hz, 3.0 Hz), 7.35-7.5 (3H, m), 7.65-7.75 (1H, m), 7.89 (1H, dd, J = 8.7 Hz, 5.4 Hz), 12.01 (1H, s), 14.4 (1H, s) |
| 333 | 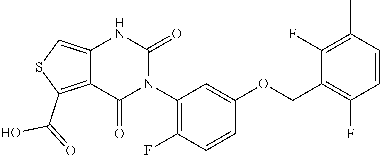 [SMILES: Cc1ccc(F)c(COc2ccc(F)c(-n3c(=O)[nH]c4csc(C(=O)O)c4c3=O)c2)c1F] | (DMSO-d6) 2.24 (3H, s), 5.08 (2H, s), 7.05-7.15 (1H, m), 7.15-7.3 (2H, m), 7.35-7.45 (3H, m), 12.02 (1H, s), 14.4 (1H, s) |
| 334 | 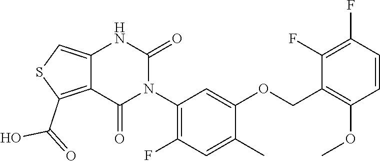 [SMILES: COc1ccc(F)c(F)c1COc1cc(-n2c(=O)[nH]c3csc(C(=O)O)c3c2=O)c(F)cc1C] | (DMSO-d6) 2.13 (3H, s), 3.82 (3H, s), 5.0 (2H, s), 6.85-6.95 (1H, m), 7.25 (1H, d, J = 10.0 Hz), 7.32 (1H, d, J = 6.4 Hz), 7.39 (1H, s), 7.4-7.55 (1H, m), 12.01 (1H, s), 14.47 (1H, s) |
| 335 | 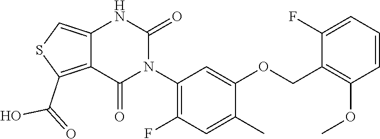 [SMILES: COc1cccc(F)c1COc1cc(-n2c(=O)[nH]c3csc(C(=O)O)c3c2=O)c(F)cc1C] | (DMSO-d6) 2.12 (3H, s), 3.84 (3H, s), 4.96 (2H, s), 6.8-7.0 (2H, m), 7.24 (1H, d, J = 10.2 Hz), 7.32 (1H, d, J = 6.3 Hz), 7.35-7.5 (2H, m), 12.04 (1H, s), 14.48 (1H, s) |
| 336 | 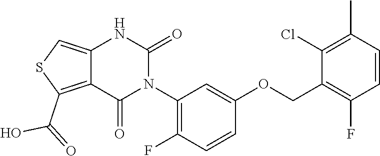 [SMILES: Cc1ccc(F)c(COc2ccc(F)c(-n3c(=O)[nH]c4csc(C(=O)O)c4c3=O)c2)c1Cl] | (DMSO-d6) 2.36 (3H, s), 5.1-5.15 (2H, m), 7.2-7.3 (3H, m), 7.35-7.45 (2H, m), 7.5 (1H, dd, J = 8.7 Hz, 6.2 Hz), 12.03 (1H, s), 14.4 (1H, s) |
| 337 | 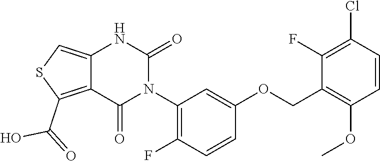 [SMILES: COc1ccc(Cl)c(F)c1COc1ccc(F)c(-n2c(=O)[nH]c3csc(C(=O)O)c3c2=O)c1] | (DMSO-d6) 3.86 (3H, s), 5.04 (2H, s), 7.0 (1H, d, J = 9.3 Hz), 7.15-7.25 (2H, m), 7.3-7.4 (2H, m), 7.55-7.65 (1H, m), 12.03 (1H, s), 14.41 (1H, s) |
| 338 | 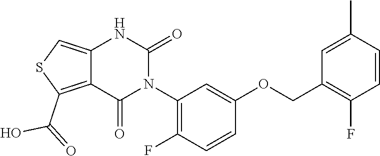 [SMILES: Cc1ccc(F)c(COc2ccc(F)c(-n3c(=O)[nH]c4csc(C(=O)O)c4c3=O)c2)c1] | (DMSO-d6) 2.3 (3H, s), 5.06 (2H, s), 7.1-7.45 (7H, m), 12.03 (1H, s), 14.43 (1H, s) |
| 339 | 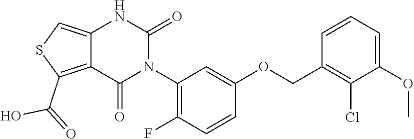 [SMILES: COc1cccc(COc2ccc(F)c(-n3c(=O)[nH]c4csc(C(=O)O)c4c3=O)c2)c1Cl] | (DMSO-d6) 3.88 (3H, s), 5.13 (2H, s), 7.15-7.25 (3H, m), 7.27 (1H, dd, J = 6.0 Hz, 3.2 Hz), 7.3-7.4 (3H, m), 12.02 (1H, s), 14.42 (1H, s) |
| 340 | 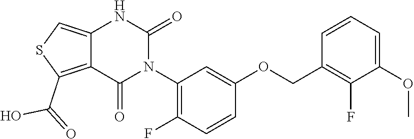 [SMILES: COc1cccc(COc2ccc(F)c(-n3c(=O)[nH]c4csc(C(=O)O)c4c3=O)c2)c1F] | (DMSO-d6) 3.85 (3H, s), 5.11 (2H, s), 7.05-7.3 (5H, m), 7.3-7.4 (2H, m), 12.02 (1H, s), 14.42 (1H, s) |
| 341 | 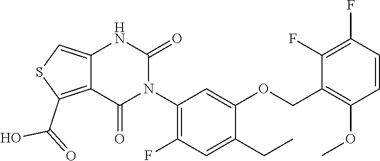 [SMILES: CCc1cc(F)c(-n2c(=O)[nH]c3csc(C(=O)O)c3c2=O)cc1OCc1c(OC)ccc(F)c1F] | (DMSO-d6) 1.1 (3H, t, J = 7.5 Hz), 2.45- 2.6 (2H, m), 3.82 (3H, s), 5.0 (2H, s), 6.9-6.95 (1H, m), 7.23 (1H, d, J = 10.3 Hz), 7.34 (1H, d, J = 6.3 Hz), 7.39 (1H, s), 7.4-7.55 (1H, m), 12.03 (1H, s), 14.47 (1H, s) |
| 342 | 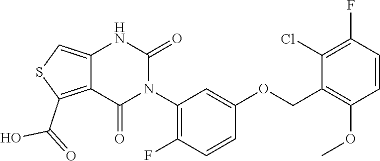 [SMILES: COc1ccc(F)c(Cl)c1COc1ccc(F)c(-n2c(=O)[nH]c3csc(C(=O)O)c3c2=O)c1] | (DMSO-d6) 3.84 (3H, s), 5.1 (2H, s), 7.13 (1H, dd, J = 9.2 Hz, 4.0 Hz), 7.15- 7.3 (2H, m), 7.3-7.4 (2H, m), 7.45-7.55 (1H, m), 12.03 (1H, s) |
| 343 | 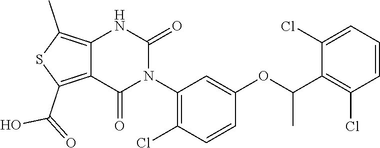 [SMILES: Cc1sc(C(=O)O)c2c(=O)n(-c3cc(OC(C)c4c(Cl)cccc4Cl)ccc3Cl)c(=O)[nH]c12] | (DMSO-d6) 1.73 (3H, d, J = 6.7 Hz), 2.5-2.55 (3H, m), 6.07 (1H, q, J = 6.7 Hz), 6.85-6.95 (1H, m), 7.15-7.2 (1H, m), 7.3-7.4 (1H, m), 7.45-7.55 (3H, m), 11.89 (1H, s), 14.37 (1H, s) |
| 344 | 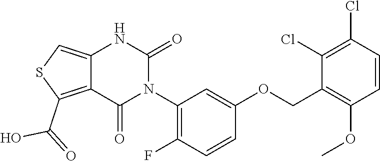 [SMILES: COc1ccc(Cl)c(Cl)c1COc1ccc(F)c(-n2c(=O)[nH]c3csc(C(=O)O)c3c2=O)c1] | (DMSO-d6) 3.86 (3H, s), 5.12 (2H, s), 7.1-7.3 (3H, m), 7.3-7.4 (2H, m), 7.69 (1H, d, J = 8.7 Hz), 12.03 (1H, s), 14.41 (1H, s) |
| 345 | 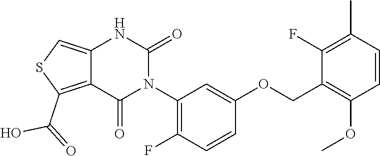 [SMILES: COc1ccc(C)c(F)c1COc1ccc(F)c(-n2c(=O)[nH]c3csc(C(=O)O)c3c2=O)c1] | (DMSO-d6) 2.15-2.2 (3H, m), 3.81 (3H, s), 5.0 (2H, s), 6.84 (1H, d, J = 8.5 Hz), 7.15-7.25 (2H, m), 7.25-7.4 (3H, m), 12.03 (1H, s), 14.42 (1H, s) |
| 346 | 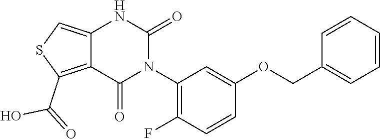 [SMILES: O=C(O)c1scc2[nH]c(=O)n(-c3cc(OCc4ccccc4)ccc3F)c(=O)c12] | (DMSO-d6) 5.08 (2H, s), 7.1-7.5 (9H, m), 12.01 (1H, s), 14.42 (1H, s) |
| 347 | 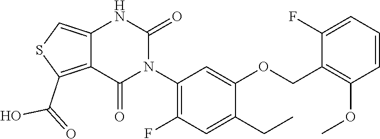 [SMILES: CCc1cc(F)c(-n2c(=O)[nH]c3csc(C(=O)O)c3c2=O)cc1OCc1c(F)cccc1OC] | (DMSO-d6) 1.1 (3H, t, J = 7.5 Hz), 2.45- 2.55 (2H, m), 3.84 (3H, s), 4.97 (2H, s), 6.85-7.0 (2H, m), 7.22 (1H, d, J = 10.2 Hz), 7.33 (1H, d, J = 6.4 Hz), 7.35-7.5 (2H, m), 12.02 (1H, s), 14.47 (1H, brs) |
| 348 | 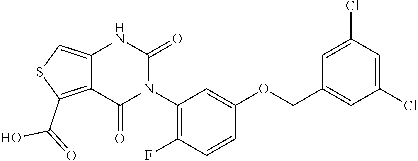 [SMILES: O=C(O)c1scc2[nH]c(=O)n(-c3cc(OCc4cc(Cl)cc(Cl)c4)ccc3F)c(=O)c12] | (DMSO-d6) 5.11 (2H, s), 7.1-7.3 (2H, m), 7.3-7.45 (2H, m), 7.5-7.65 (3H, m), 12.0 (1H, brs), 14.41 (1H, brs) |
| 349 | 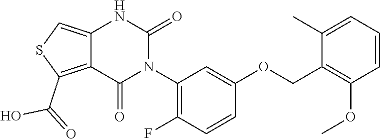 [SMILES: COc1cccc(C)c1COc1ccc(F)c(-n2c(=O)[nH]c3csc(C(=O)O)c3c2=O)c1] | (DMSO-d6) 2.33 (3H, s), 3.78 (3H, s), 5.04 (2H, s), 6.8-6.95 (2H, m), 7.1-7.3 (3H, m), 7.3-7.4 (2H, m), 12.01 (1H, s), 14.42 (1H, brs) |
| 350 | 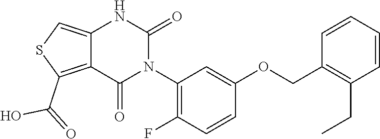 [SMILES: CCc1ccccc1COc1ccc(F)c(-n2c(=O)[nH]c3csc(C(=O)O)c3c2=O)c1] | (DMSO-d6) 1.19 (3H, t, J = 7.5 Hz), 2.68 (2H, q, J = 7.5 Hz), 5.07 (2H, s), 7.15-7.4 (7H, m), 7.4-7.5 (1H, m), 12.0 (1H, brs), 14.3-14.55 (1H, br) |
| 351 | 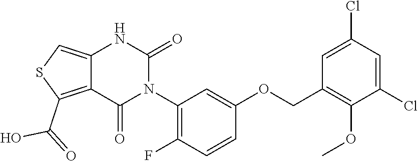 [SMILES: COc1c(Cl)cc(Cl)cc1COc1ccc(F)c(-n2c(=O)[nH]c3csc(C(=O)O)c3c2=O)c1] | (DMSO-d6) 3.84 (3H, s), 5.09 (2H, s), 7.2-7.25 (1H, m), 7.25-7.35 (1H, m), 7.35-7.45 (2H, m), 7.58 (1H, d, J = 2.7 Hz), 7.68 (1H, d, J = 2.7 Hz), 12.01 (1H, brs), 14.41 (1H, brs) |
| 352 | 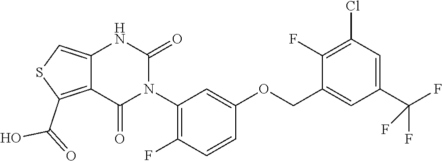 [SMILES: O=C(O)c1scc2[nH]c(=O)n(-c3cc(OCc4cc(C(F)(F)F)cc(Cl)c4F)ccc3F)c(=O)c12] | (DMSO-d6) 5.25 (2H, s), 7.2-7.35 (2H, m), 7.35-7.45 (2H, m), 7.95-8.05 (1H, m), 8.1-8.2 (1H, m), 12.01 (1H, brs), 14.4 (1H, brs) |
| 353 | 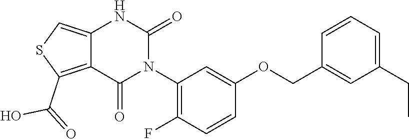 [SMILES: CCc1cccc(COc2ccc(F)c(-n3c(=O)[nH]c4csc(C(=O)O)c4c3=O)c2)c1] | (DMSO-d6) 1.19 (3H, t, J = 7.6 Hz), 2.62 (2H, q, J = 7.6 Hz), 5.05 (2H, s), 7.1-7.4 (8H, m), 12.01 (1H, brs), 14.43 (1H, brs) |
| 354 | 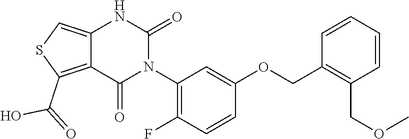 [SMILES: COCc1ccccc1COc1ccc(F)c(-n2c(=O)[nH]c3csc(C(=O)O)c3c2=O)c1] | (DMSO-d6) 3.3 (3H, s), 4.53 (2H, s), 5.13 (2H, s), 7.1-7.3 (2H, m), 7.3-7.45 (5H, m), 7.45-7.55 (1H, m), 12.01 (1H, brs), 14.42 (1H, brs) |
| 355 | 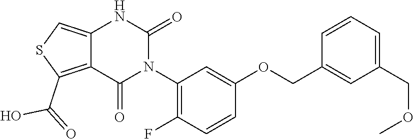 [SMILES: COCc1cccc(COc2ccc(F)c(-n3c(=O)[nH]c4csc(C(=O)O)c4c3=O)c2)c1] | (DMSO-d6) 3.29 (3H, s), 4.43 (2H, s), 5.09 (2H, s), 7.1-7.2 (1H, m), 7.2-7.45 (7H, m), 12.01 (1H, brs), 14.42 (1H, brs) |
| 356 | 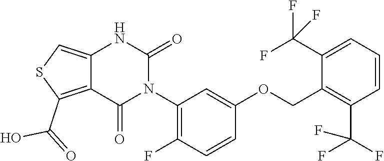 [SMILES: O=C(O)c1scc2[nH]c(=O)n(-c3cc(OCc4c(C(F)(F)F)cccc4C(F)(F)F)ccc3F)c(=O)c12] | (DMSO-d6) 5.19 (2H, s), 7.15-7.25 (1H, m), 7.25-7.3 (1H, m), 7.35-7.45 (2H, m), 7.92 (1H, t, J = 8.0 Hz), 8.21 (2H, d, J = 28.0 Hz), 12.01 (1H, s), 14.38 (1H, brs) |
| 357 | 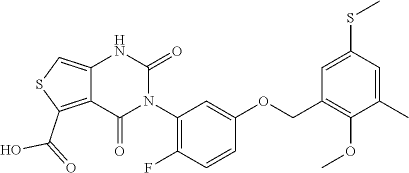 [SMILES: COc1c(C)cc(SC)cc1COc1ccc(F)c(-n2c(=O)[nH]c3csc(C(=O)O)c3c2=O)c1] | (DMSO-d6) 2.25 (3H, s), 2.44 (3H, s), 3.7 (3H, s), 5.02 (2H, s), 7.1-7.25 (3H, m), 7.25-7.3 (1H, m), 7.3-7.4 (2H, m), 12.01 (1H, brs), 14.3-14.55 (1H, br) |
| 358 | 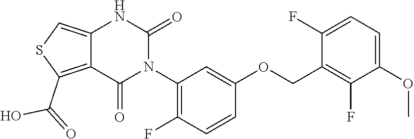 [SMILES: COc1ccc(F)c(COc2ccc(F)c(-n3c(=O)[nH]c4csc(C(=O)O)c4c3=O)c2)c1F] | (DMSO-d6) 3.85 (3H, s), 5.09 (2H, s), 7.05-7.35 (4H, m), 7.35-7.45 (2H, m), 12.02 (1H, brs), 14.39 (1H, brs) |
| 359 | 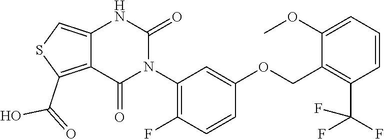 [SMILES: COc1cccc(C(F)(F)F)c1COc1ccc(F)c(-n2c(=O)[nH]c3csc(C(=O)O)c3c2=O)c1] | (DMSO-d6) 3.89 (3H, s), 5.07 (2H, s), 7.15-7.3 (2H, m), 7.3-7.5 (4H, m), 7.6- 7.7 (1H. m), 12.01 (1H, s), 14.4 (1H, brs) |
| 360 | 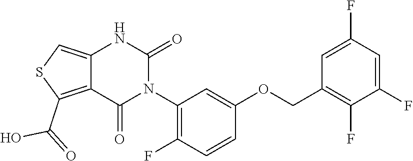 [SMILES: O=C(O)c1scc2[nH]c(=O)n(-c3cc(OCc4cc(F)cc(F)c4F)ccc3F)c(=O)c12] | (DMSO-d6) 5.18 (2H, s), 7.2-7.45 (5H, m), 7.5-7.65 (1H, m), 12.02 (1H, s), 14.4 (1H, brs) |
| 361 | 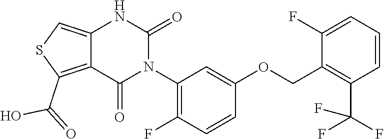 [SMILES: O=C(O)c1scc2[nH]c(=O)n(-c3cc(OCc4c(F)cccc4C(F)(F)F)ccc3F)c(=O)c12] | (DMSO-d6) 5.14 (2H, s), 7.15-7.3 (2H, m), 7.3-7.45 (2H, m), 7.6-7.8 (3H, m), 12.02 (1H, s), 14.39 (1H, s) |
| 362 | 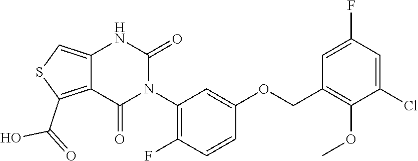 [SMILES: COc1c(Cl)cc(F)cc1COc1ccc(F)c(-n2c(=O)[nH]c3csc(C(=O)O)c3c2=O)c1] | (DMSO-d6) 3.82 (3H, s), 5.1 (2H, s), 7.15-7.45 (5H, m), 7.45-7.55 (1H, m), 12.01 (1H, s), 14.4 (1H, s) |
| 363 | 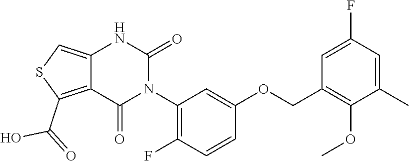 [SMILES: COc1c(C)cc(F)cc1COc1ccc(F)c(-n2c(=O)[nH]c3csc(C(=O)O)c3c2=O)c1] | (DMSO-d6) 2.27 (3H, s), 3.71 (3H, s), 5.05 (2H, s), 7.05-7.25 (3H, m), 7.25-7.3 (1H, m), 7.3-7.4 (2H, m), 12.02 (1H, s), 14.42 (1H, brs) |
| 364 | 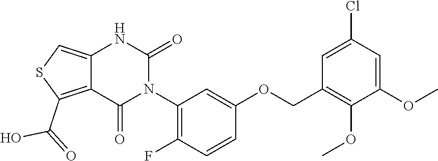 [SMILES: COc1cc(Cl)cc(COc2ccc(F)c(-n3c(=O)[nH]c4csc(C(=O)O)c4c3=O)c2)c1OC] | (DMSO-d6) 3.76 (3H, s), 3.85 (3H, s), 5.03 (2H, s), 7.05-7.3 (4H, m), 7.3-7.4 (2H, m), 12.02 (1H, brs), 14.42 (1H, brs) |
| 365 | 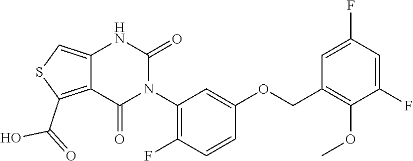 [SMILES: COc1c(F)cc(F)cc1COc1ccc(F)c(-n2c(=O)[nH]c3csc(C(=O)O)c3c2=O)c1] | (DMSO-d6) 3.8-3.9 (3H, m), 5.08 (2H, s), 7.15-7.25 (2H, m), 7.25-7.45 (4H, m), 12.02 (1H, brs), 14.41 (1H, brs) |
| 366 | 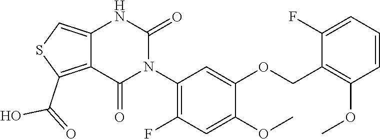 [SMILES: COc1cc(F)c(-n2c(=O)[nH]c3csc(C(=O)O)c3c2=O)cc1OCc1c(F)cccc1OC] | (DMSO-d6) 3.79 (3H, s), 3.83 (3H, s), 4.92 (2H, s), 6.8-7.0 (2H, m), 7.11 (1H, d, J = 11.2 Hz), 7.27 (1H, d, J = 27.6 Hz), 7.38 (1H, s), 7.4-7.5 (1H, m), 11.99 (1H, brs), 14.55 (1H, brs) |
| 367 | 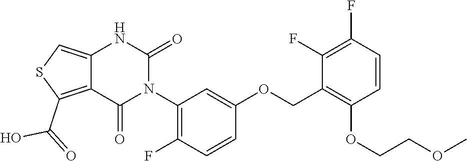 [SMILES: COCCOc1ccc(F)c(F)c1COc1ccc(F)c(-n2c(=O)[nH]c3csc(C(=O)O)c3c2=O)c1] | (DMSO-d6) 3.23 (3H, s), 3.55-3.65 (2H, m), 4.1-4.2 (2H, m), 5.09 (2H, s), 6.9-7.0 (1H, m), 7.15-7.3 (2H, m), 7.3-7.4 (2H, m), 7.4-7.5 (1H, m), 12.03 (1H, brs), 14.42 (1H, brs) |
| 368 | 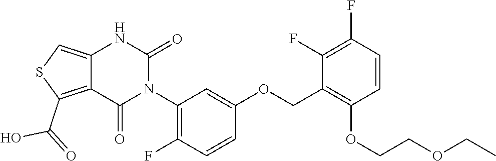 [SMILES: CCOCCOc1ccc(F)c(F)c1COc1ccc(F)c(-n2c(=O)[nH]c3csc(C(=O)O)c3c2=O)c1] | (DMSO-d6) 1.02 (3H, t, J = 6.9 Hz), 3.43 (2H, q, J = 6.9 Hz), 3.6-3.7 (2H, m), 4.1-4.2 (2H, m), 5.08 (2H, s), 6.9-7.0 (1H, m), 7.15- 7.25 (2H, m), 7.3-7.4 (2H, m), 7.4-7.5 (1H, m), 12.02 (1H, s), 14.42 (1H, brs) |
| 369 | 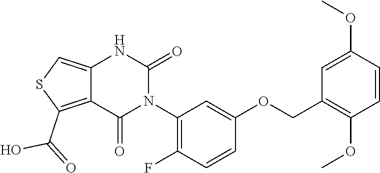 [SMILES: COc1ccc(OC)c(COc2ccc(F)c(-n3c(=O)[nH]c4csc(C(=O)O)c4c3=O)c2)c1] | (DMSO-d6) 3.71 (3H, s), 3.77 (3H, s), 4.99 (2H, s), 6.85-6.95 (1H, m), 6.95- 7.05 (2H, m), 7.1-7.2 (1H, m), 7.2-7.3 (1H, m), 7.3-7.4 (2H, m), 12.01 (1H, s), 14.44 (1H, brs) |
| 370 | 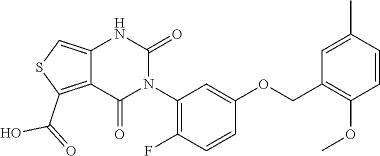 [SMILES: COc1ccc(C)cc1COc1ccc(F)c(-n2c(=O)[nH]c3csc(C(=O)O)c3c2=O)c1] | (DMSO-d6) 2.25 (3H, s), 3.78 (3H, s), 4.98 (2H, s), 6.94 (1H, d, J = 7.9 Hz), 7.1-7.2 (2H, m), 7.2-7.3 (2H, m), 7.3-7.4 (2H, m), 12.01 (1H, brs), 14.45 (1H, brs) |
| 371 | 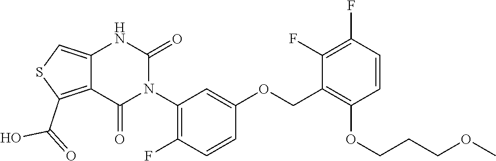 [SMILES: COCCCOc1ccc(F)c(F)c1COc1ccc(F)c(-n2c(=O)[nH]c3csc(C(=O)O)c3c2=O)c1] | (DMSO-d6) 1.85-1.95 (2H, m), 3.16 (3H, s), 3.39 (2H, t, J = 6.2 Hz), 4.08 (2H, t, J = 6.2 Hz), 5.06 (2H, s), 6.85-6.95 (1H, m), 7.15-7.3 (2H, m), 7.3-7.5 (3H, m), 12.03 (1H, s), 14.42 (1H, brs) |
| 372 | 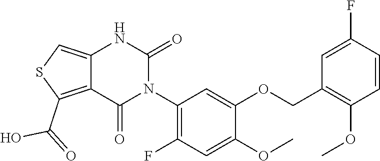 [SMILES: COc1ccc(F)cc1COc1cc(-n2c(=O)[nH]c3csc(C(=O)O)c3c2=O)c(F)cc1OC] | (DMSO-d6) 3.78 (3H, s), 3.85 (3H, s), 4.94 (2H, s), 7.0-7.1 (1H, m), 7.1-7.2 (2H, m), 7.2-7.35 (2H, m), 7.36 (1H, s), 11.96 (1H, brs), 14.56 (1H, brs) |
| 373 | 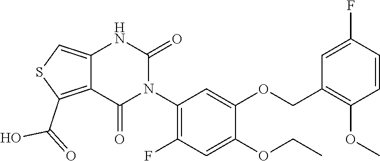 [SMILES: CCOc1cc(F)c(-n2c(=O)[nH]c3csc(C(=O)O)c3c2=O)cc1OCc1cc(F)ccc1OC] | (DMSO-d6) 1.36 (3H, t, J = 6.9 Hz), 3.78 (3H, s), 4.13 (2H, q, J = 6.9 Hz), 4.96 (2H, s), 7.0-7.1 (1H, m), 7.1-7.2 (2H, m), 7.2-7.35 (2H, m), 7.37 (1H, s) 11.97 (1H, brs), 14.56 (1H, brs) |
| 374 | 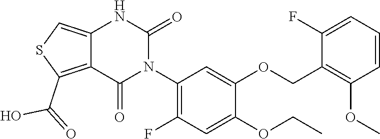 [SMILES: CCOc1cc(F)c(-n2c(=O)[nH]c3csc(C(=O)O)c3c2=O)cc1OCc1c(F)cccc1OC] | (DMSO-d6) 1.3 (3H, t, J = 7.0 Hz), 3.82 (3H, s), 4.08 (2H, q, J = 27.0 Hz), 4.94 (2H, s), 6.8-7.0 (2H, m), 7.1 (1H, d, J = 11.6 Hz), 7.26 (1H, d, J = 7.6 Hz), 7.35- 7.5 (2H, m), 11.99 (1H, s), 14.55 (1H, brs) |
| 375 | 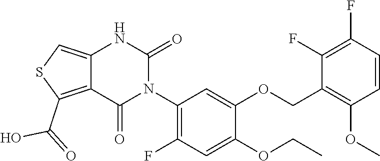 [SMILES: CCOc1cc(F)c(-n2c(=O)[nH]c3csc(C(=O)O)c3c2=O)cc1OCc1c(OC)ccc(F)c1F] | (DMSO-d6) 1.31 (3H, t, J = 7.0 Hz), 3.79 (3H, s), 4.09 (2H, q, J = 7.0 Hz), 4.95-5.05 (2H, m), 6.85-6.95 (1H, m), 7.12 (1H, d, J = 11.7 Hz), 7.23 (1H, d, J = 7.3 Hz), 7.38 (1H, s), 7.4-7.5 (1H, m), 11.99 (1H, s), 14.54 (1H, brs) |
| 376 | 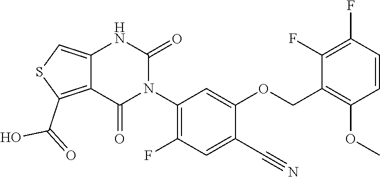 [SMILES: COc1ccc(F)c(F)c1COc1cc(-n2c(=O)[nH]c3csc(C(=O)O)c3c2=O)c(F)cc1C#N] | (DMSO-d6) 3.83 (3H, s), 5.18 (2H, s), 6.9-7.0 (1H, m), 7.38 (1H, s), 7.45-7.6 (1H, m), 7.7-7.85 (1H, m), 8.0-8.1 (1H, m), 12.08 (1H, s), 14.16 (1H, brs) |
| 377 | 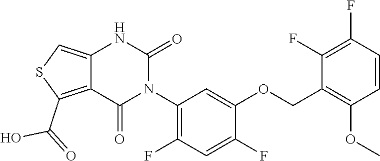 [SMILES: COc1ccc(F)c(F)c1COc1cc(-n2c(=O)[nH]c3csc(C(=O)O)c3c2=O)c(F)cc1F] | (DMSO-d6) 3.81 (3H, s), 5.07 (2H, s), 6.9-6.95 (1H, m), 7.39 (1H, s), 7.45- 7.55 (1H, m), 7.55-7.65 (2H, m), 12.06 (1H, s), 14.37 (1H, brs) |
| 378 | 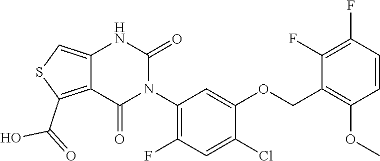 [SMILES: COc1ccc(F)c(F)c1COc1cc(-n2c(=O)[nH]c3csc(C(=O)O)c3c2=O)c(F)cc1Cl] | (DMSO-d6) 3.82 (3H, s), 5.08 (2H, s), 6.9-7.0 (1H, m), 7.39 (1H, s), 7.45-7.6 (1H, m), 7.61 (1H, d, J = 6.7 Hz), 7.73 (1H, d J = 9.1 Hz), 12.07 (1H, s), 14.33 (1H, brs) |
| 379 | 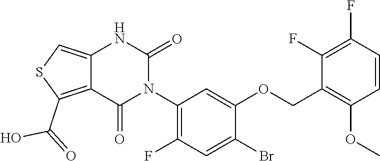 [SMILES: COc1ccc(F)c(F)c1COc1cc(-n2c(=O)[nH]c3csc(C(=O)O)c3c2=O)c(F)cc1Br] | (DMSO-d6) 3.82 (3H, s), 5.07 (2H, s), 6.9-7.0 (1H, m), 7.39 (1H, s), 7.45-7.55 (1H, m), 7.58 (1H, d, J = 6.5 Hz), 7.84 (1H, d, J = 8.8 Hz), 12.07 (1H, s), 14.33 (1H, s) |
| 380 | 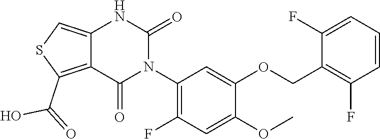 [SMILES: COc1cc(F)c(-n2c(=O)[nH]c3csc(C(=O)O)c3c2=O)cc1OCc1c(F)cccc1F] | (DMSO-d6) 3.81 (3H, s), 5.0 (2H, s), 7.1-7.25 (3H, m), 7.29 (1H, d, J = 7.4 Hz), 7.39 (1H, s), 7.45-7.6 (1H, m), 12.01 (1H, s), 14.54 (1H, s) |
| 381 | 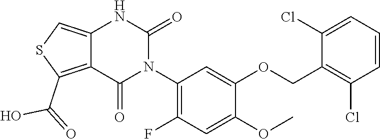 [SMILES: COc1cc(F)c(-n2c(=O)[nH]c3csc(C(=O)O)c3c2=O)cc1OCc1c(Cl)cccc1Cl] | (DMSO-d6) 3.82 (3H, s), 5.13 (2H, s), 7.15 (1H, d, J = 11.4 Hz), 7.33 (1H, d, J = 7.3 Hz), 7.39 (1H, s), 7.45-7.6 (3H, m), 12.01 (1H, s), 14.54 (1H, s) |
| 382 | 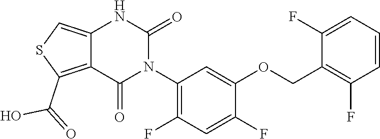 [SMILES: O=C(O)c1scc2[nH]c(=O)n(-c3cc(OCc4c(F)cccc4F)c(F)cc3F)c(=O)c12] | (DMSO-d6) 5.12 (2H, s), 7.15-7.25 (2H, m), 7.39 (1H, s), 7.5-7.7 (3H, m), 12.06 (1H, s), 14.36 (1H, brs) |
| 383 | 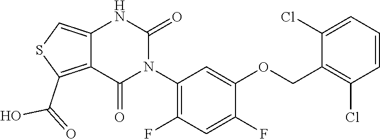 [SMILES: O=C(O)c1scc2[nH]c(=O)n(-c3cc(OCc4c(Cl)cccc4Cl)c(F)cc3F)c(=O)c12] | (DMSO-d6) 5.24 (2H, s), 7.39 (1H, s), 7.45-7.55 (1H, m), 7.55-7.75 (4H, m), 12.06 (1H, s), 14.36 (1H, brs) |
| 384 | 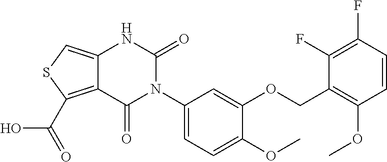 [SMILES: COc1ccc(-n2c(=O)[nH]c3csc(C(=O)O)c3c2=O)cc1OCc1c(OC)ccc(F)c1F] | (DMSO-d6) 3.78 (3H, s), 3.81 (3H, s), 4.96 (2H, s), 6.85-7.0 (2H, m), 7.07 (1H, d, J = 8.4 Hz), 7.18 (1H, d, J = 2.4 Hz), 7.37 (1H, s), 7.4-7.55 (1H, m), 11.87 (1H, s), 14.97 (1H, s) |
| 385 | 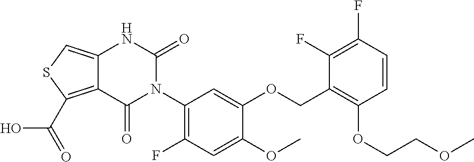 [SMILES: COCCOc1ccc(F)c(F)c1COc1cc(-n2c(=O)[nH]c3csc(C(=O)O)c3c2=O)c(F)cc1OC] | (DMSO-d6) 3.2 (3H, s), 3.5-3.6 (2H, m), 3.81 (3H, s), 4.05-4.15 (2H, m), 5.01 (2H, s); 6.85-6.95 (1H, m), 7.13 (1H, d, J = 11.2 Hz), 7.23 (1H, d, J = 7.0 Hz), 7.39 (1H, s), 7.4-7.5 (1H, m), 11.99 (1H, s) 14.52 (1H, s) |
| 386 | 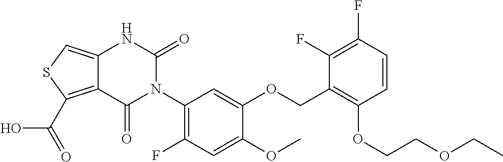 [SMILES: CCOCCOc1ccc(F)c(F)c1COc1cc(-n2c(=O)[nH]c3csc(C(=O)O)c3c2=O)c(F)cc1OC] | (DMSO-d6) 1.0 (3H, t, J = 7.0 Hz), 3.4 (2H, q, J = 7.0 Hz), 3.55-3.65 (2H, m), 3.81 (3H, s), 4.05-4.15 (2H, m), 5.0 (2H, s), 6.85-6.95 (1H, m), 7.13 (1H, d, J = 11.4 Hz), 7.23 (1H, d, J = 7.7 Hz), 7.38 (1H, s), 7.4-7.5 (1H, m), 11.99 (1H, s), 14.53 (1H, brs) |
| 387 | 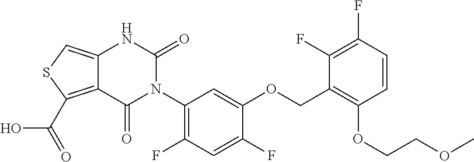 [SMILES: COCCOc1ccc(F)c(F)c1COc1cc(-n2c(=O)[nH]c3csc(C(=O)O)c3c2=O)c(F)cc1F] | (DMSO-d6) 3.19 (3H, s), 3.5-3.65 (2H, m), 4.05-4.2 (2H, m), 5.13 (2H, s), 6.9- 7.0 (1H, m), 7.38 (1H, s), 7.4-7.65 (3H, m), 12.03 (1H, s), 14.34 (1H, brs) |
| 388 | 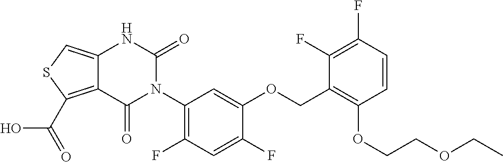 [SMILES: CCOCCOc1ccc(F)c(F)c1COc1cc(-n2c(=O)[nH]c3csc(C(=O)O)c3c2=O)c(F)cc1F] | (DMSO-d6) 0.99 (3H, t, J = 7.0 Hz), 3.4 (2H, q, J = 7.0 Hz), 3.55-3.7 (2H, m), 4.05-4.2 (2H, m), 5.11 (2H, s), 6.9-7.0 (1H, m), 7.38 (1H, s), 7.4-7.65 (3H, m), 12.03 (1H, s), 14.35 (1H, brs) |
| 389 | 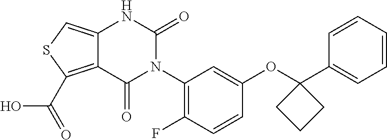 [SMILES: O=C(O)c1scc2[nH]c(=O)n(-c3cc(OC4(c5ccccc5)CCC4)ccc3F)c(=O)c12] | (DMSO-d6) 1.75-1.9 (1H, m), 1.9-2.05 (1H, m), 2.5-2.7 (4H, m), 6.55-6.6 (1H, m), 6.9- 6.95 (1H, m), 7.1-7.2 (1H, m), 7.25-7.45 (4H, m), 7.45-7.55 (2H, m), 11.96 (1H, s), 14.41 (1H, s) |
| 390 | 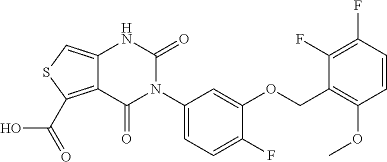 [SMILES: COc1ccc(F)c(F)c1COc1cc(-n2c(=O)[nH]c3csc(C(=O)O)c3c2=O)ccc1F] | (DMSO-d6) 3.82 (3H, s), 5.08 (2H, s), 6.9-7.0 (1H, m), 7.0-7.1 (1H, m), 7.3- 7.4 (2H, m), 7.4-7.55 (2H, m), 11.91 (1H, brs), 14.82 (1 H, brs) |
| 391 | 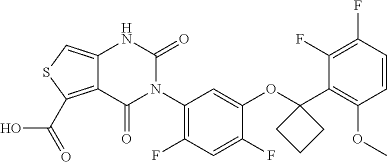 [SMILES: COc1ccc(F)c(F)c1C1(Oc2cc(-n3c(=O)[nH]c4csc(C(=O)O)c4c3=O)c(F)cc2F)CCC1] | (DMSO-d6) 1.8-1.95 (1H, m), 2.25-2.4 (1H, m), 2.6-2.8 (4H, m), 3.6 (3H, s), 6.65-6.75 (1H, m), 7.05-7.15 (1H, m), 7.25-7.35 (2H, m), 7.35-7.5 (1H, m), 11.7-12.2 (1H, br), 14.1-14.8 (1H, br) |
| 392 | 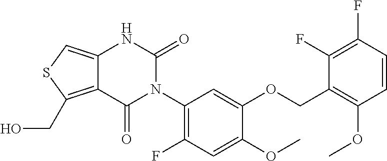 [SMILES: COc1cc(F)c(-n2c(=O)[nH]c3csc(CO)c3c2=O)cc1OCc1c(OC)ccc(F)c1F] | (DMSO-d6) 3.78 (3H, s), 3.81 (3H, s), 4.9-5.1 (4H, m), 5.95 (1H, t, J = 5.6 Hz), 6.71 (1H, s), 6.85-6.95 (1H, m), 7.05 (1H, d, J = 11.2 Hz), 7.17 (1H, d, J = 7.4 Hz), 7.4-7.55 (1H, m), 11.32 (1H, s) |
| 393 | 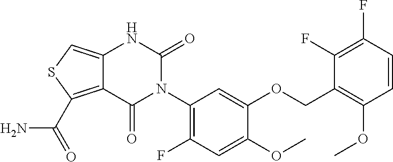 [SMILES: COc1cc(F)c(-n2c(=O)[nH]c3csc(C(N)=O)c3c2=O)cc1OCc1c(OC)ccc(F)c1F] | (DMSO-d6) 3.8 (3H, s), 3.81 (3H, s), 4.95 (2H, s), 6.85-6.95 (1H, m), 7.1 (1H, d, J = 11.5 Hz), 7.2-7.3 (2H, m), 7.4- 7.55 (1H, m), 8.05-8.15 (1H, m), 9.65- 9.75 (1H, m), 11.77 (1H, s) |
| 394 | 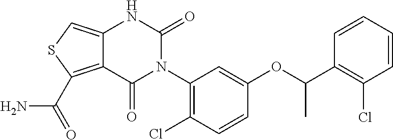 [SMILES: CC(Oc1ccc(Cl)c(-n2c(=O)[nH]c3csc(C(N)=O)c3c2=O)c1)c1ccccc1Cl] | (DMSO-d6) 1.58 (3H, d, J = 6.4 Hz), 5.65-5.75 (1H, m), 6.85-6.9 (1H, m), 7.2-7.25 (2H, m), 7.3-7.45 (2H, m), 7.45-7.55 (3H, m), 8.05-8.15 (1H, m), 9.6-9.7 (1H, m), 11.7-11.8 (1H, m) |
| 395 | 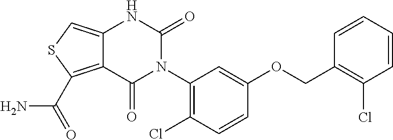 [SMILES: NC(=O)c1scc2[nH]c(=O)n(-c3cc(OCc4ccccc4Cl)ccc3Cl)c(=O)c12] | (DMSO-d6) 5.16 (2H, s), 7.19 (1H, dd, J = 8.9 Hz, 3.2 Hz), 7.24 (1H, s), 7.35- 7.45 (3H, m), 7.5-7.7 (3H, m), 8.11 (1H, d, J = 2.1 Hz), 9.65-9.7 (1H, m), 11.8 (1H, s) |
| 396 | 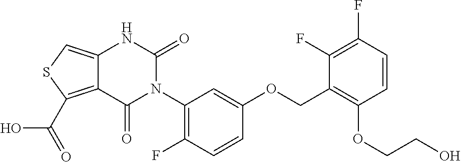 [SMILES: O=C(O)c1scc2[nH]c(=O)n(-c3cc(OCc4c(OCCO)ccc(F)c4F)ccc3F)c(=O)c12] | (DMSO-d6) 3.6-3.75 (2H, m), 4.04 (2H, t, J = 4.9 Hz), 4.8-4.95 (1H, m), 5.13 (2H, s), 6.9-6.95 (1H, m), 7.15-7.3 (2H, m), 7.3-7.4 (2H, m), 7.4-7.5 (1H, m), 12.01 (1H, s), 14.4 (1H, brs) |
| 397 | 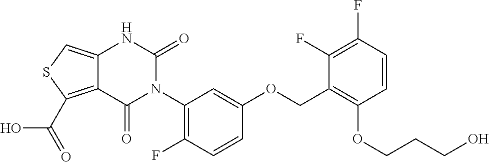 [SMILES: O=C(O)c1scc2[nH]c(=O)n(-c3cc(OCc4c(OCCCO)ccc(F)c4F)ccc3F)c(=O)c12] | (DMSO-d6) 1.75-1.9 (2H, m), 3.45-3.55 (2H, m), 4.1 (2H, t, J = 6.2 Hz), 4.9-5.0 (1H, m), 5.06 (2H, s), 6.85-6.95 (1H, m), 7.15-7.25 (2H, m), 7.3-7.4 (2H, m), 7.4- 7.5 (1H, m), 12.02 (1H, s), 14.4 (1H, brs) |
| 398 | 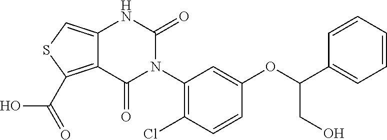 [SMILES: O=C(O)c1scc2[nH]c(=O)n(-c3cc(OC(CO)c4ccccc4)ccc3Cl)c(=O)c12] | (DMSO-d6) 3.55-3.65 (1H, m), 3.7-3.8 (1H, m), 5.15-5.25 (1H, m), 5.25-5.35 (1H, m), 7.0-7.05 (1H, m), 7.2-7.5 (8H, m), 11.95-12.05 (1H, m), 14.42 (1H, s) |
| 399 | 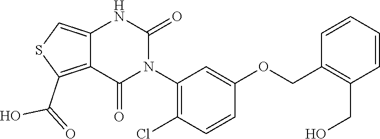 [SMILES: O=C(O)c1scc2[nH]c(=O)n(-c3cc(OCc4ccccc4CO)ccc3Cl)c(=O)c12] | (DMSO-d6) 4.61 (2H, s), 5.16 (2H, s), 5.21 (1H, brs), 7.15-7.5 (7H, m), 7.57 (1H, d, J = 8.6 Hz), 12.0 (1H, s) |
| 400 | 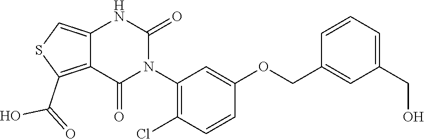 [SMILES: O=C(O)c1scc2[nH]c(=O)n(-c3cc(OCc4cccc(CO)c4)ccc3Cl)c(=O)c12] | (DMSO-d6) 4.51 (2H, d, J = 5.2 Hz), 5.1 (2H, s), 5.22 (1H, t, J = 5.2 Hz), 7.15-7.2 (1H, m), 7.25-7.45 (6H, m), 7.57 (1H, d, J = 8.6 Hz), 12.03 (1H, s), 14.44 (1H, s) |
| 401 | 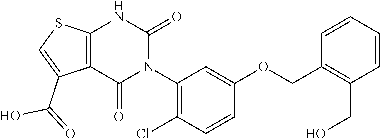 [SMILES: O=C(O)c1csc2[nH]c(=O)n(-c3cc(OCc4ccccc4CO)ccc3Cl)c(=O)c12] | (DMSO-d6) 4.61 (2H, s), 5.15 (2H, s), 7.15-7.5 (6H, m), 7.57 (1H, d, J = 9.2 Hz), 7.95 (1H, s), 13.95 (1H, s) |
| 402 | 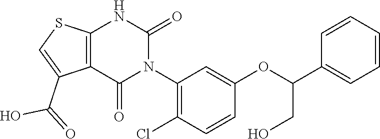 [SMILES: O=C(O)c1csc2[nH]c(=O)n(-c3cc(OC(CO)c4ccccc4)ccc3Cl)c(=O)c12] | (DMSO-d6) 3.55-3.65 (1H, m), 3.7-3.8 (1H, m), 5.0-5.4 (2H, m), 7.0-7.1 (1H, m), 7.15-7.5 (7H, m), 7.95 (1H, d, J = 5.9 Hz), 12.98 (1H, brs), 13.95 (1H, s) |
| 403 | 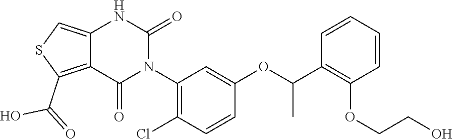 [SMILES: CC(Oc1ccc(Cl)c(-n2c(=O)[nH]c3csc(C(=O)O)c3c2=O)c1)c1ccccc1OCCO] | (DMSO-d6) 1.55 (3H, d, J = 6.3 Hz), 3.6-3.8 (2H, m), 3.95-4.15 (2H, m), 4.85-4.95 (1H, m), 5.75-5.9 (1H, m), 6.9-7.05 (3H, m), 7.15-7.5 (5H, m), 11.95-12.05 (1H, m), 14.43 (1H, s) |
| 404 | 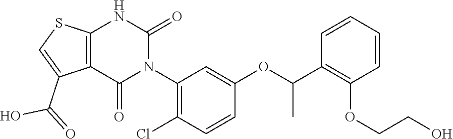 [SMILES: CC(Oc1ccc(Cl)c(-n2c(=O)[nH]c3scc(C(=O)O)c3c2=O)c1)c1ccccc1OCCO] | (DMSO-d6) 1.55 (3H, d, J = 6.3 Hz), 3.6-3.8 (2H, m), 3.95-4.15 (2H, m), 4.8- 5.0 (1H, br), 5.75-5.85 (1H, m), 6.9-7.05 (3H, m), 7.15-7.35 (3H, m), 7.43 (1H, d, J = 8.9 Hz), 7.9-8.0 (1H, m), 12.8-13.2 (1H, br), 13.99 (1H, s) |
| 405 | 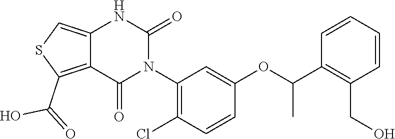 [SMILES: CC(Oc1ccc(Cl)c(-n2c(=O)[nH]c3csc(C(=O)O)c3c2=O)c1)c1ccccc1CO] | (DMSO-d6) 1.57 (3H, d, J = 6.2 Hz), 4.55-4.65 (1H, m), 4.65-4.75 (1H, m), 5.3-5.35 (1H, m), 5.7-5.8 (1H, m), 6.95- 7.05 (1H, m), 7.2-7.5 (7H, m), 11.95- 12.05 (1H, m), 14.42 (1H, s) |
| 406 | 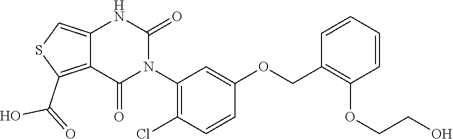 [SMILES: O=C(O)c1scc2[nH]c(=O)n(-c3cc(OCc4ccccc4OCCO)ccc3Cl)c(=O)c12] | (DMSO-d6) 3.65-3.75 (2H, m), 4.0-4.1 (2H, m), 4.87 (1H, brs), 5.12 (2H, s), 6.95-7.0 (1H, m), 7.05 (1H, d, J = 8.1 Hz), 7.19 (1H, dd, J = 9.0 Hz, 3.0 Hz), 7.25-7.45 (4H, m), 7.56 (1H, d, J = 9.0 Hz), 12.04 (1H, s), 14.46 (1H, s) |
| 407 | 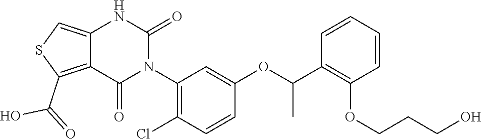 [SMILES: CC(Oc1ccc(Cl)c(-n2c(=O)[nH]c3csc(C(=O)O)c3c2=O)c1)c1ccccc1OCCCO] | (DMSO-d6) 1.54 (3H, d, J = 6.3 Hz), 1.85-1.95 (2H, m), 3.55-3.65 (2H, m), 4.05-4.2 (2H, m), 4.54 (1H, brs), 5.65- 5.75 (1H, m), 6.85-7.0 (2H, m), 7.0-7.1 (1H, m), 7.15-7.2 (1H, m), 7.2-7.35 (2H, m), 7.4 (1H, d, J = 4.6 Hz), 7.47 (1H, d, J = 9.0 Hz), 11.95-12.05 (1H, m), 14.42 (1H, s) |
| 408 | 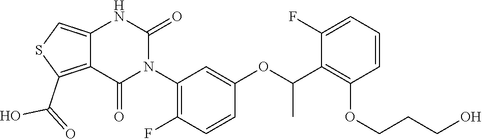 [SMILES: CC(Oc1ccc(F)c(-n2c(=O)[nH]c3csc(C(=O)O)c3c2=O)c1)c1c(F)cccc1OCCCO] | (DMSO-d6) 1.66 (3H, d, J = 6.4 Hz), 1.85-1.95 (2H, m), 3.5-3.6 (2H, m), 4.05-4.2 (2H, m), 5.75-5.85 (1H, m), 6.7-7.1 (4H, m), 7.2-7.4 (3H, 7), 11.95- 12.05 (1H, m), 14.35-14.45 (1H, m) |
| 409 | 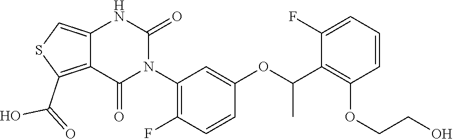 [SMILES: CC(Oc1ccc(F)c(-n2c(=O)[nH]c3csc(C(=O)O)c3c2=O)c1)c1c(F)cccc1OCCO] | (DMSO-d6) 1.66 (3H, d, J = 6.6 Hz), 3.55-3.8 (2H, m), 3.85-4.0 (1H, m), 4.05-4.2 (1H, m), 4.9-5.05 (1H, m), 5.9- 6.0 (1H, m), 6.7-6.9 (2H, m), 7.0-7.4 (5H, m), 11.95-12.05 (1H, m), 14.44 (1H, s) |
| 410 | 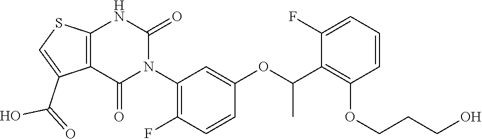 [SMILES: CC(Oc1ccc(F)c(-n2c(=O)[nH]c3scc(C(=O)O)c3c2=O)c1)c1c(F)cccc1OCCCO] | (DMSO-d6) 1.66 (3H, d, J = 6.4 Hz), 1.8-1.95 (2H, m), 3.5-3.6 (2H, m), 4.0- 4.2 (2H, m), 5.8 (1H, q, J = 6.4 Hz), 6.7- 6.8 (1H, m), 6.8-7.0 (2H, m), 7.0-7.05 (1H, m), 7.2-7.35 (2H, m), 7.9-7.95 (1H, m), 13.93 (1H, s) |
| 411 | 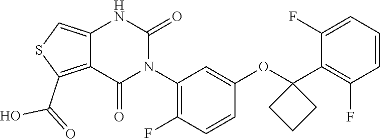 [SMILES: O=C(O)c1scc2[nH]c(=O)n(-c3cc(OC4(c5c(F)cccc5F)CCC4)ccc3F)c(=O)c12] | (DMSO-d6) 1.85-2.0 (1H, m), 2.2-2.35 (1H, m), 2.65-2.85 (4H, m), 6.75-6.85 (1H, m), 7.0-7.1 (3H, m), 7.24 (1H, t, J = 9.3 Hz), 7.3-7.45 (2H, m), 11.97 (1H, brs), 13.5-15.0 (1H, br) |
| 412 | 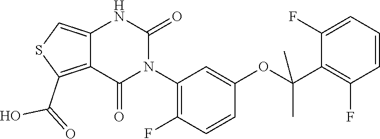 [SMILES: CC(C)(Oc1ccc(F)c(-n2c(=O)[nH]c3csc(C(=O)O)c3c2=O)c1)c1c(F)cccc1F] | (DMSO-d6) 1.820 (3H, s), 1.823 (3H, s), 6.75-6.8 (1H, m), 6.95-7.1 (3H, m), 7.15-7.25 (1H, m), 7.3-7.45 (2H, m), 11.96 (1H, brs), 14.43 (1H, brs) |
| 413 | 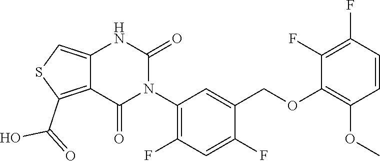 [SMILES: COc1ccc(F)c(F)c1OCc1cc(-n2c(=O)[nH]c3csc(C(=O)O)c3c2=O)c(F)cc1F] | (DMSO-d6) 3.79 (3H, s), 5.05-5.2 (2H, m), 6.8-6.8 (1H, m), 7.05-7.2 (1H, m), 7.37 (1H, s), 7.5-7.6 (1H, m), 7.65-7.75 (1H, m), 12.02 (1H, s), 14.35 (1H, s) |
| 414 | 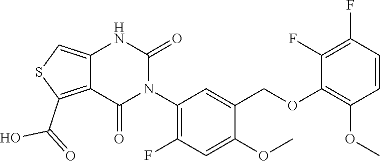 [SMILES: COc1cc(F)c(-n2c(=O)[nH]c3csc(C(=O)O)c3c2=O)cc1COc1c(OC)ccc(F)c1F] | (DMSO-d6) 3.79 (3H, s), 3.85 (3H, s), 5.0-5.1 (2H, m), 6.8-6.9 (1H, m), 7.05- 7.2 (2H, m), 7.38 (1H, s), 7.51 (1H, d, J = 8.5 Hz), 11.98 (1H, s), 14.54 (1H, brs) |
| 415 | 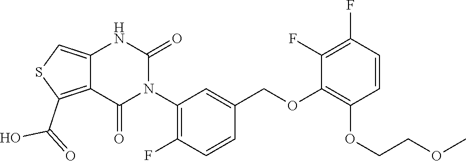 [SMILES: COCCOc1ccc(F)c(F)c1OCc1ccc(F)c(-n2c(=O)[nH]c3csc(C(=O)O)c3c2=O)c1] | (DMSO-d6) 3.27 (3H, s), 3.6-3.7 (2H, m), 4.1-4.2 (2H, m), 5.13 (2H, s), 6.85- 6.95 (1H, m), 7.05-7.2 (1H, m), 7.39 (1H, s), 7.4-7.5 (1H, m), 7.6-7.7 (2H, m), 12.05 (1H, s), 14.42 (1H, s) |
| 416 | 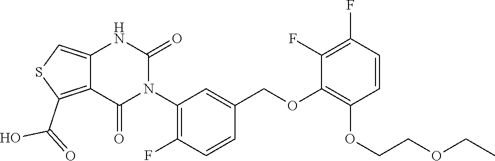 [SMILES: CCOCCOc1ccc(F)c(F)c1OCc1ccc(F)c(-n2c(=O)[nH]c3csc(C(=O)O)c3c2=O)c1] | (DMSO-d6) 1.05 (3H, t, J = 7.1 Hz), 3.46 (2H, q, J = 7.1 Hz), 3.65-3.75 (2H, m), 4.1-4.2 (2H, m), 5.14 (2H, s), 6.85- 6.95 (1H, m), 7.05-7.2 (1H, m), 7.38 (1H, s), 7.4-7.5 (1H, m), 7.55-7.7 (2H, m), 12.05 (1H, s), 14.41 (1H, s) |
| 417 | 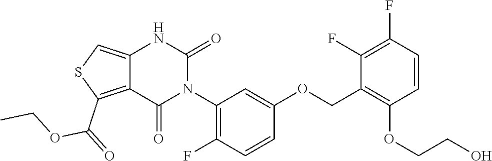 [SMILES: CCOC(=O)c1scc2[nH]c(=O)n(-c3cc(OCc4c(OCCO)ccc(F)c4F)ccc3F)c(=O)c12] | (DMSO-d6) 1.27 (3H, t, J = 7.1 Hz), 3.68 (2H, t, J = 4.8 Hz), 4.05 (2H, t, J = 4.8 Hz), 4.3 (2H, q, J = 7.1 Hz), 5.12 (2H, s), 6.85-6.95 (1H, m), 7.1-7.25 (3H, m), 7.25-7.35 (1H, m), 7.4-7.5 (1H, m), 11.63 (1H, s) |
| 418 | 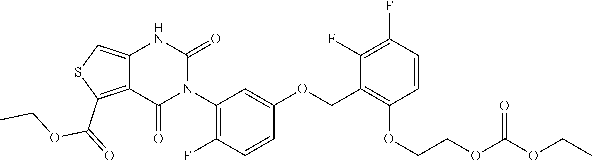 [SMILES: CCOC(=O)OCCOc1ccc(F)c(F)c1COc1ccc(F)c(-n2c(=O)[nH]c3csc(C(=O)OCC)c3c2=O)c1] | (DMSO-d6) 1.13 (3H, t, J = 7.0 Hz), 1.27 (3H, t, J = 7.1 Hz), 4.03 (2H, q, J = 7.0 Hz), 4.25-4.35 (4H, m), 4.35-4.45 (2H, m), 5.07 (2H, s), 6.9-7.0 (1H, m), 7.1-7.2 (3H, m), 7.25-7.35 (1H, m), 7.4- 7.55 (1H, m), 11.63 (1H, s) |
| 419 | 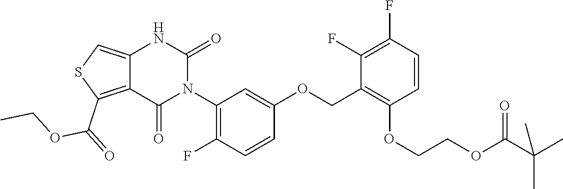 [SMILES: CCOC(=O)c1scc2[nH]c(=O)n(-c3cc(OCc4c(OCCOC(=O)C(C)(C)C)ccc(F)c4F)ccc3F)c(=O)c12] | (DMSO-d6) 1.04 (9H, s), 1.27 (3H, t, J = 7.1 Hz), 4.2-4.35 (6H, m), 5.05 (2H, s), 6.9-7.05 (1H, m), 7.05-7.2 (3H, m), 7.25-7.35 (1H, m), 7.4-7.55 (1H, m), 11.63 (1H, s) |
| 420 | 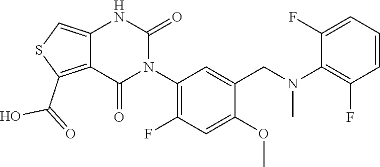 [SMILES: COc1cc(F)c(-n2c(=O)[nH]c3csc(C(=O)O)c3c2=O)cc1CN(C)c1c(F)cccc1F] | (DMSO-d6) 2.7-2.75 (3H, m), 3.71 (3H, s), 4.1-4.25 (2H, m), 6.95-7.15 (4H, m), 7.38 (1H, s), 7.41 (1H, d, J = 8.6 Hz), 11.95 (1H, s), 14.57 (1H, brs) |
| 421 | 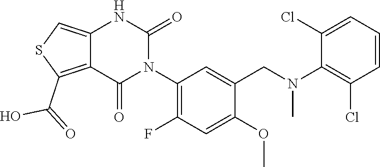 [SMILES: COc1cc(F)c(-n2c(=O)[nH]c3csc(C(=O)O)c3c2=O)cc1CN(C)c1c(Cl)cccc1Cl] | (DMSO-d6) 2.73 (3H, s), 3.74 (3H, s), 4.15-4.25 (2H, m), 7.08 (1H, d, J = 12.4 Hz), 7.15-7.2 (1H, m), 7.36 (1H, s), 7.4- 7.45 (2H, m), 7.56 (1H, d, J = 8.7 Hz), 11.96 (1H, brs), 14.61 (1H, brs) |
| 422 | 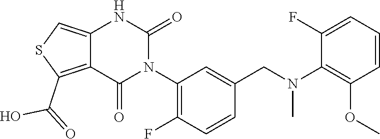 [SMILES: COc1cccc(F)c1N(C)Cc1ccc(F)c(-n2c(=O)[nH]c3csc(C(=O)O)c3c2=O)c1] | (DMSO-d6) 2.68 (3H, s), 3.81 (3H, s), 4.15 (2H, s), 6.7-6.85 (2H, m), 7.0-7.1 (1H, m), 7.3-7.4 (2H, m), 7.45-7.55 (2H, m), 11.98 (1H, s), 14.45 (1H, s) |
| 423 | 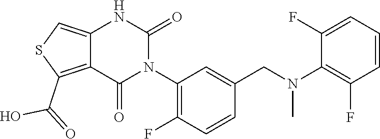 [SMILES: CN(Cc1ccc(F)c(-n2c(=O)[nH]c3csc(C(=O)O)c3c2=O)c1)c1c(F)cccc1F] | (DMSO-d6) 2.75 (3H, s), 4.22 (2H, s), 6.95-7.15 (3H, m), 7.35-7.45 (2H, m), 7.45-7.5 (2H, m), 12.02 (1H, s) |
| 424 | 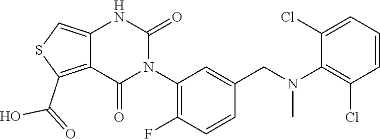 [SMILES: CN(Cc1ccc(F)c(-n2c(=O)[nH]c3csc(C(=O)O)c3c2=O)c1)c1c(Cl)cccc1Cl] | (DMSO-d6) 2.72 (3H, s), 4.3 (2H, s), 7.15-7.25 (1H, m), 7.35-7.5 (4H, m), 7.55-7.65 (2H, m), 12.03 (1H, s) |
| 425 | 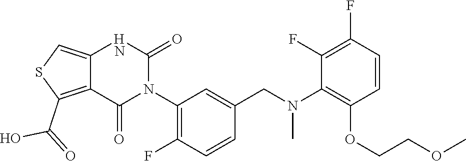 | (DMSO-d6) 2.7-2.75 (3H, m), 3.23 (3H, s), 3.6-3.7 (2H, m), 4.05-4.1 (2H, m), 4.22 (2H, s), 6.7-6.8 (1H, m), 7.0-7.1 (1H, m), 7.3-7.4 (2H, m), 7.45-7.55 (2H, m), 12.03 (1H, s), 14.46 (1H, brs) |
| 426 | 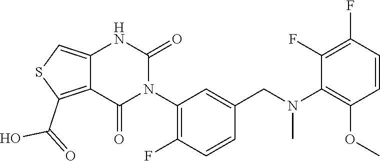 [SMILES: COc1ccc(F)c(F)c1N(C)Cc1ccc(F)c(-n2c(=O)[nH]c3csc(C(=O)O)c3c2=O)c1] | (DMSO-d6) 2.7-2.75 (3H, m), 3.79 (3H, s), 4.2 (2H, s), 6.7-6.8 (1H, m), 7.0-7.15 (1H, m), 7.3-7.4 (2H, m), 7.45-7.55 (2H, m), 12.02 (1H, s), 14.47 (1H, brs) |
| 427 | 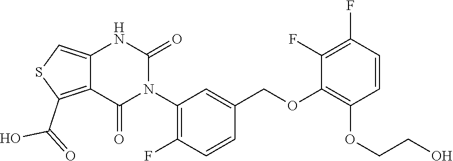 [SMILES: O=C(O)c1scc2[nH]c(=O)n(-c3cc(COc4c(OCCO)ccc(F)c4F)ccc3F)c(=O)c12] | (DMSO-d6) 3.7-3.8 (2H, m), 4.04 (2H, t, J = 4.6 Hz), 4.85-4.95 (1H, m), 5.17 (2H, s), 6.85-6.95 (1H, m), 7.05-7.15 (1H, m), 7.39 (1H, s), 7.4-7.5 (1H, m), 7.6-7.7 (2H, m), 12.05 (1H, s), 14.44 (1H, s) |
| 428 | 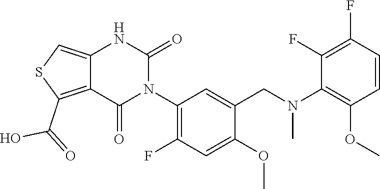 [SMILES: COc1cc(F)c(-n2c(=O)[nH]c3csc(C(=O)O)c3c2=O)cc1CN(C)c1c(OC)ccc(F)c1F] | (DMSO-d6) 2.7-2.75 (3H, m), 3.7-3.8 (6H, m), 4.1-4.2 (2H, m), 6.7-6.75 (1H, m), 6.95-7.1 (2H, m), 7.37 (1H, s), 7.41 (1H, d, J = 8.8 Hz), 11.95 (1H, s), 14.57 (1H, s) |

For example, the GnRH antagonist may be 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, or a pharmaceutically acceptable salt thereof. The salt may be, for instance, the choline salt thereof, represented by formula (VIa), below.

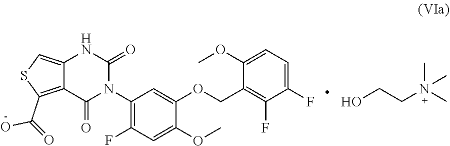

Compound (VI) and pharmaceutically acceptable salts thereof, such as the choline salt thereof (compound (VIa)), can be synthesized, for example, using the methodology described in WO 2014/042176, the disclosure of which is incorporated herein by reference in its entirety. An exemplary synthetic scheme that may be used for the preparation of compound (VI) and the choline salt thereof is shown in Scheme 1, below.

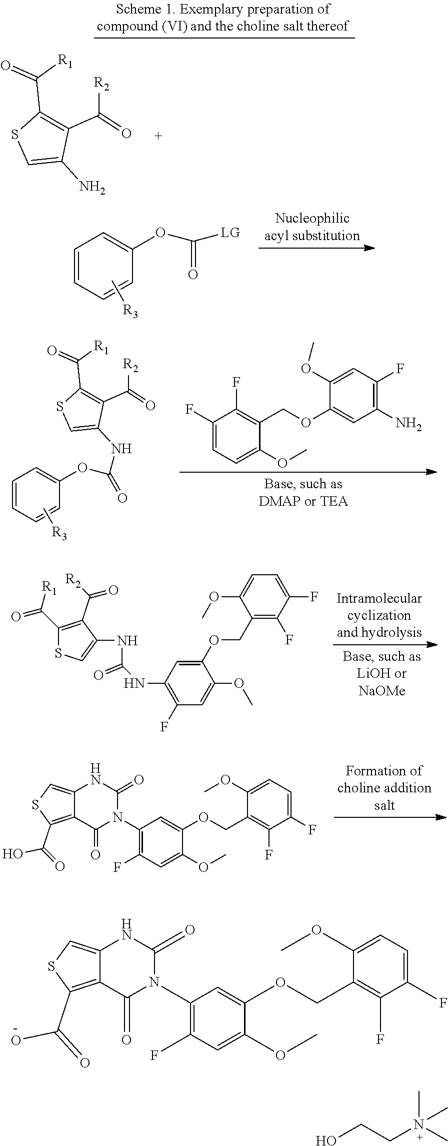

wherein R₁ and R₂ are each independently C₁₋₆ alkoxy groups; LG is a nucleofugal leaving group, such as chlorine or bromine, among others; R₃ represents an optional substituent, such as halogen, acyl group, C₁₋₆ alkyl group, or a nitro substituent; DMAP denotes N-dimethylaminopyridine; and TEA denotes trimethylamine.

Crystalline compound (VIa) has been characterized spectroscopically, for instance, in U.S. Pat. No. 9,169,266, the disclosure of which is incorporated herein by reference in its entirety. The foregoing crystalline form has been shown to exhibit characteristic X-ray powder diffraction peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ. Additionally, this crystalline form exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm. This crystalline form further exhibits ¹⁹F solid-state NMR peaks centered at about −151.8 ppm, −145.2 ppm, and −131.6 ppm.

Compound (VI), as well as pharmaceutically acceptable salts thereof, such as the choline salt thereof, exhibit a high affinity for human GnRH receptor (27.4 nM). Using the compositions and methods described herein, a patient that is presenting with or has been diagnosed as having an estrogen-dependent disease (e.g., uterine fibroids, endometriosis, such as rectovaginal endometriosis, and/or adenomyosis, among others described herein) may be administered a compound of formula (VI), or a pharmaceutically acceptable salt thereof, such as the choline salt thereof, to treat the disease or ameliorate one or more symptoms of the disease. Exemplary doses of compound (VI) and pharmaceutically acceptable salts thereof, such as the choline salt thereof, include doses of from 25 mg to 500 mg daily, such as doses of 100 mg per day and 200 mg per day. Additional dosing information is provided below.

### 3-Aminoalkyl pyrimidine-2,4(1H,3H)-diones

Additional GnRH antagonists that may be used in conjunction with the compositions and methods described herein include optionally substituted 3-aminoalkyl pyrimidine-2,4(1H,3H)-dione derivatives, such as compounds represented by formula (VII)

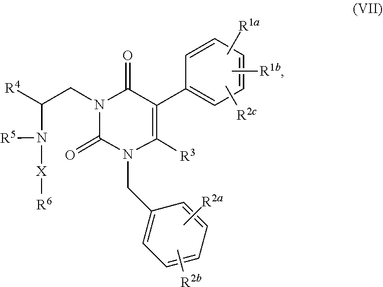

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

For example, the GnRH antagonist may be the conjugate acid of elagolix, which is represented by formula (VIII),

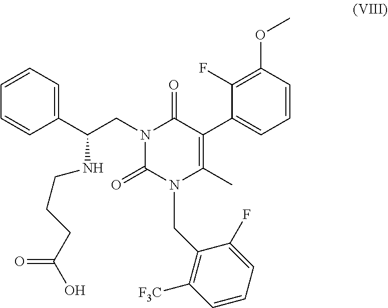 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

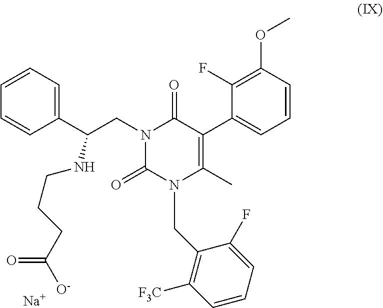 [SMILES: COc1cccc(-c2c(C)n(Cc3c(F)cccc3C(F)(F)F)c(=O)n(C[C@H](NCCCC(=O)[O-])c3ccccc3)c2=O)c1F ; [Na+]]

Compound (IX), also referred to as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, is known as elagolix. Other GnRH antagonists of this chemical class that may be used for the treatment of estrogen-dependent diseases in accordance with the compositions and methods of the disclosure include compounds described in U.S. Pat. No. 7,056,927, the contents of which are incorporated herein by reference. Exemplary GnRH antagonists of this chemical class that may be used for the treatment of estrogen-dependent diseases in accordance with the present disclosure include the compounds set forth in Table 2, below.

TABLE 2
Exemplary 3-Aminoalkyl pyrimidine-2,4(1H,3H)-dione GnRH Antagonists Useful for the
Treatment of Estrogen-Dependent Diseases

| No. | Compound | Reported spectral properties |
|---|---|---|
| 1 | 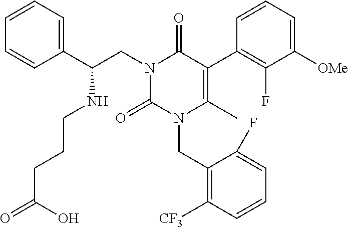 [SMILES: COc1cccc(-c2c(C)n(Cc3c(F)cccc3C(F)(F)F)c(=O)n(C[C@H](NCCCC(=O)O)c3ccccc3)c2=O)c1F] | HPLC MS (Cl) m/z 632.2 (MH+), tR = 26.45 |
| 2 | 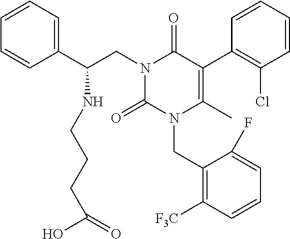 [SMILES: Cc1c(-c2ccccc2Cl)c(=O)n(C[C@H](NCCCC(=O)O)c2ccccc2)c(=O)n1Cc1c(F)cccc1C(F)(F)F] | MS (Cl) m/z 618.2 (MH+) tR = 1.005 |
| 3 | 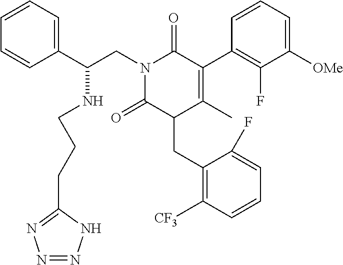 [SMILES: COc1cccc(C2=C(C)C(Cc3c(F)cccc3C(F)(F)F)C(=O)N(C[C@H](NCCCc3nnn[nH]3)c3ccccc3)C2=O)c1F] | HPLC-MS (Cl) m/z 656.2 (MH+), tR = 2.128 min |
| 4 | 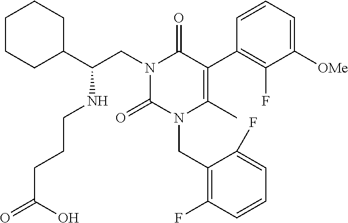 [SMILES: COc1cccc(-c2c(C)n(Cc3c(F)cccc3F)c(=O)n(C[C@H](NCCCC(=O)O)C3CCCCC3)c2=O)c1F] | MS (Cl) m/z 588.3 (MH+) |
| 5 | 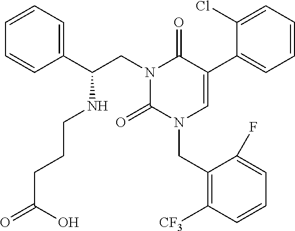 [SMILES: O=C(O)CCCN[C@@H](Cn1c(=O)c(-c2ccccc2Cl)cn(Cc2c(F)cccc2C(F)(F)F)c1=O)c1ccccc1] | HPLC-MS (Cl) m/z 604.1, 606.1 (MH+), tR = 2.511 |
| 6 | 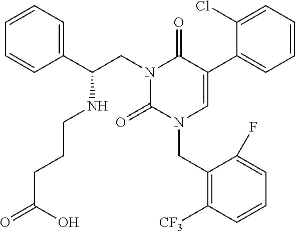 [SMILES: O=C(O)CCCN[C@@H](Cn1c(=O)c(-c2ccccc2Cl)cn(Cc2c(F)cccc2C(F)(F)F)c1=O)c1ccccc1] | HPLC-MS (Cl) m/z 604.1, 606.1 (MH+), tR = 26.98 |
| 7 | 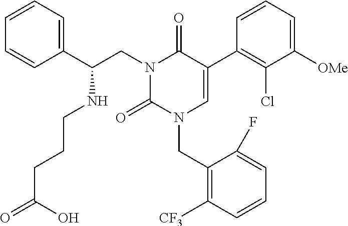 [SMILES: COc1cccc(-c2cn(Cc3c(F)cccc3C(F)(F)F)c(=O)n(C[C@H](NCCCC(=O)O)c3ccccc3)c2=O)c1Cl] | HPLC-MS (Cl) m/z 634.2, 636.2 (MH+), tR = 24.925 |
| 8 | 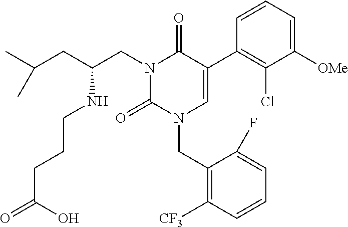 [SMILES: COc1cccc(-c2cn(Cc3c(F)cccc3C(F)(F)F)c(=O)n(C[C@@H](CC(C)C)NCCCC(=O)O)c2=O)c1Cl] | MS (Cl) m/z 614.1, 616.1 (MH+), tR = 6.550 min |
| 9 | 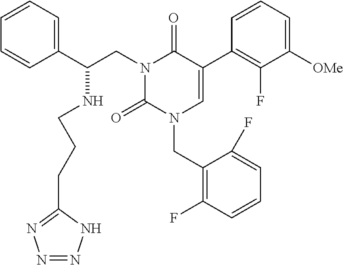 [SMILES: COc1cccc(-c2cn(Cc3c(F)cccc3F)c(=O)n(C[C@H](NCCCc3nnn[nH]3)c3ccccc3)c2=O)c1F] | HPLC-MS (Cl) m/z 592.3 (MH+), tR = 2.150 |
| 10 | 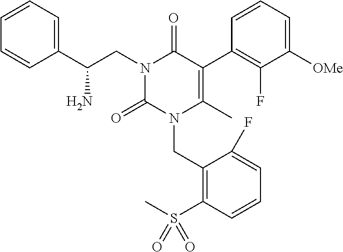 [SMILES: COc1cccc(-c2c(C)n(Cc3c(F)cccc3S(C)(=O)=O)c(=O)n(C[C@H](N)c3ccccc3)c2=O)c1F] | HPLC-MS (Cl) m/z = 556.2 (M + H+), tR = 2.354 |
| 11 | 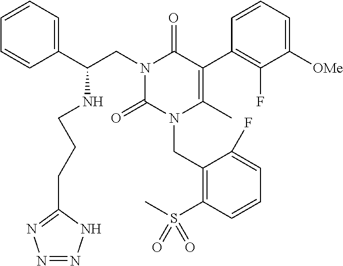 [SMILES: COc1cccc(-c2c(C)n(Cc3c(F)cccc3S(C)(=O)=O)c(=O)n(C[C@H](NCCCc3nnn[nH]3)c3ccccc3)c2=O)c1F] | |

### Thieno[2,3d]pyrimidines

Additional GnRH antagonists that may be used in conjunction with the compositions and methods described herein include optionally substituted thieno[2,3d]pyrimidine derivatives, such as compounds represented by formula (X)

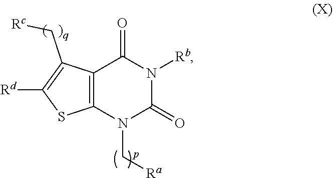

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a thieno[2,3d]pyrimidine compound represented by formula (XI)

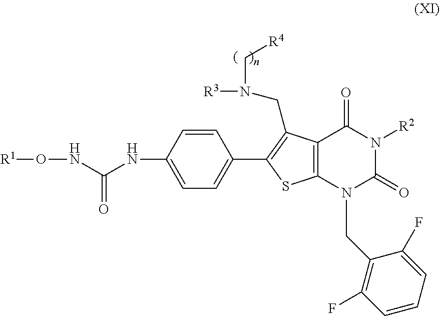

wherein R¹ is C₁₋₄alkyl;

R² is (1) a C₁₋₆alkyl which may have a substituent selected from the group consisting of (1) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1) a halogen, (2′) a hydroxy group, (3′) a C₁₋₄alkyl and (4′) a C₁₋₄alkoxy, (4) a phenyl which may have a substituent selected from the group consisting of (1) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4) N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. For example, the GnRH antagonist may be a compound represented by formula (XII), below.

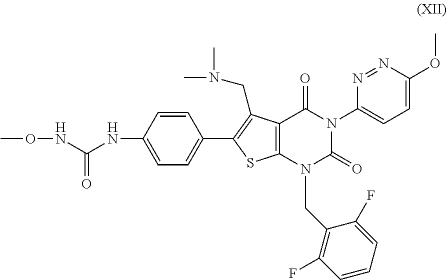

Compound (XII), also referred to as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, is known as relugolix. Other GnRH antagonists of this chemical class that may be used for the treatment of estrogen-dependent diseases in accordance with the compositions and methods of the disclosure include compounds described in U.S. Pat. No. 7,300,935, the contents of which are incorporated herein by reference. Exemplary GnRH antagonists of this chemical class that may be used for the treatment of estrogen-dependent diseases in accordance with the present disclosure include the compounds set forth in Table 3, below.

TABLE 3
Exemplary Thieno[2,3d]pyrimidine GnRH Antagonists Useful for the Treatment of Estrogen-
Dependent Diseases

| No. | Compound | Reported spectral properties |
|---|---|---|
| 1 | 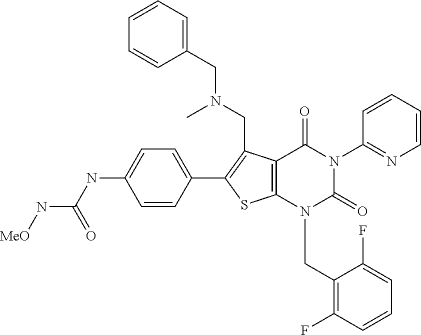 [SMILES: CONC(=O)Nc1ccc(-c2sc3c(c2CN(C)Cc2ccccc2)c(=O)n(-c2ccccn2)c(=O)n3Cc2c(F)cccc2F)cc1] | ¹H-NMR (CDCl₃) δ: 2.05 (3H, s), 3.56 (2H, s), 3.82 (3H, s), 3.89 (2H, s), 5.34 (2H, brs), 6.91 (2H, t, J = 8.0 Hz), 7.1- 7.45 (9H, m), 7.56 (2H, d, J = 8.8 Hz), 7.65 (1H, s), 7.75 (2H, d, J = 8.8 Hz), 7.91 (1H, dt, J = 2.0, 7.7 Hz), 8.7-8.75 (1H, m). |
| 2 | 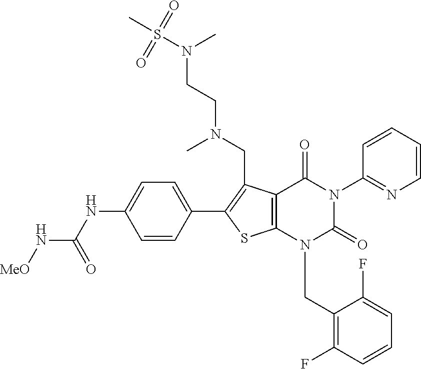 [SMILES: CONC(=O)Nc1ccc(-c2sc3c(c2CN(C)CCN(C)S(C)(=O)=O)c(=O)n(-c2ccccn2)c(=O)n3Cc2c(F)cccc2F)cc1] | ¹H-NMR (CDCl₃) δ: 2.11 (3H, s), 2.45-2.6 (2H, m), 2.70 (3H, s), 2.75 (3H, s), 3.1-3.25 (2H, m), 3.80 (2H, s), 3.83 (3H, s), 5.36 (2H, brs), 6.93 (2H, t, J = 8.2 Hz), 7.14 (1H, s), 7.2- 7.6 (7H, m), 7.65 (1H, s), 7.85- 7.95 (1H, m), 8.65-8.75 (1H, m). |
| 3 | 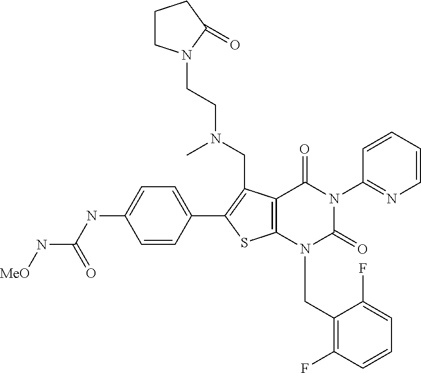 [SMILES: CONC(=O)Nc1ccc(-c2sc3c(c2CN(C)CCN2CCCC2=O)c(=O)n(-c2ccccn2)c(=O)n3Cc2c(F)cccc2F)cc1] | ¹H-NMR (CDCl₃) δ: 1.7-1.85 (2H, m), 2.17 (3H, s), 2.15-2.3 (2H, m), 2.5-2.6 (2H, m), 3.15 (2H, t, J = 7.0 Hz), 3.2-3.4 (2H, m), 3.76 (2H, s), 3.83 (3H, s), 5.36 (2H, brs), 6.93 (2H, t, J = 8.4 Hz), 7.16 (1H, s), 7.2- 7.7 (8H, m), 7.85-7.95 (1H, m), 8.65-8.75 (1H, m). |
| 4 | 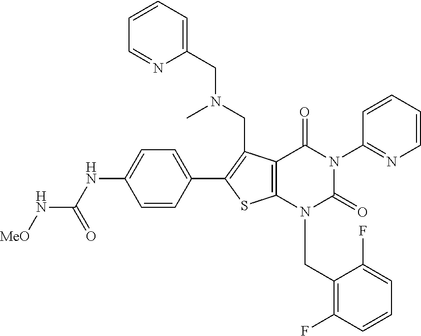 [SMILES: CONC(=O)Nc1ccc(-c2sc3c(c2CN(C)Cc2ccccn2)c(=O)n(-c2ccccn2)c(=O)n3Cc2c(F)cccc2F)cc1] | ¹H-NMR (CDCl₃) δ: 2.10 (3H, s), 3.70 (2H, s), 3.82 (3H, s), 3.96 (3H, s), 5.34 (2H, brs), 6.85-7.7 (14H, m), 7.85-7.95 (1H, m), 8.4-8.5 (1H, m), 8.65- 8.75 (1H, m). |
| 5 | 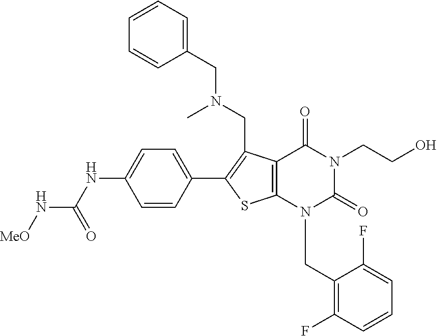 [SMILES: CONC(=O)Nc1ccc(-c2sc3c(c2CN(C)Cc2ccccc2)c(=O)n(CCO)c(=O)n3Cc2c(F)cccc2F)cc1] | ¹H-NMR (CDCl₃) δ: 2.04 (3H, s), 2.5-2.65 (1H, m), 3.58 (2H, s), 3.83 (3H, s), 3.91 (2H, s), 3.9-4.0 (2H, m), 4.37 (2H, t, J = 5.0 Hz), 5.34 (2H, s), 6.92 (2H, t, J = 8.2 Hz), 7.1-7.4 (7H, m), 7.54 (2H, d, J = 8.6 Hz), 7.66 (2H, d, J = 8.6 Hz), 7.6- 7.7 (1H, m). |
| 6 | 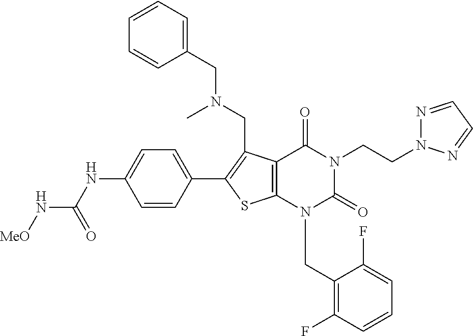 [SMILES: CONC(=O)Nc1ccc(-c2sc3c(c2CN(C)Cc2ccccc2)c(=O)n(CCn2nccn2)c(=O)n3Cc2c(F)cccc2F)cc1] | ¹H-NMR (CDCl₃) δ: 2.04 (3H, s), 3.51 (2H, s), 3.82 (3H, s), 3.86 (2H, s), 4.57 (2H, t, J = 6.2 Hz), 4.81 (2H, t, J = 6.2 Hz), 5.28 (2H, s), 6.91 (2H, t, J = 8.4 (2H, s), 7.53 (2H, d, J = 8.6 Hz), 7.62 (1H, s), 7.70 (2H, t, J = 8.6 Hz), 7.75 (1H, s). |
| 7 | 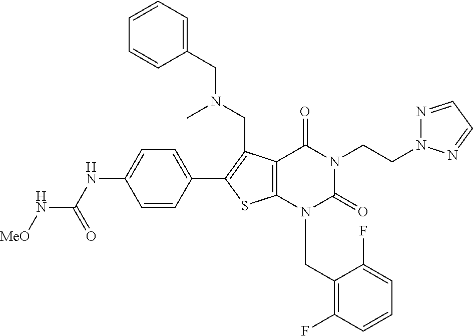 [SMILES: CONC(=O)Nc1ccc(-c2sc3c(c2CN(C)Cc2ccccc2)c(=O)n(CCn2nccn2)c(=O)n3Cc2c(F)cccc2F)cc1] | 2-yl Form (1) ¹H-NMR (CDCl₃) δ: 2.02 (3H, s), 3.51 (2H, s), 3.83 (5H, s), 4.6-4.7 (2H, m), 5.0-5.1 (2H, m), 5.28 (2H, s), 6.92 (2H, t, J = 8.2 Hz), 7.12 (1H, s), 7.2- 7.75 (11H, m), 8.38 (1H, s). |
| | 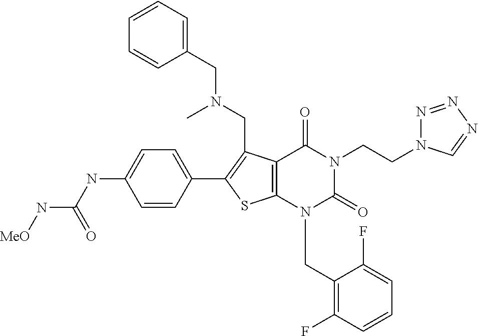 [SMILES: CONC(=O)Nc1ccc(-c2sc3c(c2CN(C)Cc2ccccc2)c(=O)n(CCn2cnnn2)c(=O)n3Cc2c(F)cccc2F)cc1] | 1-yl Form (2) ¹H-NMR (CDCl₃) δ: 2.01 (3H, s), 3.51 (2H, s), 3.83 (3H, s), 3.8-4.0 (2H, m), 4.6-4.7 (2H, m), 4.8-4.9 (2H, m), 5.30 (2H, s), 6.65-6.75 (1H, m), 6.85-7.0 (2H, m), 7.1-7.7 (11H, m), 8.68 (1H, s). |
| 8 | 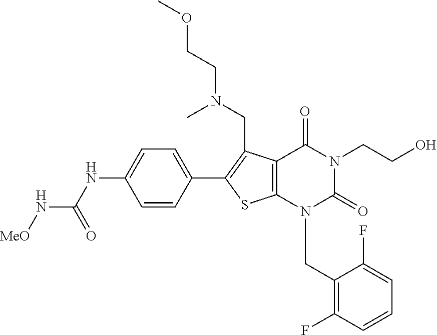 [SMILES: COCCN(C)Cc1c(-c2ccc(NC(=O)NOC)cc2)sc2c1c(=O)n(CCO)c(=O)n2Cc1c(F)cccc1F] | ¹H-NMR (CDCl₃) δ: 2.11 (3H, s), 2.65 (2H, t, J = 5.8 Hz), 3.30 (3H, s), 3.46 (2H, t, J = 5.8 Hz), 3.82 (5H, s), 3.9-4.0 (2H, m), 4.35 (2H, t, J = 5.2 Hz), 5.34 (2H, s), 6.92 (2H, t, J = 8.0 Hz), 7.14 (1H, s), 7.2-7.35 (1H, m), 7.5-7.65 (5H, m). |
| 9 | 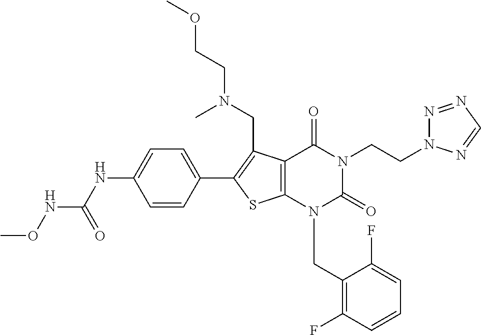 [SMILES: COCCN(C)Cc1c(-c2ccc(NC(=O)NOC)cc2)sc2c1c(=O)n(CCn1ncnn1)c(=O)n2Cc1c(F)cccc1F] | 2-yl Form (1) ¹H-NMR (CDCl₃) δ: 2.10 (3H, brs), 2.55-2.65 (2H, m), 3.31 (3H, s), 3.4-3.5 (2H, m), 3.76 (2H, s), 3.82 (3H, s), 4.62 (2H, t, J = 5.8 Hz), 5.02 (2H, t, J = 5.8 Hz), 5.27 (2H, s), 6.92 (2H, t, J = 8.2 Hz), 7.13 (1H, s), 7.25- 7.4 (1H, m), 7.5-7.65 (5H, m), 8.43 (1H, s). |
| | 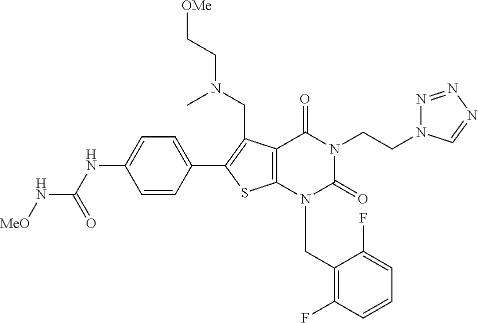 [SMILES: COCCN(C)Cc1c(-c2ccc(NC(=O)NOC)cc2)sc2c1c(=O)n(CCn1cnnn1)c(=O)n2Cc1c(F)cccc1F] | 1-yl Form (2) ¹H-NMR (CDCl₃) δ: 2.07 (3H, s), 2.55-2.65 (2H, m), 3.30 (3H, s), 3.4-3.5 (2H, m), 3.74 (2H, s), 3.82 (3H, s), 4.55-4.65 (2H, m), 4.8-4.9 (2H, m), 5.30 (2H, s), 6.93 (2H, t, J = 7.8 Hz), 7.10 (1H, s), 7.2-7.4 (1H, m), 7.5-7.65 (5H, m), 8.69 (1H, s). |
| 10 | 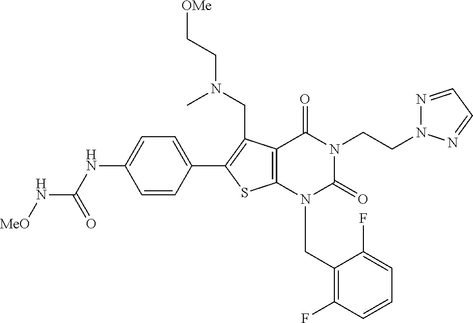 [SMILES: COCCN(C)Cc1c(-c2ccc(NC(=O)NOC)cc2)sc2c1c(=O)n(CCn1nccn1)c(=O)n2Cc1c(F)cccc1F] | ¹H-NMR (CDCl₃) δ: 2.11 (3H, s), 2.62 (2H, t, J = 5.8 Hz), 3.31 (3H, s), 3.44 (2H, t, J = 5.8 Hz), 3.78 (2H, s), 3.82 (3H, s), 4.55 (2H, t, J = 5.6 Hz), 4.79 (2H, t, J = 5.6 Hz), 5.27 (2H, s), 6.91 (2H, t, J = 8.2 Hz), 7.10 (1H, s), 7.2-7.4 (2H, m), 7.51 (2H, s), 7.45-7.65 (4H, m). |
| 11 | 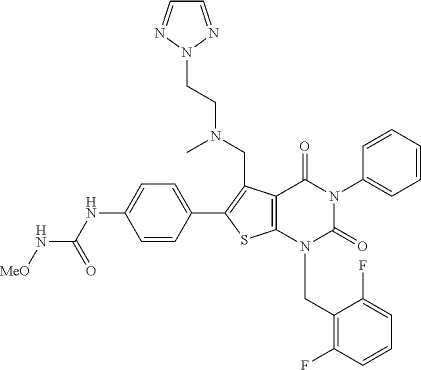 [SMILES: CONC(=O)Nc1ccc(-c2sc3c(c2CN(C)CCn2nccn2)c(=O)n(-c2ccccc2)c(=O)n3Cc2c(F)cccc2F)cc1] | 2-yl Form (1) ¹H-NMR (CDCl₃) δ: 2.23 (3H, s), 3.00 (2H, t, J = 6.3 Hz), 3.78 (2H, s), 3.82 (3H, s), 4.45 (2H, t, J = 6.3 Hz), 5.37 (2H, s), 6.92 (2H, t, J = 8.2 Hz), 6.85-6.95 (1H, m), 7.11 (1H, s), 7.2-7.6 (12H, m). |
| | 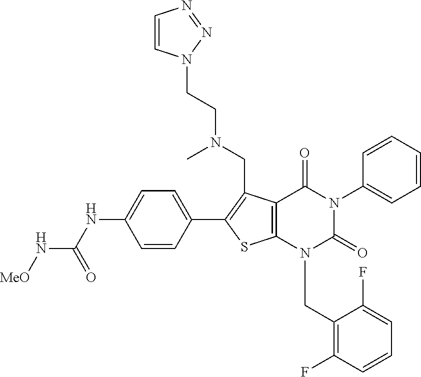 [SMILES: CONC(=O)Nc1ccc(-c2sc3c(c2CN(C)CCn2ccnn2)c(=O)n(-c2ccccc2)c(=O)n3Cc2c(F)cccc2F)cc1] | 1-yl Form (2) ¹H-NMR (CDCl₃) δ: 2.14 (3H, s), 2.82 (2H, t, J = 6.0 Hz), 3.80 (2H, s), 3.82 (3H, s), 4.39 (2H, t, J = 6.0 Hz), 5.37 (2H, s), 6.92 (2H, t, J = 8.2 Hz), 6.85-6.95 (1H, m), 7.14 (1H, s), 7.2-7.55 (11H, m), 7.63 (1H, s). |
| 12 | 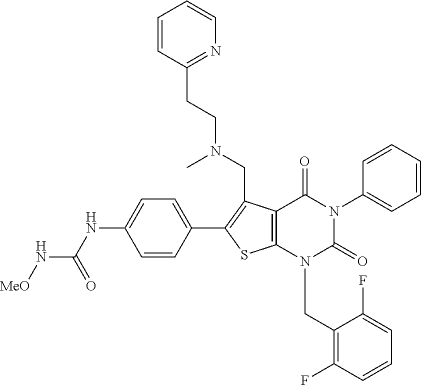 [SMILES: CONC(=O)Nc1ccc(-c2sc3c(c2CN(C)CCc2ccccn2)c(=O)n(-c2ccccc2)c(=O)n3Cc2c(F)cccc2F)cc1] | ¹H-NMR (CDCl₃) δ: 2.20 (3H, s), 2.85 (4H, s), 3.82 (5H, s), 5.37 (2H, s), 6.93 (2H, t, J = 8.2 Hz), 6.95-7.1 (2H, m), 7.14 (1H, s), 7.2-7.55 (11H, m), 7.60 (1H, s), 8.43 (1H, d, J = 4.0 Hz). |
| 13 | 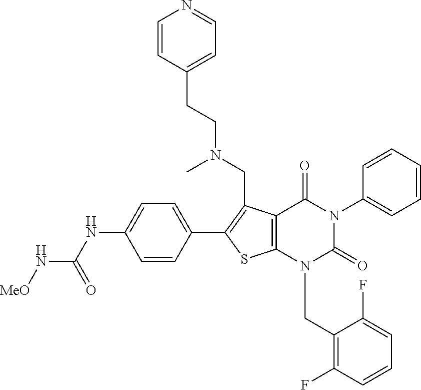 [SMILES: CONC(=O)Nc1ccc(-c2sc3c(c2CN(C)CCc2ccncc2)c(=O)n(-c2ccccc2)c(=O)n3Cc2c(F)cccc2F)cc1] | ¹H-NMR (CDCl₃) δ: 2.18 (3H, s), 2.68 (4H, s), 3.83 (5H, s), 5.37 (2H, s), 6.85-7.0 (5H, m), 7.16 (1H, s), 7.2-7.65 (10H, m), 8.35-8.4 (2H, m). |
| 14 | 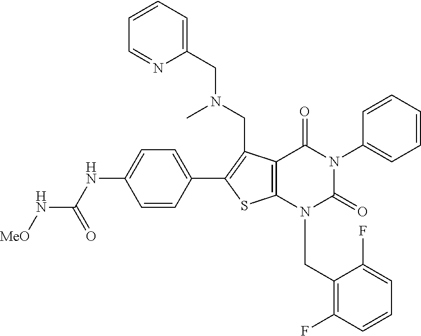 [SMILES: CONC(=O)Nc1ccc(-c2sc3c(c2CN(C)Cc2ccccn2)c(=O)n(-c2ccccc2)c(=O)n3Cc2c(F)cccc2F)cc1] | ¹H-NMR (CDCl₃) δ: 2.10 (3H, s), 3.71 (2H, s), 3.83 (3H, s), 3.99 (2H, s), 5.36 (2H, s), 6.92 (2H, t, J = 8.2 Hz), 7.0-7.1 (1H, m), 7.15 (1H, s), 7.2-7.35 (4H, m), 7.4-7.65 (9H, m), 8.4-8.5 (1H, m). |
| 15 | 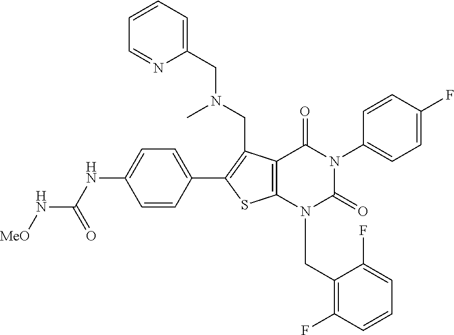 [SMILES: CONC(=O)Nc1ccc(-c2sc3c(c2CN(C)Cc2ccccn2)c(=O)n(-c2ccc(F)cc2)c(=O)n3Cc2c(F)cccc2F)cc1] | ¹H-NMR (CDCl₃) δ: 2.10 (3H, s), 3.71 (2H, s), 3.83 (3H, s), 3.97 (2H, s), 5.35 (2H, s), 6.93 (2H, t, J = 8.2 Hz), 7.0-7.7 (14H, m), 8.4-8.5 (1H, m). |
| 16 | 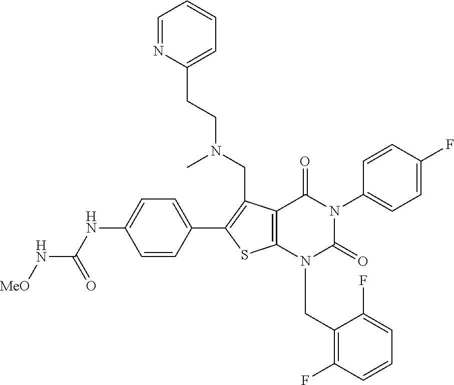 [SMILES: CONC(=O)Nc1ccc(-c2sc3c(c2CN(C)CCc2ccccn2)c(=O)n(-c2ccc(F)cc2)c(=O)n3Cc2c(F)cccc2F)cc1] | ¹H-NMR (CDCl₃) δ: 2.19 (3H, s), 2.85 (4H, s), 3.82 (5H, s), 5.36 (2H, s), 6.9-7.55 (13H, m), 7.60 (1H, s), 8.4-8.45 (1H, m). |
| 17 | 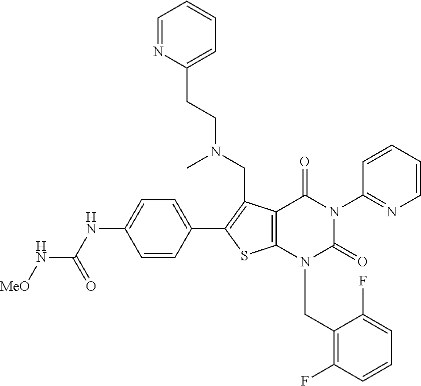 [SMILES: CONC(=O)Nc1ccc(-c2sc3c(c2CN(C)CCc2ccccn2)c(=O)n(-c2ccccn2)c(=O)n3Cc2c(F)cccc2F)cc1] | ¹H-NMR (CDCl₃) δ: 2.21 (3H, s), 2.8-2.9 (4H, m), 3.82 (5H, s), 5.34 (2H, brs), 6.85-7.75 (13H, m), 7.60 (1H, s), 7.85- 7.95 (1H, m), 8.4-8.5 (1H, m), 8.65-8.75 (1H, m). |
| 18 | 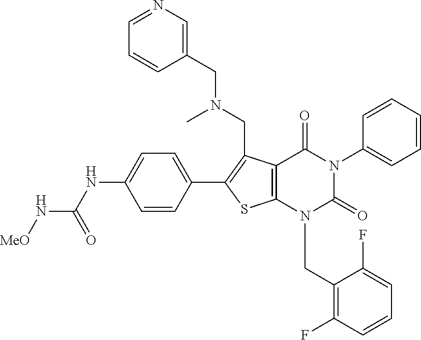 [SMILES: CONC(=O)Nc1ccc(-c2sc3c(c2CN(C)Cc2cccnc2)c(=O)n(-c2ccccc2)c(=O)n3Cc2c(F)cccc2F)cc1] | ¹H-NMR (CDCl₃) δ: 2.04 (3H, s), 3.58 (2H, s), 3.83 (3H, s), 3.92 (2H, s), 5.37 (2H, s), 6.92 (2H, t, J = 8.2 Hz), 7.05-7.7 (14H, m), 8.35-8.45 (2H, m). |
| 19 | 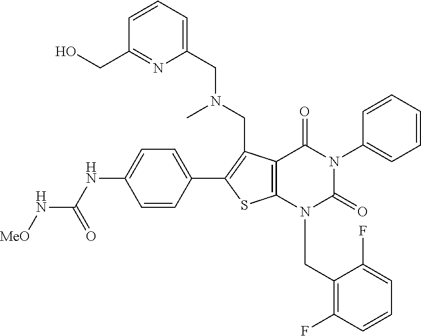 [SMILES: CONC(=O)Nc1ccc(-c2sc3c(c2CN(C)Cc2cccc(CO)n2)c(=O)n(-c2ccccc2)c(=O)n3Cc2c(F)cccc2F)cc1] | ¹H-NMR (CDCl₃) δ: 2.13 (3H, s), 3.72 (2H, s), 3.83 (3H, s), 3.97 (2H, s), 4.05-4.1 (1H, m), 4.65-4.75 (2H, m), 5.37 (2H, s), 6.93 (2H, t, J = 8.2 Hz), 6.9- 7.05 (1H, m), 7.1-7.2 (2H, m), 7.2-7.7 (12H, m). |
| 20 | 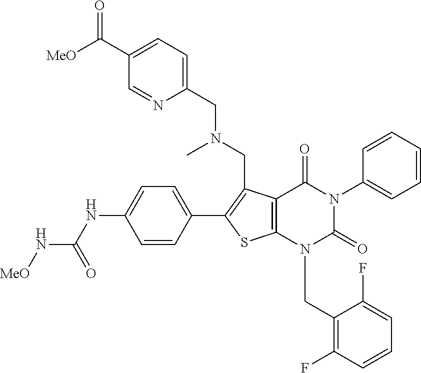 [SMILES: CONC(=O)Nc1ccc(-c2sc3c(c2CN(C)Cc2ccc(C(=O)OC)cn2)c(=O)n(-c2ccccc2)c(=O)n3Cc2c(F)cccc2F)cc1] | ¹H-NMR (CDCl₃) δ: 2.09 (3H, s), 3.76 (2H, s), 3.83 (3H, s), 3.93 (3H, s), 3.99 (2H, s), 5.35 (2H, s), 6.92 (2H, t, J = 8.2 Hz), 7.26 (1H, s), 7.2-7.6 (11H, m), 7.64 (1H, s), 8.05-8.15 (1H, m), 9.0-9.05 (1H, s). |
| 21 | 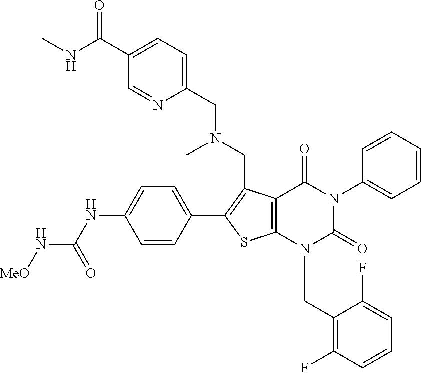 [SMILES: CNC(=O)c1ccc(CN(C)Cc2c(-c3ccc(NC(=O)NOC)cc3)sc3c2c(=O)n(-c2ccccc2)c(=O)n3Cc2c(F)cccc2F)nc1] | ¹H-NMR (CDCl₃) δ: 2.21 (3H, s), 3.00 (3H, d, J = 4.6 Hz), 3.74 (2H, s), 3.84 (3H, s), 3.92 (2H, s), 5.36 (2H, s), 6.5-6.65 (1H, m), 6.92 (2H, t, J = 8.0 Hz), 7.15-7.6 (12H, m), 7.71 (1H, s), 7.9-8.0 (1H, m), 8.80-8.85 (1H, m). |
| 22 | 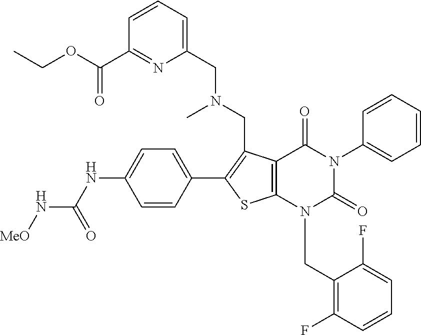 [SMILES: CCOC(=O)c1cccc(CN(C)Cc2c(-c3ccc(NC(=O)NOC)cc3)sc3c2c(=O)n(-c2ccccc2)c(=O)n3Cc2c(F)cccc2F)n1] | ¹H-NMR (CDCl₃) δ: 1.40 (3H, t, J = 7.2 Hz), 2.10 (3H, s), 3.79 (2H, s), 3.83 (3H, s), 4.01 (2H, s), 4.43 (2H, q, J = 7.2 Hz), 5.35 (2H, s), 6.92 (2H, t, J = 8.2 Hz), 7.15 (1H, s), 7.2-7.7 (13H, m), 7.88 (1H, d, J = 7.4 Hz). |
| 23 | 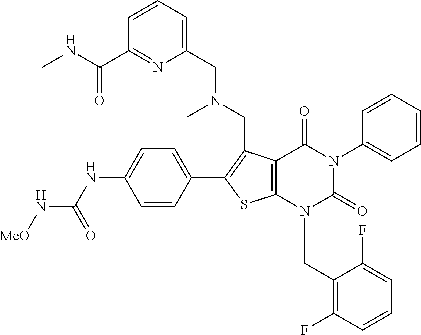 [SMILES: CNC(=O)c1cccc(CN(C)Cc2c(-c3ccc(NC(=O)NOC)cc3)sc3c2c(=O)n(-c2ccccc2)c(=O)n3Cc2c(F)cccc2F)n1] | ¹H-NMR (CDCl₃) δ: 2.13 (3H, s), 2.95 (3H, d, J = 5.2 Hz), 3.74 (2H, s), 3.84 (3H, s), 3.99 (2H, s), 5.36 (2H, s), 6.93 (2H, t, J = 8.2 Hz), 7.15-7.7 (13H, m), 7.9-8.1 (3H, m). |
| 24 | 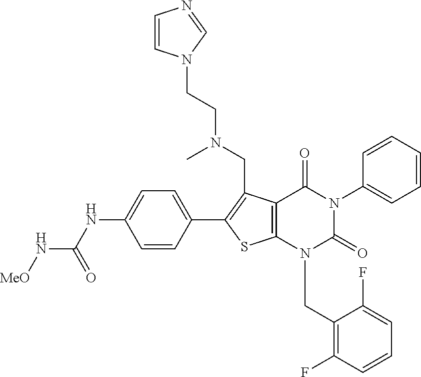 [SMILES: CONC(=O)Nc1ccc(-c2sc3c(c2CN(C)CCn2ccnc2)c(=O)n(-c2ccccc2)c(=O)n3Cc2c(F)cccc2F)cc1] | ¹H-NMR (CDCl₃) δ: 2.17 (3H, s), 2.6-2.8 (2H, m), 3.80 (2H, s), 3.83 (3H, s), 3.9-4.0 (2H, m), 5.38 (2H, s), 6.78 (1H, s), 6.85-7.0 (3H, m), 7.2-7.6 (12H, m), 7.71 (1H, s). |
| 25 | 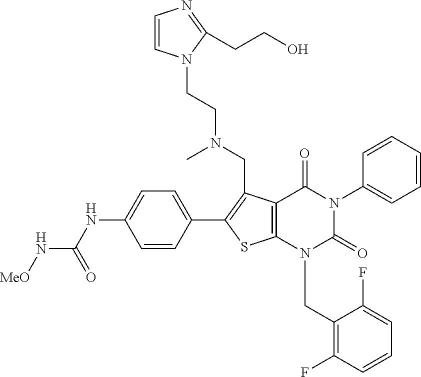 [SMILES: CONC(=O)Nc1ccc(-c2sc3c(c2CN(C)CCn2ccnc2CCO)c(=O)n(-c2ccccc2)c(=O)n3Cc2c(F)cccc2F)cc1] | ¹H-NMR (CDCl₃) δ: 1.9-2.1 (2H, m), 2.16 (3H, s), 2.6-2.8 (4H, m), 3.78 (2H, s), 3.83 (2H, s), 3.9-4.0 (2H, m), 5.38 (2H, s), 6.70 (1H, s), 6.80 (1H, s), 6.94 (2H, t, J = 8.0 Hz), 7.2- 7.6 (11H, m), 7.73 (1H, s). |
| 26 | 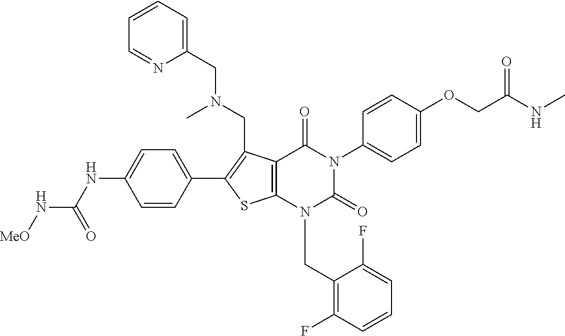 [SMILES: CNC(=O)COc1ccc(-n2c(=O)c3c(CN(C)Cc4ccccn4)c(-c4ccc(NC(=O)NOC)cc4)sc3n(Cc3c(F)cccc3F)c2=O)cc1] | ¹H-NMR (CDCl₃) δ: 2.09 (3H, s), 2.93 (3H, d, J = 4.8 Hz), 3.72 (2H, s), 3.83 (3H, s), 3.97 (2H, s), 4.55 (2H, s), 5.35 (2H, s), 6.55-6.65 (1H, m), 6.92 (2H, t, J = 8.0 Hz), 7.0-7.65 (14H, m), 8.44 (1H, d, J = 6.0 Hz). |
| 27 | 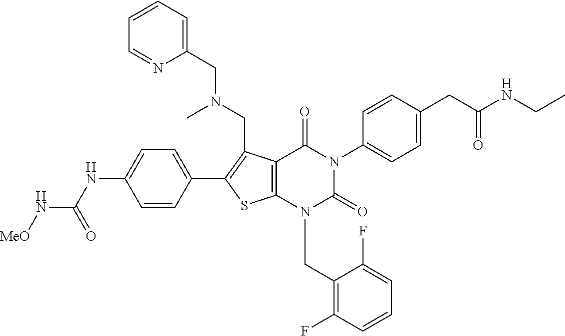 [SMILES: CCNC(=O)Cc1ccc(-n2c(=O)c3c(CN(C)Cc4ccccn4)c(-c4ccc(NC(=O)NOC)cc4)sc3n(Cc3c(F)cccc3F)c2=O)cc1] | ¹H-NMR (CDCl₃) δ: 1.10 (3H, t, J = 7.2 Hz), 2.10 (3H, s), 3.2- 3.4 (2H, m), 3.64 (2H, s), 3.72 (2H, s), 3.83 (3H, s), 3.98 (2H, s), 5.36 (2H, s), 5.45-5.55 (1H, m), 6.93 (2H, t, J = 8.0 Hz), 7.0- 7.1 (1H, m), 7.16 (1H, s), 7.25- 7.7 (12H, m), 8.44 (1H, d, J = 4.0 Hz). |
| 28 | 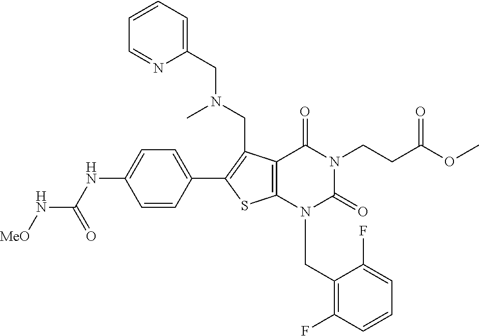 [SMILES: CONC(=O)Nc1ccc(-c2sc3c(c2CN(C)Cc2ccccn2)c(=O)n(CCC(=O)OC)c(=O)n3Cc2c(F)cccc2F)cc1] | ¹H-NMR (CDCl₃) δ: 2.10 (3H, s), 2.65-2.8 (2H, m), 3.68 (3H, s), 3.72 (2H, s), 3.83 (3H, s), 3.98 (2H, s), 4.3-4.5 (2H, m), 5.31 (2H, s), 6.91 (2H, t, J = 7.6 Hz), 7.0-7.4 (5H, m), 7.45-7.65 (5H, m), 8.4-8.5 (1H, m). |
| 29 | 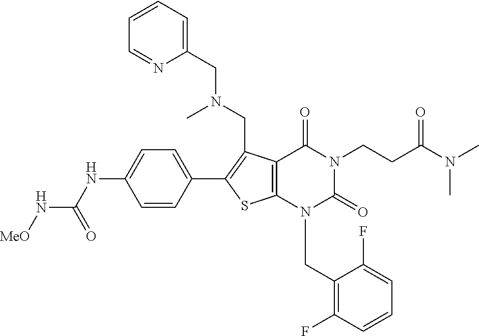 [SMILES: CONC(=O)Nc1ccc(-c2sc3c(c2CN(C)Cc2ccccn2)c(=O)n(CCC(=O)N(C)C)c(=O)n3Cc2c(F)cccc2F)cc1] | ¹H-NMR (CDCl₃) δ: 2.09 (3H, s), 2.75 (3H, d, J = 7.8 Hz), 2.94 (3H, s), 3.02 (3H, s), 3.73 (2H, s), 3.83 (3H, s), 4.07 (2H, s), 4.40 (2H, t, J = 7.8 Hz), 5.33 (2H, s), 6.91 (2H, t, J = 8.2 Hz), 7.0-7.35 (5H, m), 7.5-7.65 (5H, m), 8.45 (1H, d, J = 4.0 Hz). |
| 30 | 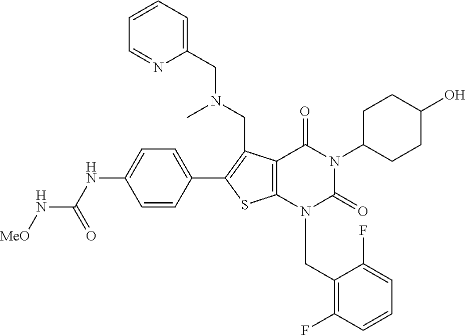 [SMILES: CONC(=O)Nc1ccc(-c2sc3c(c2CN(C)Cc2ccccn2)c(=O)n(C2CCC(O)CC2)c(=O)n3Cc2c(F)cccc2F)cc1] | ¹H-NMR (CDCl₃) δ: 1.4-1.8 (4H, m), 2.0-2.1 (2H, m), 2.12 (3H, s), 2.55-2,75 (2H, m), 3.73 (2H, s), 3.7-3.8 (1H, m), 3.82 (3H, s), 3.98 (2H, s), 4.9- 5.1 (1H, brm), 5.29 (2H, s), 6.91 (2H, t, J = 8.2 Hz), 7.0-7.4 (7H, m), 7.45-7.65 (5H, m), 8.45 (1H, d, J = 4.8 Hz). |
| 31 | 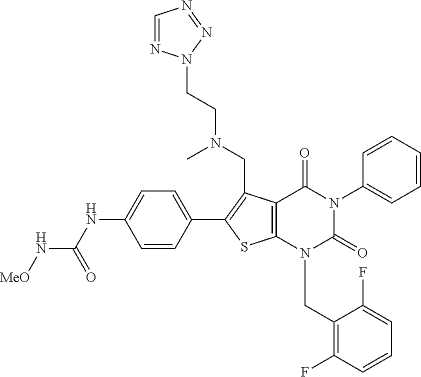 [SMILES: CONC(=O)Nc1ccc(-c2sc3c(c2CN(C)CCn2ncnn2)c(=O)n(-c2ccccc2)c(=O)n3Cc2c(F)cccc2F)cc1] | 2-yl Form (1) ¹H-NMR (CDCl₃) δ: 2.25 (3H, s), 3.04 (2H, t, J = 6.2 Hz), 3.78 (2H, s), 3.83 (3H, s), 4.66 (2H, t, J = 6.2 Hz), 5.38 (2H, s), 6.94 (2H, t, J = 8.0 Hz), 7.16 (1H, s), 7.2-7.6 (10H, m), 7.64 (1H, s), 8.30 (1H, s). |
| | 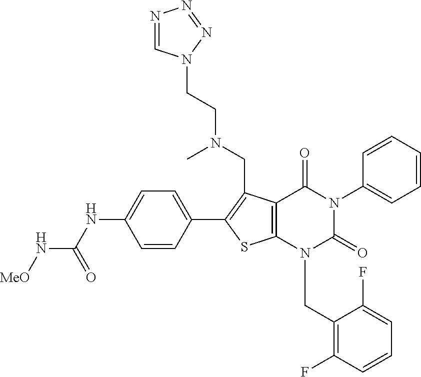 [SMILES: CONC(=O)Nc1ccc(-c2sc3c(c2CN(C)CCn2cnnn2)c(=O)n(-c2ccccc2)c(=O)n3Cc2c(F)cccc2F)cc1] | 1-yl Form (2) ¹H-NMR (CDCl₃) δ: 2.02 (3H, s), 2.7-2.8 (2H, m), 3.78 (2H, s), 3.83 (3H, s), 4.4-4.5 (2H, m), 5.38 (2H, s), 6.92 (2H, t, J = 8.0 Hz), 7.17 (1H, s), 7.25- 7.65 (10H, m), 7.66 (1H, s). |
| 32 | 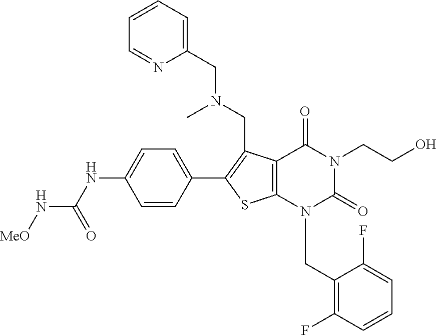 [SMILES: CONC(=O)Nc1ccc(-c2sc3c(c2CN(C)Cc2ccccn2)c(=O)n(CCO)c(=O)n3Cc2c(F)cccc2F)cc1] | ¹H-NMR (CDCl₃) δ: 2.22 (3H, s), 3.3-3.5 (1H, br), 3.78 (2H, s), 3.83 (3H, s), 3.9-4.05 (2H, m), 3.99 (2H, s), 4.35-4.4 (2H, m), 5.27 (2H, s), 6.91 (2H, t, J = 8.0 Hz), 6.9-7.1 (1H, m), 7.15 (1H, s), 7.2-7.65 (8H, m), 8.35-8.4 (1H, m). |
| 33 | 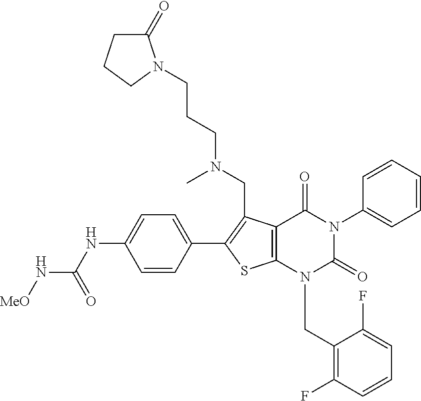 [SMILES: CONC(=O)Nc1ccc(-c2sc3c(c2CN(C)CCCN2CCCC2=O)c(=O)n(-c2ccccc2)c(=O)n3Cc2c(F)cccc2F)cc1] | ¹H-NMR (CDCl₃) δ: 1.5-1.7 (2H, m), 1.8-2.0 (2H, m), 2.05 (3H, s), 2.25-2.45 (4H, m), 3.15 (2H, t, J = 7.8 Hz), 3.23 (2H, t, J = 7.2 Hz), 3.76 (2H, s), 3.83 (3H, s), 5.37 (2H, s), 6.93 (2H, t, J = 8.0 Hz), 7.15 (1H, s), 7.2-7.6 (10H, m), 7.67 (1H, s). |
| 34 | 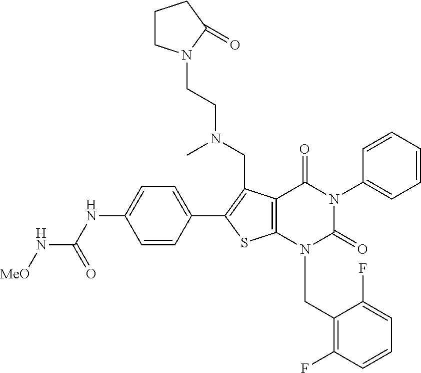 [SMILES: CONC(=O)Nc1ccc(-c2sc3c(c2CN(C)CCN2CCCC2=O)c(=O)n(-c2ccccc2)c(=O)n3Cc2c(F)cccc2F)cc1] | ¹H-NMR (CDCl₃) δ: 1.7-1.85 (2H, m), 2.14 (3H, s), 2.21 (2H, t, J = 8.1 Hz), 2.54 (2H, t, J = 6.2 Hz), 3.19 (2H, t, J = 7.0 Hz), 3.29 (2H, t, J = 6.2 Hz), 3.77 (2H, s), 3.83 (3H, s), 5.38 (2H, s), 6.93 (2H, t, J = 8.1 Hz), 7.18 (1H, s), 7.25-7.7 (11H, m). |
| 35 | 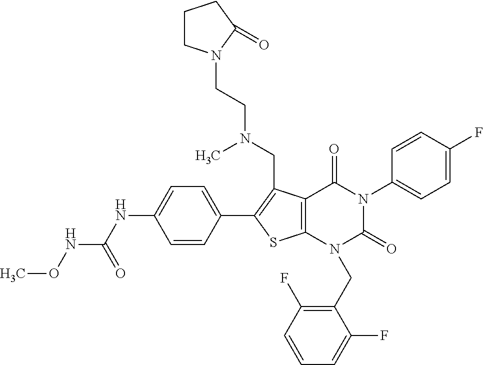 [SMILES: CONC(=O)Nc1ccc(-c2sc3c(c2CN(C)CCN2CCCC2=O)c(=O)n(-c2ccc(F)cc2)c(=O)n3Cc2c(F)cccc2F)cc1] | ¹H-NMR (CDCl₃) δ: 1.7-1.9 (2H, m), 2.15 (3H, s), 2.15-2.3 (2H, m), 2.52 (2H, t, J = 6.2 Hz), 3.20 (2H, t, J = 6.8 Hz), 3.29 (2H, t, J = 6.2 Hz), 3.77 (2H, s), 3.83 (3H, s), 5.37 (2H, s), 6.93 (2H, t, J = 8.2 Hz), 7.1-7.35 (6H, m), 7.5-7.65 (4H, m), 7.64 (1H, s). |
| 36 | 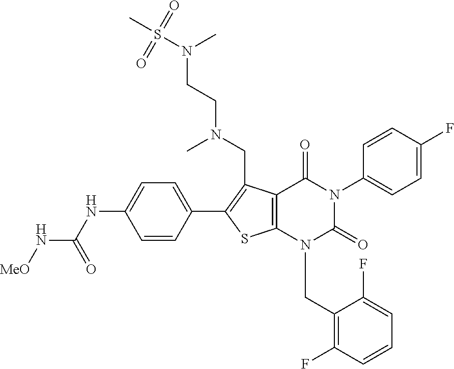 [SMILES: CONC(=O)Nc1ccc(-c2sc3c(c2CN(C)CCN(C)S(C)(=O)=O)c(=O)n(-c2ccc(F)cc2)c(=O)n3Cc2c(F)cccc2F)cc1] | ¹H-NMR (CDCl₃) δ: 2.13 (3H, s), 2.55 (2H, t, J = 6.4 Hz), 2.70 (3H, s), 2.71 (3H, s), 3.15 (2H, t, J = 6.4 Hz), 3.81 (2H, s), 3.83 (3H, s), 5.37 (2H, s), 6.93 (2H, t, J = 8.2 Hz), 7.1-7.7 (11H, m). |
| 37 | 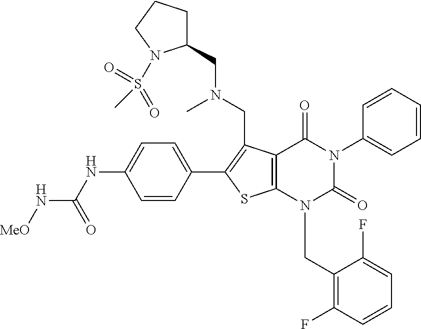 [SMILES: CONC(=O)Nc1ccc(-c2sc3c(c2CN(C)C[C@@H]2CCCN2S(C)(=O)=O)c(=O)n(-c2ccccc2)c(=O)n3Cc2c(F)cccc2F)cc1] | ¹H-NMR (CDCl₃) δ: 1.7-1.85 (4H, m), 2.08 (3H, s), 2.2-2.4 (1H, m), 2.5-2.65 (1H, m), 2.72 (3H, s), 3.15-3.3 (2H, m), 3.7- 3.9 (3H, m), 3.83 (3H, s), 5.37 (2H, s), 6.92 (2H, t, J = 8.2 Hz), 7.13 (1H, s), 7.2-7.7 (11H, m). |
| 38 | 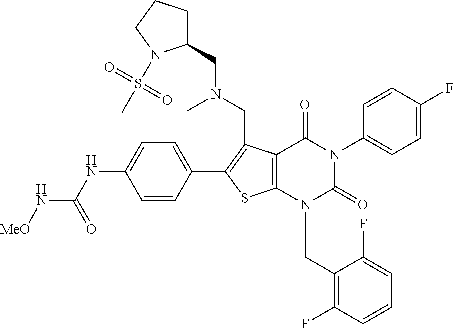 [SMILES: CONC(=O)Nc1ccc(-c2sc3c(c2CN(C)C[C@@H]2CCCN2S(C)(=O)=O)c(=O)n(-c2ccc(F)cc2)c(=O)n3Cc2c(F)cccc2F)cc1] | ¹H-NMR (CDCl₃) δ: 1.7-1.85 (4H, m), 2.09 (3H, s), 2.25- 2.35 (1H, m), 2.55-2.65 (1H, m), 2.71 (3H, s), 3.15-3.3 (2H, m), 3.65-3.7 (2H, m), 3.74 (1H, d, J = 12.0 Hz), 3.83 (3H, s), 3.87 (1H, d, J = 12.0 Hz), 5.36 (2H, s), 6.92 (2H, t, J = 8.2 Hz), 6.85-6.95 (1H, m), 7.1-7.35 (5H, m), 7.49 (2H, d, J = 8.4 Hz), 7.55 (2H, d, J = 8.4 Hz), 7.62 (1H, s). |
| 39 | 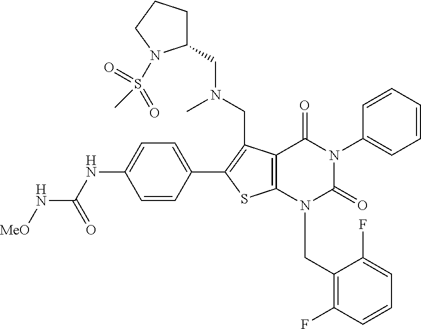 [SMILES: CONC(=O)Nc1ccc(-c2sc3c(c2CN(C)C[C@H]2CCCN2S(C)(=O)=O)c(=O)n(-c2ccccc2)c(=O)n3Cc2c(F)cccc2F)cc1] | ¹H-NMR (CDCl₃) δ: 1.7-1.85 (4H, m), 2.08 (3H, s), 2.2-2.4 (1H, m), 2.5-2.65 (1H, m), 2.72 (3H, s), 3.15-3.3 (2H, m), 3.7- 3.9 (3H, m), 3.83 (3H, s), 5.37 (2H, s), 6.92 (2H, t, J = 8.2 Hz), 7.13 (1H, s), 7.2-7.7 (11H, m). |
| 40 | 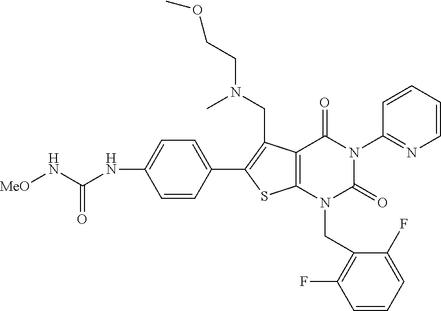 [SMILES: COCCN(C)Cc1c(-c2ccc(NC(=O)NOC)cc2)sc2c1c(=O)n(-c1ccccn1)c(=O)n2Cc1c(F)cccc1F] | ¹H-NMR (CDCl₃) δ: 2.15 (3H, s), 2.62 (2H, t, J = 5.9 Hz), 3.26 (3H, s), 3.41 (2H, t, J = 5.9 Hz), 3.80 (3H, s), 3.81 (2H, brs), 5.34 (2H, brs), 6.91 (2H, t, J = 8.1 Hz), 7.24-7.40 (4H, m), 7.53 (2H, d, J = 8.4 Hz), 7.62 (2H, d, J = 8.4 Hz), 7.65 (1H, s), 7.88 (1H, dt, J = 1.5 Hz, 7.8 Hz), 8.67-8.69 (1H, m). |
| 41 | 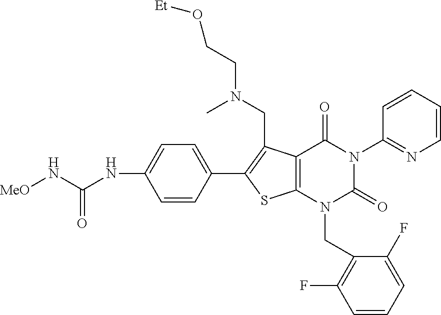 [SMILES: CCOCCN(C)Cc1c(-c2ccc(NC(=O)NOC)cc2)sc2c1c(=O)n(-c1ccccn1)c(=O)n2Cc1c(F)cccc1F] | ¹H-NMR (CDCl₃) δ: 1.13 (3H, t, J = 6.9 Hz), 2.15 (3H, s), 2.63 (2H, t, J = 6.2 Hz), 3.39 (2H, q, J = 6.9 Hz), 3.44 (2H, t, J = 6.2 Hz), 3.80 (2H, brs), 3.81 (3H, s), 5.34 (2H, brs), 6.91 (2H, t, J = 8.1 Hz), 7.19 (1H, s), 7.27- 7.32 (1H, m), 7.35-7.41 (2H, m), 7.53 (2H, d, J = 8.4 Hz), 7.63 (1H, s), 7.64 (2H, d, J = 8.4 Hz), 7.88 (1H, dt, J = 1.2 Hz, 7.5 Hz), 8.68 (1H, dt, J = 0.9 Hz, 4.8 Hz). |
| 42 | 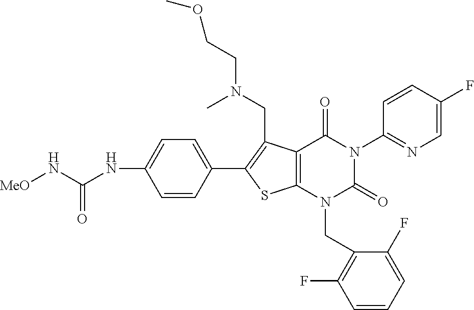 [SMILES: COCCN(C)Cc1c(-c2ccc(NC(=O)NOC)cc2)sc2c1c(=O)n(-c1ccc(F)cn1)c(=O)n2Cc1c(F)cccc1F] | ¹H-NMR (CDCl₃) δ: 2.13 (3H, s), 2.62 (2H, t, J = 5.9 Hz), 3.26 (3H, s), 3.41 (2H, t, J = 5.9 Hz), 3.80 (2H, brs), 3.82 (3H, s), 5.33 (2H, brs), 6.92 (2H, t, J = 8.3 Hz), 7.19 (1H, s), 7.28- 7.38 (2H, m), 7.52-7.63 (6H, m), 8.51 (1H, d, J = 3.0 Hz). |
| 43 | 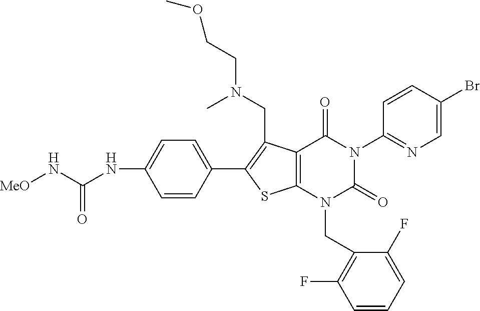 [SMILES: COCCN(C)Cc1c(-c2ccc(NC(=O)NOC)cc2)sc2c1c(=O)n(-c1ccc(Br)cn1)c(=O)n2Cc1c(F)cccc1F] | ¹H-NMR (CDCl₃) δ: 2.13 (3H, s), 2.62 (2H, t, J = 5.9 Hz), 3.26 (3H, s), 3.41 (2H, t, J = 5.9 Hz), 3.78 (2H, brs), 3.80 (3H, s), 5.32 (2H, brs), 6.92 (2H, t, J = 8.1 Hz), 7.27 (1H, d, J = 8.4 Hz), 7.27-7.33 (1H, m), 7.37 (1H, s), 7.98 (1H, dd, J = 2.7 Hz, 8.4 Hz), 8.72 (1H, d, J = 2.7 Hz). |
| 44 | 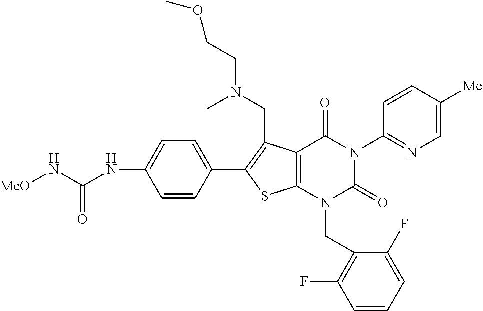 [SMILES: COCCN(C)Cc1c(-c2ccc(NC(=O)NOC)cc2)sc2c1c(=O)n(-c1ccc(C)cn1)c(=O)n2Cc1c(F)cccc1F] | ¹H-NMR (CDCl₃) δ: 2.14 (3H, s), 2.39 (3H, s), 2.62 (2H, t, J = 5.7 Hz), 3.26 (3H, s), 3.41 (2H, t, J = 5.7 Hz), 3.77 (2H, brs), 3.80 (3H, s), 5.26 (1H, brs), 5.38 (1H, brs), 6.91 (2H, t, J = 8.3 Hz), 7.23-7.34 (2H, m), 7.42 (1H, s), 7.53 (2H, d, J = 8.7 Hz), 7.62 (2H, d, J = 8.7 Hz), 7.66 (1H, s), 7.66-7.69 (1H, m), 8.48 (1H, d, J = 2.4 Hz). |
| 45 | 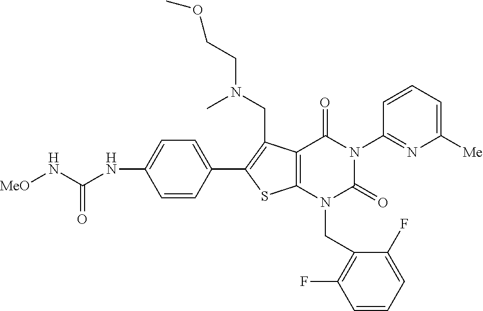 [SMILES: COCCN(C)Cc1c(-c2ccc(NC(=O)NOC)cc2)sc2c1c(=O)n(-c1cccc(C)n1)c(=O)n2Cc1c(F)cccc1F] | ¹H-NMR (CDCl₃) δ: 2.15 (3H, s), 2.60 (3H, s), 2.62 (2H, t, J = 5.8 Hz), 3.27 (3H, s), 3.41 (2H, t, J = 5.8 Hz), 3.66-3.94 (2H, m), 3.81 (3H, s), 5.15 (1H, d, J = 15.3 Hz), 5.48 (1H, d, J = 15.3 Hz), 6.91 (2H, t, J = 8.1 Hz), 7.16 (1H d, J = 7.8 Hz), 7.21 (1H, d, J = 7.8 Hz), 7.25 (1H, s), 7.26-7.35 (1H, m), 7.53 (2H, d, J = 8.7 Hz), 7.63 (1H, s), 7.53 (2H, d, J = 8.7 Hz), 7.64 (2H, d, J = 8.7 Hz), 7.76 (1H, t, J = 7.8 Hz). |
| 46 | 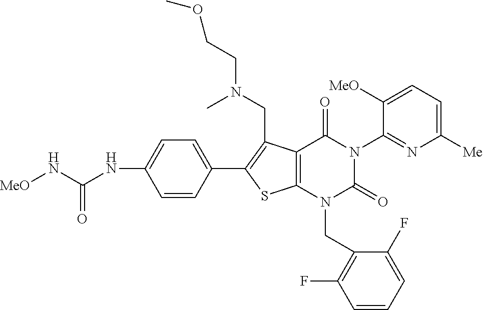 [SMILES: COCCN(C)Cc1c(-c2ccc(NC(=O)NOC)cc2)sc2c1c(=O)n(-c1nc(C)ccc1OC)c(=O)n2Cc1c(F)cccc1F] | ¹H-NMR (CDCl₃) δ: 2.13 (3H, s), 2.51 (3H, s), 2.62 (2H, t, J = 5.9 Hz), 3.26 (3H, s), 3.40 (2H, t, J = 5.9 Hz), 3.77 (1H, d, J = 12.3 Hz), 3.77 (3H, s), 3.79 (3H, s), 3.86 (1H, d, J = 12.3 Hz), 5.24 (1H, d, J = 15.6 Hz), 5.40 (1H, d, J = 15.6 Hz), 6.90 (2H, t, J = 8.1 Hz), 7.19 (1H, d, J = 8.4 Hz), 7.23-7.34 (1H, m), 7.27 (1H, d, J = 8.4 Hz), 7.51 (2H, d, J = 8.7 Hz), 7.58 (2H, d, J = 8.7 Hz), 7.65 (1H, s), 7.69 (1H, s). |
| 47 | 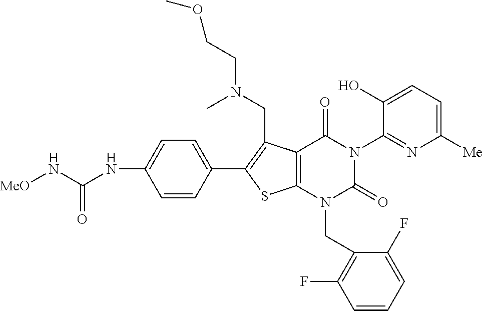 [SMILES: COCCN(C)Cc1c(-c2ccc(NC(=O)NOC)cc2)sc2c1c(=O)n(-c1nc(C)ccc1O)c(=O)n2Cc1c(F)cccc1F] | ¹H-NMR (CDCl₃) δ: 2.11 (3H, s), 2.48 (3H, s), 2.51-2.59 (2H, m), 3.20 (3H, s), 3.30-3.46 (4H, m), 3.60 (1H, d, J = 12.3 Hz), 3.79 (3H, s), 4.05 (1H, d, J = 12.3 Hz), 5.21 (1H, d, J = 15.6 Hz), 5.31 (1H, d, J = 15.6 Hz), 6.88 (2H, t, J = 8.1 Hz), 7.07 (1H, d, J = 8.1 Hz), 7.21-7.31 (2H, m), 7.43-7.51 (4H, m), 7.69 (1H, s). |
| 48 | 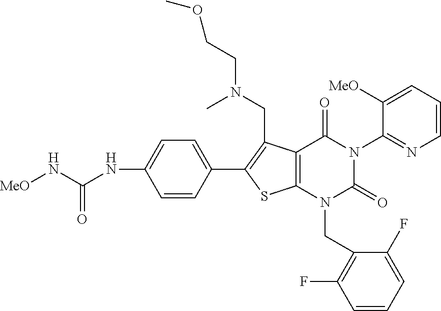 [SMILES: COCCN(C)Cc1c(-c2ccc(NC(=O)NOC)cc2)sc2c1c(=O)n(-c1ncccc1OC)c(=O)n2Cc1c(F)cccc1F] | ¹H-NMR (CDCl₃) δ: 2.20 (3H, s), 2.63 (2H, t, J = 6.2 Hz), 3.28 (3H, s), 3.41 (2H, t, J = 6.2 Hz), 3.78 (1H, d, J = 12.3 Hz), 3.82 (3H, s), 3.84 (3H, s), 3.88 (1H, d, J = 12.3 Hz), 5.35 (2H, s), 6.92 (2H, t, J = 8.1 Hz), 7.17 (1H, s), 7.23-7.39 (3H, m), 7.54 (2H, d, J = 8.8 Hz), 7.57 (1H, s), 7.69 (2H, d, J = 8.8 Hz), 8.23-8.27 (1H, m). |
| 49 | 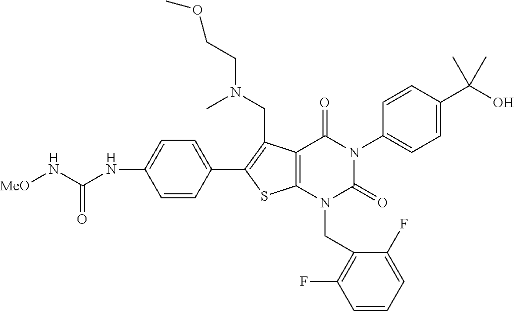 [SMILES: COCCN(C)Cc1c(-c2ccc(NC(=O)NOC)cc2)sc2c1c(=O)n(-c1ccc(C(C)(C)O)cc1)c(=O)n2Cc1c(F)cccc1F] | ¹H-NMR (CDCl₃) δ: 1.60 (6H, s), 1.79 (1H, s), 2.14 (3H, s), 2.63 (2H, t, J = 5.9 Hz), 3.27 (3H, s), 3.41 (2H, t, J = 5.9 Hz), 3.81 (3H, s), 3.82 (2H, s), 5.36 (2H, s), 6.92 (2H, t, J = 8.3 Hz), 7.20-7.34 (4H, m), 7.53 (2H, d, J = 8.7 Hz), 7.60-7.63 (5H, m). |
| 50 | 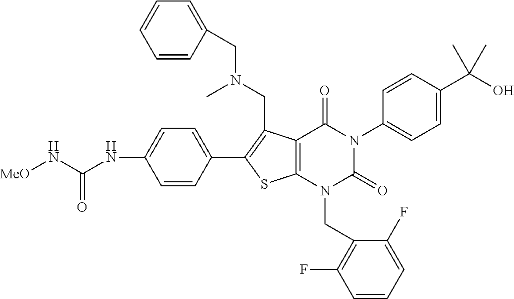 [SMILES: CONC(=O)Nc1ccc(-c2sc3c(c2CN(C)Cc2ccccc2)c(=O)n(-c2ccc(C(C)(C)O)cc2)c(=O)n3Cc2c(F)cccc2F)cc1] | ¹H-NMR (CDCl₃) δ: 1.62 (6H, s), 1.77 (1H, s), 2.05 (3H, s), 3.56 (2H, s), 3.82 (3H, s), 3.90 (2H, s), 5.36 (2H, s), 6.91 (2H, t, J = 8.1 Hz), 7.14-7.38 (9H, m), 7.55 (2H, d, J = 9.0 Hz), 7.62 (1H, s), 7.64 (2H, d, J = 8.7 Hz), 7.72 (2H, d, J = 8.4 Hz). |
| 51 | 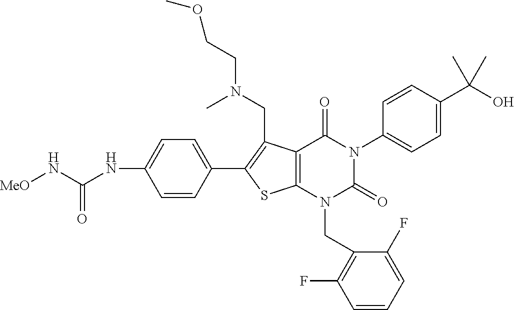 [SMILES: COCCN(C)Cc1c(-c2ccc(NC(=O)NOC)cc2)sc2c1c(=O)n(-c1ccc(C(C)(C)O)cc1)c(=O)n2Cc1c(F)cccc1F] | ¹H-NMR (CDCl₃) δ: 1.55 (6H, s), 2.15 (3H, s), 2.64 (2H, t, J = 5.9 Hz), 3.11 (3H, s), 3.27 (3H, s), 3.41 (2H, t, J = 5.9 Hz), 3.82 (3H, s), 3.83 (2H, s), 5.36 (2H, s), 6.92 (2H, t, J = 8.3 Hz), 7.16 (1H, s), 7.24-7.36 (4H, m), 7.51-7.63 (6H, m). |
| 52 | 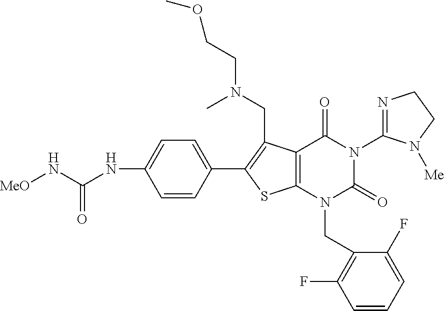 [SMILES: COCCN(C)Cc1c(-c2ccc(NC(=O)NOC)cc2)sc2c1c(=O)n(C1=NCCN1C)c(=O)n2Cc1c(F)cccc1F] | ¹H-NMR (CDCl₃) δ: 2.15 (3H, s), 2.61 (2H, dt, J = 1.8 Hz, 6.90 Hz), 3.27 (3H, s), 3.40 (2H, dt, J = 1.8 Hz, 6.0 Hz), 3.53 (3H, s), 3.75 (1H, d, J = 12.3 Hz), 3.80 (3H, s), 3.81 (1H, d, J = 12.3 Hz), 5.12 (1H, d, J = 15.9 Hz), 5.57 (1H, d, J = 15.9 Hz), 6.91 (2H, t, J = 8.1 Hz), 6.99 (1H, d, J = 1.5 Hz), 7.14 (1H, d, J = 1.5 Hz), 7.28 (1H, s), 7.25-7.34 (1H, m), 7.53 (2H, d, J = 9.0 Hz), 7.60 (2H, d, J = 9.0 Hz), 7.70 (1H, s). |
| 53 | 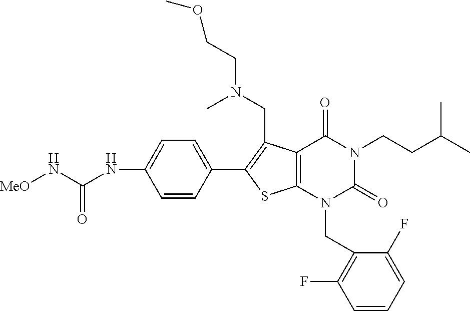 [SMILES: COCCN(C)Cc1c(-c2ccc(NC(=O)NOC)cc2)sc2c1c(=O)n(CCC(C)C)c(=O)n2Cc1c(F)cccc1F] | ¹H-NMR (CDCl₃) δ: 0.98 (6H, d, J = 6.3 Hz), 1.52-1.58 (2H, m), 1.64-1.71 (1H, m), 2.14 (3H, s), 2.66 (2H, t, J = 5.9 Hz), 3.30 (3H, s), 3.45 (2H, t, J = 5.9 Hz), 3.81 (3H, s), 3.85 (2H, s), 4.04-4.09 (2H, m), 5.33 (2H, s), 6.90 (2H, t, J = 8.3 Hz), 7.17 (1H, s), 7.24-7.35 (1H, m), 7.51 (2H, d, J = 8.7 Hz), 7.57 (2H, d, J = 8.7 Hz), 7.60 (1H, s). |
| 54 | 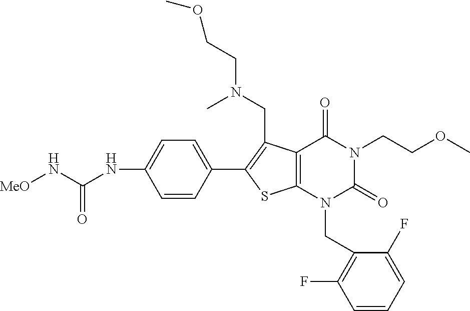 [SMILES: COCCN(C)Cc1c(-c2ccc(NC(=O)NOC)cc2)sc2c1c(=O)n(CCOC)c(=O)n2Cc1c(F)cccc1F] | ¹H-NMR (CDCl₃) δ: 2.14 (3H, s), 2.65 (2H, t, J = 5.9 Hz), 3.30 (3H, s), 3.36 (3H, s), 3.45 (2H, t, J = 5.9 Hz), 3.66 (2H, t, J = 5.9 Hz), 3.81 (3H, s), 3.84 (2H, s), 4.30 (2H, t, J = 5.9 Hz), 5.33 (2H, s), 6.90 (2H, t, J = 8.3 Hz), 7.15 (1H, s), 7.24-7.34 (1H, m), 7.51 (2H, d, J = 9.0 Hz), 7.56 (2H, d, J = 9.0 Hz), 7.60 (1H, m). |
| 55 | 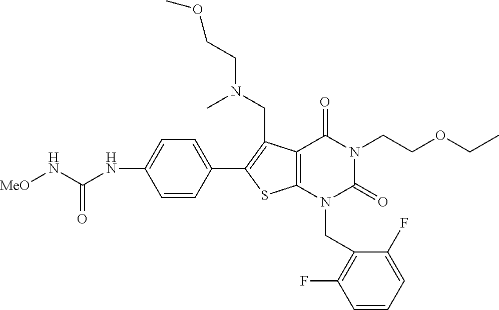 [SMILES: CCOCCn1c(=O)c2c(CN(C)CCOC)c(-c3ccc(NC(=O)NOC)cc3)sc2n(Cc2c(F)cccc2F)c1=O] | ¹H-NMR (CDCl₃) δ: 1.15 (3H, t, J = 6.9 Hz), 2.14 (3H, s), 2.66 (2H, t, J = 6.0 Hz), 3.30 (3H, s), 3.45 (2H, t, J = 6.0 Hz), 3.54 (2H, q, J = 6.9 Hz), 3.69 (2H, t, J = 6.0 Hz), 3.81 (3H, s), 3.84 (2H, s), 4.29 (2H, t, J = 6.0 Hz), 5.32 (2H, s), 6.89 (2H, t, J = 8.1 Hz), 7.17 (1H, s), 7.23-7.34 (1H, m), 7.52 (2H, d, J = 8.7 Hz), 7.57 (2H, d, J = 8.7 Hz), 7.60 (1H, m). |
| 56 | 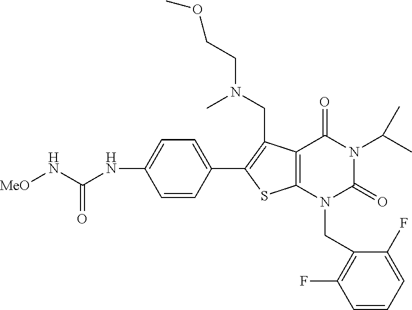 [SMILES: COCCN(C)Cc1c(-c2ccc(NC(=O)NOC)cc2)sc2c1c(=O)n(C(C)C)c(=O)n2Cc1c(F)cccc1F] | ¹H-NMR (CDCl₃) δ: 1.52 (6H, d, J = 6.9 Hz), 2.13 (3H, s), 2.66 (2H, t, J = 5.9 Hz), 3.31 (3H, s), 3.46 (2H, t, J = 5.9 Hz), 3.82 (3H, s), 3.84 (2H, s), 5.31 (2H, s), 5.34 (1H, m), 6.90 (2H, t, J = 8.1 Hz), 7.16 (1H, s), 7.24- 7.35 (1H, m), 7.52 (2H, d, J = 8.4 Hz), 7.55 (2H, d, J = 8.4 Hz), 7.60 (1H, m). |
| 57 | 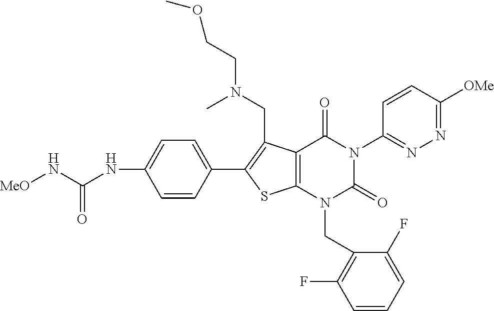 [SMILES: COCCN(C)Cc1c(-c2ccc(NC(=O)NOC)cc2)sc2c1c(=O)n(-c1ccc(OC)nn1)c(=O)n2Cc1c(F)cccc1F] | ¹H-NMR (CDCl₃) δ: 2.13 (3H, s), 2.62 (2H, t, J = 5.7 Hz), 3.26 (3H, s), 3.41 (2H, t, J = 5.7 Hz), 3.74 (2H, brs), 3.82 (3H, s), 4.18 (3H, s), 5.32 (2H, brs), 6.92 (2H, t, J = 8.3 Hz), 7.12 (1H, d, J = 9.3 Hz), 7.24 (1H, s), 7.29-7.35 (1H, m), 7.41 (2H, d, J = 9.3 Hz), 7.54 (2H, d, J = 9.0 Hz), 7.59 (2H, d, J = 8.7 Hz), 7.66 (1H, s). |
| 58 | 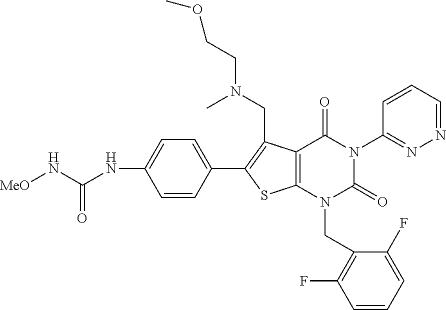 [SMILES: COCCN(C)Cc1c(-c2ccc(NC(=O)NOC)cc2)sc2c1c(=O)n(-c1cccnn1)c(=O)n2Cc1c(F)cccc1F] | ¹H-NMR (CDCl₃) δ: 2.12 (3H, s), 2.61 (2H, t, J = 5.7 Hz), 3.26 (3H, s), 3.39 (2H, t, J = 5.7 Hz), 3.78 (2H, brs), 3.82 (3H, s), 5.34 (2H, brs), 6.93 (2H, t, J = 8.1 Hz), 7.26 (1H, s), 7.29- 7.37 (1H, m), 7.53-7.61 (5H, m), 7.67 (1H, s), 7.69 (1H, dd, J = 4.8 Hz, 8.4 Hz), 9.28 (1H, dd, J = 1.8 Hz, 4.8 Hz). |
| 59 | 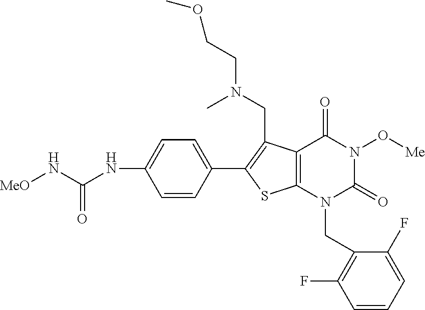 [SMILES: COCCN(C)Cc1c(-c2ccc(NC(=O)NOC)cc2)sc2c1c(=O)n(OC)c(=O)n2Cc1c(F)cccc1F] | ¹H-NMR (CDCl₃) δ: 2.14 (3H, s), 2.68 (2H, t, J = 6.0 Hz), 3.31 (3H, s), 3.47 (2H, t, J = 6.0 Hz), 3.82 (3H, s), 3.83 (2H, s), 4.06 (3H, s), 5.35 (2H, s), 6.92 (2H, t, J = 8.3 Hz), 7.20 (1H, s), 7.29-7.35 (1H, m), 7.55 (4H, s), 7.63 (1H, s). |
| 60 | 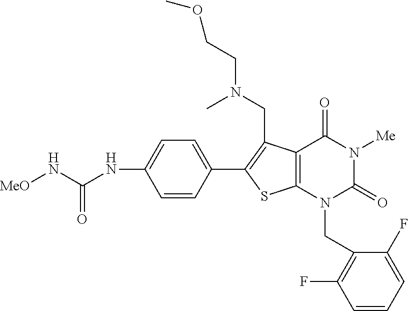 [SMILES: COCCN(C)Cc1c(-c2ccc(NC(=O)NOC)cc2)sc2c1c(=O)n(C)c(=O)n2Cc1c(F)cccc1F] | ¹H-NMR (CDCl₃) δ: 2.13 (3H, s), 2.66 (2H, t, J = 5.9 Hz), 3.31 (3H, s), 3.45 (3H, s), 3.48 (2H, t, J = 5.9 Hz), 3.82 (3H, s), 3.84 (2H, s), 5.33 (2H, s), 6.91 (2H, t, J = 8.3 Hz), 7.17 (1H, s), 7.25-7.35 (1H, m), 7.55 (4H, s), 7.62 (1H, s). |
| 61 | 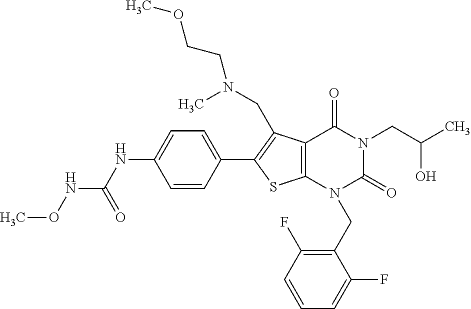 [SMILES: COCCN(C)Cc1c(-c2ccc(NC(=O)NOC)cc2)sc2c1c(=O)n(CC(C)O)c(=O)n2Cc1c(F)cccc1F] | ¹H-NMR (CDCl₃) δ: 1.27 (3H, d, J = 5.6 Hz), 2.12 (3H, s), 2.64 (2H, t, J = 5.8 Hz), 2.9-3.05 (1H, m), 3.30 (3H, s), 3.45 (2H, d, J = 5.8 Hz), 3.82 (5H, s), 4.05- 4.25 (1H, m), 4.18 (2H, s), 5.34 (2H, s), 6.91 (2H, t, J = 8.2 Hz), 7.2-7.4 (1H, m), 7.5-7.6 (3H, m), 7.63 (1H, s). |
| 62 | 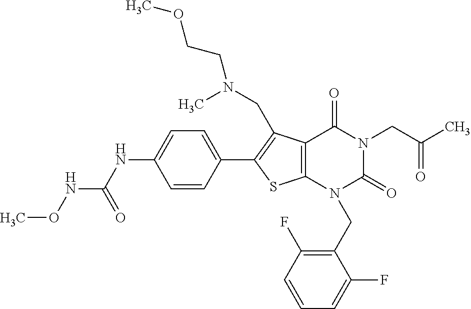 [SMILES: COCCN(C)Cc1c(-c2ccc(NC(=O)NOC)cc2)sc2c1c(=O)n(CC(C)=O)c(=O)n2Cc1c(F)cccc1F] | ¹H-NMR (CDCl₃) δ: 2.13 (3H, s), 2.27 (3H, s), 2.55-2.65 (2H, m), 3.29 (3H, s), 3.4-3.5 (2H, m), 3.82 (5H, s), 4.88 (2H, s), 5.33 (2H, s), 6.91 (2H, t, J = 8.0 Hz), 7.2-7.35 (1H, m), 7.5-7.65 (4H, m). |
| 63 | 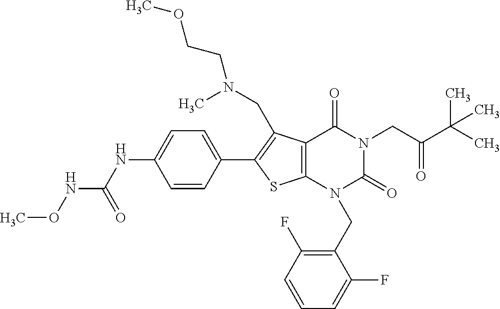 [SMILES: COCCN(C)Cc1c(-c2ccc(NC(=O)NOC)cc2)sc2c1c(=O)n(CC(=O)C(C)(C)C)c(=O)n2Cc1c(F)cccc1F] | ¹H-NMR (CDCl₃) δ: 1.30 (9H, s), 2.12 (3H, s), 2.62 (2H, t, J = 5.8 Hz), 3.29 (3H, s), 3.44 (2H, d, J = 5.8 Hz), 3.80 (2H, s), 3.82 (3H, s), 5.04 (2H, s), 5.33 (2H, s), 6.91 (2H, t, J = 8.2 Hz), 7.14 (1H, s), 7.2-7.3 (1H, m), 7.5-7.6 (4H, m), 7.61 (1H, s). |
| 64 | 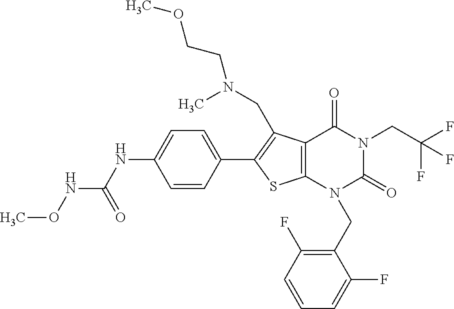 [SMILES: COCCN(C)Cc1c(-c2ccc(NC(=O)NOC)cc2)sc2c1c(=O)n(CC(F)(F)F)c(=O)n2Cc1c(F)cccc1F] | ¹H-NMR (CDCl₃) δ: 2.12 (3H, s), 2.65 (2H, t, J = 6.0 Hz), 3.31 (3H, s), 3.45 (2H, d, J = 6.0 Hz), 3.82 (5H, s), 4.75-4.85 (2H, m), 5.36 (2H, s), 6.92 (2H, t, J = 8.2 Hz), 7.13 (1H, s), 7.2- 7.35 (1H, m), 7.55-7.6 (4H, m), 7.62 (1H, s). |
| 65 | 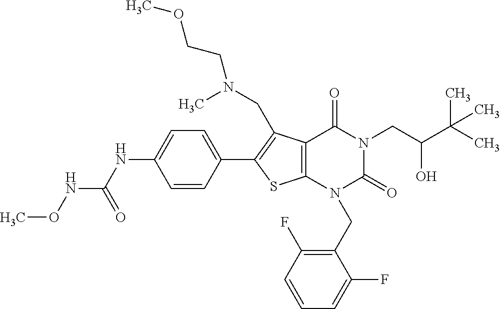 [SMILES: COCCN(C)Cc1c(-c2ccc(NC(=O)NOC)cc2)sc2c1c(=O)n(CC(O)C(C)(C)C)c(=O)n2Cc1c(F)cccc1F] | ¹H-NMR (CDCl₃) δ: 1.04 (9H, s), 2.12 (3H, s), 2.64 (2H, t, J = 6.0 Hz), 2.96 (1H, d, J = 6.0 Hz), 3.31 (3H, s), 3.46 (2H, d, J = 6.0 Hz), 3.5-3.6 (1H, m), 3.82 (3H, s), 3.75-3.9 (2H, m), 4.05-4.2 (1H, m), 4.3-4.45 (1H, m), 5.25-5.45 (2H, m), 6.91 (2H, t, J = 8.2 Hz), 7.14 (1H, s), 7.2-7.35 (1H, m), 7.5-7.6 (4H, m), 7.61 (1H, s). |
| 66 | 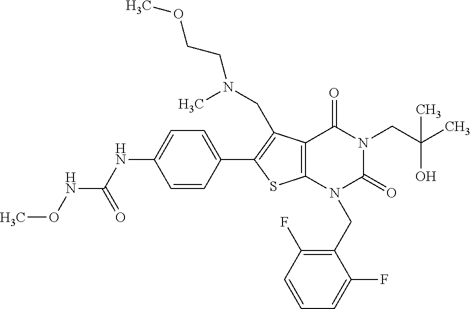 [SMILES: COCCN(C)Cc1c(-c2ccc(NC(=O)NOC)cc2)sc2c1c(=O)n(CC(C)(C)O)c(=O)n2Cc1c(F)cccc1F] | ¹H-NMR (CDCl₃) δ: 1.28 (6H, s), 2.3 (3H, s), 2.64 (2H, t, J = 5.8 Hz), 3.30 (3H, s), 3.45 (2H, t, J = 5.8 Hz), 3.82 (5H, s), 3.99 (1H, s), 4.25 (2H, s), 5.36 (2H, s), 6.91 (2H, t, J = 8.2 Hz), 7.12 (1H, s), 7.2-7.4 (1H, m), 7.5-7.6 (4H, m), 7.61 (1H, s). |
| 67 | 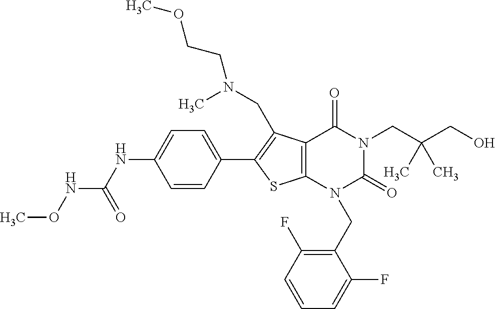 [SMILES: COCCN(C)Cc1c(-c2ccc(NC(=O)NOC)cc2)sc2c1c(=O)n(CC(C)(C)CO)c(=O)n2Cc1c(F)cccc1F] | ¹H-NMR (CDCl₃) δ: 0.96 (6H, s), 2.13 (3H, s), 2.64 (2H, t, J = 6.2 Hz), 3.13 (2H, s), 3.30 (3H, s), 3.45 (2H, t, J = 6.2 Hz), 3.82 (5H, s), 3.95-4.15 (2H, brm), 5.1-5.5 (2H, br), 6.91 (2H, t, J = 8.2 Hz), 7.14 (1H, s), 7.2-7.4 (1H, m), 7.5-7.6 (4H, m), 7.61 (1H, s). |
| 68 | 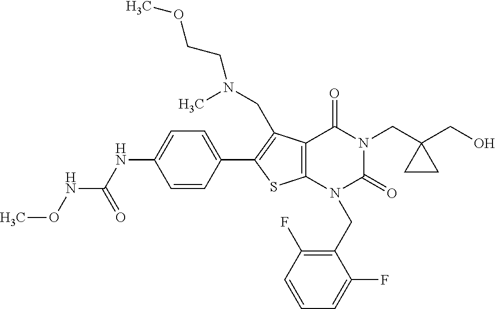 [SMILES: COCCN(C)Cc1c(-c2ccc(NC(=O)NOC)cc2)sc2c1c(=O)n(CC1(CO)CC1)c(=O)n2Cc1c(F)cccc1F] | ¹H-NMR (CDCl₃) δ: 0.46 (2H, t, J = 5.4 Hz), 0.85 (2H, t, J = 5.4 Hz), 2.11 (3H, s), 2.64 (2H, t, J = 6.0 Hz), 3.25 (2H, s), 3.31 (3H, s), 3.46 (2H, t, J = 6.0 Hz), 3.82 (5H, s), 3.95-4.15 (1H, br), 4.14 (2H, s), 5.37 (2H, s), 6.91 (2H, t, J = 8.0 Hz), 7.12 (1H, s), 7.2-7.4 (1H, m), 7.54 (4H, s), 7.61 (1H, s). |
| 69 | 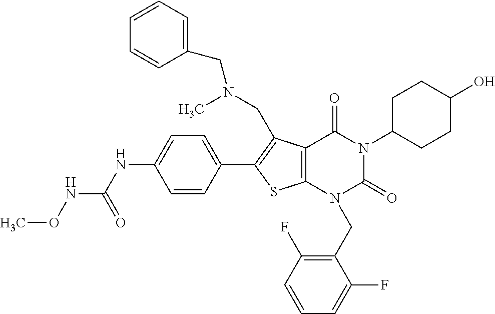 [SMILES: CONC(=O)Nc1ccc(-c2sc3c(c2CN(C)Cc2ccccc2)c(=O)n(C2CCC(O)CC2)c(=O)n3Cc2c(F)cccc2F)cc1] | ¹H-NMR (CDCl₃) δ: 1.35-1.65 (3H, m), 1.65-1.8 (2H, m), 2.07 (3H, s), 2.5-2.8 (2H, m), 3.58 (2H, s), 3.7-3.9 (1H, m), 3.82 (3H, s), 3.91 (2H, s), 4.9-5.1 (1H, m), 5.29 (2H, s), 6.90 (2H, t, J = 7.8 Hz), 7.13 (1H, s), 7.15-7.35 (6H, m), 7.53 (2H, d, J = 8.6 Hz), 7.61 (1H, s), 7.66 (2H, d, J = 8.6 Hz). |
| 70 | 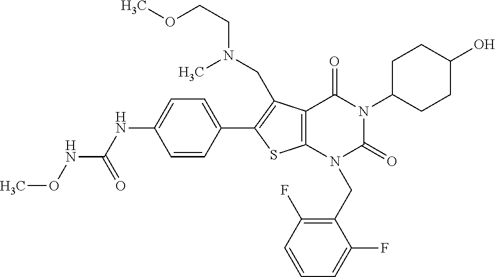 [SMILES: COCCN(C)Cc1c(-c2ccc(NC(=O)NOC)cc2)sc2c1c(=O)n(C1CCC(O)CC1)c(=O)n2Cc1c(F)cccc1F] | ¹H-NMR (CDCl₃) δ: 1.2-1.6 (3H, m), 1.6-1.8 (2H, m), 2.0- 2.1 (2H, m), 2.14 (3H, s), 2.5- 2.75 (4H, m), 3.31 (3H, s), 3.45 (2H, t, J = 5.8 Hz), 3.65- 3.85 (3H, m), 3.82 (3H, s), 4.9- 5.05 (1H, br), 5.30 (2H, s), 6.90 (2H, t, J = 8.0 Hz), 7.12 (1H, s), 7.25-7.4 (1H, m), 7.5- 7.6 (4H, m), 7.60 (1H, s). |
| 71 | 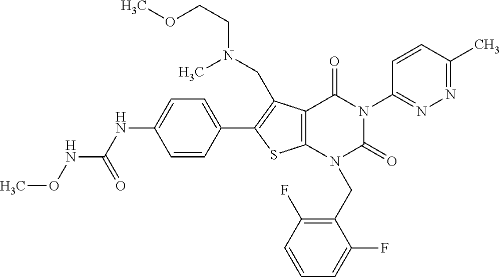 | ¹H-NMR (CDCl₃) δ: 2.13 (3H, s), 2.61 (2H, t, J = 5.8 Hz), 2.79 (3H, s), 3.26 (3H, s), 3.41 (2H, t, J = 5.8 Hz), 3.75-3.85 (2H, m), 3.82 (3H, s), 5.25-5.45 (2H, brm), 6.92 (2H, t, J = 8.2 Hz), 7.18 (1H, s), 7.2-7.7 (8H, m). |
| 72 | 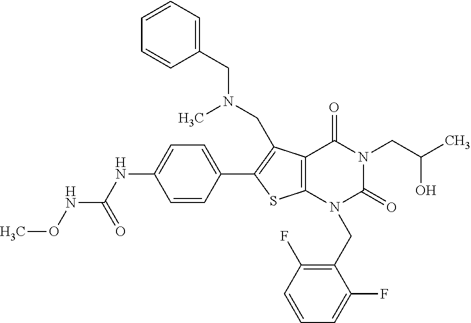 [SMILES: CONC(=O)Nc1ccc(-c2sc3c(c2CN(C)Cc2ccccc2)c(=O)n(CC(C)O)c(=O)n3Cc2c(F)cccc2F)cc1] | ¹H-NMR (CDCl₃) δ: 1.30 (3H, d, J = 6.0 Hz), 2.06 (3H, s), 2.90 (1H, d, J = 5.2 Hz), 3.57 (2H, s), 3.82 (3H, s), 3.91 (2H, s), 4.1- 4.25 (1H, m), 4.20 (2H, s), 5.34 (2H, s), 6.91 (2H, t, J = 8.0 Hz), 7.16 (1H, s), 7.2-7.4 (6H, m), 7.54 (2H, d, J = 8.8 Hz), 7.62 (1H, s), 7.67 (2H, d, J = 8.8 Hz). |
| 73 | 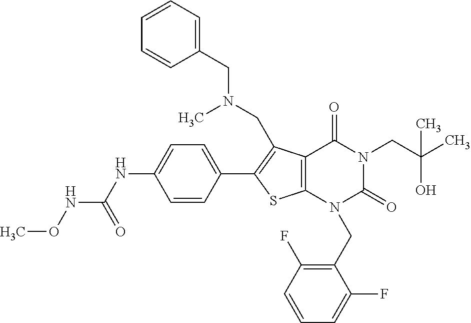 [SMILES: CONC(=O)Nc1ccc(-c2sc3c(c2CN(C)Cc2ccccc2)c(=O)n(CC(C)(C)O)c(=O)n3Cc2c(F)cccc2F)cc1] | ¹H-NMR (CDCl₃) δ: 1.29 (6H, s), 2.06 (3H, s), 3.56 (2H, s), 3.83 (3H, s), 3.91 (2H, s), 3.96 (1H, s), 4.28 (2H, s), 5.36 (2H, s), 6.91 (2H, t, J = 8.2 Hz), 7.13 (1H, s), 7.2-7.35 (6H, m), 7.54 (2H, d, J = 8.8 Hz), 7.62 (1H, s), 7.67 (2H, d, J = 8.8 Hz). |
| 74 | 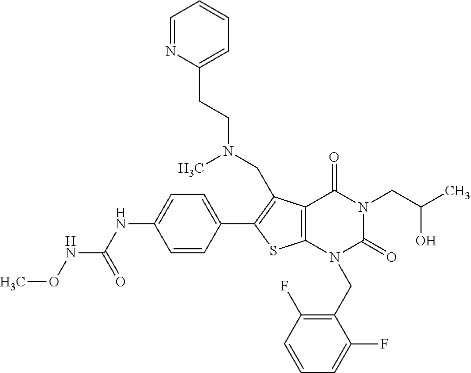 [SMILES: CONC(=O)Nc1ccc(-c2sc3c(c2CN(C)CCc2ccccn2)c(=O)n(CC(C)O)c(=O)n3Cc2c(F)cccc2F)cc1] | ¹H-NMR (CDCl₃) δ: 1.28 (3H, d, J = 5.8 Hz), 2.21 (3H, s), 2.75-2.95 (4H, m), 3.0-3.1 (1H, m), 3.82 (5H, s), 4.1-4.2 (1H, m), 4.17 (2H, s), 5.34 (2H, s), 6.91 (2H, t, J = 8.2 Hz), 6.95- 7.1 (2H, m), 7.14 (1H, s), 7.2- 7.4 (1H, m), 7.4-7.55 (5H, m), 7.59 (1H, s), 8.43 (1H, d, J = 5.0 Hz). |
| 75 | 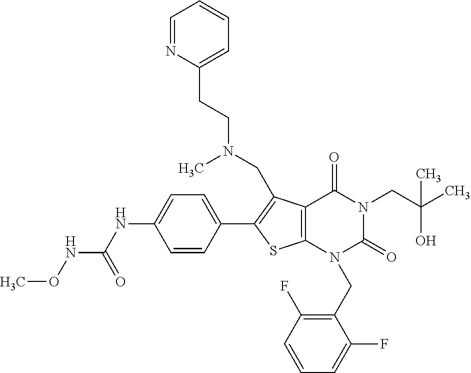 [SMILES: CONC(=O)Nc1ccc(-c2sc3c(c2CN(C)CCc2ccccn2)c(=O)n(CC(C)(C)O)c(=O)n3Cc2c(F)cccc2F)cc1] | ¹H-NMR (CDCl₃) δ: 1.26 (6H, s), 2.21 (3H, s), 2.75-2.95 (4H, m), 3.82 (5H, s), 3.99 (1H, s), 4.24 (2H, s), 5.36 (2H, s), 6.91 (2H, t, J = 8.2 Hz), 7.0-7.1 (2H, m), 7.13 (1H s), 7.2-7.35 (1H m), 7.45-7.55 (5H, m), 7.59 (1H, s), 8.43 (1H, d, J = 4.0 Hz). |
| 76 | 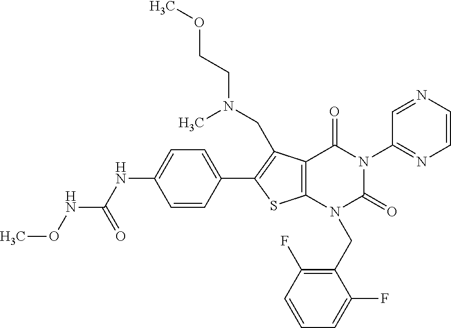 [SMILES: COCCN(C)Cc1c(-c2ccc(NC(=O)NOC)cc2)sc2c1c(=O)n(-c1cnccn1)c(=O)n2Cc1c(F)cccc1F] | ¹H-NMR (CDCl₃) δ: 2.13 (3H, s), 2.62 (2H, t, J = 6.0 Hz), 3.26 (3H, s), 3.41 (2H, t, J = 6.0 Hz), 3.79 (2H, s), 3.83 (1H, s), 5.36 (2H, s), 6.94 (2H, t, J = 8.0 Hz), 7.12 (1H, s), 7.2-7.4 (1H, m), 7.5-7.65 (5H, m), 8.65-8.7 (3H, m). |
| 77 | 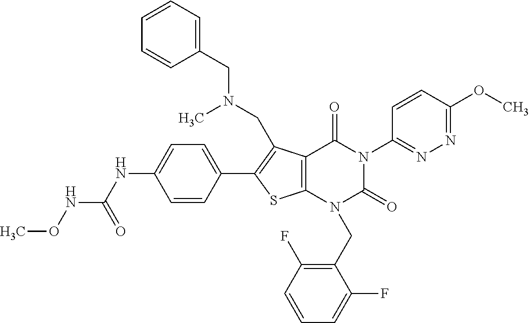 | ¹H-NMR (CDCl₃) δ: 2.06 (3H, s), 3.55 (2H, s), 3.83 (3H, s), 3.87 (2H, s), 4.19 (3H, s), 5.35 (2H, s), 6.92 (2H, t, J = 8.2 Hz), 7.1-7.45 (9H, m), 7.55 (2H, d, J = 8.4 Hz), 7.63 (1H, s), 7.72 (2H, d, J = 8.4 Hz). |
| 78 | 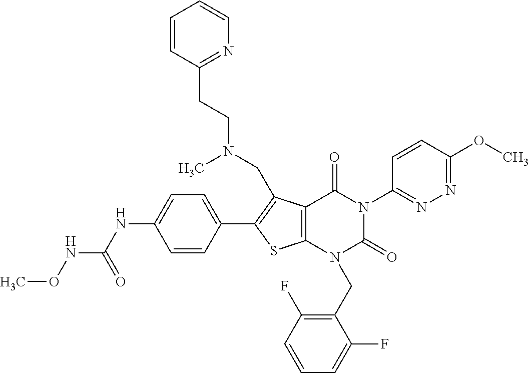 | ¹H-NMR (CDCl₃) δ: 2.20 (3H, s), 2.7-2.9 (4H, m), 3.78 (2H, s), 3.82 (3H, s), 4.19 (3H, s), 5.34 (2H, s), 6.85-7.2 (5H, m), 7.25-7.45 (2H, m), 7.45-7.7 (7H, m), 8.42 (1H, d, J = 4.0 Hz). |
| 79 | 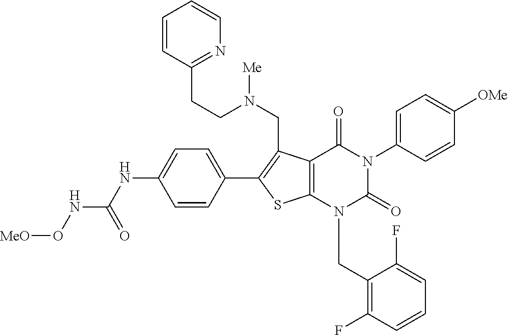 [SMILES: COONC(=O)Nc1ccc(-c2sc3c(c2CN(C)CCc2ccccn2)c(=O)n(-c2ccc(OC)cc2)c(=O)n3Cc2c(F)cccc2F)cc1] | ¹H NMR (CDCl₃) δ 2.20 (3H, s), 2.86 (4H, m), 3.82-3.84 (8H, m), 5.36 (2H, s), 6.92 (2H, d, J = 8.3 Hz), 7.00-7.06 (4H, m), 7.14-7.33 (4H, in), 7.46-7.51 (5H, m), 7.61 (1H, s), 8.42 (1H, d, J = 5.7 Hz). |
| 80 | 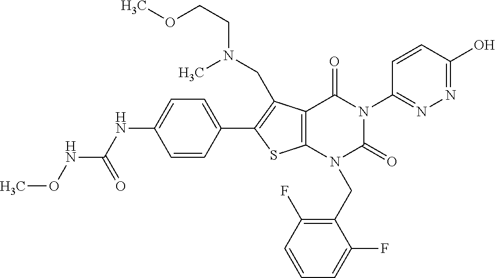 [SMILES: COCCN(C)Cc1c(-c2ccc(NC(=O)NOC)cc2)sc2c1c(=O)n(-c1ccc(O)nn1)c(=O)n2Cc1c(F)cccc1F] | ¹H-NMR (CDCl₃) δ: 2.12 (3H, s), 2.63 (2H, t, J = 5.8 Hz), 3.28 (3H, s), 3.43 (2H, t, J = 5.8 Hz), 3.79 (2H, s), 3.83 (3H, s), 5.35 (2H, s), 6.94 (2H, t, J = 8.2 Hz), 7.0-7.1 (1H, m), 7.2-7.4 (3H, m), 7.5-7.65 (4H, m), 7.63 (1H, s), 10.5-10.6 (1H, brs). |
| 81 | 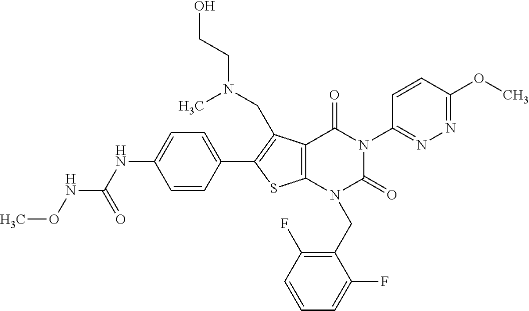 | ¹H-NMR (CDCl₃) δ: 1.98 (3H, s), 2.45-2.5 (2H, m), 2.9-3.2 (1H, m), 3.5-3.55 (2H, m), 3.65-3.85 (2H, brm), 3.82 (3H, s), 4.18 (3H, s), 5.34 (2H, s), 6.93 (2H, t, J = 8.0 Hz), 7.11 (1H, d, J = 9.0 Hz), 7.18 (1H, s), 7.25-7.35 (1H, m), 7.35-7.45 (3H, m), 7.57 (2H, d, J = 8.7 Hz), 7.66 (1H, s). |
| 82 | 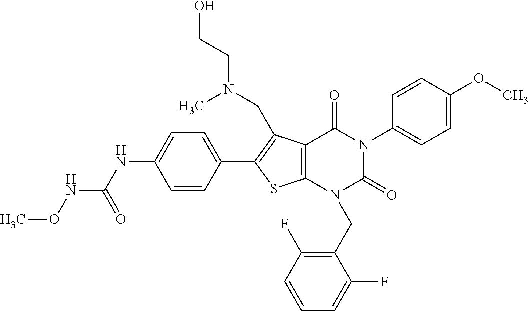 | ¹H-NMR (CDCl₃) δ: 1.99 (3H, s), 2.45-2.55 (2H, m), 3.5-3.6 (2H, m), 3.79 (2H, s), 3.82 (3H, s), 3.83 (3H, s), 5.36 (2H, s), 6.92 (2H, t, J = 8.0 Hz), 6.99 (2H, d, J = 8.8 Hz), 7.1-7.3 (4H, m), 7.39 (2H, d, J = 8.8 Hz), 7.56 (2H, d, J = 8.8 Hz), 7.64 (1H, s). |
| 83 | 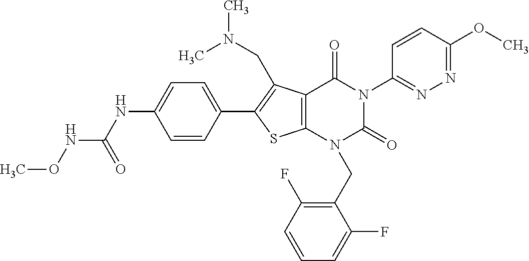 | ¹H-NMR (CDCl₃) δ: 2.15 (6H, s), 3.6-3.8 (2H, m), 3.82 (3H, s), 4.18 (3H, s), 5.35 (2H), 6.92 (2H, t, J = 8.2 Hz), 7.12 (1H, d, J = 8.8 Hz), 7.2-7.65 (7H, m), 7.69 (1H, s). |
| 84 | 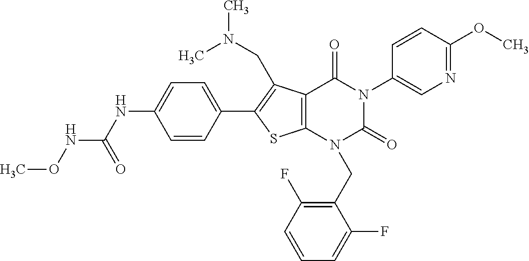 | ¹H-NMR (CDCl₃) δ: 2.13 (6H, s), 3.68 (2H, s), 3.83 (3H, s), 3.96 (3H, s), 5.36 (2H, s), 6.8- 7.0 (3H, m), 7.13 (1H, s), 7.2- 7.4 (1H, m), 7.45-7.65 (6H, m), 8.10 (1H, d, J = 2.6 Hz). |

### Propane-1,3-diones

Additional GnRH antagonists that may be used in conjunction with the compositions and methods described herein include optionally substituted propane-1,3-dione derivatives, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Other GnRH antagonists of this chemical class that may be used for the treatment of estrogen-dependent diseases in accordance with the compositions and methods of the disclosure include compounds described in U.S. Pat. No. 6,960,591, the contents of which are incorporated herein by reference.

### Add-Back Therapy

Among the potential side-effects of GnRH antagonist therapy is a reduction in bone mineral density due to excessive depletion of estrogen (Newhall-Perry et al., American Journal of Obstetrics and Gynecology 173:824-829 (1995)). To combat this potential side effect, a patient undergoing GnRH antagonist therapy using the compositions and methods described herein can be administered add-back therapy. Add-back therapy may contain an estrogen (such as β17-estradiol, ethinyl estradiol, or a conjugated estrogen, such as a conjugated equine estrogen) optionally in combination with a progestin (such as norethindrone or an ester thereof, e.g., norethindrone acetate, or another agent such as progesterone, norgestimate, medroxyprogesterone, or drospirenone).

Endogenous estrogens are largely responsible for the development and maintenance of the female reproductive system and secondary sexual characteristics. Although circulating estrogens exist in a dynamic equilibrium of metabolic interconversions, estradiol is the principal intracellular human estrogen and is substantially more potent than its metabolites, estrone and estriol, at the receptor level. The primary source of estrogen in normally cycling adult women is the ovarian follicle, which secretes 70 to 500 μg of estradiol daily, depending on the phase of the menstrual cycle. After menopause, most endogenous estrogen is produced by conversion of androstenedione, secreted by the adrenal cortex, to estrone by peripheral tissues. Thus, estrone and the sulfate conjugated form, estrone sulfate, are the most abundant circulating estrogens in postmenopausal women. Circulating estrogens modulate the pituitary secretion of the gonadotropins, LH and FSH, through a negative feedback mechanism. Estrogens act to reduce the elevated levels of these hormones seen in postmenopausal women.

Progestin compounds, such as norethindrone and esters thereof (e.g., norethindrone acetate), as well as progesterone, norgestimate, medroxyprogesterone, and drospirenone, enhance cellular differentiation and generally oppose the actions of estrogens by decreasing estrogen receptor levels, increasing local metabolism of estrogens to less active metabolites, or inducing gene products that blunt cellular responses to estrogen. Progestins exert their effects in target cells by binding to specific progesterone receptors that interact with progesterone response elements in target genes. Progesterone receptors have been identified in the female reproductive tract, breast, pituitary, hypothalamus, and central nervous system. Progestins produce similar endometrial changes to those of the naturally occurring hormone progesterone. Progestins may be included in combination with estrogen in add-back therapy. For instance, according to the methods described herein, one can administer estrogen (e.g., E2) in conjunction with a progestin (e.g., norethindrone or an ester thereof, such as norethindrone acetate) to a patient undergoing GnRH antagonist therapy as to counteract the hypoestrogenemia that may be induced by the antagonist. In this way, add-back therapy can be used to mitigate or prevent potentially deleterious side effects, such as a reduction in bone mineral density.

Add-back therapy may be formulated for oral administration. For instance, add-back therapy administered in conjunction with the compositions and methods described herein may be formulated as a tablet, capsule, gel cap, powder, liquid solution, or liquid suspension. In some embodiments, the add-back therapy includes both an estrogen, such as β17-estradiol, and a progestin, such as norethindrone or norethindrone acetate. The estrogen and progestin may be administered separately or admixed in a single composition, such as a single tablet, capsule, gel cap, powder, liquid solution, or liquid suspension. For example, add-back therapy may feature a co-formulation containing estrogen (e.g., in the form of E2) and an additional agent such as a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered to a patient in the form of a single tablet, capsule, gel cap, powder, liquid solution, or liquid suspension that contains both estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate) in the form of a single pharmaceutical composition, such as a single tablet, capsule, gel cap, powder, liquid solution, or liquid suspension.

### Methods of Treating Estrogen-Dependent Diseases

Using the compositions and methods described herein, a patient having an estrogen-dependent diseases, such as uterine fibroids, endometriosis (e.g., rectovaginal endometriosis), and/or adenomyosis, among others, may be administered a GnRH antagonist as described herein. Additional examples of estrogen-dependent diseases that may be treated using the compositions and methods of the disclosure include, without limitation, benign prostatic hypertrophy, precocious puberty, premenstrual syndrome, polycystic ovary syndrome, hirsutism, short stature, a sleep disorder, acne, baldness, and irritable bowel syndrome.

A variety of methods known in the art and described herein can be used to determine whether a patient is responding favorably to GnRH antagonist treatment. For instance, beneficial clinical results in response to GnRH antagonist therapy include, without limitation, alleviation of symptoms of the endometrial growth disorder. Indications of successful treatment of a uterine fibroids patient include, for example, (i) a reduction in serum concentration of FSH, LH, and/or E2 following administration of the GnRH antagonist to the patient, (ii) a reduction in uterine blood loss following administration of the GnRH antagonist to the patient, (iii) elimination of heavy menstrual bleeding following administration of the GnRH antagonist to the patient, and (iv) induction of amenorrhea in the patient following administration of the GnRH antagonist to the patient.

Similarly, clinical indicators of successful treatment of an endometriosis patient (e.g., a rectovaginal endometriosis patient) that is administered a GnRH antagonist include, without limitation, (i) a reduction in serum concentration of FSH, LH, and/or E2 following administration of the GnRH antagonist to the patient, (ii) a reduction in the volume of one or more endometriosis nodes (e.g., rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and (x) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

Exemplary indicia of successful treatment of an adenomyosis patient that is administered a gonadotropin-releasing hormone (GnRH) antagonist include, without limitation, (i) a reduction in serum concentration of FSH, LH, and/or E2 following administration of the GnRH antagonist to the patient, (ii) a reduction in uterine volume following administration of the GnRH antagonist to the patient, (iii) a reduction in the thickness of the anterior and/or posterior region of the uterine myometrium following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine tenderness following administration of the GnRH antagonist to the patient; (ix) a reduction in uterine bleeding following administration of the GnRH antagonist to the patient; (x) achievement of amenorrhea following administration of the GnRH antagonist to the patient; (xi) a reduction in the diameter of a junctional zone of adenomyosis following administration of the GnRH antagonist to the patient; and (xii) an improvement in the patient's overall well-being as determined by an improvement in the patient's EHP-30 score following administration of the GnRH antagonist to the patient and/or by way of a positive PGIC score following administration of the GnRH antagonist to the patient.

### Modified Biberoglu and Behrman Symptom Severity Scale

Exemplary methods for assessing a patient's response to GnRH antagonist therapy for the treatment of estrogen-dependent diseases, such as uterine fibroids, endometriosis (e.g., rectovaginal endometriosis) and/or adenomyosis, among others described herein, include administration of a modified Biberoglu and Behrman questionnaire, as described herein. An exemplary mB&B questionnaire for use in conjunction with the compositions and methods described herein is shown in Table 4, below.

### Endometriosis Health Profile Questionnaire

Additional methods for assessing patient respond to GnRH antagonist therapy for the treatment of estrogen-dependent diseases, such as uterine fibroids, endometriosis (e.g., rectovaginal endometriosis) and/or adenomyosis, among others described herein, include analyzing the patient's score on an Endometriosis Health Profile questionnaire. An exemplary Endometriosis Health Profile questionnaire for use in conjunction with the compositions and methods described herein is the EHP-30 questionnaire shown in Table 5, below.

### Patient Global Impression of Change Score

Additional methods for assessing patient response to GnRH antagonist therapy for the treatment of an estrogen-dependent disease described herein, such as uterine fibroids, endometriosis (e.g., rectovaginal endometriosis, and/or adenomyosis, among others) include analyzing the patient's score on a Patient Global Impression of Change (PGIC) scale. An exemplary PGIC questionnaire for use in conjunction with the compositions and methods described herein is shown in Table 6, below.

### Quantitation of Uterine Blood Loss by the Alkaline Hematin Method

Techniques for quantifying uterine blood loss are known in the art and include, for instance, the alkaline hematin method, as described, for instance, in Hallberg et al., Scand. J. Clin. Lab. Invest. 16:244-248 (1964), the disclosure of which is incorporated herein by reference as it pertains to techniques for assessing the volume of blood lost by a patient. In the alkaline hematin approach, uterine blood soaked into, for example, a sanitary napkin, vaginal tampon, or cotton pad, is reconstituted in a basic aqueous solution, such as a solution of 5% (w/v) sodium hydroxide. This incubation enables (i) extraction of the iron-containing porphyrin of hemoglobin and (ii) oxidation of the ferrous ion to a hydroxy-coordinated ferric ion in each chelate, thus forming hematin. Hematin is a detectable chromophore, absorbing light at between 550 and 546 nm. By comparing the concentration of hematin obtained from incubation of a soaked menstrual blood sample with aqueous sodium hydroxide to the concentration of hematin obtained from incubation of a sample of venous blood with aqueous sodium hydroxide, one can stoichiometrically determine the volume of menstrual blood lost by a patient, such as a patient having an estrogen-dependent disease. Improvements to the original alkaline hematin method are known in the art and are described, for example, in Newton et al., Contraception 16:269-282 (1977), and in van Eijkeren et al., Eur. J. Obstet. Gynecol. Reprod. Biol. 22:345-351 (1986), the disclosures of each of which are incorporated herein by reference as they pertain to methods of determining the volume of blood lost by a patient.

### Routes of Administration and Dosing of GnRH Antagonists

The GnRH antagonists described herein may be administered to a patient in need thereof (e.g., a patient suffering from an estrogen-dependent disease, such as uterine fibroids, endometriosis (e.g., rectovaginal endometriosis) and/or adenomyosis, among others described herein) by a variety of routes of administration. For instance, the GnRH antagonists described herein may be formulated for oral administration, among other routes. Exemplary non-oral routes of administration of the GnRH antagonists described herein include, without limitation, intravenous administration, parenteral administration, subcutaneous administration, intramuscular administration, and intradermal administration, among others.

In some embodiments, the GnRH antagonist is a compound of any one of formulas (I)-(VIa), above, and is administered to the patient in an amount of from about 25 mg to about 500 mg per dose, and may be administered in one or more doses per day, for example, in accordance with a dosing schedule described above. For instance, the GnRH antagonist may a compound of any one of formulas (I)-(VIa), above, and may be administered to the patient in an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose, and may be administered in one or more doses per day (e.g., in a single daily dose). In some embodiments, the GnRH antagonist is a compound of any one of formulas (I)-(VIa), above, and is administered to the patient once daily in an amount of from 100 mg to 300 mg, per dose, such as from 105 mg to 295 mg, 110 mg to 290 mg, 115 mg to 285 mg, 120 mg to 280 mg, 125 mg to 275 mg, 130 mg to 270 mg, 135 mg to 265 mg, 140 mg to 260 mg, 145 mg to 255 mg, 150 mg to 250 mg, 155 mg to 245 mg, 160 mg to 240 mg, 165 mg to 235 mg, 170 mg to 230 mg, 175 mg to 225 mg, 180 mg to 220 mg, 185 mg to 215 mg, 190 mg to 210 mg, or 195 mg to 205 mg, per dose. In some embodiments, the GnRH antagonist is a compound of any one of formulas (I)-(VIa), above, and is administered to the patient once daily in an amount of about 50 mg, 75 mg, 100 mg, or 200 mg per dose.

The GnRH antagonists described herein may be administered to a patient a plurality of times over the course of a treatment period. For instance, the GnRH antagonists described herein may be administered to a patient periodically over a treatment period of at least two weeks (e.g., a treatment period of from about two weeks to about six months, about three weeks to about five months, about four weeks to about four months, or about one month to about three months). The GnRH antagonist may be administered to the patient, for example, over a treatment period of from about four weeks to about six months (e.g., from about 28 days to about 180 days, about 30 days to about 175 days, about 35 days to about 170 days, about 40 days to about 165 days, about 45 days to about 160 days, about 50 days to about 155 days, about 55 days to about 150 days, about 60 days to about 145 days, about 65 days to about 140 days, about 70 days to about 135 days, about 75 days, to about 130 days, about 80 days to about 125 days, about 85 days, to about 120 days, or about 90 days to about 115 days). In some embodiments, the GnRH antagonist is periodically administered to the patient over the course of from about eight weeks to about sixteen weeks (e.g., from about 60 days to about 110 days, about 65 days to about 105 days, about 70 days to about 100 days, about 75 days to about 95 days, or about 80 days, to about 90 days). In some embodiments, the GnRH antagonist is periodically administered to the patient over a treatment period of about 12 weeks. In some embodiments, the GnRH antagonist is periodically administered to the patient over a treatment period of about 24 weeks.

Additional dosing schedules for the treatment of estrogen-dependent diseases, such as uterine fibroids, endometriosis (e.g., rectovaginal endometriosis) and/or adenomyosis, among others described herein, using other GnRH antagonists disclosed herein are described in detail above.

### Pharmaceutical Compositions

GnRH antagonists suitable for use with the compositions and methods described herein can be formulated into a pharmaceutical composition for administration to a patient, such as a female human patient, in a biologically compatible form suitable for administration in vivo. A pharmaceutical composition containing a GnRH antagonist, such as a compound described herein (e.g., 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof, such as the choline salt thereof), may additionally contain a suitable diluent, carrier, or excipient. GnRH antagonists can be administered to a patient, for example, orally or by intravenous injection. Under ordinary conditions of storage and use, a pharmaceutical composition may contain a preservative, e.g., to prevent the growth of microorganisms. Conventional procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33).

Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a patient in need of treatment.

A pharmaceutical composition may be administered to a patient, e.g., a human patient, alone or in combination with one or more pharmaceutically acceptable carriers, e.g., as described herein, the proportion of which may be determined by the solubility and/or chemical nature of the compound, chosen route of administration, and standard pharmaceutical practice.

### Compound for Use

In another aspect, the disclosure provides a GnRH antagonist (e.g., a GnRH antagonist described herein) for use in any of the methods described herein. For example, the disclosure features a GnRH antagonist, such as a GnRH antagonist described herein, for use in a method of treating an estrogen-dependent disease, such as uterine fibroids, endometriosis, adenomyosis, or rectovaginal endometriosis. The method may feature, for example, any one or more of the method steps recited herein.

### Medicament

In another aspect, the disclosure provides a GnRH antagonist (e.g., a GnRH antagonist described herein) for use in the manufacture of a medicament for performing any of the methods described herein. For example, the disclosure features a GnRH antagonist, such as a GnRH antagonist described herein, for use in the manufacture of a medicament for use in a method of treating an estrogen-dependent disease, such as uterine fibroids, endometriosis, adenomyosis, or rectovaginal endometriosis.

The method may feature, for example, any one or more of the method steps recited herein.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example 1. Administration of a GnRH Antagonist Effectuates a Sustained Alleviation of Estrogen-Dependent Disease Symptoms Even after Treatment is Halted

### Methods

This example describes the results of a Phase 2b human clinical trial undertaken to evaluate the efficacy of a GnRH antagonist represented by formula (VI) in a series of patients suffering from an estrogen-dependent disease. In this trial, a series of human female patients diagnosed as having endometriosis were administered the choline salt of 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid periodically over the course of a 24-week treatment period. After 24 weeks of treatment, various patients entered a 24-week post-treatment follow-up period during which the patients were monitored for serum β17-estradiol (E2) concentration, severity of disease symptoms, and bone mineral density.

### Results

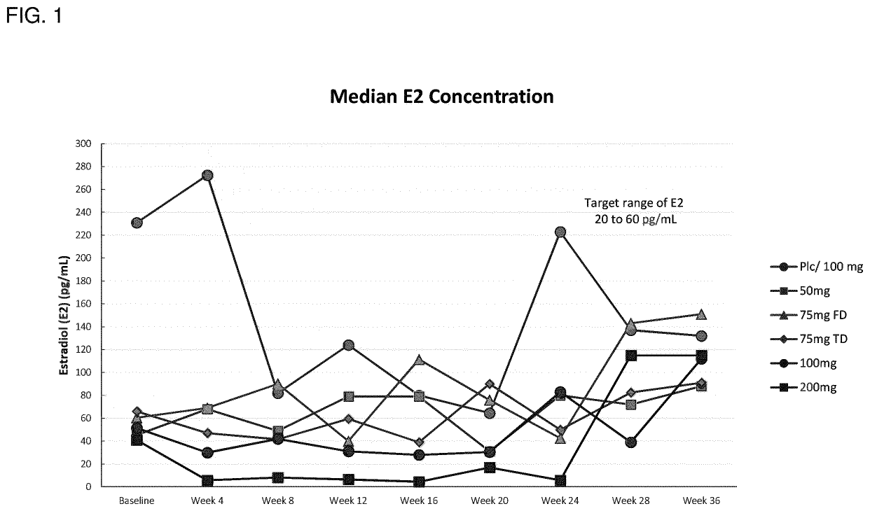

A series of n=328 human female patients having clinically-diagnosed endometriosis were randomly assigned to treatment arms in which patients were to be administered placebo or the GnRH antagonist in a once-daily amount of 50 mg/day, 100 mg/day, 75 mg/day, or 200 mg/day. Patients initially treated with placebo (plc) were administered the GnRH antagonist in an amount of 100 mg/day after 12 weeks. Also, after 12 weeks, patients that were initially administered 75 mg/day were provided a higher (100 mg/day) or lower (50 mg/day) dosage of the GnRH antagonist based on the patients' E2 levels at the 12-week point. The E2 levels for this group of patients is represented by “75 mg (TD)” in FIGS. 1-3. Patients that were administered 75 mg through the end of the treatment period are represented by “75 mg (FD)” in FIGS. 1-3.

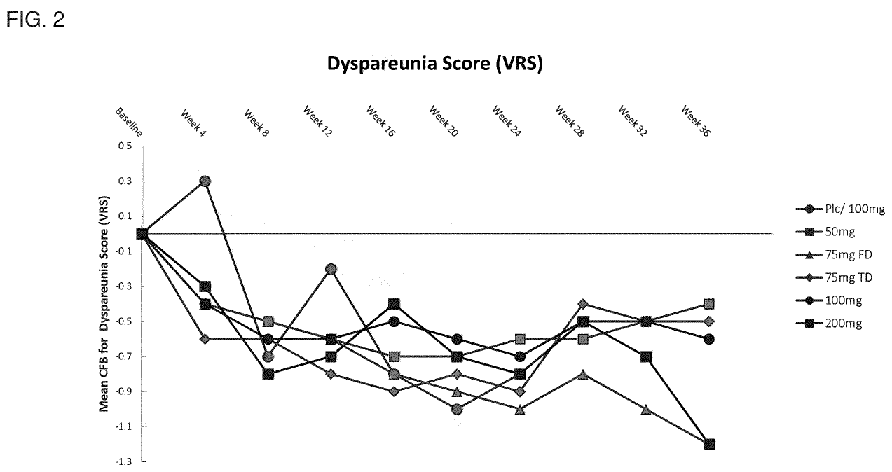

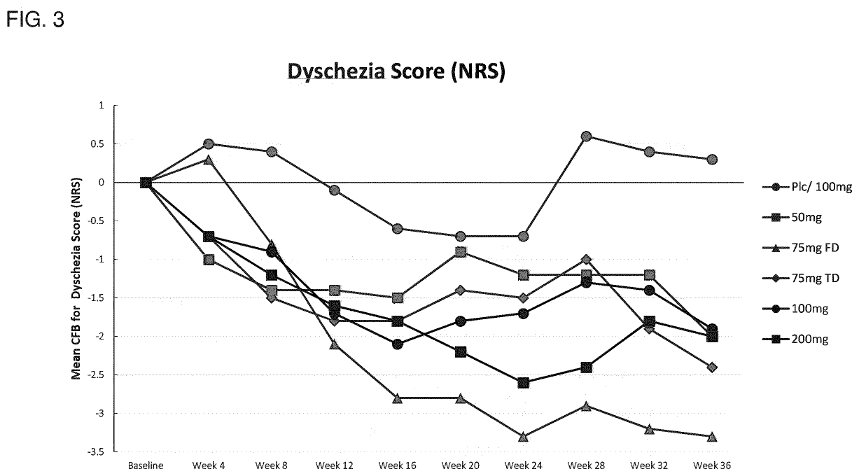

After 24 weeks of continuous, once-daily treatment, administration of the GnRH antagonist ceased. A set of n=65 patients entered the 24-week post-treatment follow-up study. Following the 24-week treatment period, these patients were periodically monitored. The median E2 levels of patients that were administered the GnRH antagonist during the original 24-week treatment period and that subsequently entered the 24-week post-treatment follow-up study are shown in FIG. 1. FIGS. 2 and 3 show the mean change in dyspareunia and dyschezia scores for such patients over the 24-week treatment period and a 12-week portion of the post-treatment follow-up period. As shown in FIGS. 2 and 3, despite cessation of GnRH antagonist administration, patients continued to exhibit a sustained reduction in dyspareunia, as assessed using the Verbal Rating Score (VRS), as well as a sustained reduction in dyschezia, as assessed using the Numerical Rating Score (NRS).

In addition to demonstrating a continuous reduction in dyspareunia and dyschezia, patients that entered the 24-week post-treatment follow-up period exhibited a reduction in overall pelvic pain, as shown in Table 7, below. This table depicts the proportion of patients that exhibited an overall pelvic pain score reduction of greater than 30% from baseline using a VRS scale at 12 weeks following the cessation of GnRH antagonist treatment.

TABLE 7
Overall Pelvic Pain Responder Rates (Defined has patients
exhibiting an overall pelvic pain VRS score reduction
of > 30% from baseline VRS score)
Overall Pelvic Pain Responder Rate (%)

| Dose (Once-daily) | 75 mg | 200 mg |
|---|---|---|
| After 24 weeks of GnRH | 70.8 | 77.3 |
| antagonist treatment | (n = 48) | (n = 44) |
| After 12 weeks following | 87.5 | 70.0 |
| cessation of GnRH antagonist | (n = 7) | (n = 10) |
| treatment | | |

Surprisingly, in addition to exhibiting a sustained reduction in disease symptomology, patients that were monitored during the post-treatment follow-up period exhibited an increase in bone mineral density. Table 8, below, depicts the mean change in bone mineral density from a baseline, pre-treatment measurement following completion of the 24-week treatment period and at 12 weeks following the cessation of the GnRH antagonist. All bone mineral density measurements shown below were obtained by assessing bone mineral density at each patient's spine.

TABLE 8
Bone mineral density following 24-week treatment period
and 12 weeks after cessation of treatment
Bone Mineral Density (Mean % change from baseline)

| Dose (Once-daily) | 75 mg | 200 mg |
|---|---|---|
| After 24 weeks of GnRH | −0.798% | −2.602% |
| antagonist treatment | | |
| After 12 weeks following | +0.313% | +1.135% |
| cessation of GnRH antagonist | | |
| treatment | | |

As shown in Table 8, patients that exhibited a reduction in bone mineral density during the 24-week treatment period effectively recovered bone mineral density upon cessation of GnRH antagonist treatment.

### Conclusions

Taken together, the results of these experiments demonstrate that patients administered a GnRH antagonist represented by formula (VI) over the course of a treatment period exhibit a sustained alleviation in symptoms of an estrogen-dependent disease, even after administration of the GnRH antagonist is halted. Additionally, patients that exhibited a reduction in bone mineral density during treatment with the GnRH antagonist demonstrated a recovery in bone mineral density once the treatment was discontinued. Surprisingly, this recovery in bone mineral density occurred without the detriment of a return in disease symptoms.

### Example 2. Use of a GnRH Antagonist for the Treatment of a Patient Having Uterine Fibroids or Endometriosis

Using the compositions and methods described herein, a patient may be administered a GnRH antagonist so as to treat, and/or ameliorate the symptoms of, uterine fibroids or endometriosis, among other estrogen-dependent diseases. The GnRH antagonist (e.g., a compound of formula (I), above, such as compound (VI) or the choline salt thereof) may be administered to the patient in an amount sufficient to reduce the serum concentration of LH, FSH, and/or E2 in circulation. The GnRH antagonist may be administered, for example, in an amount of from about 25 mg to about 500 mg per dose. Exemplary doses of the GnRH antagonist include, without limitation, an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose. For example, when the GnRH antagonist is compound (VI) or the choline salt thereof, the compound may be administered at a dose of 200 mg.

The GnRH antagonist may be administered to the patient periodically over a first treatment period of, for example, two or more weeks (e.g., a first treatment period of 2 weeks, 3 weeks, 4 weeks, 5 weeks, 6 weeks, 7 weeks, 8 weeks, 9 weeks, 10 weeks, 1 month, 2 months, 3 months, 4 months, 5 months, 6 months, 7 months, 8 months, 9 months, 10 months, 11 months, 12 months, or longer). The GnRH antagonist may be provided to the patient in combination with add-back therapy. The patient may then be monitored for bone mineral density loss, for example, using dual energy X-ray absorptiometry. If the patient is determined to exhibit a reduction in bone mineral density relative to a measurement of the patient's bone mineral density obtained during, or prior to commencement of, the first treatment period, administration of the GnRH antagonist may be temporarily halted. After a time, such as once the patient has demonstrated an increase in bone mineral density, administration of the GnRH antagonist may commence again.

To determine the responsiveness of the patient to the GnRH antagonist, a physician may monitor the time required for the patient to achieve a reduction in uterine blood loss in the case of a patient having uterine fibroids. For example, the physician may observe one or more of the following responses of the patient as an indicator of successful treatment: (i) a reduction in menstrual blood loss to less than 80 ml per menstrual cycle, such that the patient no longer exhibits heavy menstrual blood loss, the volume of one or more rectovaginal endometriosis nodes following administration of the GnRH antagonist to the patient, and/or (ii) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

In the case of a patient having endometriosis, the physician may monitor the patient to assess whether the patient exhibits a reduction in pain, such as overall pelvic pain, dysmenorrhea, dyspareunia, or dyschezia. For example, the physician may observe one or more of the following responses of the patient as an indicator of successful treatment: (i) a reduction in serum concentration of FSH, LH, and/or E2 following administration of the GnRH antagonist to the patient, (ii) a reduction in the volume of one or more endometriosis nodes (e.g., rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and/or (x) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

### Example 3. Use of a GnRH Antagonist for the Treatment of a Patient Having Adenomyosis

This examples describes the results of a human clinical trial conducted to evaluate the ability of a GnRH antagonist, 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid (represented by formula (VI)), or a pharmaceutically acceptable salt thereof, to treat adenomyosis, an estrogen-dependent disease, in human patients diagnosed as having this condition.

One aim of this study was to investigate the ability of the GnRH antagonist to maintain its therapeutic effects despite being administered to patients in a progressively lower dosage throughout the trial. To this end, the patients treated in this study was first administered the GnRH antagonist of formula (VI) in an amount of 200 mg/day during an initial 12-week period. The GnRH antagonist was then administered to the patients in a reduced amount of 100 mg/day during a subsequent 12-week period. The patients' responsiveness to the GnRH antagonist was monitored throughout the study.

In addition to assessing the effectiveness of the GnRH antagonist when administered at lower doses, another aim of this study was to evaluate the ability of the GnRH antagonist to sustain its therapeutic effects even after treatment was ceased altogether. Accordingly, after being periodically administered the GnRH antagonist of formula (VI) over the course of 24 weeks, first in an amount of 200 mg/day and then in an amount of 100 mg/day, treatment with the GnRH antagonist was halted entirely. The patients were then monitored during a post-treatment follow-up period to analyze the extent to which the therapeutic effects of the compound were sustained.

To test the effects of compound (VI), a total of n=6 human female patients diagnosed as having adenomyosis were administered compound (VI) in accordance with the dosing schedule described above. One of these patients in particular was closely monitored beginning well before her treatment with compound (VI) was initiated. The specific results of this patient's therapy are described in the section entitled “Single-Patient Longitudinal Case Study,” below. The cumulative set of results for all 6 patients is reported in the section entitled “Multi-Patient Analysis,” below.

### Introduction

Adenomyosis is a commonly encountered estrogen-dependent disease characterized by the presence of endometrial glands and stroma in the myometrium having a depth of greater than 2.5 mm. The endometrial glands and stroma are surrounded by hyperplastic and hypertrophic smooth muscle. Affecting 19.5% of women of reproductive age, uterine adenomyosis is responsible for heavy menstrual bleeding, infertility, and pelvic pain. The symptomology adenomyosis may overlap with that of other estrogen-dependent diseases, including endometriosis and uterine fibroids. Despite its prevalence and severity of symptoms, there has been a paucity of treatment options available for treating the underlying pathology of this disease.

### Single-Patient Longitudinal Case Study

Methods

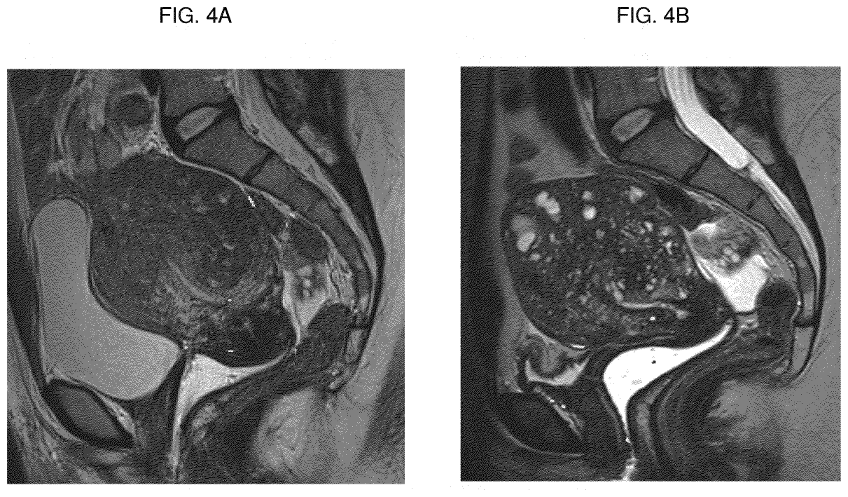

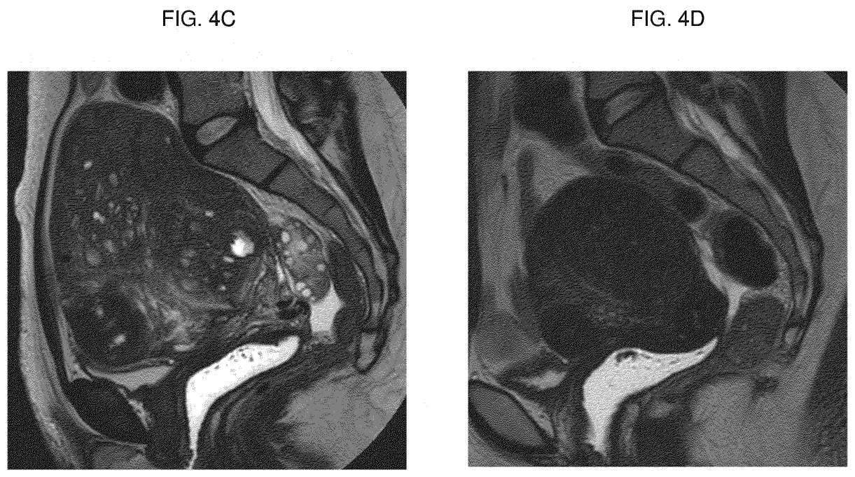

As part of the clinical trial, a case study was conducted in which a single, female patient of reproductive age having clinically diagnosed adenomyosis was monitored during the course of treatment with compound (VI). The patient, born in 1981, was nulliparous. Two years before the study began, the patient presented with heavy menstrual bleeding, pelvic pain, and dysmenorrhea. On clinical examination, her uterine volume was equivalent to 12 weeks of gestation and magnetic resonance imaging (MRI) revealed an enlarged uterus with diffuse and disseminated adenomyosis (FIG. 4A). Her gynecologist prescribed ulipristal acetate (UPA), a selective progesterone receptor modulator (SPRM), to be administered in an amount of 5 mg/day over the course of a treatment period lasting three months.

As is described in further detail below, treatment with UPA was largely unsuccessful. After being administered UPA in an amount of 5 mg/day for three months, treatment with UPA was discontinued.

The patient was then recommended for treatment with the GnRH antagonist represented by formula (VI) herein. Accordingly, the patient was orally administered compound (VI) in an amount of 200 mg/day for three months. Following this initial three-month period, the patient was orally administered compound (VI) in a reduced amount of 100 mg/day for a subsequent three-period. During both treatment periods, compound (VI) was administered to the patient in the form of a choline salt.

Results

During the course of the patient's treatment with UPA in an amount of 5 mg/day for three months, the patient's symptoms of pelvic pain and dysmenorrhea worsened. Moreover, the patient experienced breakthrough bleeding and spotting. At the end of the UPA course, MRI examination revealed that the patient exhibited a significantly increased quantity of adenomyotic lesions. Moreover, the lesions were substantially aggravated relative to the state of the lesions prior to treatment with UPA (FIG. 4B). Particularly, the results of the patient's MRI obtained following UPA treatment showed numerous spots typical of adenomyosis in the myometrium. The MRI also revealed an extension of the lesions, featuring enlarged, asymmetric, heterogeneous myometrial tissue with multiple myometrial cysts. The presence of this cystic tissue is indicated of dilated islets of ectopic endometrium (see, e.g., Bazot et al., Fertil Steril. 109:389-397 (2018)). In sum, the patient's MRI results clearly revealed a worsened state of the myometrium after UPA treatment, reflecting the exacerbation of symptoms. Accordingly, after the patient's three-month UPA treatment period, treatment with UPA was discontinued.

The patient subsequently sought another form of treatment for her pelvic pain, heavy bleeding, and infertility. On clinical examination, the patient's uterus was enlarged, now showing the equivalence of 14-15 weeks of gestation. Treatment with a GnRH agonist was proposed, but the patient rejected this form of therapy because of the mode of administration and the risk of symptom aggravation due to the likelihood of a transient increase in GnRH activity at the beginning of the treatment period. One year later, the patient again sought treatment for her still-worsening adenomyosis condition. At this point, upon clinical examination, the patient's uterus had a volume equivalent to 16-17 weeks of gestation.

The patient was recommended for once-daily treatment with the GnRH antagonist of formula (VI). Accordingly, the patient was administered the GnRH antagonist of formula (VI) in an amount of 200 mg/day during a first treatment period, which lasted three months. At the conclusion of the first treatment period, the patient began a second treatment period in which she was administered compound (VI) in a reduced amount of 100 mg/day for three months. During both treatment periods, compound (VI) was administered to the patient in the form of a choline salt.

At baseline, prior to treatment with compound (VI), the patient's hemoglobin (Hb) level was 10 g/dL. This level of Hb was indicative of moderate anemia. Additionally, at baseline, the patient exhibited an estrogen (E2) level of 45 pg/mL. The patient's concentration of E2 rapidly decreased to less than 20 pg/mL after four weeks of treatment with the GnRH antagonist of formula (VI). The results of the patient's MRI at baseline (FIG. 4C) revealed a very large uterus, with multiple images typical of severe full-thickness adenomyosis. The patient's baseline uterine volume was estimated to be approximately 875 cm³. The patient's baseline quality of life was profoundly affected by her adenomyosis, according to the Endometriosis Health Profile (EHP)-30 questionnaire. Specifically, the patient exhibit a pain score of 97.8 and an emotional well-being score of 58.3. The patient's scores on each of the three remaining scales (control and powerlessness, social support, and self-image) were 100.

During the initial 12-week period in which compound (VI) was administered once-daily in an amount of 200 mg, the patient remained in amenorrhea and, at week 12, noted a significant improvement in symptoms. Particularly, the patient's 5-scaled EHP-30 score at the conclusion of the first 12-week period was 0, and her Hb level was 12.2 g/dL. The results of the patient's MRI after the conclusion of the first 12 weeks of treatment with compound (VI) showed a reduction in uterine volume had to 290 cm³. Additionally, the patient's adenomyotic lesions had significantly regressed (FIG. 4D). Clinical evaluation revealed that the patient's uterus now had a volume equivalent to 10 weeks of gestation. Both the clinical evaluation and the MRI illustrated the highly significant response to compound (VI) administered in an amount of 200 mg/day.

Following the initial 12-week period of treatment with compound (VI), the patient was administered compound (VI) at a reduced amount of 100 mg/day for the next 12 weeks (weeks 13-24). During this subsequent treatment period, the patient's E2 levels were 38 pg/ml, 26 pg/ml, and 52 pg/mL at weeks 16, 20, and 24, respectively. After the conclusion of 24 weeks of treatment with compound (VI), the patient was still in amenorrhea and had an Hb level of 13.6 g/dL. She reported continued alleviation of symptoms even after treatment ceased. Moreover, she reported a high quality of life, scoring 4.2 on the EHP-30 scale for emotional well-being and scoring 0 on all of the remaining EHP-30 scales. The patient's endometrial thickness, which was evaluated by vaginal ultrasound, was 3 mm, and her uterine volume was 440 cm³, as assessed by MRI. After treatment with the GnRH antagonist of formula (VI) ceased, the patient's adenomyotic lesions remained significantly smaller compared to the MRI done at baseline.

With regard to the patient's bone mineral density (BMD), at baseline, her femoral neck T-score and Z-score were +1.6 and +0.3, respectively, and her baseline lumbar spine T-score and Z-score were +0.7 and −0.5, respectively. These values were found to be unchanged at week 24. All BMD values were measured using Dual Energy X-ray Absorptiometry (DEXA).

As for side effects, the patient experienced a few instances of hot flushes and vaginal dryness during the first treatment period in which she was administered compound (VI) in an amount of 200 mg/day. These side effects disappeared upon reducing the amount of compound (VI) administered to the patient to 100 mg/day.

Taken together, these findings demonstrate that a partial suppression of E2 can be achieved using reduced dosages of compound (VI), and that this therapeutic effect can be maintained even after treatment ceases. This partial suppression of E2 simultaneously resolves adenomyotic lesions in the endometrium while avoiding potentially harmful side effects associated with excessive E2 depletion.

### Multi-Patient Analysis

Methods

As was the case for the patient described in the “Single-Patient Longitudinal Case Study,” above, all n=6 patients in the clinical trial were diagnosed as having adenomyosis. All were orally administered compound (VI) in an amount of 200 mg/day for three months. Following this initial three-month period, all patients were orally administered compound (VI) in a reduced amount of 100 mg/day for a subsequent three-period. During both treatment periods, compound (VI) was administered to the patient in the form of a choline salt.

Results

At baseline, all n=6 patients presented with pelvic pain, severe dysmenorrhea, heavy menstrual bleeding, and anemia. MRI analysis at baseline revealed, for all patients, an enlarged uterus with severe adenomyosis, characterized by heterogenous myometrial tissue with multiple myometrial cysts. The patients exhibited a mean uterine volume of 320 cm³ and a range of from 83 cm³ to 875 cm³. The uterine volumes of the n=6 patients observed at various timepoints throughout the study are summarized in Table 9, below.

TABLE 9
Uterine volumes of n = 6 adenomyosis
patients treated with compound (VI)

| | Baseline Uterine | Week 12 Uterine | Week 24 Uterine |
|---|---|---|---|
| Patient | Volume | Volume | Volume |
| 1 | 875 cm³ | 290 cm³ | 440 cm³ |
| 2 | 198 cm³ | 84 cm³ | 75 cm³ |
| 3 | 228 cm³ | 138 cm³ | 266 cm³ |
| 4 | 354 cm³ | 81 cm³ | NR |
| 5 | 183 cm³ | 70 cm³ | NR |
| 6 | 83 cm³ | 70 cm³ | NR |

*NR: Not reported

After 12 weeks of treatment, MRI analysis showed significant reduction in uterine volume to a mean of 122 cm³ and a range of 70 cm³ to 290 cm³. Significant regression of adenomyotic lesions was also observed for all patients. After 12 weeks, the patients exhibited sustained amenorrhea, substantially reduced pelvic pain, and anemia was resolved for all patients. In all cases, serum E2 decreased rapidly to less than 20 pg/ml.

During the subsequent 12 weeks (weeks 13-24), patients remained in amenorrhea and reported continued alleviation of symptoms. Serum E2 values were between 25 pg/ml and 55 pg/ml.

After 24 weeks, all patients' adenomyotic lesions remained smaller than those observed at baseline by MRI analysis. Each patient's BMD, as assessed by DEXA, showed either minimal change or no change from baseline. Side effects of hot flushes and vaginal dryness observed during the 200 mg/day treatment period resolved following the reduction to the 100 mg/day regime.

The results of all n=6 patients reinforce the findings of the single-patient longitudinal case study and demonstrate that the GnRH antagonist of formula (VI) maintains its therapeutic effect on patients suffering from an estrogen-dependent disease even when administered at reduced dosages and even after treatment has ceased.

### Discussion

The results of this study demonstrate the effectiveness of the GnRH antagonist of formula (VI) for treating the pathology that underlies adenomyosis, an estrogen-dependent disorder. Particularly, the results show that compound (VI) sustains its therapeutic effect when administered at reduced dosages and even after treatment is halted.

Additionally, the results of the single-patient longitudinal case study show the ability of GnRH antagonists, such as the compound of formula (VI), to effectively treat adenomyosis in patients who previously failed to respond to treatment with SPRMs, such as UPA. As the results of this study demonstrate, GnRH antagonists, such as the compound of formula (VI), are efficacious in patients suffering from uterine adenomyosis and whose adenomyotic condition was exacerbated by SPRM treatment.

In sum, the results of treatment of all n=6 patients with compound (VI), including the specific results of the singe patient for whom a longitudinal study was conducted, demonstrate that GnRH antagonists, such as compound (VI), can be used to effectively treat the etiology that gives rise to estrogen-dependent diseases and alleviate associated symptoms. Importantly, GnRH antagonists, such as compound (VI), can maintain their therapeutic effect despite reductions in dosage and after treatment has ceased, providing patients with the flexibility to down-titrate doses and/or pause their therapy as needed without experiencing a setback in estrogen-dependent disease progression.

### Example 4. The GnRH Antagonist Represented by Formula (VI) Effectuates a Sustained Reduction in Uterine Fibroids Symptomology and can Safely be Administered to Patients Over Extended Treatment Periods

### Objective

Uterine fibroids is a common estrogen-dependent disease characterized by the growth of benign tumors of the muscular tissue of the uterus. Uterine fibroids affect women of childbearing age and can vary in size from undetectable to a large bulky mass. The symptoms of uterine fibroids are wide-ranging and include heavy menstrual bleeding, anemia, pelvic pressure and bloating, urinary frequency, and pain that can be extremely debilitating with a significant impact on quality of life. These symptoms can also have an impact on mental health, creating the additional burden of anxiety and distress. The purpose of the experiments described in this example was to evaluate the safety and efficacy of the GnRH antagonist represented by formula (VI), herein, in patients suffering from uterine fibroids.

### Methods

This example describes the results of two Phase 3, double-blind, randomized, placebo-controlled, multicenter, human clinical trials undertaken to evaluate the efficacy of the GnRH antagonist represented by formula (VI) in a series of patients suffering from uterine fibroids. In these trials, referred to herein as “PRIMROSE 1” and “PRIMROSE 2,” human female patients diagnosed as having uterine fibroids were periodically administered compound (VI), 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, in the form of a choline salt over the course of a 12-month treatment period. Patients were administered compound (VI) once daily in an amount of either 100 mg or 200 mg. Patients receiving compound (VI) in a daily amount of 200 mg also received once daily hormonal add-back therapy (ABT), which consisted of 1 0.0 mg of β17-estradiol (E2) per day and 0.5 mg of norethindrone acetate (NETA) per day. Patients participating in the PRIMROSE 1 and PRIMROSE 2 studies did not receive Vitamin D or calcium supplementation at any point during the treatment period.

The PRIMROSE 1 study, conducted in the United States, enrolled 526 women. The PRIMROSE 2 study, conducted in the United States and Europe, enrolled 535 women. Baseline characteristics of randomized and treated patients are shown in Table 10, below.

TABLE 10
Demographics and baseline characteristics

| | PRIMROSE 1 | | | | | | PRIMROSE 2 | | | | | |
|---|---|---|---|---|---|---|---|---|---|---|---|---|
| | | | | | 200 mg + | | | | | | 200 mg + | |
| | Placebo | | 100 mg | | ABT | | Placebo | | 100 mg | | ABT | |
| | n = 103 | | n = 94 | | n = 102 | | n = 102 | | n = 97 | | n = 98 | |
| Age (years): | 42.0 | (5.7) | 41.3 | (5.9) | 41.8 | (5.9) | 42.9 | (5.3) | 43.4 | (5.4) | 43.1 | (4.8) |
| mean (SD) | | | | | | | | | | | | |
| White/ | 33/63/4 | | | | | | 95/5/0 | | | | | |
| Black/ | | | | | | | | | | | | |
| Other (%) | | | | | | | | | | | | |
| BMI (kg/m²): | 32.2 | (6.8) | 33.3 | (7.4) | 33.0 | (7.3) | 26.8 | (5.4) | 27.4 | (5.7) | 26.8 | (5.5) |
| mean (SD) | | | | | | | | | | | | |
| Weight (kg): | 87.4 | (18.9) | 90.4 | (22.4) | 88.6 | (20.2) | 74.4 | (16.0) | 75.2 | (15.6) | 72.7 | (15.2) |
| mean (SD) | | | | | | | | | | | | |
| Hb < 10 | 26 | (25.2) | 31 | (33.0) | 31 | (30.4) | 14 | (13.7) | 21 | (21.6) | 24 | (24.5) |
| g/dL: n (%) | | | | | | | | | | | | |
| Hb < 12 | 76 | (73.8) | 68 | (72.3) | 72 | (70.6) | 51 | (50.0) | 61 | (62.9) | 56 | (57.1) |
| g/dL: n (%)* | | | | | | | | | | | | |
| MBL** (mL) | 195 | (110) | 197 | (110) | 195 | (117) | 218 | (128) | 247 | (162) | 213 | (143) |

mean (SD)

*Patients were characterized as anemic if they exhibited a hemoglobin (Hb) value of less than 12.0 g/dL
**Menstrual blood loss (MBL) was characterized as “heavy” if total blood lost per menstrual cycle exceeded 80 mL, as assessed by way of the alkaline hematin method, described herein.

### Dosing Regimens

Patients were randomized to one of several cohorts: (i) placebo, (ii) 100 mg/day of compound (VI), or (iii) 200 mg/day of compound (VI). Patients receiving 200 mg/day of compound (VI) also received once-daily ABT containing 1.0 mg E2 and 0.5 mg NETA.

### Endpoints

Patients were assessed for uterine fibroid symptomology at baseline, after 24 weeks of treatment, and again after 52 weeks of treatment. Particularly, to monitor the efficacy of compound (VI), patients were evaluated to determine the extent to which they exhibited a reduction in menstrual blood loss relative to their menstrual blood loss patterns at baseline. Patients were also assessed in order to determine the number of days of uterine bleeding during their last 28-day interval prior to week 24 of treatment with placebo or compound (VI).

Apart from analyzing the ability of compound (VI) to engender a reduction in uterine blood loss, patients were also assessed to determine the proportion of subjects that exhibited sustained amenorrhea after 24 weeks of treatment with compound (VI). Patients that exhibited anemia at baseline were evaluated to determine whether they exhibited an improvement in their condition, particularly by assessing whether they exhibited an increase in hemoglobin level during the studies. Pelvic pain is yet another symptom of uterine fibroids; accordingly, patients were also assessed to determine the extent to which they exhibited a reduction in pain during the studies. Pain levels were measured using a Verbal Rating Score (VRS), as described herein. Patients were also assessed to determine the proportion that reported an improvement in quality of life using the Uterine Fibroids Symptoms and Health-Related Quality of Life questionnaire.

To evaluate the safety of compound (VI), patients' bone mineral density levels were measured at baseline and at various timepoints during the studies.

### Results

Patients treated with compound (VI), either in an amount of 100 mg/day without ABT or in an amount of 200 mg/day with once-daily ABT, exhibited a sustained reduction in menstrual blood loss and pain symptoms throughout the duration of the PRIMROSE 1 and PRIMROSE 2 studies. Specifically, in the PRIMROSE 1 study, after 24 weeks of treatment, patients receiving compound (VI) experienced a clinically and statistically significant reduction in menstrual bleeding, defined as ≤80 mL of menstrual blood loss and a ≥50% reduction in menstrual blood loss from baseline. Among women receiving 200 mg of compound (VI) per day in combination with ABT, 75.5% (p<0.001) achieved a reduction in menstrual blood loss to a level of ≤80 mL and a ≥50% reduction in menstrual blood loss from baseline. Among patients receiving 100 mg of compound (VI) without ABT, 56.4% (p=0.003) achieved a reduction in menstrual blood loss to a level of ≤80 mL and a ≥50% reduction in menstrual blood loss from baseline. In contrast, among patients receiving placebo, 35% exhibited a reduction in menstrual blood loss to a level of ≤80 mL and a ≥50% reduction in menstrual blood loss from baseline.

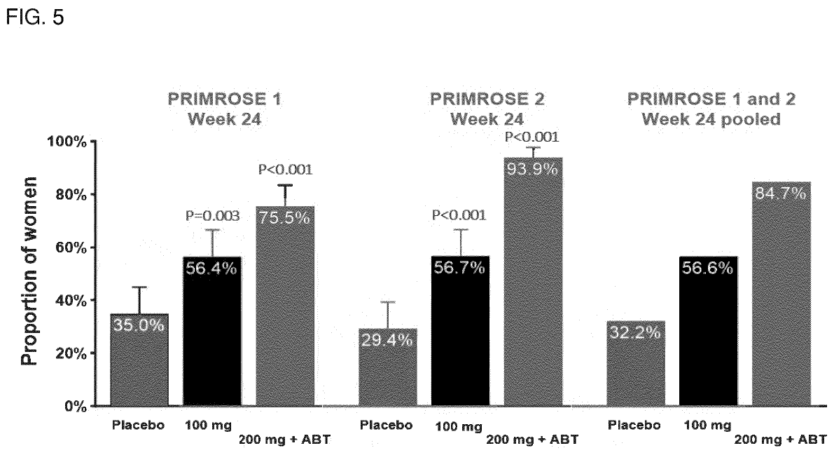

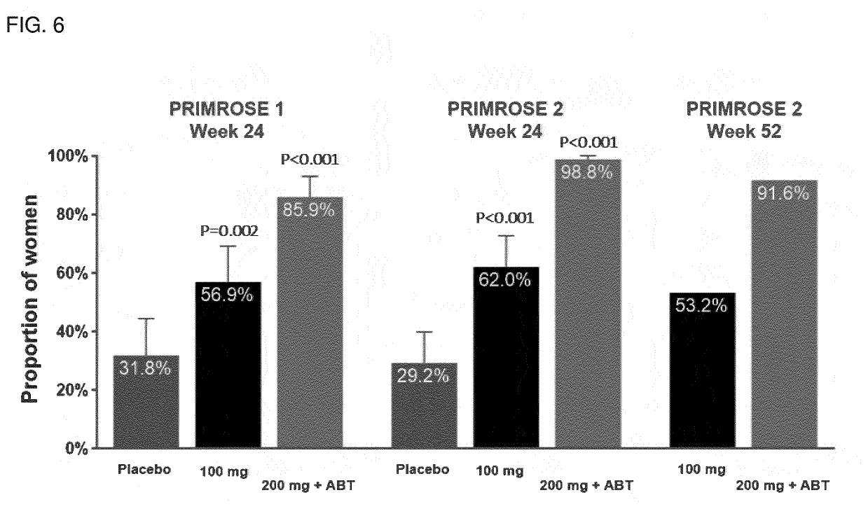

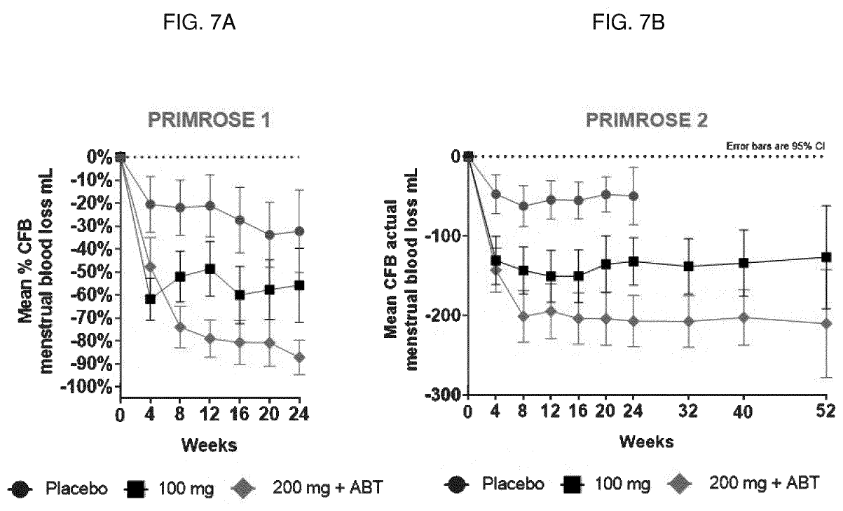

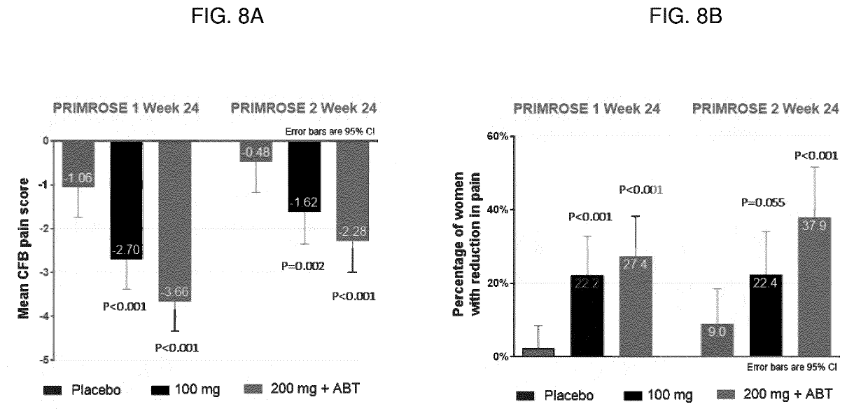

Importantly, the therapeutic effects of compound (VI) were sustained for up to 52 weeks, as demonstrated by the data obtained from the PRIMROSE 2 study. In the PRIMROSE2 trial, among patients being administered 200 mg of compound (VI) per day in combination with ABT for 52 weeks, 91.6% achieved a reduction in menstrual blood loss to a level of ≤80 mL and a ≥50% reduction in menstrual blood loss from baseline. Similarly, among patients receiving 100 mg of compound (VI) without ABT for 52 weeks, 53.2% achieved a reduction in menstrual blood loss to a level of 80 mL and a 50% reduction in menstrual blood loss from baseline. Both of these values mirror the response rates observed after 24 weeks of treatment in the PRIMROSE 2 study. These results are shown graphically in FIGS. 5-8.

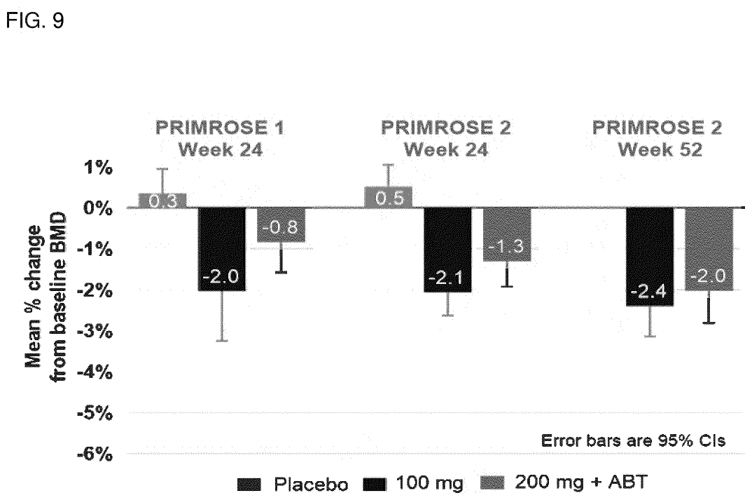

Not only was compound (VI) capable of attenuating uterine blood loss, reducing pain, and improving patients' quality of life, the data from the PRIMROSE 1 and PRIMROSE 2 studies demonstrate that compound (VI) does not induce a substantial decrease in bone mineral density. This is shown, for example, in FIG. 9, which depicts the lumbar spine bone mineral density of patients treated with compound (VI) after 24 weeks and 52 weeks of once-daily treatment with compound (VI).

The efficacy of compound (VI) in the PRIMROSE 1 and PRIMROSE 2 studies is summarized in Table 11, below.

TABLE 11
Efficacy of compound (VI) in reducing uterine fibroids
symptomology and improving quality of
life in PRIMROSE 1 and PRIMROSE 2 studies

| | | | 200 mg/day of | |
|---|---|---|---|---|
| | | | compound (VI) + | |
| | | | once-daily ABT | |
| | 100 mg/day of | | (1.0 mg E2 + 0.5 | |
| | compound (VI) | | mg NETA) | |
| | PRIMROSE | PRIMROSE | PRIMROSE | PRIMROSE |
| Endpoint | 1 | 2 | 1 | 2 |
| Reduction in | p = 0.002 | p < 0.001 | p < 0.001 | p < 0.001 |
| menstrual | p = 0.001 | p < 0.001 | p < 0.001 | p < 0.001 |
| blood loss | | | | |
| • Time to | | | | |
| reduced | | | | |
| menstrual | | | | |
| blood loss (≤80 | | | | |
| mL and ≥ 50% | | | | |
| reduction from | | | | |
| baseline) up to | | | | |
| Week 24 | | | | |
| • Number | | | | |
| of days | | | | |
| of uterine | | | | |
| bleeding for the | | | | |
| last 28-day | | | | |
| interval prior to | | | | |
| Week 24 | | | | |
| Amenorrhea | p = 0.009 | p < 0.001 | p < 0.001 | p < 0.001 |
| • Percentage at | p = 0.007 | p < 0.001 | p < 0.001 | p < 0.001 |
| Week 24 | | | | |
| • Time to | | | | |
| amenorrhea up | | | | |
| to Week 24 | | | | |
| Improvement in | p = 0.019 | p = 0.002 | p < 0.001 | p < 0.001 |
| anemia | | | | |
| Hemoglobin | | | | |
| level | | | | |
| at week 24 in | | | | |
| anemic | | | | |
| subjects⁺ | | | | |
| Reduction | p < 0.001 | p = 0.002 | p < 0.001 | p < 0.001 |
| in pain | | | | |
| Change from | | | | |
| baseline | | | | |
| pain score | | | | |
| at week 24 | | | | |
| Reduction in | p = 0.149 | p = 0.055 | p = 0.671 | p = 0.008 |
| volume | p = 0.014 | p = 0.003 | p = 0.223 | p < 0.001 |
| • Fibroid | | | | |
| volume | | | | |
| change from | | | | |
| baseline at | | | | |
| Week 24 | | | | |
| • Uterine | | | | |
| volume | | | | |
| change from | | | | |
| baseline at | | | | |
| Week 24 | | | | |
| Improvement in | p = 0.002 | p = 0.004 | p < 0.001 | p < 0.001 |
| quality of life | | | | |
| Change from | | | | |
| baseline | | | | |
| in quality | | | | |
| of life (QoL) | | | | |
| symptom | | | | |
| severity | | | | |
| score | | | | |
| (LS mean) | | | | |

⁺Defined as subjects with Hb < 12 g/dL at baseline

### CONCLUSION

As the results of the PRIMROSE 1 and PRIMROSE 2 studies show, compound (VI) can safely be administered to patients over the course of extended treatment periods, such as those having a duration of one year or longer, so as to reduce estrogen-dependent disease symptomology and treat the underlying etiology of the disease. The therapeutic effects of compound (VI) that are observed after initial treatment periods (e.g., of 24 weeks) are observed over extended periods of time (e.g., for 52 weeks or longer). Advantageously, compound (VI) achieves these desirable treatment outcomes without inducing a substantial reduction in bone mineral density.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating an estrogen-dependent disease in a human patient in need thereof, the method comprising:
a) periodically administering to the patient a therapeutically effective amount of a gonadotropin-releasing hormone (GnRH) antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

2. The method of claim 1, wherein the estrogen-dependent disease is uterine fibroids.

3. A method of reducing the volume of menstrual blood loss in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

4. A method of inducing amenorrhea in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

5. A method of reducing the volume of one or more uterine fibroids in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

6. A method of preserving or increasing hemoglobin in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

7. The method of claim 1, wherein the estrogen-dependent disease is endometriosis.

8. A method of reducing the volume of one or more endometriosis nodes in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

9. A method of reducing pelvic pain in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

10. A method of reducing dysmenorrhea in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

11. A method of reducing dyspareunia in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

12. A method of reducing dyschezia in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

13. A method of reducing uterine bleeding in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

14. A method of inducing amenorrhea in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

15. The method of claim 1, wherein the estrogen-dependent disease is adenomyosis.

16. A method of reducing uterine volume in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

17. A method of reducing the thickness of the anterior and/or posterior region of the uterine myometrium in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

18. A method of reducing pelvic pain in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

19. A method of reducing dysmenorrhea in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

20. A method of reducing dyspareunia in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

21. A method of reducing dyschezia in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

22. A method of reducing uterine tenderness in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

23. A method of reducing uterine bleeding in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

24. A method of inducing amenorrhea in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

25. The method of claim 1, wherein the estrogen-dependent disease is rectovaginal endometriosis.

26. A method of reducing the volume of one or more rectovaginal endometriosis nodes in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

27. A method of reducing the volume of one or more type III rectovaginal endometriosis nodes in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

28. A method of reducing pelvic pain in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

29. A method of reducing dysmenorrhea in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

30. A method of reducing dyspareunia in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

31. A method of reducing dyschezia in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

32. A method of reducing uterine bleeding in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

33. A method of inducing amenorrhea in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the second treatment period commences at least one week after the end of the first treatment period, and wherein the patient is not administered a GnRH antagonist during the time elapsed between the end of the first treatment period and commencement of the second treatment period.

34. The method of any one of claims 1-33, wherein the amount of the GnRH antagonist that is periodically administered to the patient during the second treatment period is less than the amount of the GnRH antagonist that is periodically administered to the patient during the first treatment period.

35. The method of any one of claims 1-34, wherein the GnRH antagonist that is periodically administered to the patient during the first treatment period is not a compound represented by formula (I), and the GnRH antagonist that is periodically administered to the patient during the second treatment period is a compound represented by formula (I) 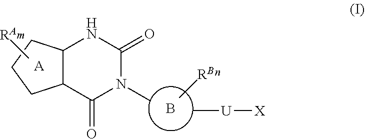
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

36. A method of treating an estrogen-dependent disease in a human patient in need thereof, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

37. The method of claim 36, wherein the estrogen-dependent disease is uterine fibroids.

38. A method of reducing the volume of menstrual blood loss in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

39. A method of inducing amenorrhea in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

40. A method of reducing the volume of one or more uterine fibroids in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

41. A method of preserving or increasing hemoglobin in a human patient diagnosed as having uterine fibroids, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

42. The method of claim 36, wherein the estrogen-dependent disease is endometriosis.

43. A method of reducing the volume of one or more endometriosis nodes in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

44. A method of reducing pelvic pain in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

45. A method of reducing dysmenorrhea in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

46. A method of reducing dyspareunia in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

47. A method of reducing dyschezia in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

48. A method of reducing uterine bleeding in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

49. A method of inducing amenorrhea in a human patient diagnosed as having endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

50. The method of claim 36, wherein the estrogen-dependent disease is adenomyosis.

51. A method of reducing uterine volume in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

52. A method of reducing the thickness of the anterior and/or posterior region of the uterine myometrium in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

53. A method of reducing pelvic pain in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

54. A method of reducing dysmenorrhea in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

55. A method of reducing dyspareunia in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

56. A method of reducing dyschezia in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

57. A method of reducing uterine tenderness in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

58. A method of reducing uterine bleeding in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

59. A method of inducing amenorrhea in a human patient diagnosed as having adenomyosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

60. The method of claim 36, wherein the estrogen-dependent disease is rectovaginal endometriosis.

61. A method of reducing the volume of one or more rectovaginal endometriosis nodes in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

62. A method of reducing the volume of one or more type III rectovaginal endometriosis nodes in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

63. A method of reducing pelvic pain in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

64. A method of reducing dysmenorrhea in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

65. A method of reducing dyspareunia in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

66. A method of reducing dyschezia in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

67. A method of reducing uterine bleeding in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

68. A method of inducing amenorrhea in a human patient diagnosed as having rectovaginal endometriosis, the method comprising:
a) periodically administering to the patient a therapeutically effective amount of a GnRH antagonist during a first treatment period,
b) monitoring the patient's bone mineral density at the end of the first treatment period, and, if the patient is determined to exhibit a reduced bone mineral density at the end of the first treatment period relative to a measurement of the patient's bone mineral density obtained during, or prior to commencement of, the first treatment period,
c) periodically administering to the patient a therapeutically effective amount of a GnRH antagonist during a second treatment period, wherein the amount of the GnRH antagonist that is periodically administered to the patient during the second treatment period is less than the amount of the GnRH antagonist that is periodically administered to the patient during the first treatment period.

69. The method of any one of claims 36-68, wherein the second treatment period commences at least one week after the end of the first treatment period.

70. The method of claim 69, wherein the patient is not administered a GnRH antagonist during the time elapsed between the end of the first treatment period and commencement of the second treatment period.

71. The method of any one of claims 36-70, wherein the GnRH antagonist that is periodically administered to the patient during the first treatment period is not a compound represented by formula (I), and the GnRH antagonist that is periodically administered to the patient during the second treatment period is a compound represented by formula (I) 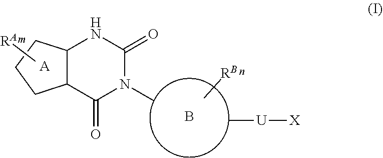
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

72. A method of treating an estrogen-dependent disease in a human patient in need thereof, the method comprising periodically administering to the patient, during a second treatment period, a therapeutically effective amount of a GnRH antagonist represented by formula (I) 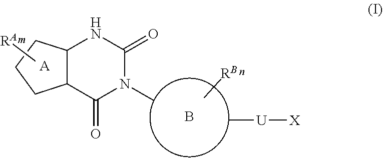
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

73. The method of claim 72, wherein the estrogen-dependent disease is uterine fibroids.

74. A method of reducing the volume of menstrual blood loss in a human patient diagnosed as having uterine fibroids, the method comprising periodically administering to the patient, during a second treatment period, a therapeutically effective amount of a GnRH antagonist represented by formula (I) 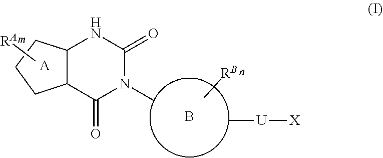
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or WV and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

75. A method of inducing amenorrhea in a human patient diagnosed as having uterine fibroids, the method comprising periodically administering to the patient, during a second treatment period, a therapeutically effective amount of a GnRH antagonist represented by formula (I) 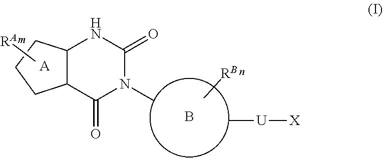
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

76. A method of reducing the volume of one or more uterine fibroids in a human patient diagnosed as having uterine fibroids, the method comprising periodically administering to the patient, during a second treatment period, a therapeutically effective amount of a GnRH antagonist represented by formula (I) 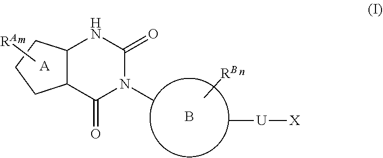
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and WV are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

77. A method of preserving or increasing hemoglobin in a human patient diagnosed as having uterine fibroids, the method comprising periodically administering to the patient, during a second treatment period, a therapeutically effective amount of a GnRH antagonist represented by formula (I) 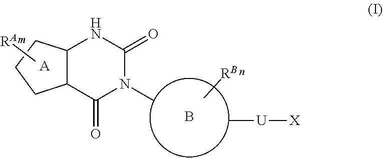
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W² to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

78. The method of claim 72, wherein the estrogen-dependent disease is endometriosis.

79. A method of reducing the volume of one or more endometriosis nodes in a human patient diagnosed as having endometriosis, the method comprising periodically administering to the patient, during a second treatment period, a therapeutically effective amount of a GnRH antagonist represented by formula (I) 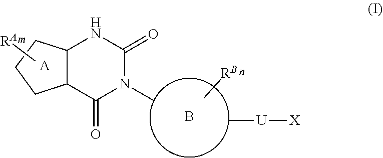
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

80. A method of reducing pelvic pain in a human patient diagnosed as having endometriosis, the method comprising periodically administering to the patient, during a second treatment period, a therapeutically effective amount of a GnRH antagonist represented by formula (I) 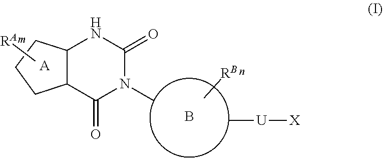
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

81. A method of reducing dysmenorrhea in a human patient diagnosed as having endometriosis, the method comprising periodically administering to the patient, during a second treatment period, a therapeutically effective amount of a GnRH antagonist represented by formula (I) 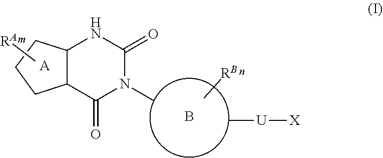
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

82. A method of reducing dyspareunia in a human patient diagnosed as having endometriosis, the method comprising periodically administering to the patient, during a second treatment period, a therapeutically effective amount of a GnRH antagonist represented by formula (I) 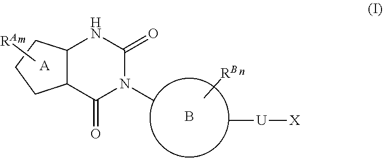
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

83. A method of reducing dyschezia in a human patient diagnosed as having endometriosis, the method comprising periodically administering to the patient, during a second treatment period, a therapeutically effective amount of a GnRH antagonist represented by formula (I) 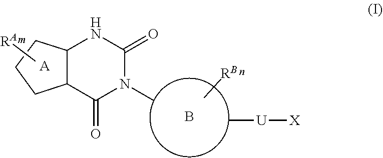
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

84. A method of reducing uterine bleeding in a human patient diagnosed as having endometriosis, the method comprising periodically administering to the patient, during a second treatment period, a therapeutically effective amount of a GnRH antagonist represented by formula (I) 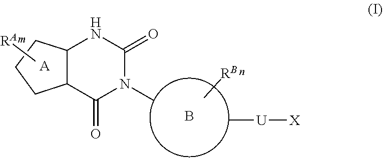
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

85. A method of inducing amenorrhea in a human patient diagnosed as having endometriosis, the method comprising periodically administering to the patient, during a second treatment period, a therapeutically effective amount of a GnRH antagonist represented by formula (I) 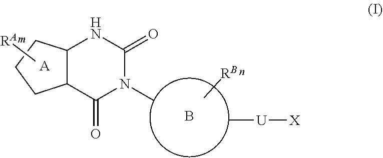
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

86. The method of claim 72, wherein the estrogen-dependent disease is adenomyosis.

87. A method of reducing uterine volume in a human patient diagnosed as having adenomyosis, the method comprising periodically administering to the patient, during a second treatment period, a therapeutically effective amount of a GnRH antagonist represented by formula (I) 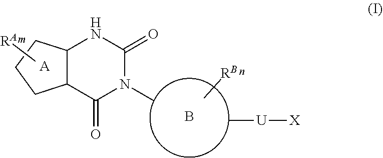
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

88. A method of reducing the thickness of the anterior and/or posterior region of the uterine myometrium in a human patient diagnosed as having adenomyosis, the method comprising periodically administering to the patient, during a second treatment period, a therapeutically effective amount of a GnRH antagonist represented by formula (I) 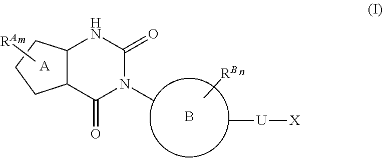
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

89. A method of reducing pelvic pain in a human patient diagnosed as having adenomyosis, the method comprising periodically administering to the patient, during a second treatment period, a therapeutically effective amount of a GnRH antagonist represented by formula (I) 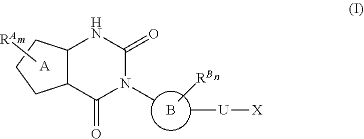
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

90. A method of reducing dysmenorrhea in a human patient diagnosed as having adenomyosis, the method comprising periodically administering to the patient, during a second treatment period, a therapeutically effective amount of a GnRH antagonist represented by formula (I) 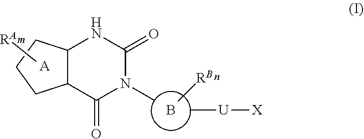
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

91. A method of reducing dyspareunia in a human patient diagnosed as having adenomyosis, the method comprising periodically administering to the patient, during a second treatment period, a therapeutically effective amount of a GnRH antagonist represented by formula (I) 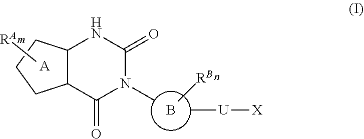
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

92. A method of reducing dyschezia in a human patient diagnosed as having adenomyosis, the method comprising periodically administering to the patient, during a second treatment period, a therapeutically effective amount of a GnRH antagonist represented by formula (I) 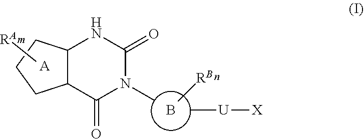
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

93. A method of reducing uterine tenderness in a human patient diagnosed as having adenomyosis, the method comprising periodically administering to the patient, during a second treatment period, a therapeutically effective amount of a GnRH antagonist represented by formula (I) 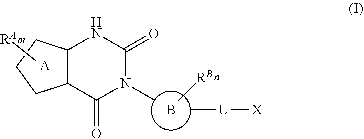
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

94. A method of reducing uterine bleeding in a human patient diagnosed as having adenomyosis, the method comprising periodically administering to the patient, during a second treatment period, a therapeutically effective amount of a GnRH antagonist represented by formula (I) 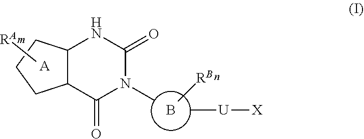
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

95. A method of inducing amenorrhea in a human patient diagnosed as having adenomyosis, the method comprising periodically administering to the patient, during a second treatment period, a therapeutically effective amount of a GnRH antagonist represented by formula (I) 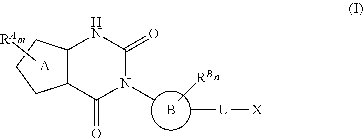
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

96. The method of claim 72, wherein the estrogen-dependent disease is rectovaginal endometriosis.

97. A method of reducing the volume of one or more rectovaginal endometriosis nodes in a human patient diagnosed as having rectovaginal endometriosis, the method comprising periodically administering to the patient, during a second treatment period, a therapeutically effective amount of a GnRH antagonist represented by formula (I) 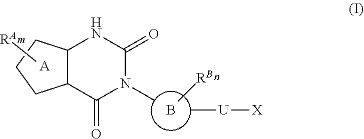
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

98. A method of reducing the volume of one or more type III rectovaginal endometriosis nodes in a human patient diagnosed as having rectovaginal endometriosis, the method comprising periodically administering to the patient, during a second treatment period, a therapeutically effective amount of a GnRH antagonist represented by formula (I) 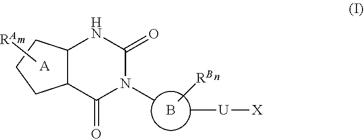
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

99. A method of reducing pelvic pain in a human patient diagnosed as having rectovaginal endometriosis, the method comprising periodically administering to the patient, during a second treatment period, a therapeutically effective amount of a GnRH antagonist represented by formula (I) 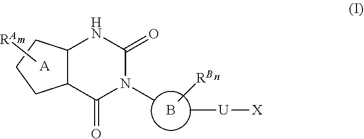
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

100. A method of reducing dysmenorrhea in a human patient diagnosed as having rectovaginal endometriosis, the method comprising periodically administering to the patient, during a second treatment period, a therapeutically effective amount of a GnRH antagonist represented by formula (I) 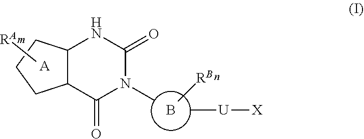
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

101. A method of reducing dyspareunia in a human patient diagnosed as having rectovaginal endometriosis, the method comprising periodically administering to the patient, during a second treatment period, a therapeutically effective amount of a GnRH antagonist represented by formula (I) 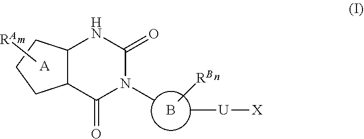
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

102. A method of reducing dyschezia in a human patient diagnosed as having rectovaginal endometriosis, the method comprising periodically administering to the patient, during a second treatment period, a therapeutically effective amount of a GnRH antagonist represented by formula (I) 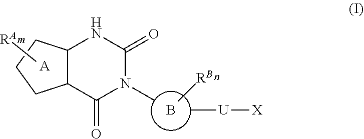
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

103. A method of reducing uterine bleeding in a human patient diagnosed as having rectovaginal endometriosis, the method comprising periodically administering to the patient, during a second treatment period, a therapeutically effective amount of a GnRH antagonist represented by formula (I) 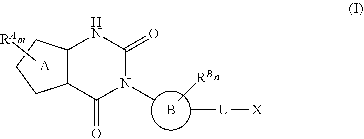
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

104. A method of inducing amenorrhea in a human patient diagnosed as having rectovaginal endometriosis, the method comprising periodically administering to the patient, during a second treatment period, a therapeutically effective amount of a GnRH antagonist represented by formula (I) 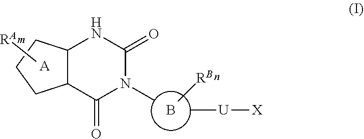
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof,
wherein the patient has previously been administered, during a first treatment period, a GnRH antagonist that is not a compound represented by formula (I).

105. The method of any one of claims 72-104, wherein the GnRH antagonist that is not a compound represented by formula (I) has been periodically administered to the patient during the first treatment period.

106. The method of any one of claims 72-105, wherein the patient has been determined to exhibit a reduced bone mineral density at the end of the first treatment period relative to a measurement of the patient's bone mineral density obtained during, or prior to commencement of, the first treatment period.

107. The method of any one of claims 72-106, wherein the second treatment period commences at least one week after the end of the first treatment period.

108. The method of claim 107, wherein the patient is not administered a GnRH antagonist during the time elapsed between the end of the first treatment period and commencement of the second treatment period.

109. The method of any one of claims 72-108, wherein the amount of the GnRH antagonist that is periodically administered to the patient during the second treatment period is less than the amount of the GnRH antagonist that is periodically administered to the patient during the first treatment period.

110. The method of any one of claims 1-34 and 36-70, wherein the GnRH antagonist administered during the first treatment period is a compound represented by formula (I) 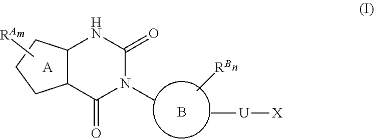
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

111. The method of claim 110, wherein the ring A is a thiophene ring represented by formula (IIa)

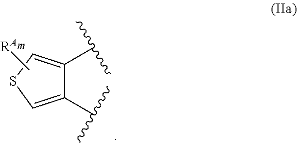

112. The method of claim 110 or 111, wherein m is 1.

113. The method of claim 112, wherein the ring A is an optionally substituted thiophene ring represented by formula (IIb)

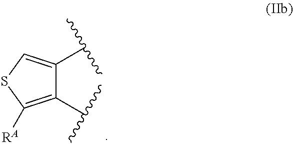

114. The method of any one of claims 110-113, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

115. The method of claim 114, wherein each R^{A} is COOH or pharmaceutically acceptable salt thereof.

116. The method of any one of claims 110-115, wherein the ring B is an optionally substituted benzene ring, pyridine ring, or thiophene ring.

117. The method of claim 116, wherein the ring B is represented by a formula selected from the group consisting of:

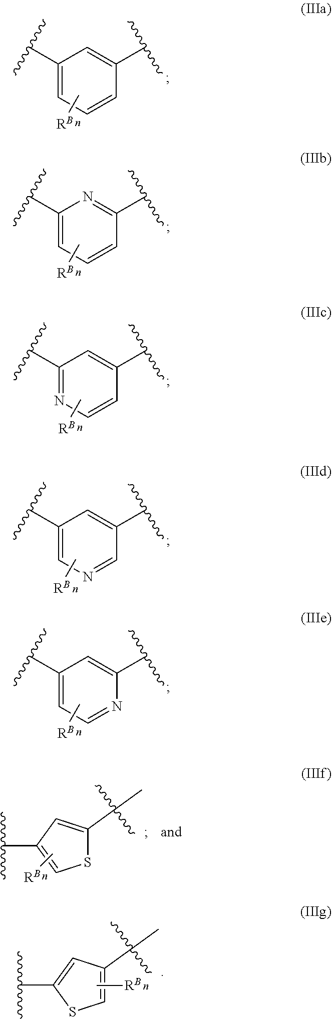

118. The method of any one of claims 110-117, wherein n is 2.

119. The method of claim 118, wherein the ring B is represented by a formula selected from the group consisting of:

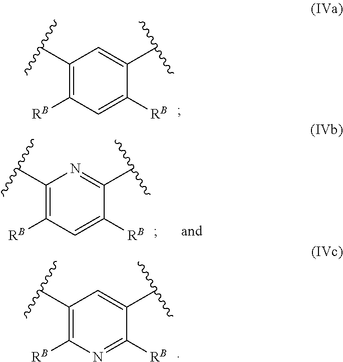

120. The method of any one of claims 110-119, wherein each R^{B} is independently a halogen atom, an optionally substituted lower alkyl group, or OW⁴, wherein each W⁴ is independently a hydrogen atom or an optionally substituted lower alkyl group.

121. The method of claim 120, wherein each R^{B} is independently a fluorine atom, chlorine atom, bromine atom, methyl group, or methoxy group.

122. The method of any one of claims 110-121, wherein U is a single bond.

123. The method of any one of claims 110-122, wherein X is a group represented by —O-L-Y.

124. The method of any one of claims 110-123, wherein L is a methylene group.

125. The method of any one of claims 110-124, wherein Y is an optionally substituted benzene ring represented by formula (V) 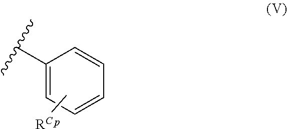
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

126. The method of claim 125, wherein Y is a substituted benzene ring represented by formula (Va)

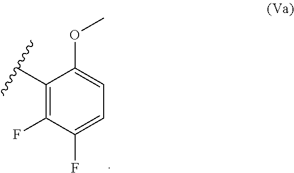 [SMILES: *c1c(OC)ccc(F)c1F]

127. The method of claim 110, wherein the compound is represented by formula (Ia) 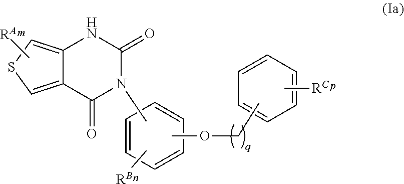
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

128. The method of claim 127, wherein the compound is represented by formula (Ib)

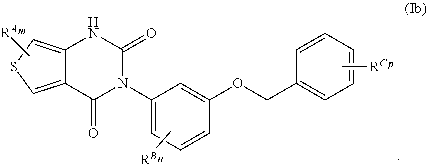

129. The method of claim 128, wherein the compound is represented by formula (Ic) 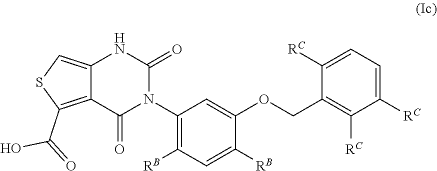
or a pharmaceutically acceptable salt thereof.

130. The method of claim any one of claims 110-129, wherein the compound is represented by formula (VI) 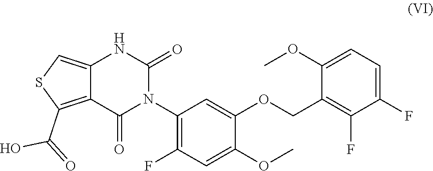 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

131. The method of claim 130, wherein the compound is the choline salt of the compound represented by formula (VI).

132. The method of claim 131, wherein the compound is in a crystalline state.

133. The method of claim 132, wherein the compound exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

134. The method of claim 132 or 133, wherein the compound exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

135. The method of any one of claims 132-134, wherein the compound exhibits ¹⁹F solid-state NMR peaks centered at about −151.8 ppm, −145.2 ppm, and −131.6 ppm.

136. The method of any one of claims 110-135, wherein the compound is orally administered to the patient during the first treatment period.

137. The method of any one of claims 110-136, wherein the compound is administered to the patient one or more times per day, week, or month during the first treatment period.

138. The method of claim 137, wherein the compound is administered to the patient one or more times daily during the first treatment period.

139. The method of claim 138, wherein the compound is administered to the patient once daily during the first treatment period.

140. The method of any one of claims 137-139, wherein the compound is administered to the patient in an amount of from about 25 mg per day to about 400 mg per day during the first treatment period.

141. The method of claim 140, wherein the compound is administered to the patient in an amount of from about 35 mg to about 65 mg per day during the first treatment period.

142. The method of claim 141, wherein the compound is administered to the patient in an amount of about 50 mg per day during the first treatment period.

143. The method of claim 140, wherein the compound is administered to the patient in an amount of from about 60 mg per day to about 90 mg per day during the first treatment period.

144. The method of claim 143, wherein the compound is administered to the patient in an amount of about 75 mg per day during the first treatment period.

145. The method of claim 140, wherein the compound is administered to the patient in an amount of from about 85 mg to about 115 mg per day during the first treatment period.

146. The method of claim 145, wherein the compound is administered to the patient in an amount of about 100 mg per day during the first treatment period.

147. The method of claim 140, wherein the compound is administered to the patient in an amount of from about 185 mg to about 215 mg per day during the first treatment period.

148. The method of claim 147, wherein the compound is administered to the patient in an amount of about 200 mg per day during the first treatment period.

149. The method of any one of claims 1-109, wherein the GnRH antagonist administered during the first treatment period is elagolix, relugolix, opigolix (ASP1707), BAY-784, or SK-2706.

150. The method of claim 149, wherein the GnRH antagonist administered during the first treatment period is elagolix.

151. The method of claim 150, wherein the GnRH antagonist is orally administered to the patient during the first treatment period.

152. The method of claim 150 or 151, wherein the GnRH antagonist is administered to the patient one or more times per day, week, or month during the first treatment period.

153. The method of claim 152, wherein the GnRH antagonist is administered to the patient one or more times daily during the first treatment period.

154. The method of claim 153, wherein the GnRH antagonist is administered to the patient once daily during the first treatment period.

155. The method of any one of claims 150-154, wherein the GnRH antagonist is administered to the patient in an amount of from about 100 mg to about 600 mg per day during the first treatment period.

156. The method of claim 155, wherein the GnRH antagonist is administered to the patient in an amount of about 150 mg per day during the first treatment period.

157. The method of claim 155, wherein the GnRH antagonist is administered to the patient in an amount of about 300 mg per day during the first treatment period.

158. The method of claim 155, wherein the GnRH antagonist is administered to the patient in an amount of about 400 mg per day during the first treatment period, optionally wherein the GnRH antagonist is administered to the patient in two daily doses of 200 mg per dose during the first treatment period.

159. The method of claim 155, wherein the GnRH antagonist is administered to the patient in an amount of about 600 mg per day during the first treatment period, optionally wherein the GnRH antagonist is administered to the patient in two daily doses of 300 mg per dose during the first treatment period.

160. The method of claim 149, wherein the GnRH antagonist administered during the first treatment period is relugolix.

161. The method of claim 160, wherein the GnRH antagonist is orally administered to the patient during the first treatment period.

162. The method of claim 160 or 161, wherein the GnRH antagonist is administered to the patient one or more times per day, week, or month during the first treatment period.

163. The method of claim 162, wherein the GnRH antagonist is administered to the patient one or more times daily during the first treatment period.

164. The method of claim 163, wherein the GnRH antagonist is administered to the patient once daily during the first treatment period.

165. The method of any one of claims 160-164, wherein the GnRH antagonist is administered to the patient in an amount of from about 10 mg to about 60 mg per day during the first treatment period.

166. The method of claim 165, wherein the GnRH antagonist is administered to the patient in an amount of from about 20 mg to about 50 mg per day during the first treatment period.

167. The method of claim 166, wherein the GnRH antagonist is administered to the patient in an amount of about 40 mg per day during the first treatment period.

168. The method of any one of claims 1-34, 36-70, and 110-167, wherein the GnRH antagonist administered during the second treatment period is a compound represented by formula (I) 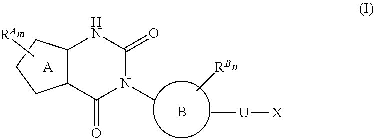
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W and v are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

169. The method of claim 168, wherein the ring A is a thiophene ring represented by formula (IIa)

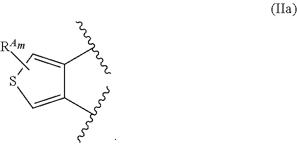

170. The method of claim 168 or 169, wherein m is 1.

171. The method of claim 170, wherein the ring A is an optionally substituted thiophene ring represented by formula (IIb)

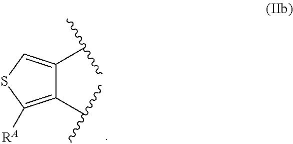

172. The method of any one of claims 168-171, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

173. The method of claim 172, wherein each R^{A} is COOH or pharmaceutically acceptable salt thereof.

174. The method of any one of claims 168-173, wherein the ring B is an optionally substituted benzene ring, pyridine ring, or thiophene ring.

175. The method of claim 174, wherein the ring B is represented by a formula selected from the group consisting of:

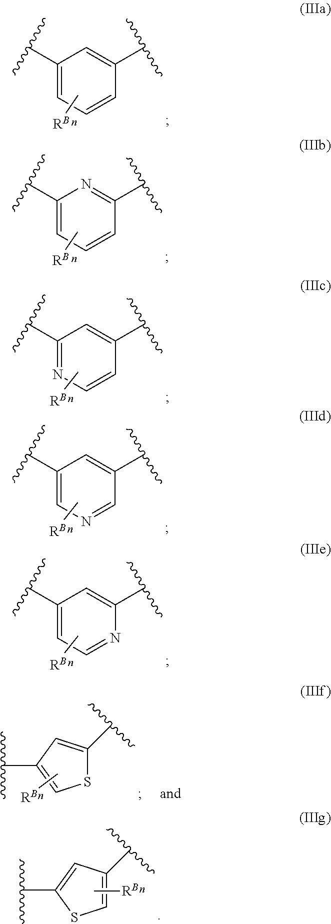

176. The method of any one of claims 168-175, wherein n is 2.

177. The method of claim 176, wherein the ring B is represented by a formula selected from the group consisting of:

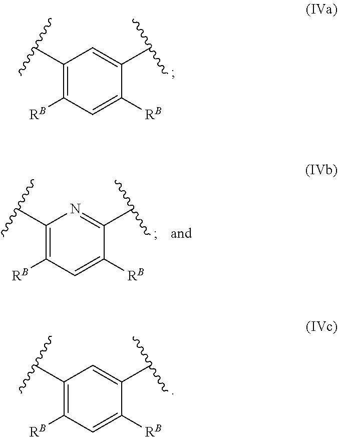

178. The method of any one of claims 168-177, wherein each R^{B} is independently a halogen atom, an optionally substituted lower alkyl group, or OW⁴, wherein each W⁴ is independently a hydrogen atom or an optionally substituted lower alkyl group.

179. The method of claim 178, wherein each R^{B} is independently a fluorine atom, chlorine atom, bromine atom, methyl group, or methoxy group.

180. The method of any one of claims 168-179, wherein U is a single bond.

181. The method of any one of claims 168-180, wherein X is a group represented by —O-L-Y.

182. The method of any one of claims 168-181, wherein L is a methylene group.

183. The method of any one of claims 168-182, wherein Y is an optionally substituted benzene ring represented by formula (V) 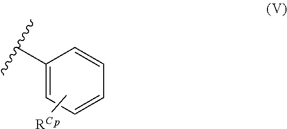
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

184. The method of claim 183, wherein Y is a substituted benzene ring represented by formula (Va)

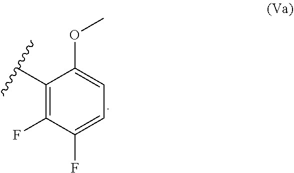 [SMILES: *c1c(OC)ccc(F)c1F]

185. The method of claim 168, wherein the compound is represented by formula (Ia) 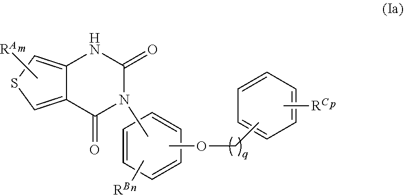
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

186. The method of claim 185, wherein the compound is represented by formula (Ib)

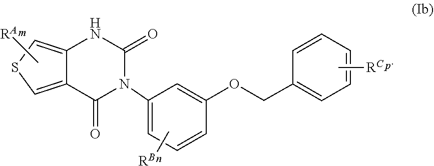

187. The method of claim 186, wherein the compound is represented by formula (Ic) 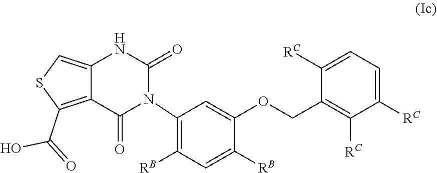
or a pharmaceutically acceptable salt thereof.

188. The method of claim any one of claims 168-187, wherein the compound is represented by formula (VI) 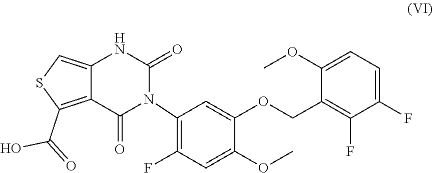 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

189. The method of claim 188, wherein the compound is the choline salt of the compound represented by formula (VI).

190. The method of claim 189, wherein the compound is in a crystalline state.

191. The method of claim 190, wherein the compound exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

192. The method of claim 190 or 191, wherein the compound exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

193. The method of any one of claims 190-192, wherein the compound exhibits ¹⁹F solid-state NMR peaks centered at about −151.8 ppm, −145.2 ppm, and −131.6 ppm.

194. The method of any one of claims 168-193, wherein the compound is orally administered to the patient during the second treatment period.

195. The method of any one of claims 168-194, wherein the compound is administered to the patient one or more times per day, week, or month during the second treatment period.

196. The method of claim 195, wherein the compound is administered to the patient one or more times daily during the second treatment period.

197. The method of claim 196, wherein the compound is administered to the patient once daily during the second treatment period.

198. The method of any one of claims 195-197, wherein the compound is administered to the patient in an amount of from about 25 mg per day to about 400 mg per day during the second treatment period.

199. The method of claim 198, wherein the compound is administered to the patient in an amount of from about 35 mg to about 65 mg per day during the second treatment period.

200. The method of claim 199, wherein the compound is administered to the patient in an amount of about 50 mg per day during the second treatment period.

201. The method of claim 198, wherein the compound is administered to the patient in an amount of from about 60 mg per day to about 90 mg per day during the second treatment period.

202. The method of claim 201, wherein the compound is administered to the patient in an amount of about 75 mg per day during the second treatment period.

203. The method of claim 198, wherein the compound is administered to the patient in an amount of from about 85 mg to about 115 mg per day during the second treatment period.

204. The method of claim 203, wherein the compound is administered to the patient in an amount of about 100 mg per day during the second treatment period.

205. The method of claim 198, wherein the compound is administered to the patient in an amount of from about 185 mg to about 215 mg per day during the second treatment period.

206. The method of claim 205, wherein the compound is administered to the patient in an amount of about 200 mg per day during the second treatment period.

207. The method of any one of claims 1-34, 36-70, and 110-167, wherein the GnRH antagonist administered during the second treatment period is elagolix, relugolix, opigolix (ASP1707), BAY-784, or SK-2706.

208. The method of claim 207, wherein the GnRH antagonist administered during the second treatment period is elagolix.

209. The method of claim 208, wherein the GnRH antagonist is orally administered to the patient during the second treatment period.

210. The method of claim 208 or 209, wherein the GnRH antagonist is administered to the patient one or more times per day, week, or month during the second treatment period.

211. The method of claim 210, wherein the GnRH antagonist is administered to the patient one or more times daily during the second treatment period.

212. The method of claim 211, wherein the GnRH antagonist is administered to the patient once daily during the second treatment period.

213. The method of any one of claims 208-212, wherein the GnRH antagonist is administered to the patient in an amount of from about 100 mg to about 600 mg per day during the second treatment period.

214. The method of claim 213, wherein the GnRH antagonist is administered to the patient in an amount of about 150 mg per day during the second treatment period.

215. The method of claim 213, wherein the GnRH antagonist is administered to the patient in an amount of about 300 mg per day during the second treatment period.

216. The method of claim 213, wherein the GnRH antagonist is administered to the patient in an amount of about 400 mg per day during the second treatment period, optionally wherein the GnRH antagonist is administered to the patient in two daily doses of 200 mg per dose during the second treatment period.

217. The method of claim 213, wherein the GnRH antagonist is administered to the patient in an amount of about 600 mg per day during the second treatment period, optionally wherein the GnRH antagonist is administered to the patient in two daily doses of 300 mg per dose during the second treatment period.

218. The method of claim 207, wherein the GnRH antagonist administered during the second treatment period is relugolix.

219. The method of claim 218, wherein the GnRH antagonist is orally administered to the patient during the second treatment period.

220. The method of claim 218 or 219, wherein the GnRH antagonist is administered to the patient one or more times per day, week, or month during the second treatment period.

221. The method of claim 220, wherein the GnRH antagonist is administered to the patient one or more times daily during the second treatment period.

222. The method of claim 221, wherein the GnRH antagonist is administered to the patient once daily during the second treatment period.

223. The method of any one of claims 218-222, wherein the GnRH antagonist is administered to the patient in an amount of from about 10 mg to about 60 mg per day during the second treatment period.

224. The method of claim 223, wherein the GnRH antagonist is administered to the patient in an amount of from about 20 mg to about 50 mg per day during the second treatment period.

225. The method of claim 224, wherein the GnRH antagonist is administered to the patient in an amount of about 40 mg per day during the second treatment period.

226. The method of any one of claims 1-225, wherein the first treatment period has a duration of at least four weeks.

227. The method of claim 226, wherein the first treatment period has a duration of at least eight weeks.

228. The method of claim 227, wherein the first treatment period has a duration of at least 10 weeks.

229. The method of claim 228, wherein the first treatment period has a duration of at least 12 weeks.

230. The method of claim 229, wherein the first treatment period has a duration of at least 24 weeks.

231. The method of any one of claims 1-225, wherein the first treatment period has a duration of from about four weeks to about 12 months.

232. The method of claim 231, wherein the first treatment period has a duration of from about four weeks to about 44 weeks.

233. The method of claim 232, wherein the first treatment period has a duration of from about four weeks to about 40 weeks.

234. The method of claim 233, wherein the first treatment period has a duration of from about four weeks to about 36 weeks.

235. The method of claim 234, wherein the first treatment period has a duration of from about four weeks to about 24 weeks.

236. The method of claim 235, wherein the first treatment period has a duration of from about five weeks to about 20 weeks.

237. The method of claim 236, wherein the first treatment period has a duration of from about six weeks to about 18 weeks.

238. The method of claim 237, wherein the first treatment period has a duration of from about eight weeks to about 16 weeks.

239. The method of claim 238, wherein the first treatment period has a duration of from about 10 weeks to about 14 weeks.

240. The method of claim 239, wherein the first treatment period has a duration of about 12 weeks.

241. The method of claim 231, wherein the first treatment period has a duration of from about 14 weeks to about 40 weeks.

242. The method of claim 241, wherein the first treatment period has a duration of from about 16 weeks to about 32 weeks.

243. The method of claim 242, wherein the first treatment period has a duration of from about 18 weeks to about 30 weeks.

244. The method of claim 243, wherein the first treatment period has a duration of from about 20 weeks to about 28 weeks.

245. The method of claim 244, wherein the first treatment period has a duration of from about 22 weeks to about 26 weeks.

246. The method of claim 245, wherein the first treatment period has a duration of about 24 weeks.

247. The method of any one of claims 1-246, wherein the second treatment period has a duration of at least four weeks.

248. The method of claim 247, wherein the second treatment period has a duration of at least eight weeks.

249. The method of claim 248, wherein the second treatment period has a duration of at least 10 weeks.

250. The method of claim 249, wherein the second treatment period has a duration of at least 12 weeks.

251. The method of claim 250, wherein the second treatment period has a duration of at least 24 weeks.

252. The method of any one of claims 1-246, wherein the second treatment period has a duration of from about four weeks to about 12 months.

253. The method of claim 252, wherein the second treatment period has a duration of from about four weeks to about 44 weeks.

254. The method of claim 253, wherein the second treatment period has a duration of from about four weeks to about 40 weeks.

255. The method of claim 254, wherein the second treatment period has a duration of from about four weeks to about 36 weeks.

256. The method of claim 255, wherein the second treatment period has a duration of from about four weeks to about 24 weeks.

257. The method of claim 256, wherein the second treatment period has a duration of from about five weeks to about 20 weeks.

258. The method of claim 257, wherein the second treatment period has a duration of from about six weeks to about 18 weeks.

259. The method of claim 258, wherein the second treatment period has a duration of from about eight weeks to about 16 weeks.

260. The method of claim 259, wherein the second treatment period has a duration of from about 10 weeks to about 14 weeks.

261. The method of claim 260, wherein the second treatment period has a duration of about 12 weeks.

262. The method of claim 252, wherein the second treatment period has a duration of from about 14 weeks to about 40 weeks.

263. The method of claim 262, wherein the second treatment period has a duration of from about 16 weeks to about 32 weeks.

264. The method of claim 263, wherein the second treatment period has a duration of from about 18 weeks to about 30 weeks.

265. The method of claim 264, wherein the second treatment period has a duration of from about 20 weeks to about 28 weeks.

266. The method of claim 265, wherein the second treatment period has a duration of from about 22 weeks to about 26 weeks.

267. The method of claim 266, wherein the second treatment period has a duration of about 24 weeks.

268. The method of any one of claims 1-267, wherein the time elapsed between the end of the first treatment period and commencement of the second treatment period is at least about two weeks.

269. The method of claim 268, wherein the time elapsed between the end of the first treatment period and commencement of the second treatment period is at least about four weeks.

270. The method of claim 269, wherein the time elapsed between the end of the first treatment period and commencement of the second treatment period is at least about eight weeks.

271. The method of claim 270, wherein the time elapsed between the end of the first treatment period and commencement of the second treatment period is at least about 12 weeks.

272. The method of claim 271, wherein the time elapsed between the end of the first treatment period and commencement of the second treatment period is at least about 24 weeks.

273. The method of any one of claims 1-267, wherein the time elapsed between the end of the first treatment period and commencement of the second treatment period is from about one week to about 48 weeks.

274. The method of claim 273, wherein the time elapsed between the end of the first treatment period and commencement of the second treatment period is from about one week to about 12 weeks.

275. The method of claim 274, wherein the time elapsed between the end of the first treatment period and commencement of the second treatment period is from about one week to about six weeks.

276. The method of claim 275, wherein the time elapsed between the end of the first treatment period and commencement of the second treatment period is from about two weeks to about four weeks.

277. The method of claim 273, wherein the time elapsed between the end of the first treatment period and commencement of the second treatment period is about two weeks, about three weeks, about four weeks, about five weeks, or about six weeks.

278. The method of claim 273, wherein the time elapsed between the end of the first treatment period and commencement of the second treatment period is from about one month to about eight months.

279. The method of claim 278, wherein the time elapsed between the end of the first treatment period and commencement of the second treatment period is from about one month to about six months.

280. The method of claim 279, wherein the time elapsed between the end of the first treatment period and commencement of the second treatment period is from about one month to about four months.

281. The method of claim 273, wherein the time elapsed between the end of the first treatment period and commencement of the second treatment period is about one month, about two months, about three months, or about four months.

282. The method of any one of claims 1-281, wherein add-back therapy is periodically administered to the patient during the first treatment period.

283. The method of any one of claims 1-282, wherein add-back therapy is periodically administered to the patient during the second treatment period.

284. The method of claim 282 or 283, wherein the add-back therapy is administered to the patient one or more times daily during the first and/or second treatment period.

285. The method of claim 284, wherein the add-back therapy is administered to the patient once daily, concurrently with the GnRH antagonist, during the first and/or second treatment period.

286. The method of claim 284, wherein the add-back therapy is administered to the patient once daily, prior to administration of the GnRH antagonist, during the first and/or second treatment period.

287. The method of claim 284, wherein the add-back therapy is administered to the patient once daily, following administration of the GnRH antagonist, during the first and/or second treatment period.

288. The method of claim 287, wherein the add-back therapy is administered to the patient in the form of a pharmaceutical composition comprising the GnRH antagonist.

289. The method of any one of claims 282-288, wherein the add-back therapy comprises an estrogen.

290. The method of claim 289, wherein the estrogen is selected from the group consisting of β17-estradiol, ethinyl estradiol, and conjugated estrogens.

291. The method of claim 290, wherein the estrogen is β17-estradiol.

292. The method of claim 291, wherein the β17-estradiol is administered to the patient in an amount of about 1.0 mg/day during the first and/or second treatment period.

293. The method of claim 291, wherein the β17-estradiol is administered to the patient in an amount of about 0.5 mg/day during the first and/or second treatment period.

294. The method of claim 290, wherein the estrogen is ethinyl estradiol.

295. The method of claim 294, wherein the ethinyl estradiol is administered to the patient in an amount of about 5.0 μg/day during the first and/or second treatment period.

296. The method of claim 294, wherein the ethinyl estradiol is administered to the patient in an amount of about 2.5 μg/day during the first and/or second treatment period.

297. The method of claim 290, wherein the estrogen is a conjugated estrogen.

298. The method of claim 297, wherein the conjugated estrogen is administered to the patient in an amount of about 0.625 mg/day during the first and/or second treatment period.

299. The method of claim 297, wherein the conjugated estrogen is administered to the patient in an amount of about 0.45 mg/day during the first and/or second treatment period.

300. The method of claim 297, wherein the conjugated estrogen is administered to the patient in an amount of about 0.3 mg/day during the first and/or second treatment period.

301. The method of any one of claims 282-300, wherein the add-back therapy comprises a progestin.

302. The method of claim 301, wherein the progestin is selected from the group consisting of norethindrone or an ester thereof, progesterone, norgestimate, medroxyprogesterone, and drospirenone.

303. The method of claim 302, wherein the progestin is norethindrone or norethindrone acetate.

304. The method of claim 303, wherein the norethindrone or norethindrone acetate is administered to the patient in an amount of about 1.0 mg/day during the first and/or second treatment period.

305. The method of claim 303, wherein the norethindrone or norethindrone acetate is administered to the patient in an amount of about 0.5 mg/day during the first and/or second treatment period.

306. The method of claim 303, wherein the norethindrone or norethindrone acetate is administered to the patient in an amount of about 0.1 mg/day during the first and/or second treatment period.

307. The method of claim 302, wherein the progestin is progesterone.

308. The method of claim 307, wherein the progesterone is administered to the patient in an amount of about 200 mg/day during the first and/or second treatment period.

309. The method of claim 307, wherein the progesterone is administered to the patient in an amount of about 100 mg/day during the first and/or second treatment period.

310. The method of claim 302, wherein the progestin is norgestimate.

311. The method of claim 310, wherein the norgestimate is administered to the patient in an amount of about 0.09 mg/day during the first and/or second treatment period.

312. The method of claim 302, wherein the progestin is medroxyprogesterone.

313. The method of claim 312, wherein the medroxyprogesterone is administered to the patient in an amount of about 5 mg/day during the first and/or second treatment period.

314. The method of claim 312, wherein the medroxyprogesterone is administered to the patient in an amount of about 2.5 mg/day during the first and/or second treatment period.

315. The method of claim 312, wherein the medroxyprogesterone is administered to the patient in an amount of about 1.5 mg/day during the first and/or second treatment period.

316. The method of claim 302, wherein the progestin is drospirenone.

317. The method of claim 316, wherein the drospirenone is administered to the patient in an amount of about 0.5 mg/day during the first and/or second treatment period.

318. The method of claim 316, wherein the drospirenone is administered to the patient in an amount of about 0.25 mg/day during the first and/or second treatment period.

319. The method of any one of claims 282-318, wherein the add-back therapy comprises about 1.0 mg of β17-estradiol and about 0.5 mg of norethindrone acetate.

320. The method of any one of claims 282-318, wherein the add-back therapy comprises about 0.5 mg of β17-estradiol and about 0.1 mg of norethindrone acetate.

321. The method of any one of claims 1-320, wherein the patient is a pre-menopausal female of from about 18 to about 48 years of age.

322. The method of any one of claims 1-321, wherein the patient has been determined to exhibit a serum concentration of follicle-stimulating hormone (FSH) of about 20 IU/L or less prior to commencement of the first and/or second treatment period.

323. The method of any one of claims 1-322, wherein the patient has been determined to exhibit a rectal (type II) and/or vaginal (type III) endometriosis node of at least 2 cm prior to commencement of the first and/or second treatment period.

324. The method of claim 323, wherein the length of the type II and/or type III endometriosis node is assessed by way of magnetic resonance imaging (MRI).

325. The method of any one of claims 1-324, wherein the patient has been determined to exhibit a junctional-zone width of about 12 mm or more prior to commencement of the first and/or second treatment period.

326. The method of claim 325, wherein the junctional-zone width is assessed by way of MRI.

327. The method of any one of claims 1-326, wherein the patient exhibits a reduction in serum concentration of luteinizing hormone (LH), follicle-stimulating hormone (FSH), and/or β17-estradiol (E2) following administration of the GnRH antagonist to the patient.

328. The method of claim 327, wherein the patient exhibits the reduction in serum concentration of LH, FSH, and/or E2 within from about one day to about 36 weeks of commencement of the second treatment period, optionally wherein the patient exhibits the reduction within from about 12 weeks to about 24 weeks of commencement of the second treatment period.

329. The method of any one of claims 1, 2, 4-12, 14-22, 24-31, 33-37, 39-47, 49-57, 59-66, 68-73, 75-83, 85-93, 95-102, and 104-328, wherein the patient exhibits a reduction in uterine bleeding following administration of the GnRH antagonist to the patient.

330. The method of any one of claims 3, 13, 23, 32, 38, 48, 58, 67, 74, 84, 94, 103, and 329, wherein the patient exhibits the reduction in uterine bleeding within from about one day to about 36 weeks of commencement of the second treatment period, optionally wherein the patient exhibits the reduction within from about 12 weeks to about 24 weeks of commencement of the second treatment period.

331. The method of any one of claims 3, 13, 23, 32, 38, 48, 58, 67, 74, 84, 94, 103, 329, and 330, wherein the reduction in uterine bleeding is assessed by way of an alkaline hematin method.

332. The method of any one of claims 1-3, 5-13, 15-23, 25-32, 34-38, 40-48, 50-58, 60-67, 69-74, 76-84, 86-94, 96-103, and 105-331, wherein the patient exhibits amenorrhea following administration of the GnRH antagonist to the patient.

333. The method of any one of claims 4, 14, 24, 33, 39, 49, 59, 68, 75, 85, 95, 104, and 332, wherein the patient exhibits the amenorrhea within from about one day to about 36 weeks of commencement of the second treatment period, optionally wherein the patient exhibits the amenorrhea within from about 12 weeks to about 24 weeks of commencement of the second treatment period.

334. The method of any one of claims 1-25, 27-60, 62-96, and 98-333, wherein the patient exhibits a reduction in the volume of one or more rectovaginal endometriosis nodes following administration of the GnRH antagonist to the patient.

335. The method of any one of claims 26, 61, 97, and 334, wherein the patient exhibits the reduction in volume of the one or more rectovaginal endometriosis nodes within from about one day to about 36 weeks of commencement of the second treatment period, optionally wherein the patient exhibits the reduction within from about 12 weeks to about 24 weeks of commencement of the second treatment period.

336. The method of any one of claims 26, 61, 97, 334, and 335, wherein the reduction in volume of the one or more rectovaginal endometriosis nodes is assessed by way of MRI or TVUS.

337. The method of any one of claims 1-336, wherein the patient exhibits a reduction in bowel involvement of one or more type III endometriosis nodes following administration of the GnRH antagonist to the patient.

338. The method of claim 337, wherein the patient exhibits the reduction in bowel involvement of the one or more type III endometriosis nodes within from about one day to about 36 weeks of commencement of the second treatment period, optionally wherein the patient exhibits the reduction within from about 12 weeks to about 24 weeks of commencement of the second treatment period.

339. The method of any one of claims 1-8, 10-17, 19-27, 29-43, 45-52, 54-62, 64-79, 81-88, 90-98, and 100-338, wherein the patient exhibits a reduction in pelvic pain following administration of the GnRH antagonist to the patient.

340. The method of any one of claims 9, 18, 28, 44, 53, 63, 80, 89, 99, and 339, wherein the patient exhibits the reduction in pelvic pain within from about one day to about 36 weeks of commencement of the second treatment period, optionally wherein the patient exhibits the reduction within from about 12 weeks to about 24 weeks of commencement of the second treatment period.

341. The method of any one of claims 9, 18, 28, 44, 53, 63, 80, 89, 99, 339, and 340, wherein the reduction in pelvic pain is assessed by way of a modified Biberoglu & Behrman (mB&B) score, Numerical Rating Scale (NRS) score, or Verbal Rating Scale (VRS) score.

342. The method of any one of claims 1-9, 11-18, 20-28, 30-44, 46-53, 55-63, 65-80, 82-89, 91-99, and 101-340, wherein the patient exhibits a reduction in dysmenorrhea following administration of the GnRH antagonist to the patient.

343. The method of any one of claims 10, 19, 29, 45, 54, 64, 81, 90, 100, and 342, wherein the patient exhibits the reduction in dysmenorrhea within from about one day to about 36 weeks of commencement of the second treatment period, optionally wherein the patient exhibits the reduction within from about 12 weeks to about 24 weeks of commencement of the second treatment period.

344. The method of any one of claims 10, 19, 29, 45, 54, 64, 81, 90, 100, 342, and 343, wherein the reduction in dysmenorrhea is assessed by way of an mB&B score, NRS score, or VRS score.

345. The method of any one of claims 1-10, 12-19, 21-29, 31-45, 47-54, 56-64, 66-81, 83-90, 92-100, and 102-344, wherein the patient exhibits a reduction in dyspareunia following administration of the GnRH antagonist to the patient.

346. The method of any one of claims 11, 20, 30, 46, 55, 65, 82, 91, 101, and 345, wherein the patient exhibits the reduction in dyspareunia within from about one day to about 36 weeks commencement of the second treatment period, optionally wherein the patient exhibits the reduction within from about 12 weeks to about 24 weeks of commencement of the second treatment period.

347. The method of any one of claims 11, 20, 30, 46, 55, 65, 82, 91, 101, 345, and 346, wherein the reduction in dyspareunia is assessed by way of an mB&B score, NRS score, or VRS score.

348. The method of any one of claims 1-11, 13-20, 22-30, 32-46, 48-55, 57-65, 67-82, 84-91, 93-101, and 103-347, wherein the patient exhibits a reduction in dyschezia following administration of the GnRH antagonist to the patient.

349. The method of any one of claims 12, 21, 31, 47, 56, 66, 83, 92, 102, and 348, wherein the patient exhibits the reduction in dyschezia within from about one day to about 36 weeks of commencement of the second treatment period, optionally wherein the patient exhibits the reduction within from about 12 weeks to about 24 weeks of commencement of the second treatment period.

350. The method of any one of claims 12, 21, 31, 47, 56, 66, 83, 92, 102, 348, and 349, wherein the reduction in dyschezia is assessed by way of an mB&B score, NRS score, or VRS score.

351. The method of any one of claims 1-15, 17-50, 52-86, and 88-349, wherein the patient exhibits a reduction in uterine volume following administration of the GnRH antagonist to the patient.

352. The method of any one of claims 16, 51, 87, and 351, wherein the patient exhibits the reduction in uterine volume within from about one day to about 36 weeks of commencement of the second treatment period, optionally wherein the patient exhibits the reduction within from about 12 weeks to about 24 weeks of commencement of the second treatment period.

353. The method of any one of claims 16, 51, 87, 351, and 352, wherein the reduction in uterine volume is assessed byway of MRI or transvaginal ultrasound (TVUS).

354. The method of any one of claims 1-16, 18-51, 53-87, and 89-353, wherein the patient exhibits a reduction in the thickness of the anterior and/or posterior region of the uterine myometrium following administration of the GnRH antagonist to the patient.

355. The method of any one of claims 17, 52, 88, and 354, wherein the patient exhibits the reduction in thickness of the anterior and/or posterior region of the uterine myometrium within from about one day to about 36 weeks of commencement of the second treatment period, optionally wherein the patient exhibits the reduction within from about 12 weeks to about 24 weeks of commencement of the second treatment period.

356. The method of any one of claims 1-21, 23-56, 58-92, and 94-355, wherein the patient exhibits a reduction in uterine tenderness following administration of the GnRH antagonist to the patient.

357. The method of any one of claims 22, 57, 93, and 356, wherein the patient exhibits the reduction in uterine tenderness within from about one day to about 36 weeks of commencement of the second treatment period, optionally wherein the patient exhibits the reduction within from about 12 weeks to about 24 weeks of commencement of the second treatment period.

358. The method of any one of claims 1-357, wherein the patient exhibits a reduction in the diameter of a junctional zone of adenomyosis following administration of the GnRH antagonist to the patient.

359. The method of claim 358, wherein the patient exhibits the reduction in the diameter of the junctional zone of adenomyosis within from about one day to about 36 weeks of commencement of the second treatment period, optionally wherein the patient exhibits the reduction within from about 12 weeks to about 24 weeks of commencement of the second treatment period.

360. The method of any one of claims 1-359, wherein the patient exhibits an improvement in Endometriosis Health Profile questionnaire (EHP-30) score following administration of the GnRH antagonist to the patient.

361. The method of claim 360, wherein the patient exhibits the improvement in the EHP-30 score within from about one day to about 36 weeks of commencement of the second treatment period, optionally wherein the patient exhibits the improvement within from about 12 weeks to about 24 weeks of commencement of the second treatment period.

362. The method of any one of claims 1-361, wherein the patient exhibits a positive Patient Global Impression of Change (PGIC) score core following administration of the GnRH antagonist to the patient.

363. The method of claim 362, wherein the patient exhibits the positive PGIC score within from about one day to about 36 weeks of commencement of the second treatment period, optionally wherein the patient exhibits the positive PGIC score within from about 12 weeks to about 24 weeks of commencement of the second treatment period.

364. The method of any one of claims 1-363, wherein the patient does not exhibit a reduction in bone mineral density (BMD) of greater than 5% at the end of the second treatment period relative to a measurement of the patient's bone mineral density obtained prior to, or during, the first treatment period.

365. The method of claim 364, wherein the patient does not exhibit a reduction in BMD of greater than 3% at the end of the second treatment period relative to a measurement of the patient's bone mineral density obtained prior to, or during, the first treatment period.

366. The method of claim 365, wherein the patient does not exhibit a reduction in BMD of greater than 2% at the end of the second treatment period relative to a measurement of the patient's bone mineral density obtained prior to, or during, the first treatment period.

367. The method of claim 366, wherein the patient does not exhibit a reduction in BMD of greater than 1% at the end of the second treatment period relative to a measurement of the patient's bone mineral density obtained prior to, or during, the first treatment period.

368. The method of any one of claims 364-367, wherein the BMD is assessed by dual energy X-ray absorptiometry.

369. The method of claim 368, wherein the BMD is assessed in the spine or femur of the patient.

370. The method of any one of claims 364-367, wherein the BMD is assessed by comparing the concentration of bone specific alkaline phosphatase (BAP) in a sample isolated from the patient following the administration to the concentration of BAP in a sample isolated from the patient prior to the administration.

371. The method of any one of claims 364-367, wherein the BMD is assessed by comparing the concentration of deoxypyridinoline (DPD) in a sample isolated from the patient following the administration to the concentration of DPD in a sample isolated from the patient prior to the administration.

372. The method of any one of claims 364-367, wherein the BMD is assessed by comparing the concentration of type I collagen C-terminal telopeptide (CTX) in a sample isolated from the patient following the administration to the concentration of CTX in a sample isolated from the patient prior to the administration.

373. The method of any one of claims 364-367, wherein the BMD is assessed by comparing the concentration of procollagen 1 N-terminal peptide (P1NP) in a sample isolated from the patient following the administration to the concentration of P1NP in a sample isolated from the patient prior to the administration.

374. A kit comprising a GnRH antagonist and a package insert instructing a user of the kit to administer the GnRH antagonist to a human patient in accordance with the method of any one of claims 1-373.

375. The kit of claim 374, wherein the GnRH antagonist is a compound represented by formula (I) 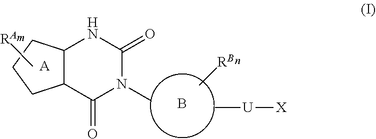
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

376. The kit of claim 375, wherein the ring A is a thiophene ring represented by formula (IIa)

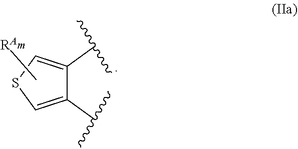

377. The kit of claim 375 or 376, wherein m is 1.

378. The kit of claim 377, wherein the ring A is an optionally substituted thiophene ring represented by formula (IIb)

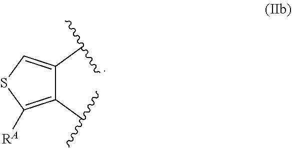

379. The kit of any one of claims 375-378, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

380. The kit of claim 379, wherein each R^{A} is COOH or pharmaceutically acceptable salt thereof.

381. The kit of any one of claims 375-380, wherein the ring B is an optionally substituted benzene ring, pyridine ring, or thiophene ring.

382. The kit of claim 381, wherein the ring B is represented by a formula selected from the group consisting of:

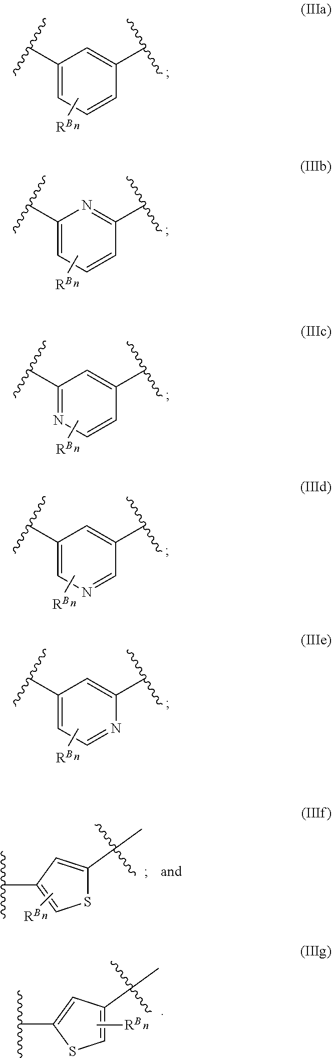

383. The kit of any one of claims 375-382, wherein n is 2.

384. The kit of claim 383, wherein ring B is represented by a formula selected from the group consisting of:

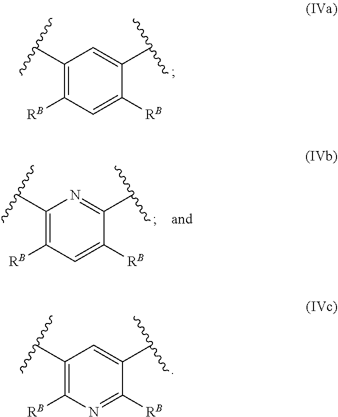

385. The kit of any one of claims 375-384, wherein each R^{B} is independently a halogen atom, an optionally substituted lower alkyl group, or OW⁴, wherein each W⁴ is independently a hydrogen atom or an optionally substituted lower alkyl group.

386. The kit of claim 385, wherein each R^{B} is independently a fluorine atom, chlorine atom, bromine atom, methyl group, or methoxy group.

387. The kit of any one of claims 375-386, wherein U is a single bond.

388. The kit of any one of claims 375-387, wherein X is a group represented by —O-L-Y.

389. The kit of any one of claims 375-388, wherein L is a methylene group.

390. The kit of any one of claims 375-389, wherein Y is an optionally substituted benzene ring represented by formula (V) 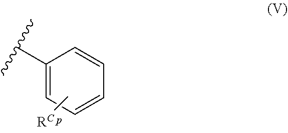
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

391. The kit of claim 390, wherein Y is a substituted benzene ring represented by formula (Va)

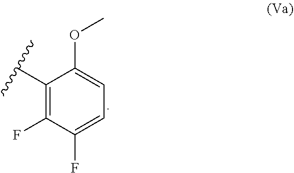 [SMILES: *c1c(OC)ccc(F)c1F]

392. The kit of claim 375, wherein the compound is represented by formula (Ia) 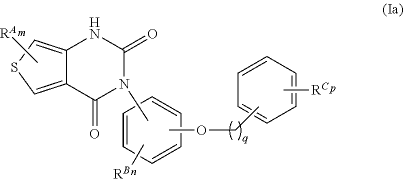
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

393. The kit of claim 392, wherein the compound is represented by formula (Ib)

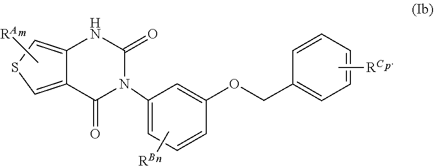

394. The kit of claim 393, wherein the compound is represented by formula (Ic) 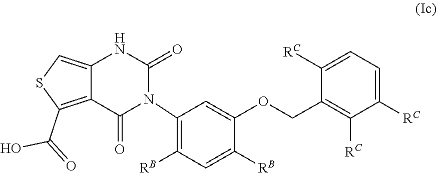
or a pharmaceutically acceptable salt thereof.

395. The kit of any one of claims 375-394, wherein the compound is represented by formula (VI) 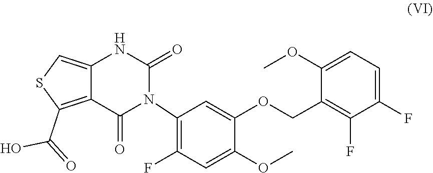 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

396. The kit of claim 395, wherein the compound is the choline salt of the compound represented by formula (VI).

397. The kit of claim 374, wherein the GnRH antagonist is elagolix, relugolix, opigolix (ASP1707), BAY-784, or SK-2706.
